(19) United States
(12) Patent Application Publication
(10) Pub. No.: US 2025/0040428 A1
(43) Pub. Date: Jan. 30, 2025

(54) NOVEL COMPOUND AND ORGANIC LIGHT EMITTING DEVICE COMPRISING THE SAME

(71) Applicant: LG CHEM, LTD., Seoul (KR)

(72) Inventors: Soyeon KIM, Daejeon (KR); Minjun KIM, Daejeon (KR); Sang Duk SUH, Daejeon (KR); Min Woo JUNG, Daejeon (KR); Young Seok KIM, Daejeon (KR); Donghee KIM, Daejeon (KR); Sunmi LEE, Daejeon (KR)

(21) Appl. No.: 18/761,098

(22) Filed: Jul. 1, 2024

(30) Foreign Application Priority Data
Jul. 3, 2023 (KR) 10-2023-0086039
Sep. 14, 2023 (KR) 10-2023-0122689

(51) Int. Cl. H10K 85/60; C07D 307/77; C07D 307/91; C07D 333/50; C07D 409/12; C09K 11/06; H10K 50/12; H10K 101/00

(52) U.S. Cl. CPC H10K 85/636; C07D 307/77; C07D 307/91; C07D 333/50; C07D 409/12; C09K 11/06; H10K 85/633; C09K 2211/1018; H10K 50/12; H10K 85/615; H10K 85/622; H10K 85/6574; H10K 2101/90

(57) ABSTRACT

A compound of Chemical Formula 1:

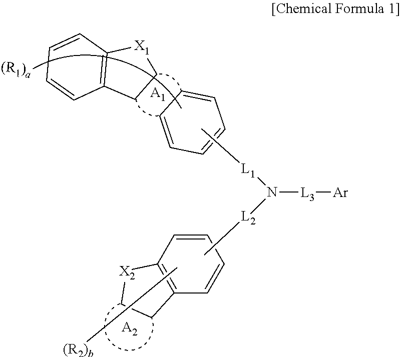

where X₁ and X₂ are each independently O or S; A₁ is a benzene ring fused with adjacent pentagonal and benzene rings; A₂ is a naphthalene ring fused with an adjacent pentagonal ring; Ar is a substituted or unsubstituted C₆₋₆₀ aryl, a substituted or unsubstituted C₂₋₆₀ heteroaryl containing a heteroatom of O or S, or a substituted or unsubstituted C₂₋₆₀ heteroaryl containing a heteroatom of N and at least one selected of O and S; R₁ and R₂ are each independently hydrogen, deuterium, halogen, cyano, or a substituted or unsubstituted C₁₋₆₀ alkyl; and the other substituents are as defined in the specification; and an organic light emitting device including the same. The organic light emitting device including the compound of Chemical Formula 1 in an organic material layer exhibits improved efficiency, low driving voltage and/or improved lifetime characteristics.

### CROSS-REFERENCE TO RELATED APPLICATIONS

This application claims priority to and the benefit of Korean Patent Application No. 10-2023-0086039 filed in the Korean Intellectual Property Office on Jul. 3, 2023, and Korean Patent Application No. 10-2023-0122689 filed in the Korean Intellectual Property Office on Sep. 14, 2023, the entire contents of each of which are incorporated herein by reference.

### TECHNICAL FIELD

The present disclosure relates to a novel compound and an organic light emitting device comprising the same.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuous need to develop a new material for the organic material used in the organic light emitting device as described above.

### PRIOR ART LITERATURE

### Patent Literature

(Patent Literature 0001) Korean Unexamined Patent Publication No. 10-2000-0051826

### DETAILED DESCRIPTION OF THE INVENTION

### Technical Problem

It is an object of the present disclosure to provide a novel compound and an organic light emitting device comprising the same.

### Technical Solution

According to the present disclosure, there is provided a compound represented by the following Chemical Formula 1:

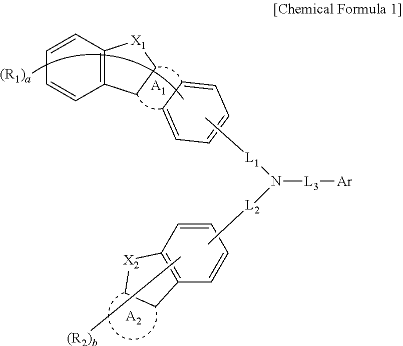

wherein in Chemical Formula 1,
X₁ and X₂ are each independently O or S,
A₁ is a benzene ring fused with adjacent pentagonal and benzene rings,
A₂ is a naphthalene ring fused with an adjacent pentagonal ring,
L₁ to L₃ are each independently a single bond; or a substituted or unsubstituted C₆₋₆₀ arylene,
Ar is a substituted or unsubstituted C₆₋₆₀ aryl; a substituted or unsubstituted C₂₋₆₀ heteroaryl containing a heteroatom of O or S; or a substituted or unsubstituted C₂₋₆₀ heteroaryl containing heteroatom of N and at least one selected of O and S,
R₁ and R₂ are each independently hydrogen; deuterium; halogen; cyano; or a substituted or unsubstituted C₁₋₆₀ alkyl, and
a and b are each independently an integer of 0 to 9.

According to another aspect of the present disclosure, there is provided an organic light emitting device comprising: a first electrode; a second electrode that is provided opposite to the first electrode; and one or more organic material layers that are provided between the first electrode and the second electrode, wherein one or more layers of the organic material layers comprises the compound represented by Chemical Formula 1.

### Advantageous Effects

The above-mentioned compound represented by Chemical Formula 1 can be used as a material of an organic material layer in an organic light emitting device, and can improve the efficiency, achieve low driving voltage and/or improve lifetime characteristics in the organic light emitting device.

### BRIEF DESCRIPTION OF THE DRAWINGS

FIG. 1 shows an example of an organic light emitting device comprising a substrate 1, an anode 2, a hole transport layer 3, a light emitting layer 4, an electron injection and transport layer 5, and a cathode 6.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6.

### DETAILED DESCRIPTION OF THE EMBODIMENTS

Hereinafter, embodiments of the present disclosure will be described in more detail to help understanding of the invention.

### Definition of Terms

In the present disclosure, the notation [symbol] or [symbol] means a bond linked to another substituent group, and “D” means deuterium.

In the present disclosure, the term “substituted or unsubstituted” means being unsubstituted or substituted with one or more substituents selected from the group consisting of deuterium; a halogen group; a cyano group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent group to which two or more substituent groups of the above-exemplified substituent groups are linked. For example, “a substituent in which two or more substituents are linked” may be a biphenylyl group. Namely, a biphenylyl group may be an aryl group, or it may be interpreted as a substituent formed by linking two phenyl groups. In one example, the term “substituted or unsubstituted” may be understood as meaning “being unsubstituted or substituted with one or more substituents selected from the group consisting of deuterium, halogen, cyano, a silyl group, a C₁₋₁₀ alkyl, a C₁₋₁₀ alkoxy and a C₆₋₂₀ aryl”, or “being unsubstituted or substituted with one or more substituents selected from the group consisting of deuterium, halogen, cyano, methyl, ethyl, phenyl, biphenylyl and naphthyl”. Further, the term “substituted with one or more substituents” as used herein may be understood as meaning “being substituted with mono to the maximum number of substitutable hydrogens”. Alternatively, the term “substituted with one or more substituents” as used herein may be understood as meaning “being substituted with 1 to 5 substituents”, or “being substituted with one or two substituents”.

In the present disclosure, the carbon number of a carbonyl group is not particularly limited, but is preferably 1 to 40. Specifically, the carbonyl group may be a substituent having the following structural formulas, but is not limited thereto.

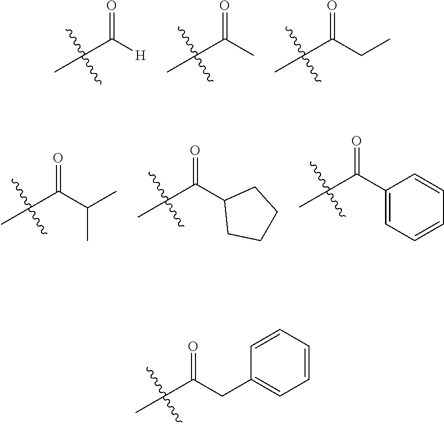

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent having the following structural formulas, but is not limited thereto.

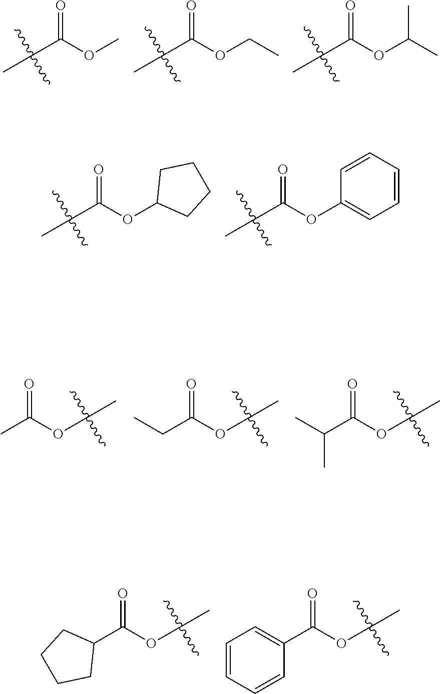

In the present disclosure, the carbon number of an imide group is not particularly limited, but is preferably 1 to 25. Specifically, the imide group may be a substituent group having the following structural formulas, but is not limited thereto.

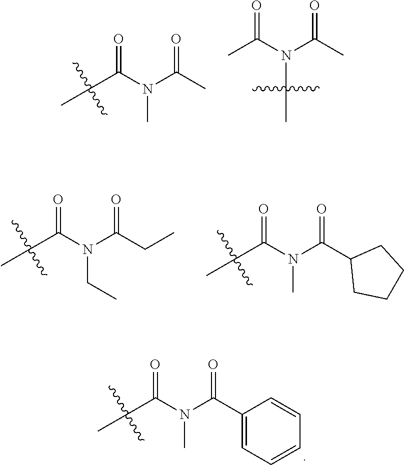

In the present disclosure, a silyl group means —Si(Z₁)(Z₂)(Z₃), wherein Z₁, Z₂ and Z₃ are each independently hydrogen, deuterium, a substituted or unsubstituted C₁₋₆₀ alkyl, a substituted or unsubstituted C₁₋₆₀ haloalkyl, a substituted or unsubstituted C₂₋₆₀ alkenyl, a substituted or unsubstituted C₂₋₆₀ haloalkenyl, or a substituted or unsubstituted C₆₋₆₀ aryl. According to one embodiment, Z₁, Z₂ and Z₃ may be each independently hydrogen, deuterium, a substituted or unsubstituted C₁₋₁₀ alkyl, a substituted or unsubstituted C₁₋₁₀ haloalkyl, or a substituted or unsubstituted C₆₋₂₀ aryl. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluoro, chloro, bromo, or iodo.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, 1-ethyl-propyl, 1,1-dimethylpropyl, hexyl, n-hexyl, 1-methylpentyl, 2-methylpentyl, 4-methyl-2-pentyl, 3,3-dimethylbutyl, 2-ethylbutyl, heptyl, n-heptyl, isohexyl, 1-methylhexyl, 2-methylhexyl, 3-methylhexyl, 4-methylhexyl, 5-methylhexyl, cyclopentylmethyl, cyclohexylmethyl, octyl, n-octyl, tert-octyl, 1-methylheptyl, 2-ethylhexyl, 2,4,4-trimethyl-1-pentyl, 2,4,4-trimethyl-2-pentyl, 2-propylpentyl, n-nonyl, 2,2-dimethylheptyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, the alicyclic group means a monovalent substituent derived from a saturated or unsaturated hydrocarbon ring compound that contains only carbon as a ring-forming atom, but does not have aromaticity, which is understood to encompass both monocyclic and fused polycyclic compounds. According to one embodiment, the carbon number of the alicyclic group is 3 to 60. According to another embodiment, the carbon number of the alicyclic group is 3 to 30. According to another embodiment, the carbon number of the alicyclic group is 3 to 20. Examples of the alicyclic group include a monocyclic group such as a cycloalkyl group, a bridged hydrocarbon group, a spiro hydrocarbon group, a substituent derived from hydrogenated derivatives of aromatic hydrocarbon compound, and the like.

Specifically, examples of the cycloalkyl group include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

Further, examples of the bridged hydrocarbon group include bicyclo[1.1.0]butyl, bicyclo[2.2.1]heptyl, bicyclo[4.2.0]octa-1,3,5-trienyl, adamantyl, decalinyl, and the like, but are not limited thereto.

Further, examples of the spiro hydrocarbon group include spiro[3.4]octyl, spiro[5.5]undecanyl, and the like, but are not limited thereto.

Further, a substituent derived from a hydrogenated derivative of the aromatic hydrocarbon compound means a substituent derived from a monocyclic or polycyclic aromatic hydrocarbon compound in which a part of the compound is hydrogenated. Examples of such a substituent include 1H-indenyl, 2H-indenyl, 4H-indenyl, 2,3-dihydro-1H-indenyl, 1,4-dihydronaphthalenyl, 1,2,3,4-tetrahydronaphthalenyl, 6,7,8,9-tetrahydro-5H-benzo[7]annulenyl, 6,7-dihydro-5H-benzocycloheptenyl, and the like, but are not limited thereto.

In the present disclosure, an aryl group is understood to mean a substituent derived from a monocyclic or fused polycyclic compound containing only carbon as a ring-forming atom and also having aromaticity, and the carbon number thereof is not particularly limited, but is preferably 6 to 60. According to one embodiment, the carbon number of the aryl group is 6 to 30. According to one embodiment, the carbon number of the aryl group is 6 to 20. The aryl group may be a phenyl group, a biphenylyl group, a terphenylyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, a fluorenyl group, or the like, but is not limited thereto.

In the present disclosure, the fluorenyl group may be substituted, and two substituent groups may be linked with each other to form a spiro structure.

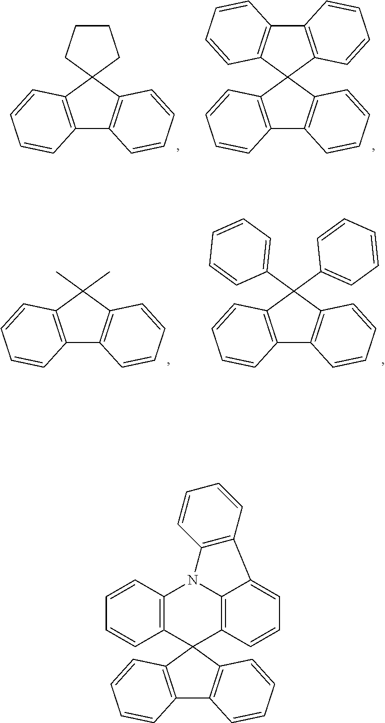

In the case where the fluorenyl group is substituted, and the like can be formed. However, the structure is not limited thereto.

In the present disclosure, a heterocyclic group means a monovalent substituent derived from a monocyclic or fused polycyclic compound that further contains at least one heteroatom selected among O, N, Si, and S in addition to carbon as a ring-forming atom, and is understood to encompass both substituents with aromaticity and substituents without aromaticity. According to one embodiment, the carbon number of the heterocyclic group is 2 to 60 carbon atoms. According to another embodiment, the carbon number of the heterocyclic group is 2 to 30. According to another embodiment, the carbon number of the heterocyclic group is 2 to 20. Examples of such a heterocyclic group include a heteroaryl group, a substituent derived from a hydrogenated derivative of the heteroaromatic compound, and the like.

Specifically, the heteroaryl group means a substituent derived from a monocyclic or fused polycyclic compound which further contains at least one heteroatom selected among N, O and S in addition to carbon as a ring forming atom, and refers to a substituent having aromaticity. According to one embodiment, the carbon number of the heteroaryl group is 2 to 60. According to another embodiment, the carbon number of the heteroaryl group is 2 to 30. According to another embodiment, the carbon number of the heteroaryl group is 2 to 20. According to another embodiment, the carbon number of the heteroaryl group is 2 to 12. According to another embodiment, the carbon number of the heteroaryl group is 2 to 10. According to another embodiment, the carbon number of the heteroaryl group is 2 to 8. Examples of the heteroaryl group include a thiophenyl group, a furanyl group, a pyrrole group, an imidazolyl group, a thiazolyl group, an oxazolyl group, an oxadiazolyl group, a triazolyl group, a pyridinyl group, a bipyridinyl group, a pyrimidinyl group, a triazinyl group, an acridinyl group, a pyridazinyl group, a pyrazinyl group, a quinolinyl group, a quinazolinyl group, a quinoxalinyl group, a phthalazinyl group, a pyridopyrimidinyl group, a pyridopyrazinyl group, an isoquinolinyl group, an indolyl group, a carbazolyl group, a benzoxazolyl group, a benzoimidazolyl group, a benzothiazolyl group, a benzocarbazolyl group, a benzothiophenyl group, a dibenzothiophenyl group, a benzofuranyl group, a dibenzofuranyl group, a phenanthrolinyl group, an isoxazolyl group, a thiadiazolyl group, a phenothiazinyl group, and the like, but are not limited thereto.

Further, a substituent derived from a hydrogenated derivative of a heteroaromatic compound means a substituent derived from a monocyclic or polycyclic heteroaromatic compound in which a part of the unsaturated bond of the compound is hydrogenated. Examples of such substituents include 1,3-dihydroisobenzofuranyl, 2,3-dihydrobenzofuranyl, 1,3-dihydrobenzo[c]thiophenyl, 2,3-dihydro[b]thiophenyl, and the like, but are not limited thereto.

In the present disclosure, the aryl group in the aralkyl group, the aralkenyl group, the alkylaryl group, the arylamine group and the arylsilyl group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heteroaryl as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heteroaryl as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heteroaryl as defined above can be applied, except that the heterocycle is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “substituted or unsubstituted with deuterium” means that “mono to the maximum number of unsubstituted or substitutable hydrogens have been substituted with deuterium.” In one example, the term “phenanthryl unsubstituted or substituted with deuterium” may be understood as meaning “phenanthryl unsubstituted or substituted with 1 to 9 deuterium atoms”, considering that the maximum number of hydrogen that can be substituted with deuterium in the phenanthryl structure is 9.

Further, “deuterated structure” means to include compounds, divalent linking groups, or monovalent substituents of all structures in which at least one hydrogen is substituted with deuterium. As an example, the deuterated structure of phenyl can be understood to refer to monovalent substituents of all structures in which at least one substitutable hydrogen in the phenyl group is substituted with deuterium, as follows.

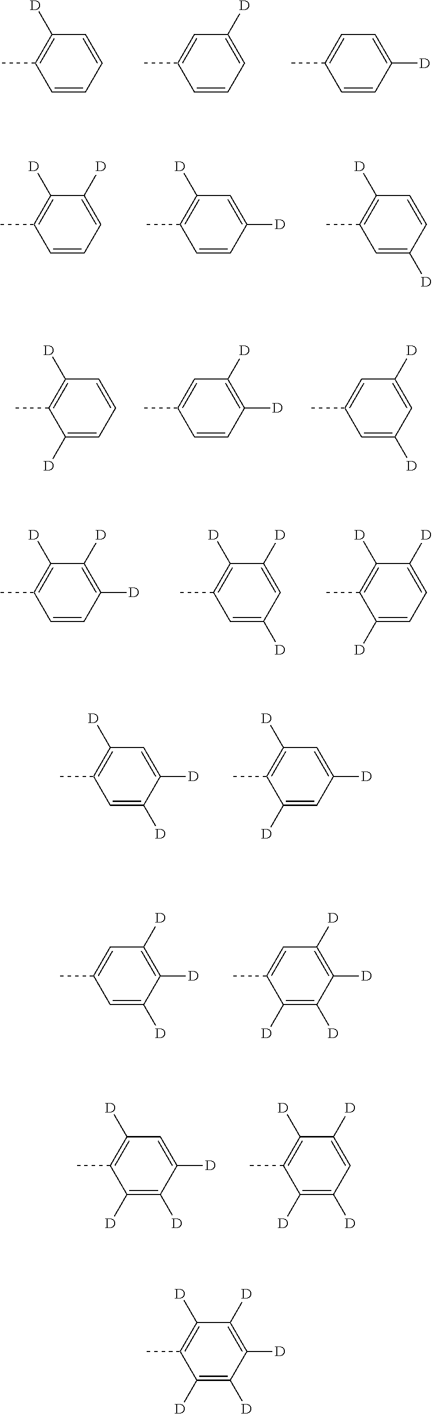

In addition, the “deuterium substitution rate” or “degree of deuteration” of a compound means that the ratio of the number of substituted deuterium atoms to the total number of hydrogen atoms (the sum of the number of hydrogen atoms substitutable with deuterium and the number of substituted deuterium atoms in a compound) that can exist in the compound is calculated as a percentage. Therefore, when the “deuterium substitution rate” or “degree of deuteration” of a compound is “K %”, it means that K % of the hydrogen atoms substitutable with deuterium in the compound are substituted with deuterium.

At this time, the “deuterium substitution rate” or “degree of deuteration” can be determined according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GC/MS (Gas Chromatography/Mass Spectrometry), or the like. More specifically, when using MALDI-TOF MS, the “deuterium substitution rate” or “degree of deuteration” may be obtained by determining the number of substituted deuterium in the compound through MALDI-TOF MS analysis, and then calculating the ratio of the number of substituted deuterium to the total number of hydrogen atoms that can exist in the compound as a percentage.

### (Compound)

Meanwhile, according to the present disclosure, there is provided the compound represented by Chemical Formula 1.

Specifically, the compound represented by Chemical Formula 1 is a tertiary amine compound having two benzonaphthofuranyl/benzonaphthothiophenyl substituents, wherein the compound has a structure in which one of the benzonaphthofuranyl/benzonaphthothiophenyl substituents is a benzene ring which is linked to the nitrogen atom of the amino group, and the other one of the benzonaphthofuranyl/benzonaphthothiophenyl substituents is a naphthalene ring which is linked to the nitrogen atom of the amino group. The compound represented by Chemical Formula 1 having such a structure not only has superior hole transport ability compared to compounds having different structures, and thus can be used as a hole transport material, but also contributes to the stabilization of excitons and simultaneously has excellent energy transfer ability to dopants, and thus can also be used as a host material.

Thereby, an organic light emitting device employing the above compound can not only exhibit a lower driving voltage compared to an organic light emitting device employing a compound having a structure different from that of the present application, but also improve efficiency and lifetime characteristics at the same time.

Meanwhile, the compound may be represented by any of the following Chemical Formulas 1A to 1C depending on the fusion position of the A₁ ring:

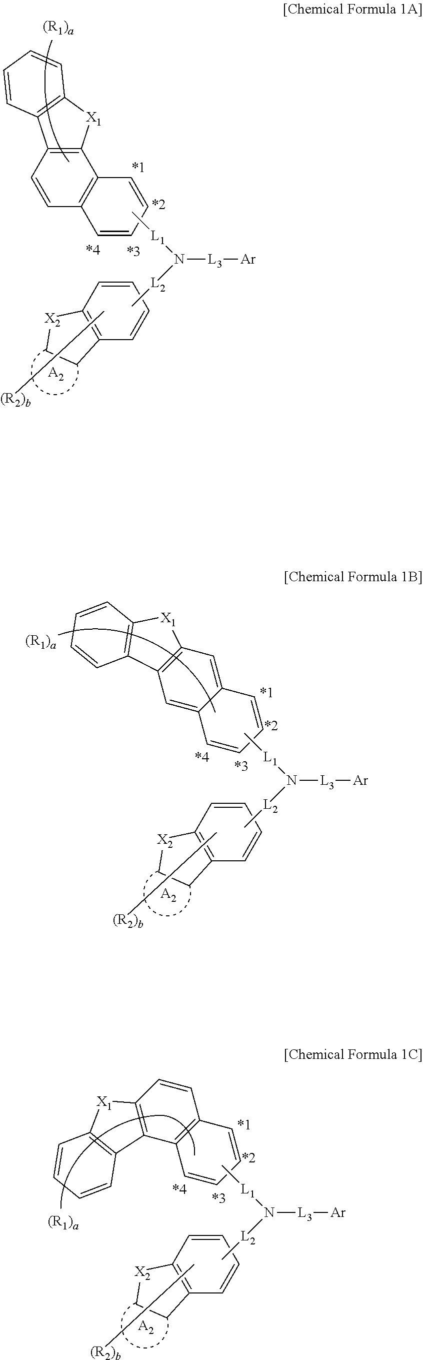

wherein in Chemical Formulas 1A to 1C,
L₁ is linked to any one of carbon at position *1, carbon at position *2, carbon at position *3, and carbon at position *4, and
X₁, X₂, A₂, L₁ to L₃, Ar, R₁, R₂, a and b are as defined in Chemical Formula 1.

In one embodiment, both X₁ and X₂ are O; or
both X₁ and X₂ are S; or
X₁ is O, and X₂ is S; or
X₁ may be S, and X₂ may be O.

Further,

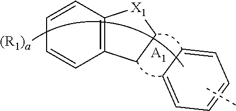

is any one of the substituents represented by the following Chemical Formulas 2a to 2l:

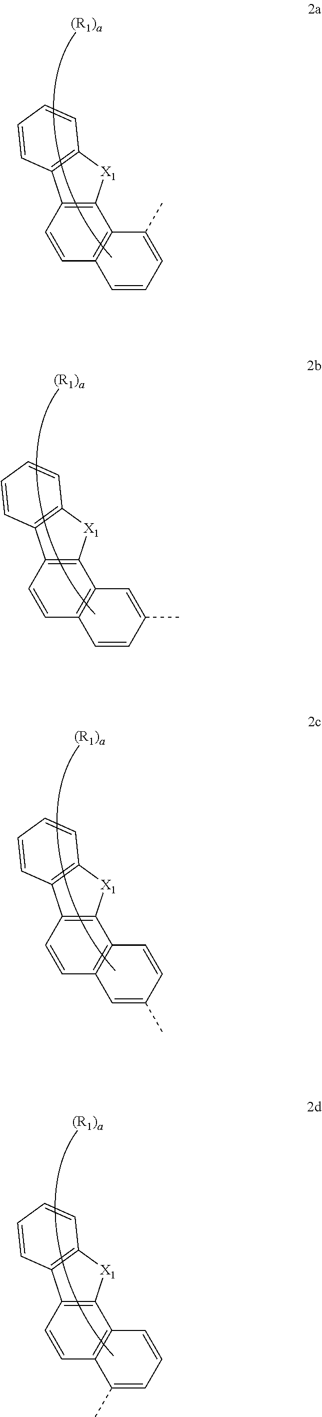

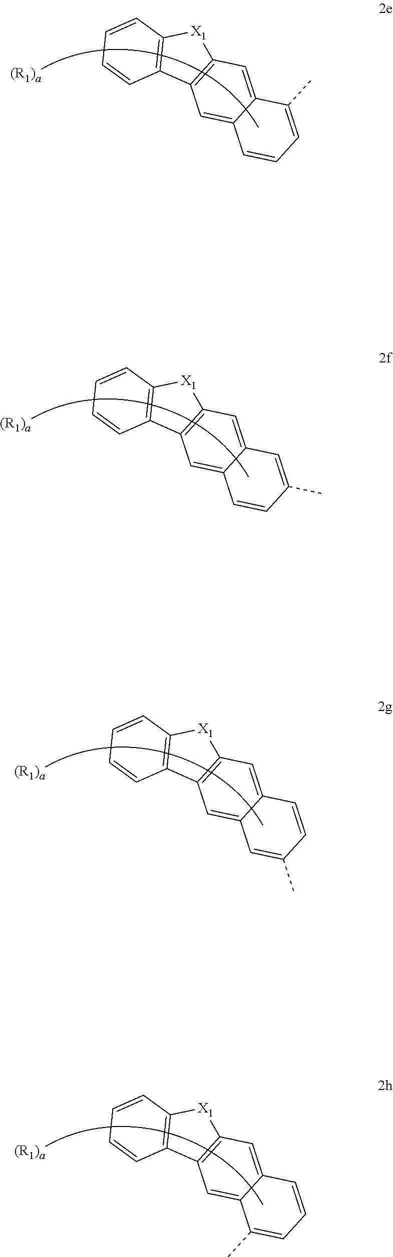

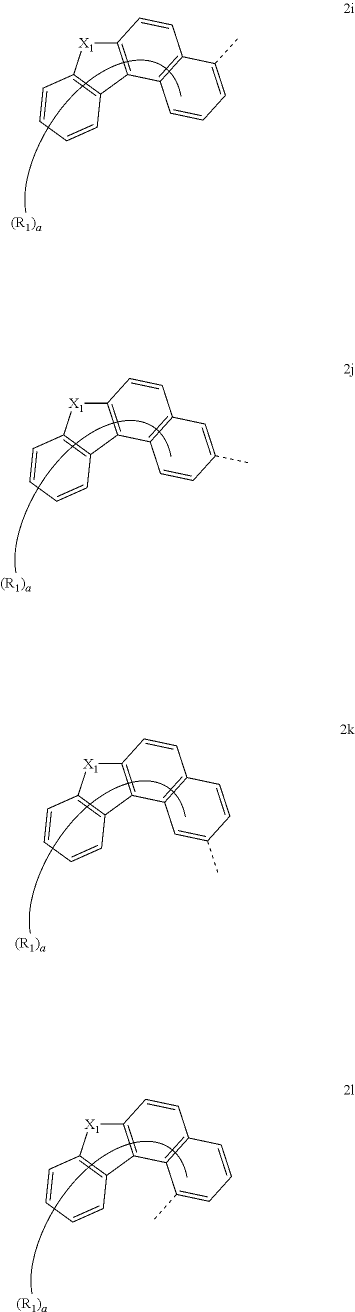

wherein in Chemical Formulas 2a to 2l,
X₁, R₁ and a are as defined in Chemical Formula 1.

Further,

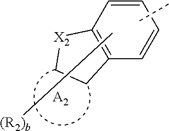

is any one of the substituents represented by the following Chemical Formulas 3a to 3l:

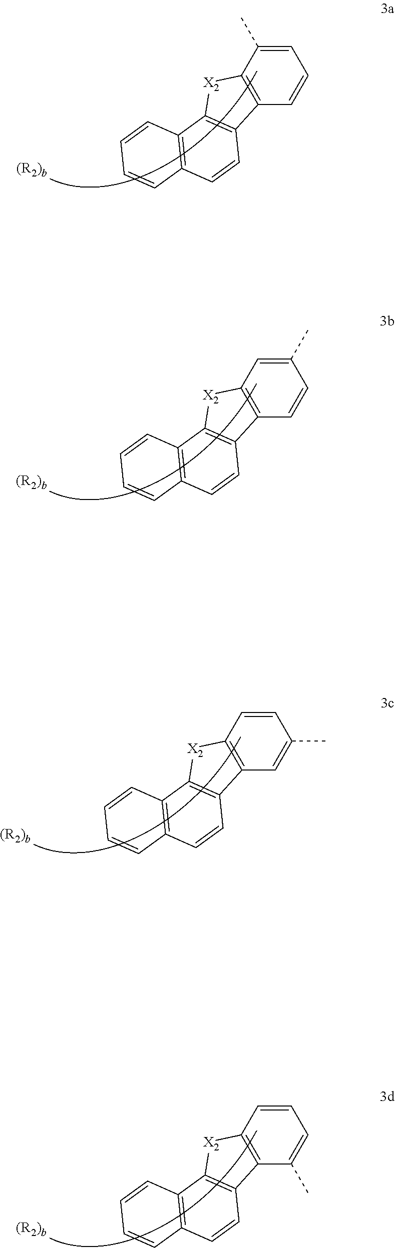

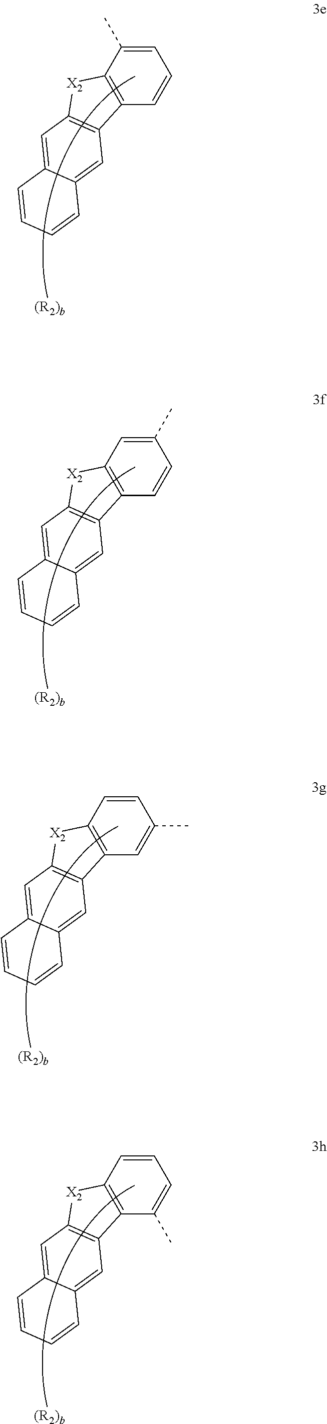

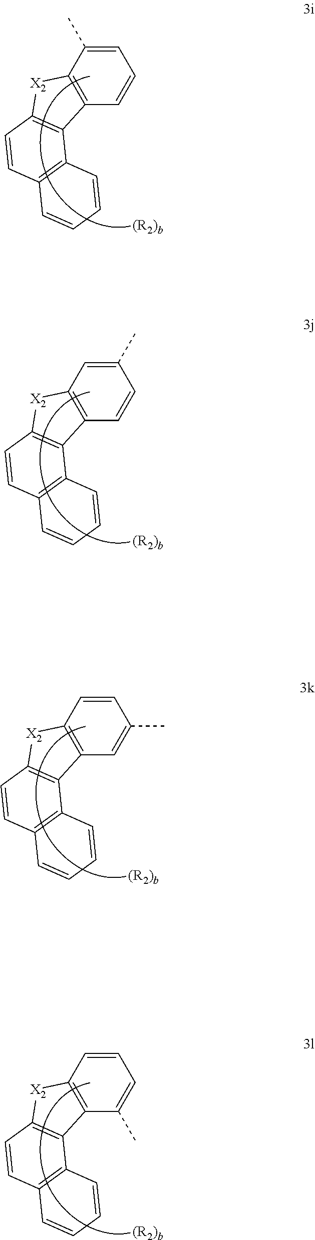

wherein in Chemical Formulas 3a to 3l,
X₂, R₂ and b are as defined in Chemical Formula 1.

In one embodiment, L₁ and L₂ may be each independently a single bond, or a substituted or unsubstituted C₆₋₂₀ arylene.

In another embodiment, L₁ and L₂ may be each independently a single bond; or a C₆₋₂₀ arylene, which is unsubstituted, or substituted with one or more substituents selected from the group consisting of deuterium, phenyl and naphthyl.

In yet another embodiment, L₁ and L₂ may be each independently a single bond, or a substituted or unsubstituted C₆₋₁₂ arylene.

In yet another embodiment, L₁ and L₂ may be each independently a single bond, a substituted or unsubstituted phenylene, a substituted or unsubstituted biphenyldiyl, or substituted or unsubstituted naphthylene.

In yet another embodiment, L₁ and L₂ may be each independently a single bond, phenylene, biphenyldiyl, or naphthylene,

wherein the phenylene, the biphenyldiyl, and the naphthylene may be unsubstituted or substituted with deuterium.

For example, L₁ and L₂ may be each independently a single bond, or any one selected from the group consisting of the following formulas and the deuterated structure thereof:

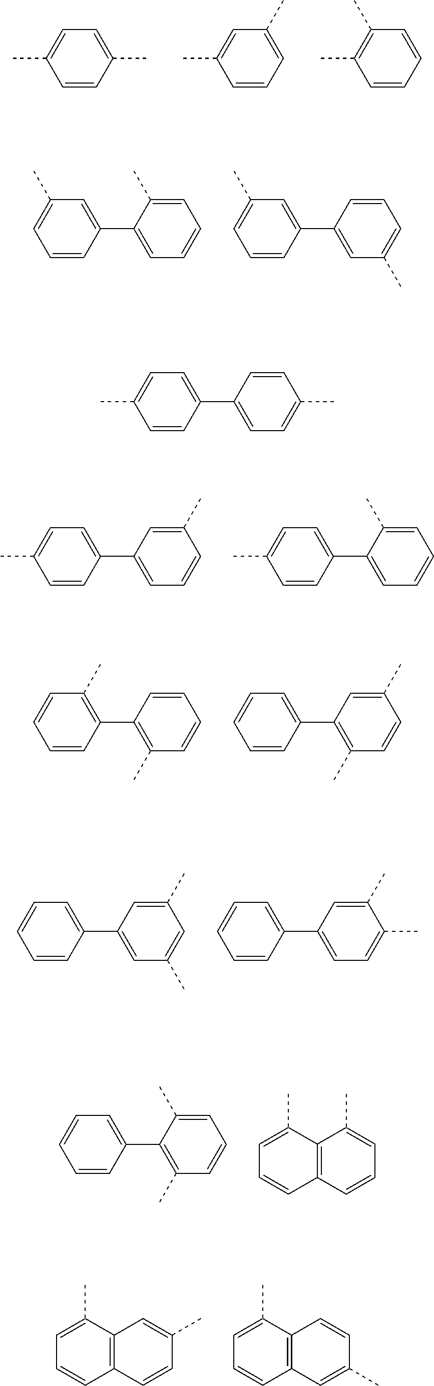

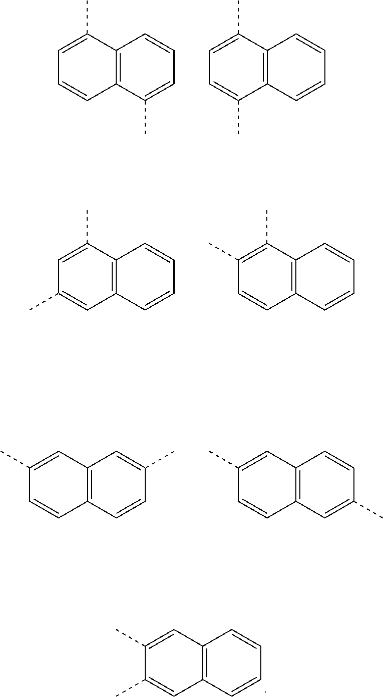

More specifically, for example, L₁ and L₂ may be each independently a single bond, or a substituted or unsubstituted 1,4-phenylene.

In yet another embodiment, at least one of L₁ and L₂ may be a single bond.

At this time, L₁ and L₂ may be identical to each other. Alternatively, L₁ and L₂ may be different from each other.

For example, both L₁ and L₂ are a single bond; or
L₁ is a single bond, and L₂ is a substituted or unsubstituted C₆₋₁₂ arylene; or
L₁ is a substituted or unsubstituted C₆₋₁₂ arylene, and L₂ may be a single bond.

For example, both L₁ and L₂ are a single bond; or
L₁ is a single bond, and L₂ is a substituted or unsubstituted C₆₋₁₀ arylene; or
L₁ is a substituted or unsubstituted C₆₋₁₀ arylene, and L₂ may be a single bond.

Further, in one embodiment, La may be a single bond, or a substituted or unsubstituted C₆₋₂₀ arylene.

In another embodiment, L₃ is a single bond; or a C₆₋₂₀ arylene which is unsubstituted, or substituted with one or more substituents selected from the group consisting of deuterium, phenyl, and naphthyl.

In yet another embodiment, L₃ may be a single bond, a substituted or unsubstituted phenylene, a substituted or unsubstituted biphenyldiyl, or a substituted or unsubstituted naphthylene.

For example, L₃ may be a single bond, or a substituted or unsubstituted phenylene.

Further, in one embodiment, Ar may be a substituted or unsubstituted C₆₋₂₀ aryl; or a substituted or unsubstituted C₂₋₂₀ heteroaryl containing a heteroatom of O or S.

In other embodiments, Ar is phenyl, biphenylyl, terphenylyl, naphthyl, phenanthryl, triphenylenyl, dibenzofuranyl, dibenzothiophenyl, benzonaphthofuranyl, or benzonaphthothiophenyl, wherein the Ar may be substituted or unsubstituted.

In yet another embodiment, Ar is phenyl, biphenylyl, terphenylyl, naphthyl, phenanthryl, triphenylenyl, dibenzofuranyl, dibenzothiophenyl, benzonaphthofuranyl, or benzonaphthothiophenyl,
wherein the Ar may be unsubstituted, or substituted with one or more substituents selected from the group consisting of deuterium, halogen, cyano, phenyl substituted or unsubstituted with deuterium, biphenylyl substituted or unsubstituted with deuterium, and naphthyl substituted or unsubstituted with deuterium.

For example, Ar may be any one selected from the group consisting of the following formulas and the deuterated structure thereof, but is not limited thereto:

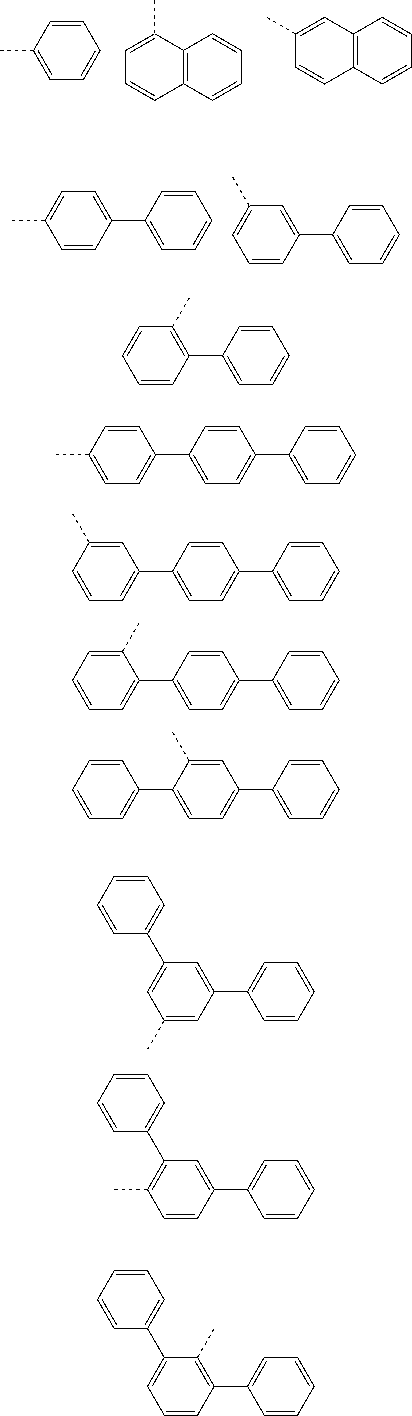

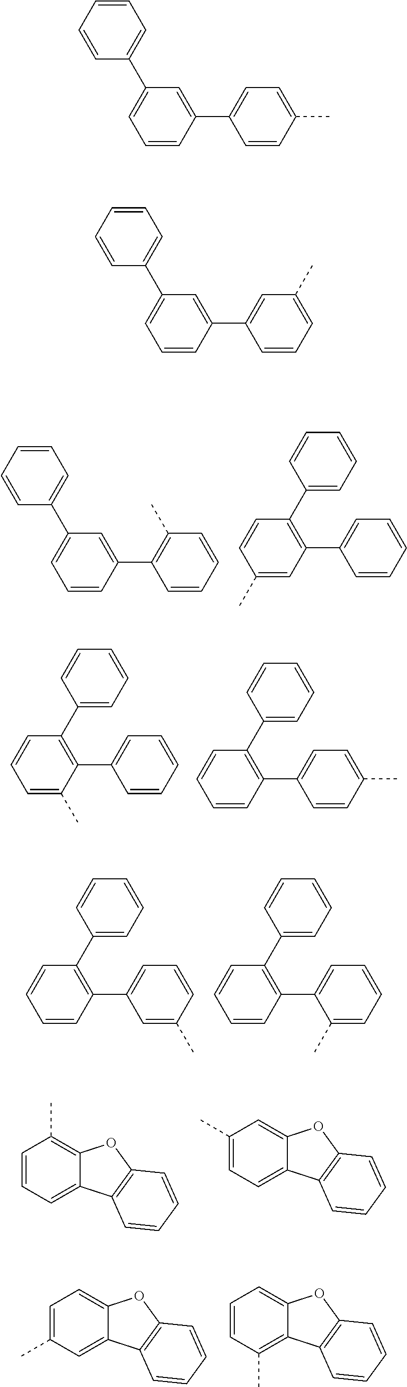

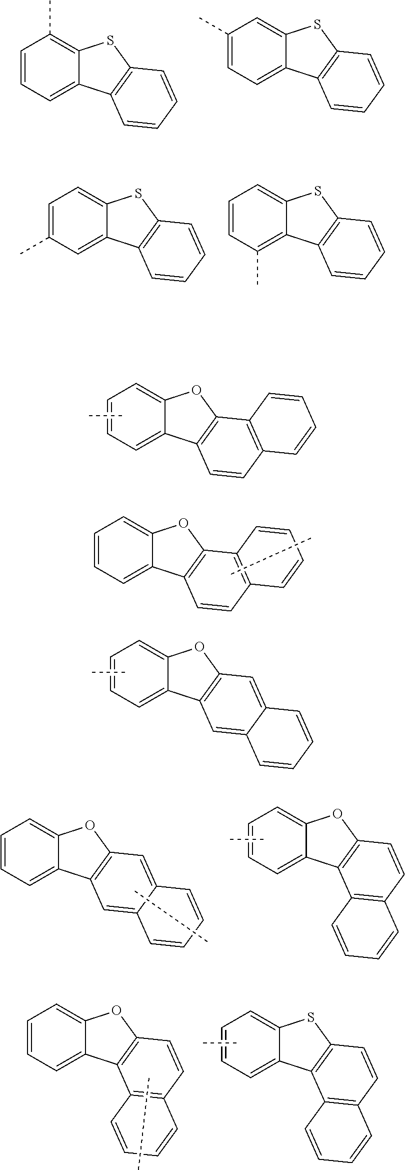

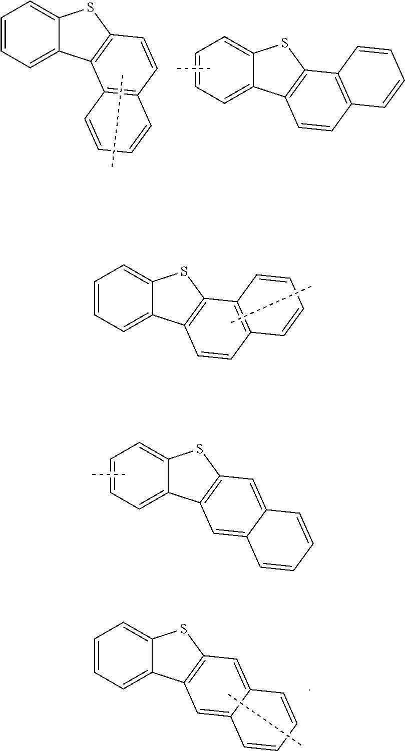

wherein a means the number of R₁, and when a is 2 or more, two or more R₁ may be the same as or different from each other. Specifically, a is 0, 1, 2, 3, 4, 5, 6, 7, 8, or 9.

Further, b means the number of R₂, and when b is 2 or more, two or more R₂ may be the same as or different from each other. Specifically, b is 0, 1, 2, 3, 4, 5, 6, 7, 8, or 9.

For example, R₁ and R₂ may be each independently hydrogen or deuterium.

For example, both R₁ and R₂ may be each independently hydrogen; or both may be deuterium.

Further, the compound may be represented by any one of the following Chemical Formulas 1-1 to 1-9:

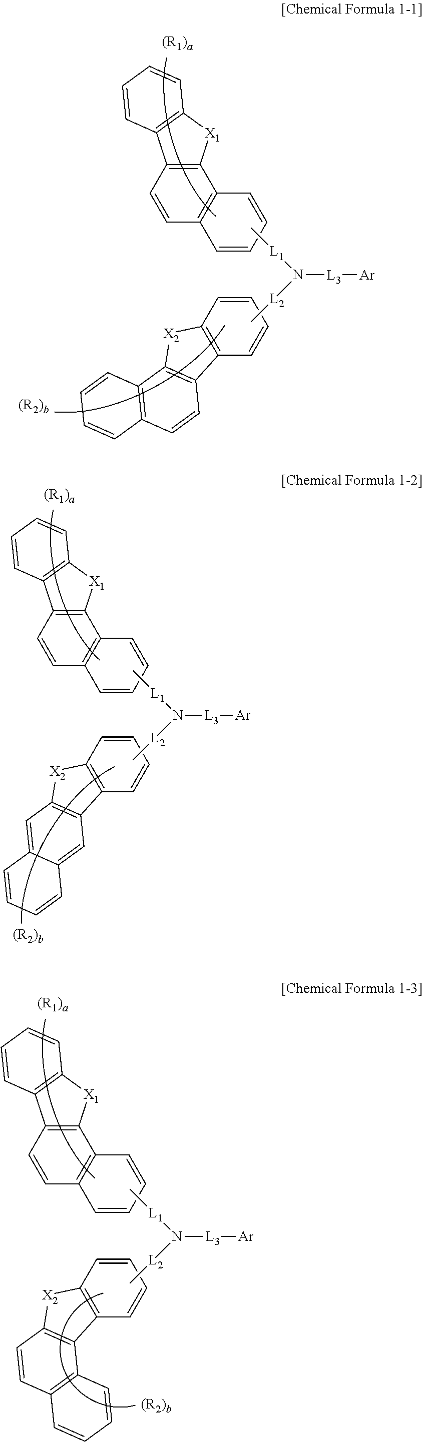

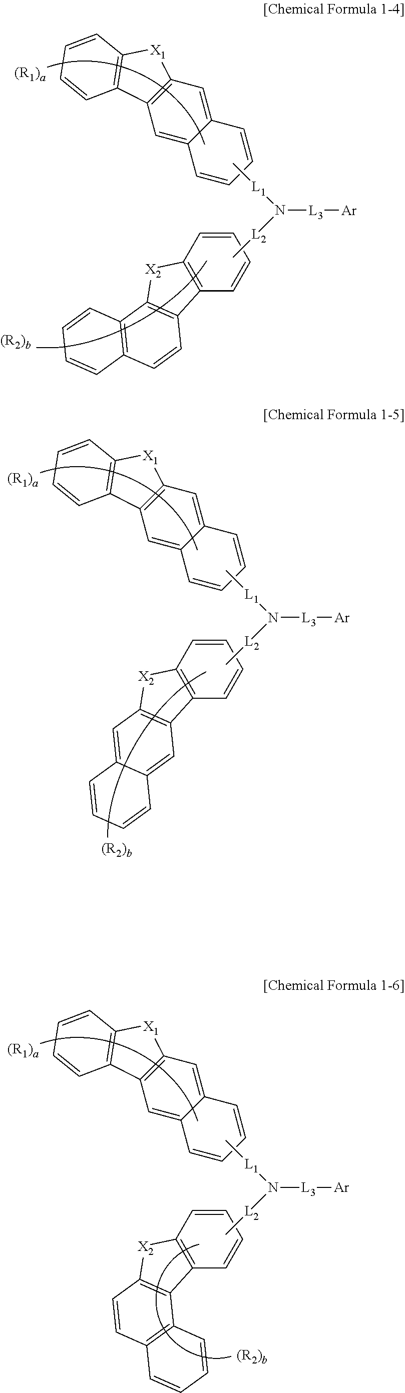

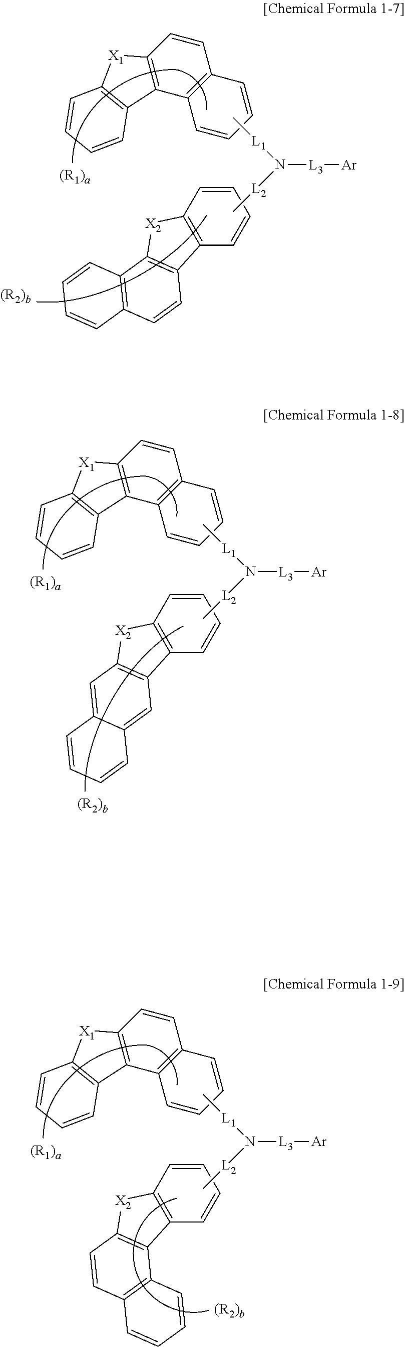

wherein in Chemical Formulas 1-1 to 1-9,
X₁, X₂, L₁ to L₃, Ar, R₁, R₂, a and b are as defined in Chemical Formula 1.

Further, the compound may not contain deuterium, or may contain at least one deuterium.

When the second compound contains deuterium, the deuterium substitution rate of the compound may be 1% to 100%. Specifically, the deuterium substitution rate of the compound may be 5% or more, 10% or more, 20% or more, 30% or more, 40% or more, 50% or more, 60% or more, 70% or more, 75% or more, 80% or more, or 90% or more, and 100% or less.

In one embodiment, the compound may not contain deuterium, or may contain 1 to 50 deuterium atoms. More specifically, the compound may not contain deuterium, or may contain 1 or more, 2 or more, 3 or more, 4 or more, 5 or more, 6 or more, 7 or more, 8 or more, or 9 or more, and 50 or less, 40 or less, 30 or less, 28 or less, 26 or less, 24 or less, 22 or less, 20 or less, 18 or less, 16 or less, 14 or less, 13 or less, 12 or less, 11 or less, or 10 or less deuterium atoms.

In this case, when the number of deuterium substitution of the compound is to be represented, it may be represented by the following Chemical Formula 1D:

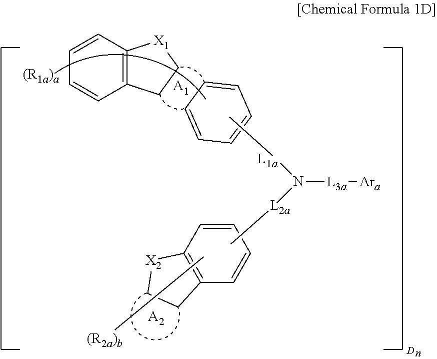

wherein in Chemical Formula 1D,
Dn means that n hydrogens have been replaced by deuterium,
wherein n is an integer of 0 or more,
L₁ₐ to L₃ₐ, Arₐ, R₁ₐ and R₂ₐ mean L₁ to L₃, Ar, R₁ and R₂ substituents which are not substituted with deuterium, respectively.

Meanwhile, representative examples of the compound represented by Chemical Formula 1 are as follows:

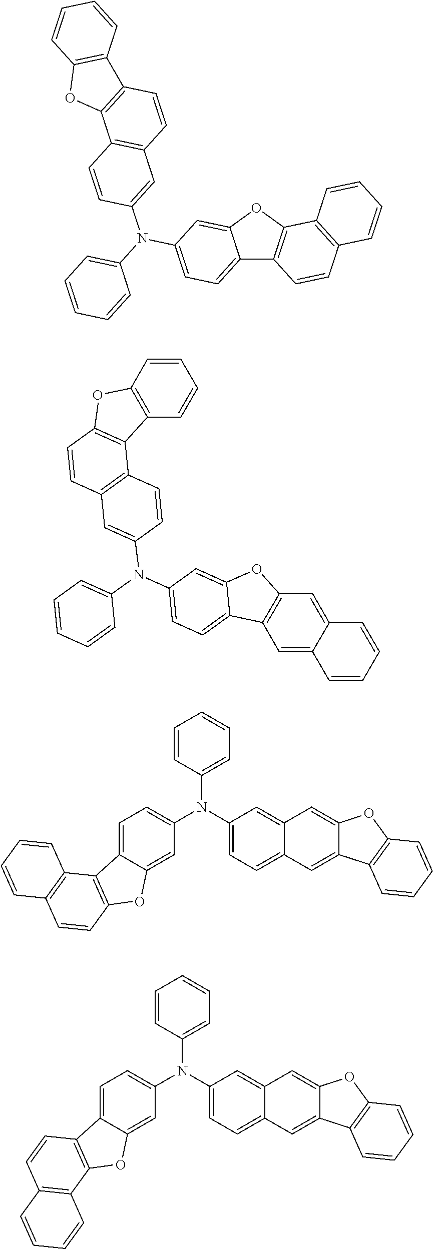

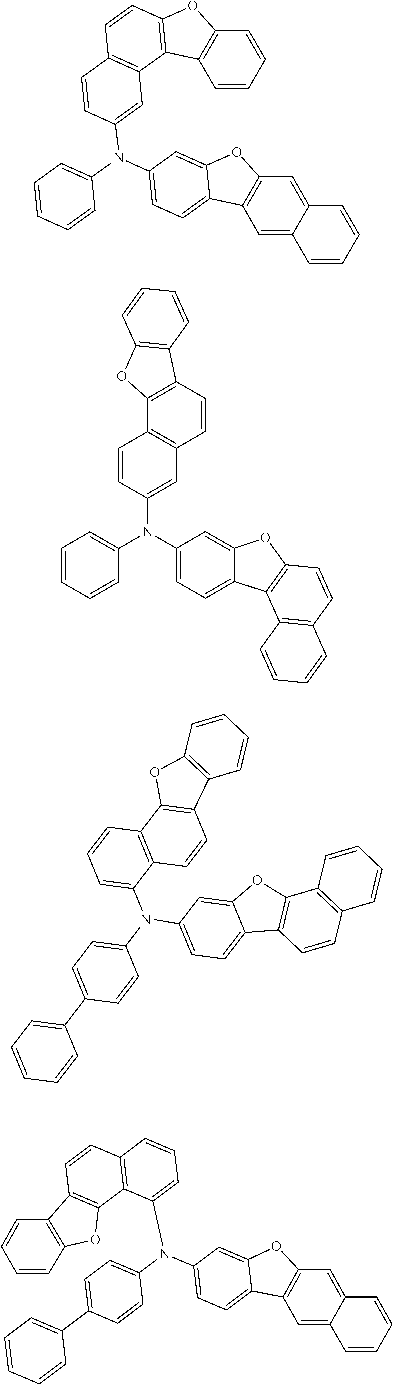

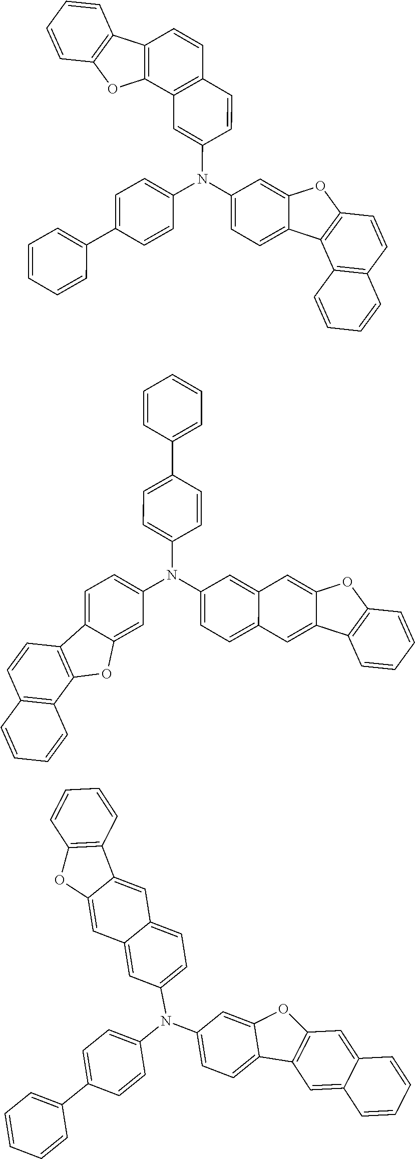

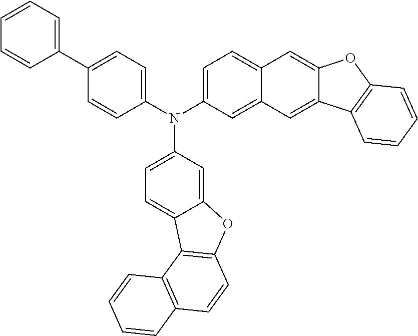

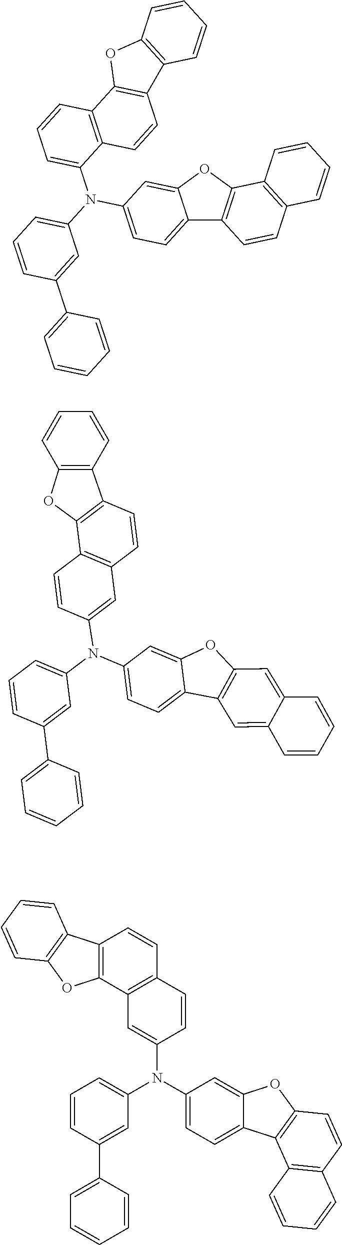

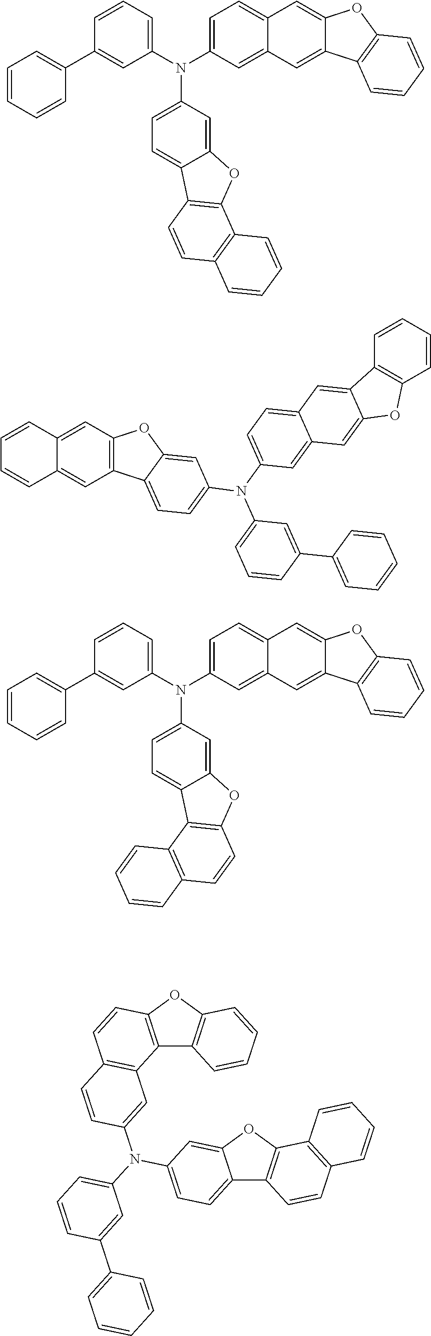

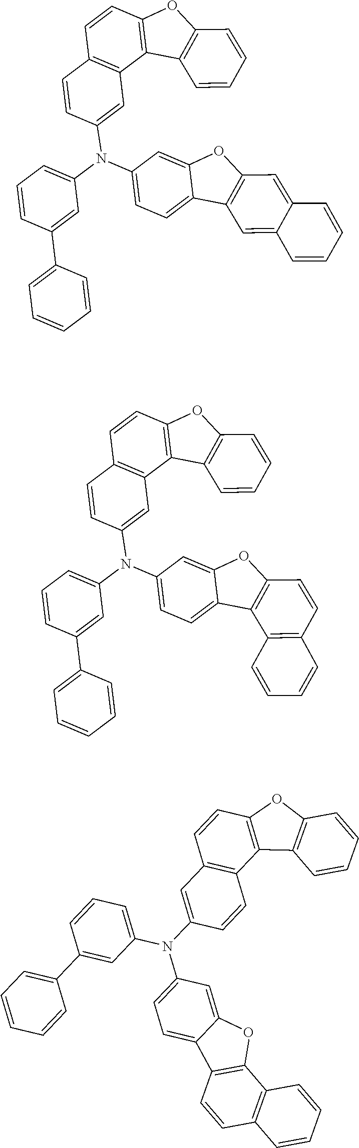

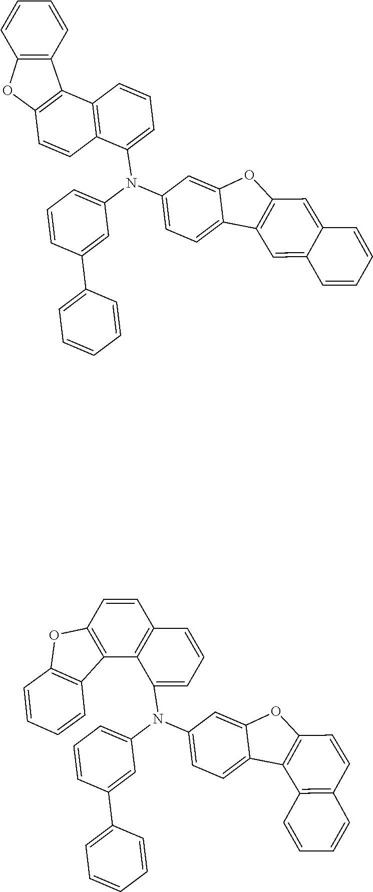

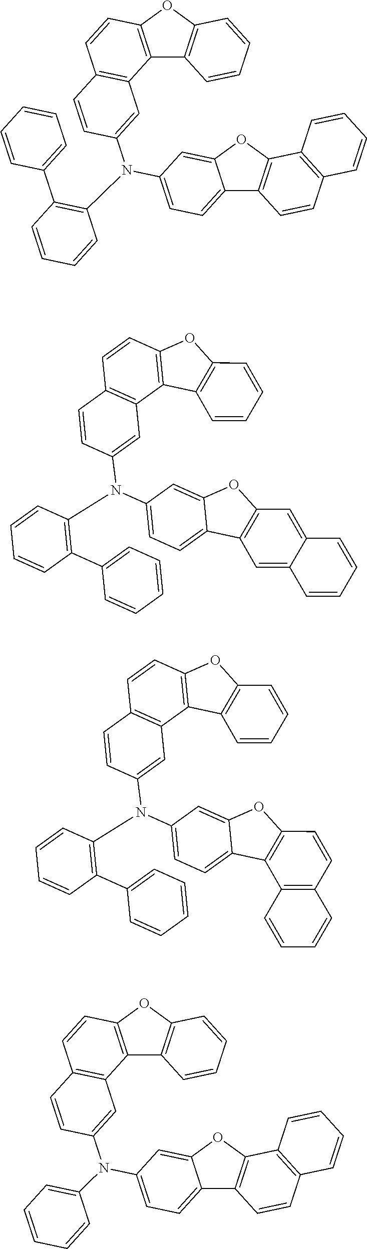

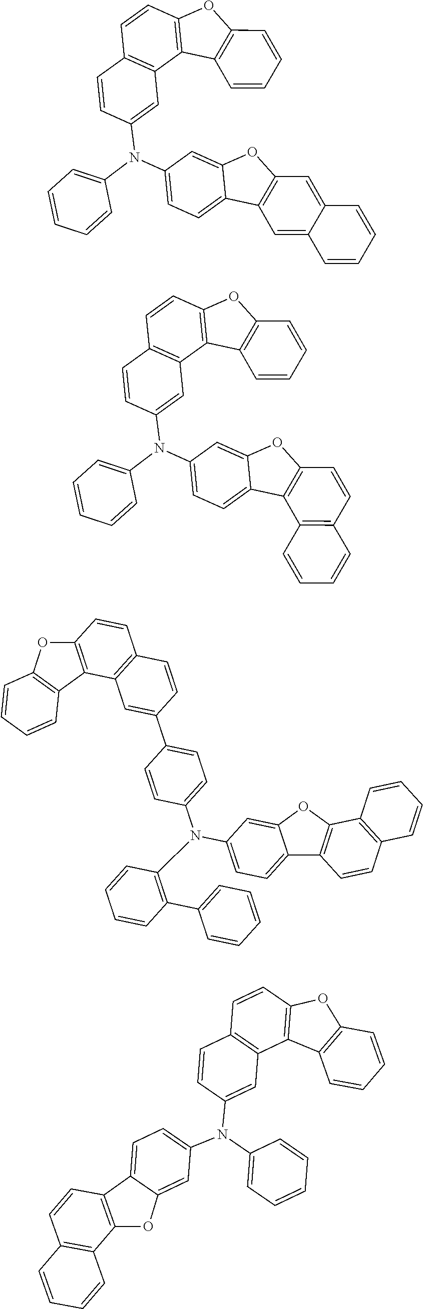

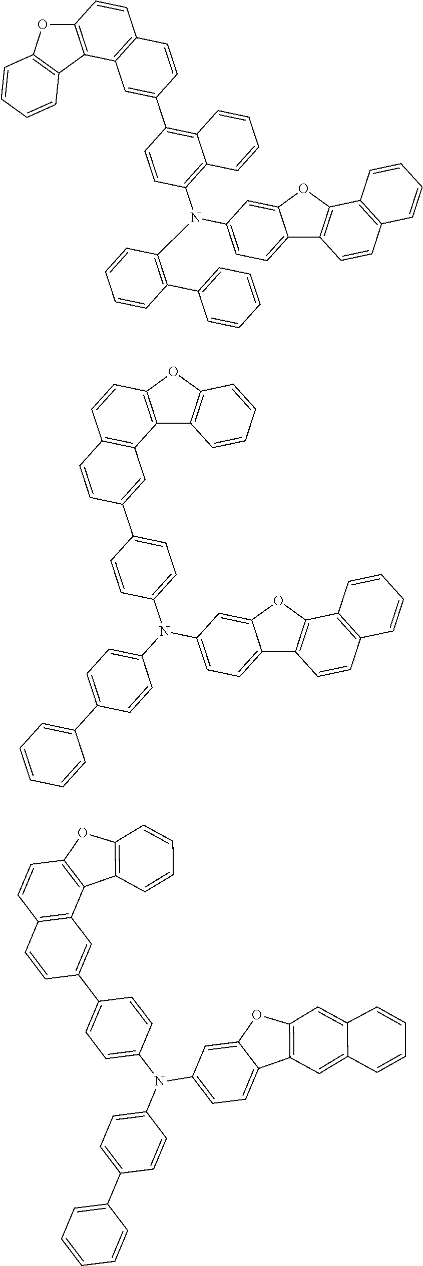

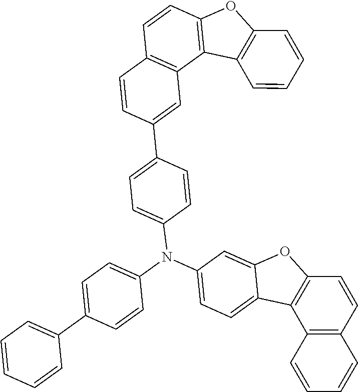

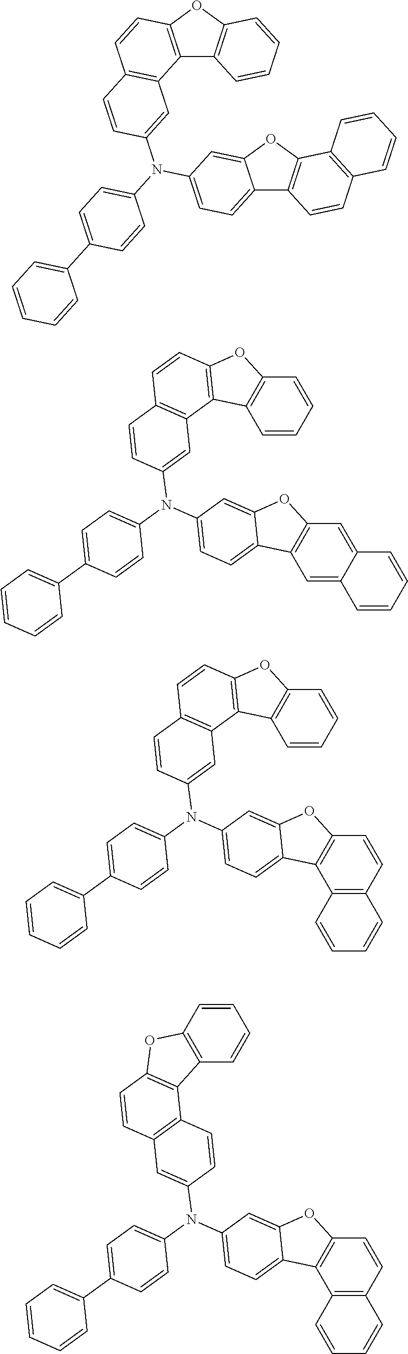

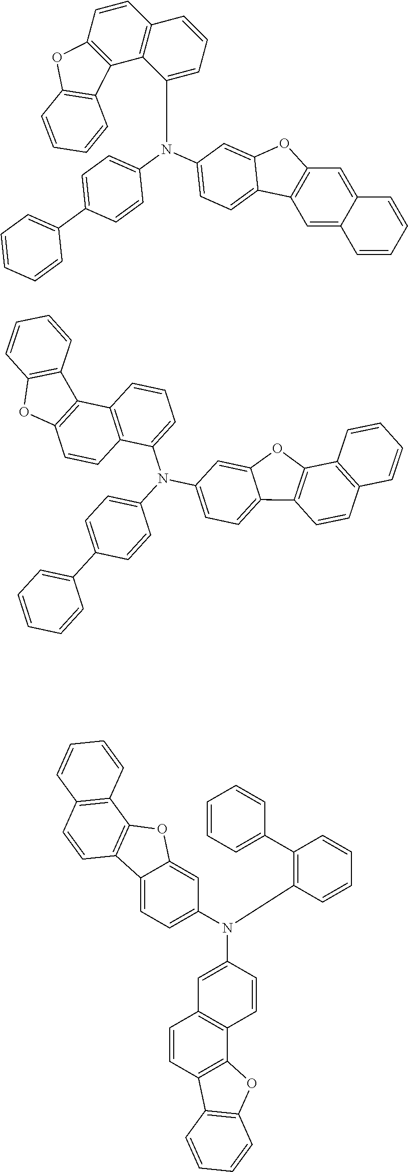

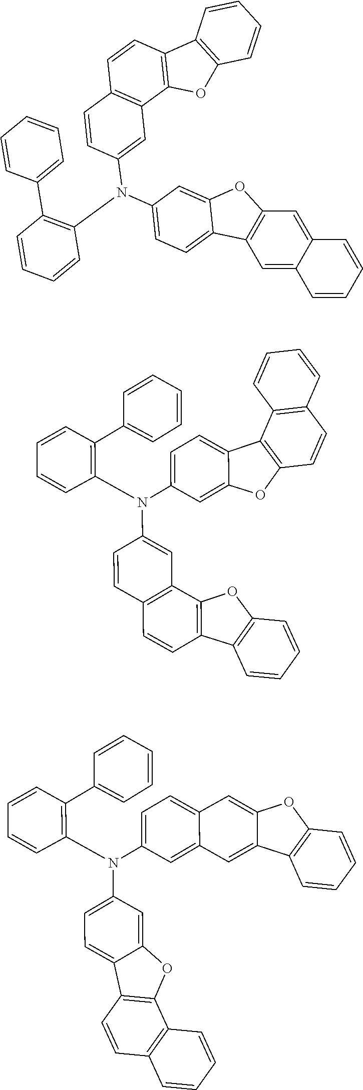

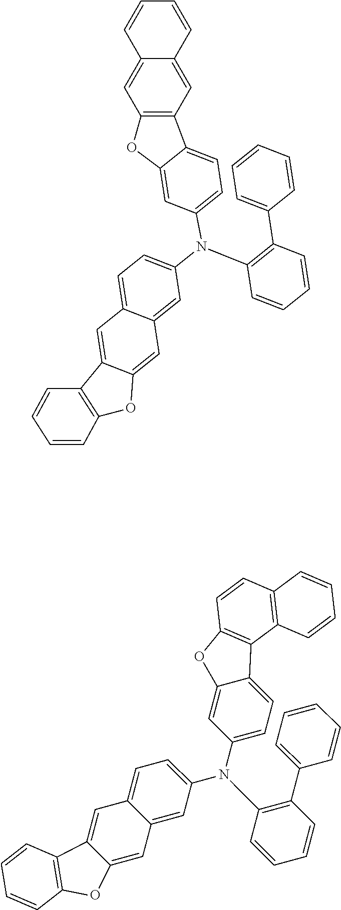

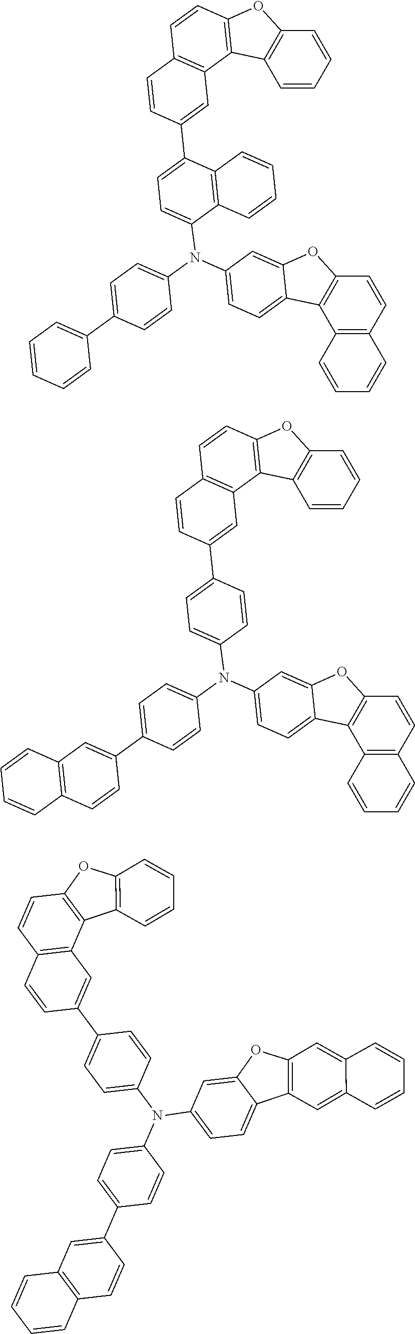

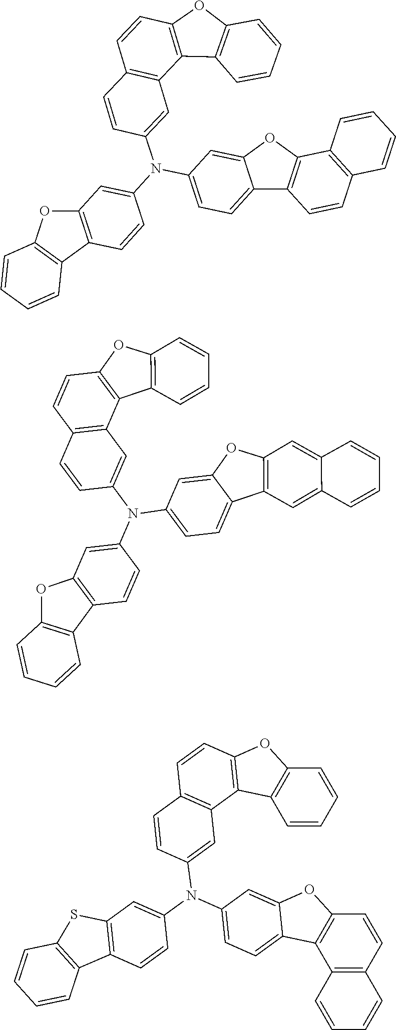

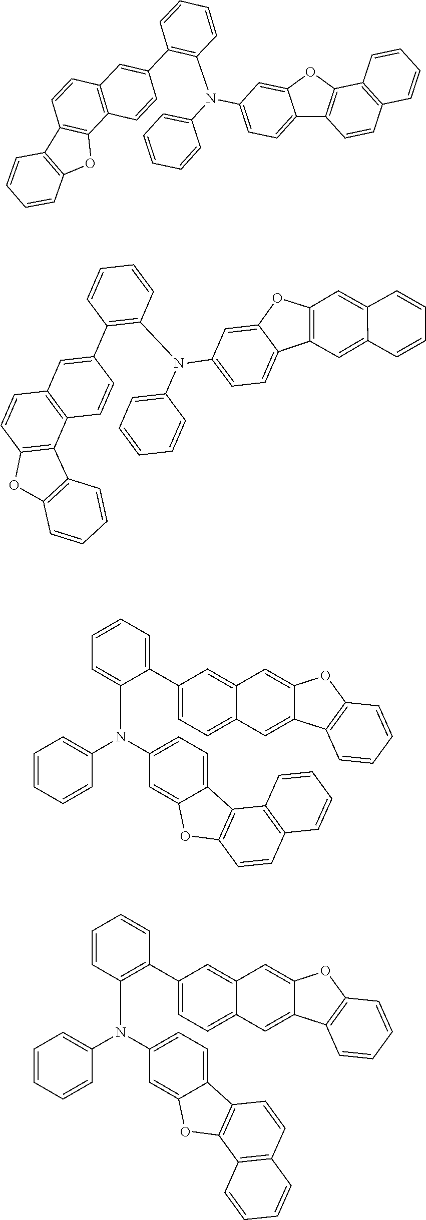

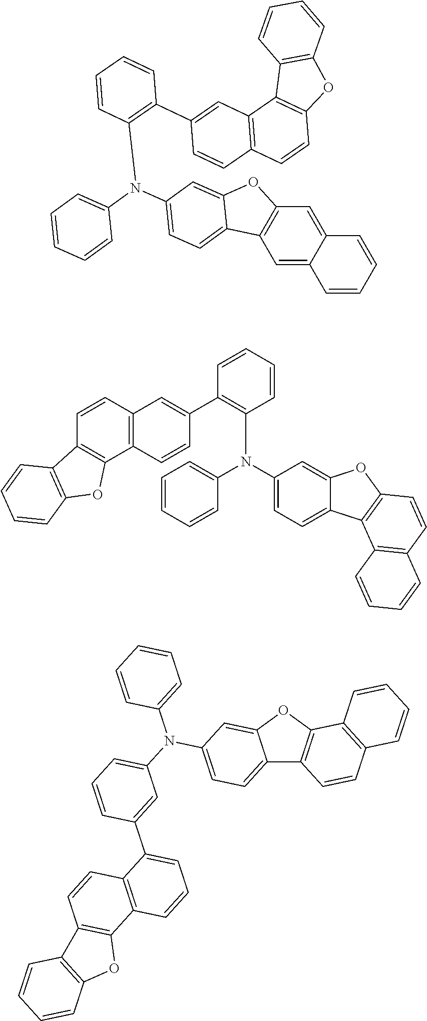

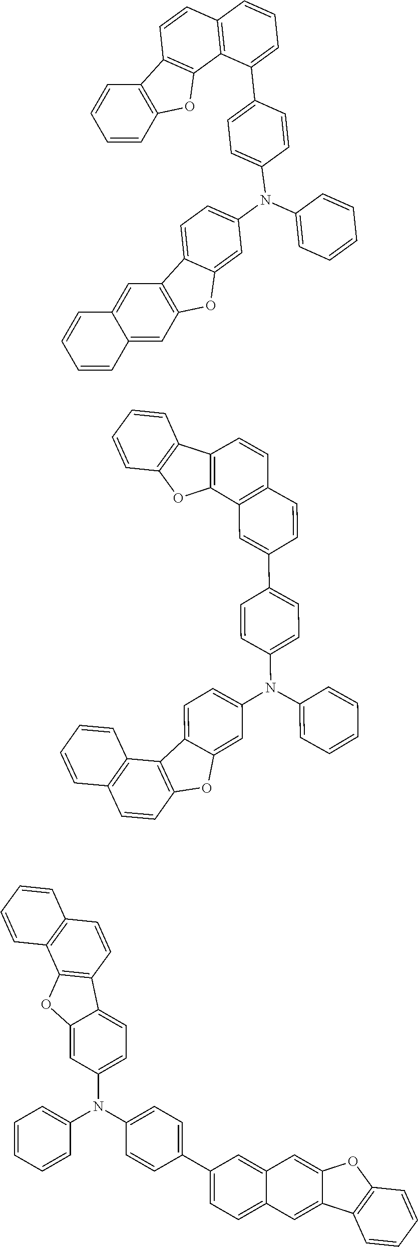

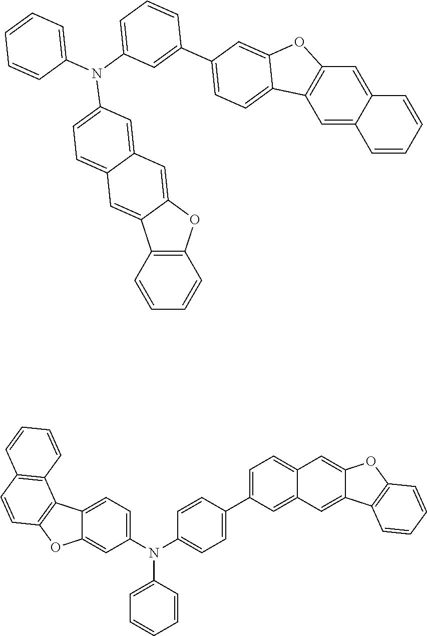

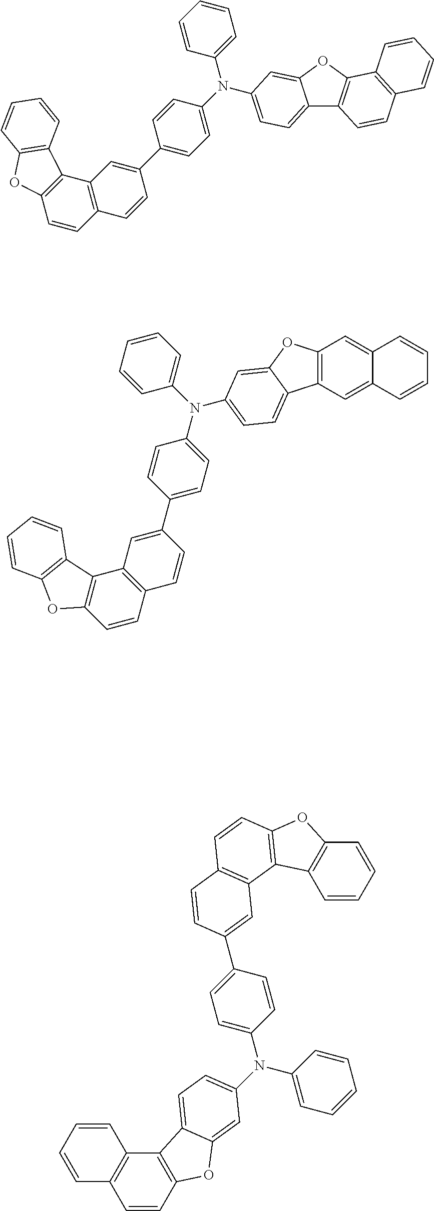

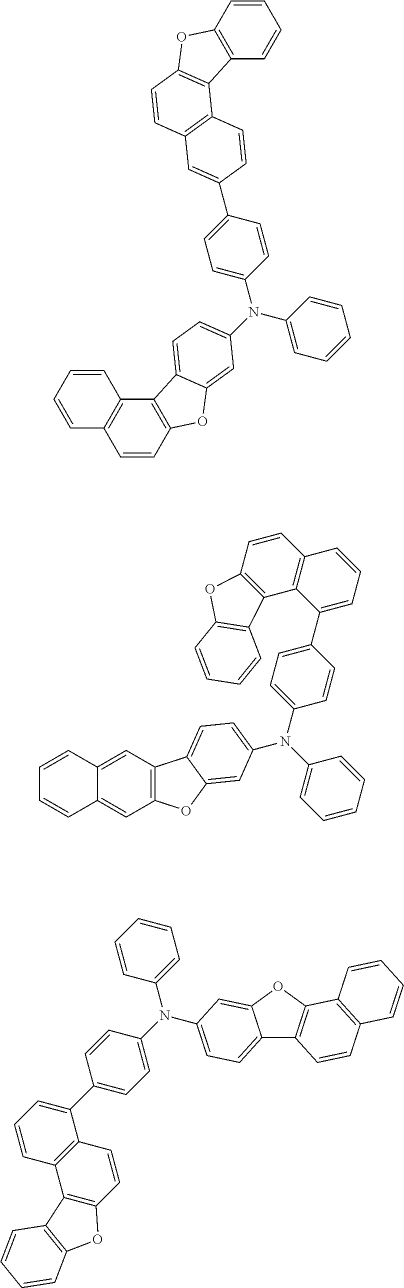

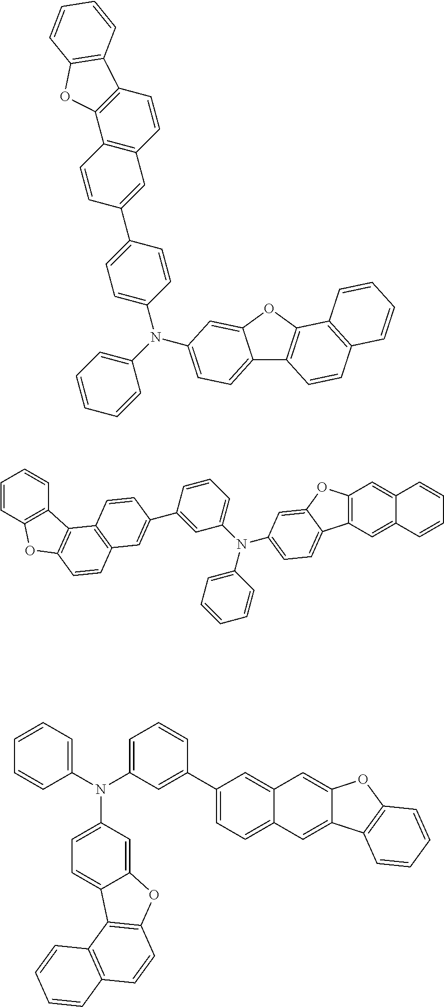

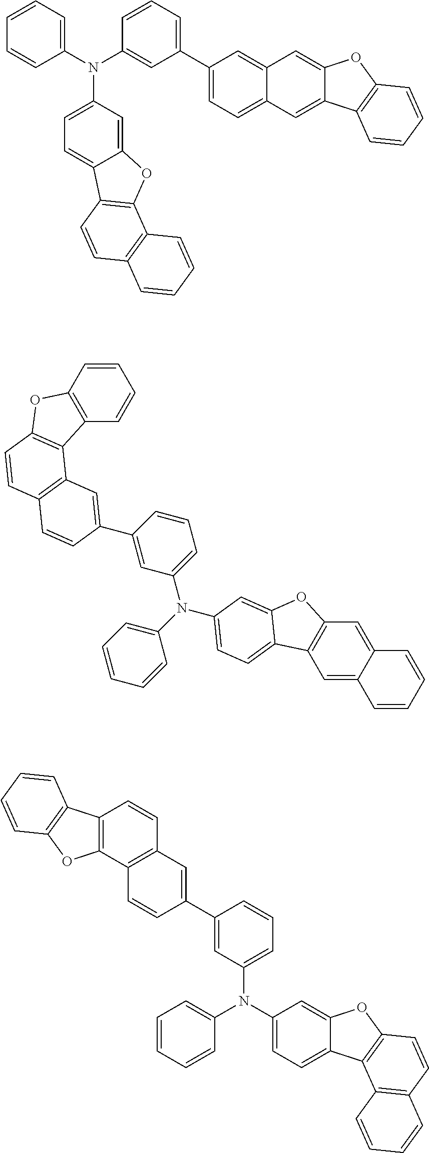

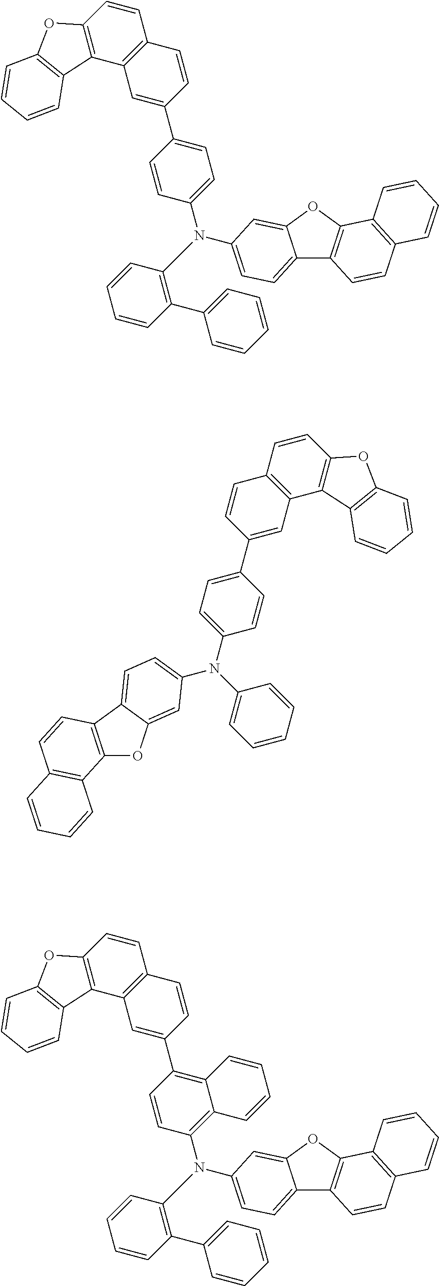

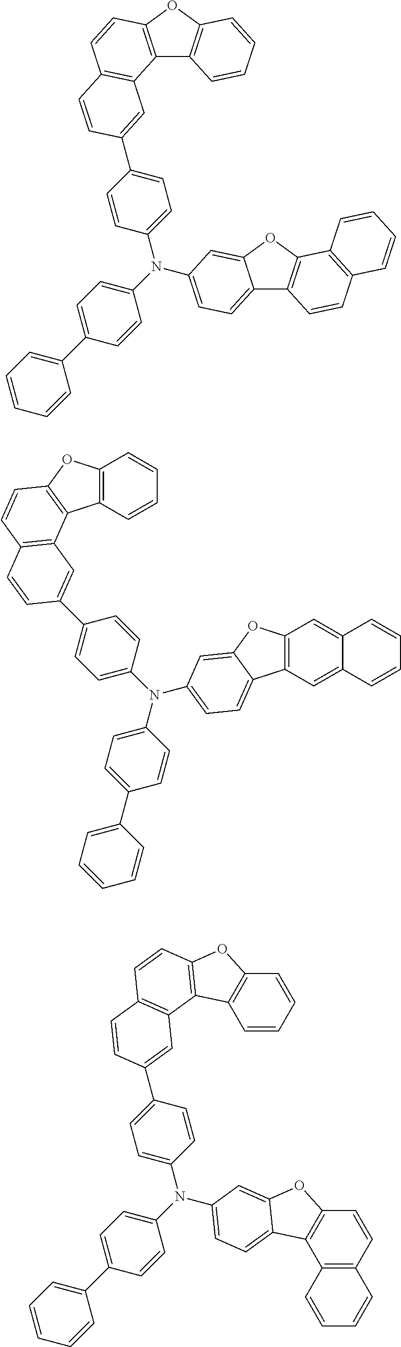

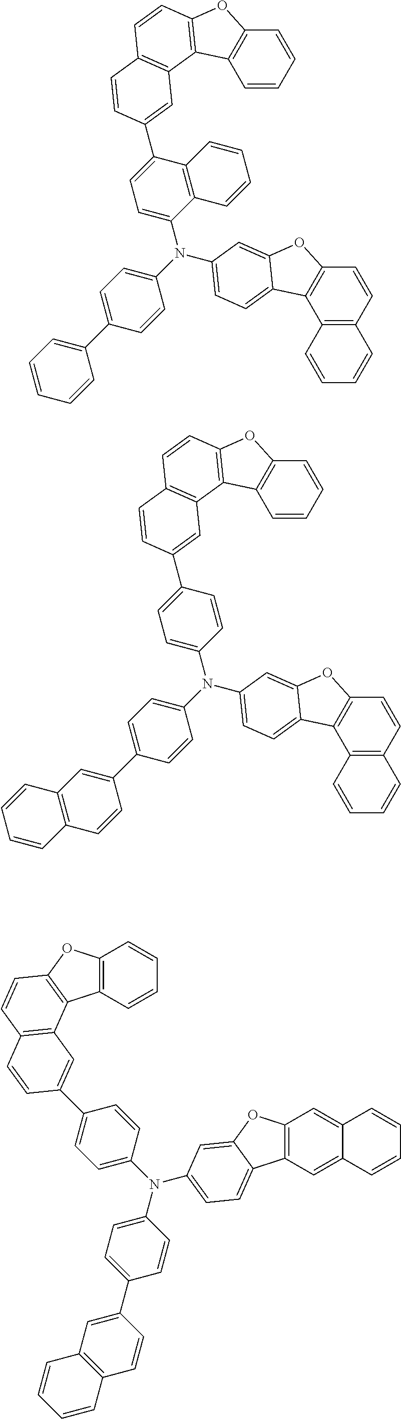

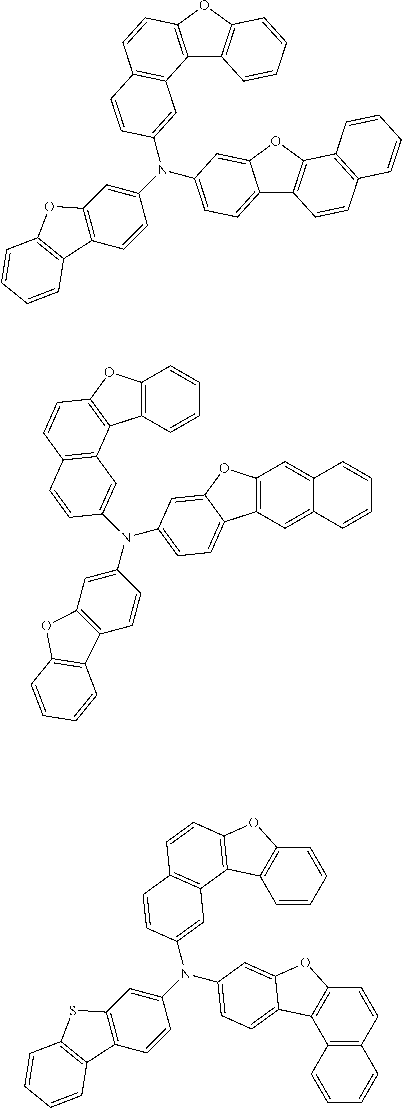

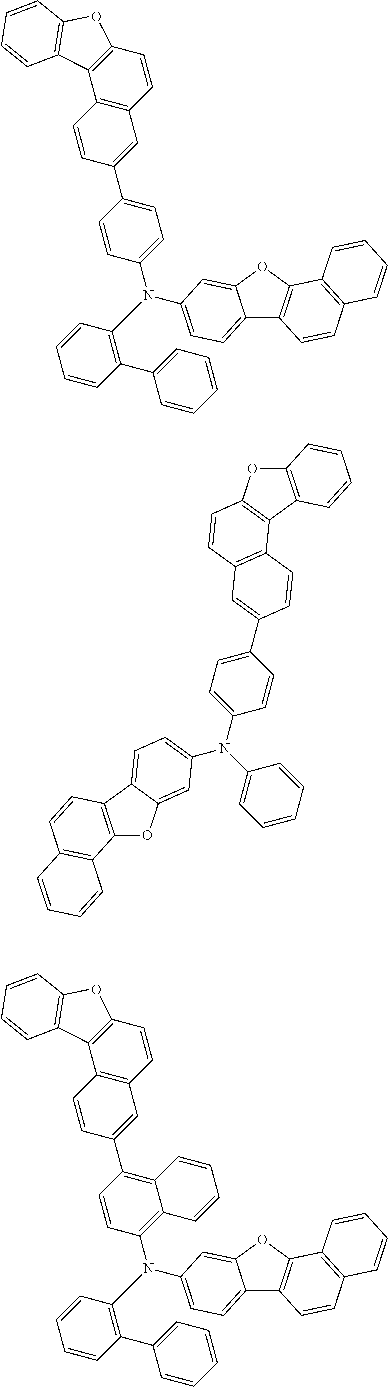

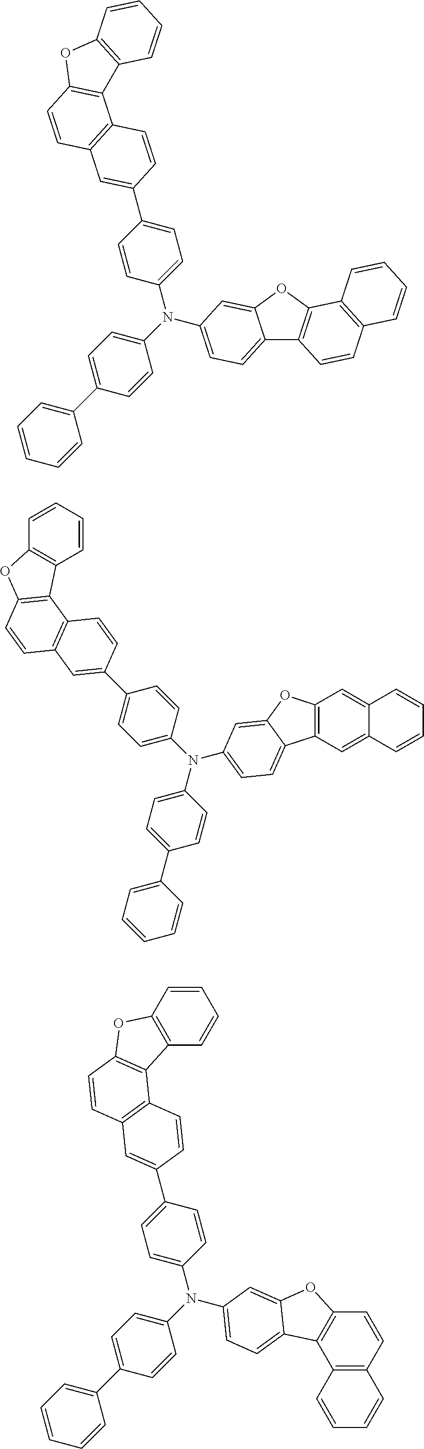

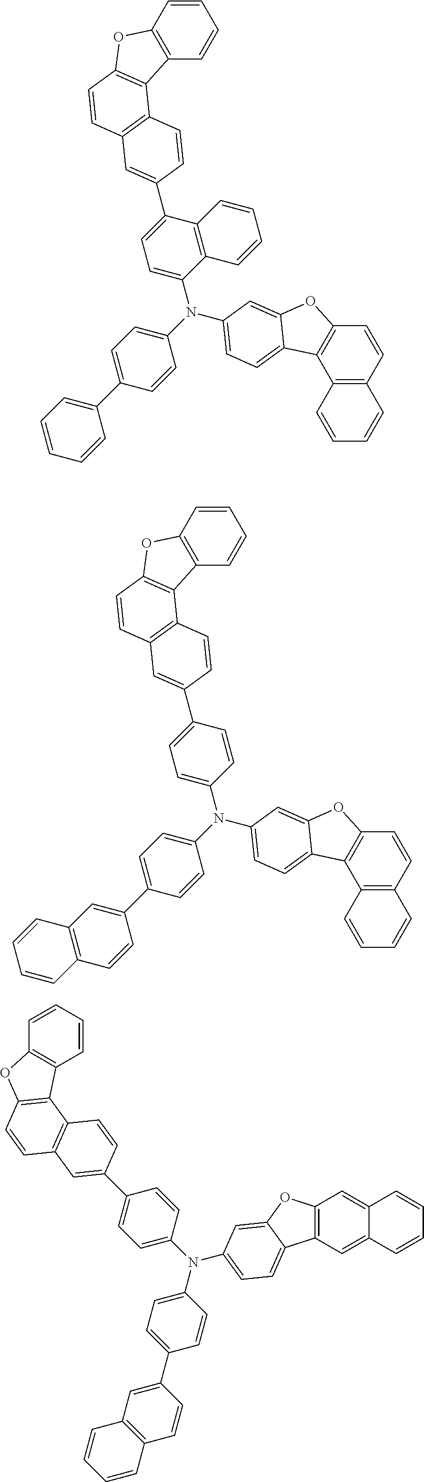

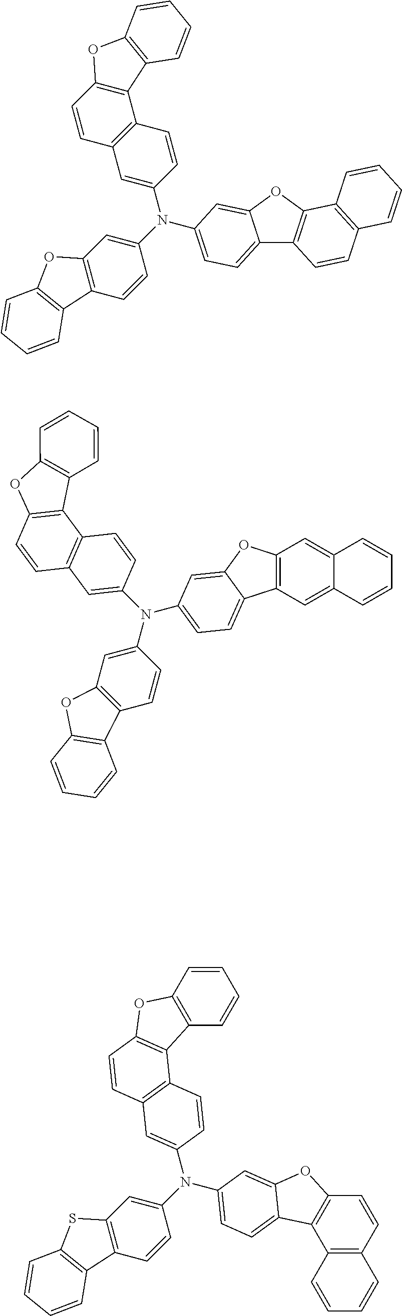

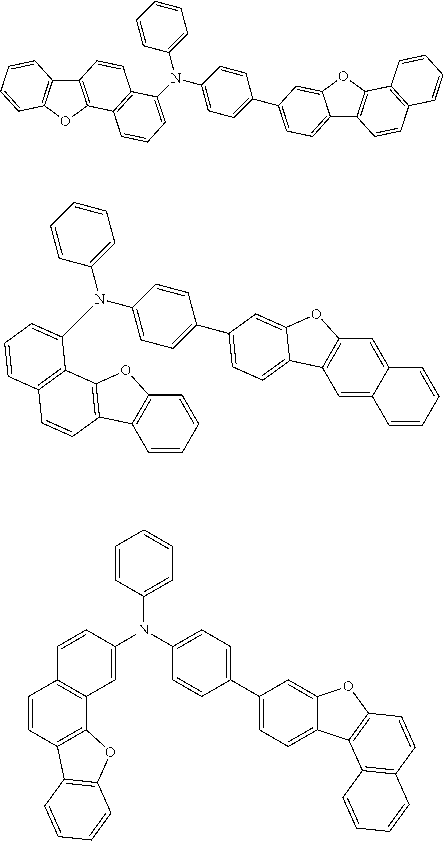

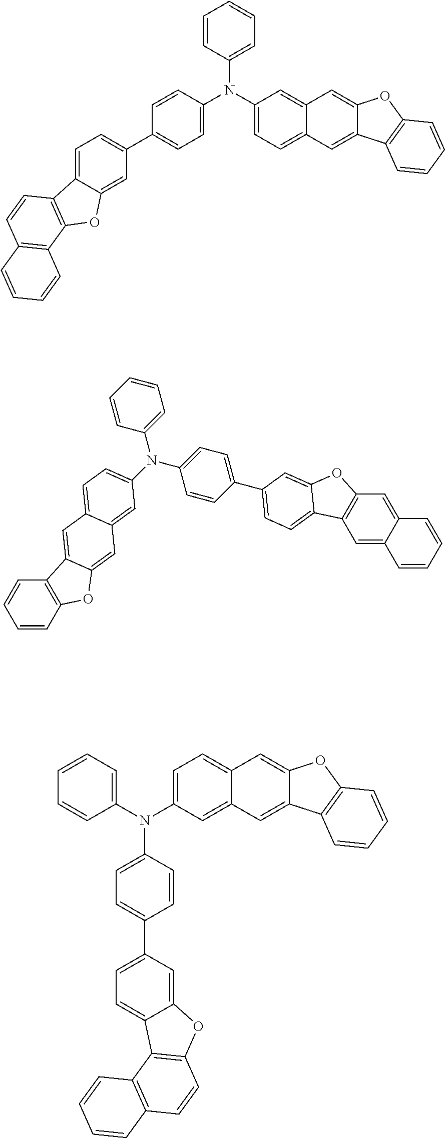

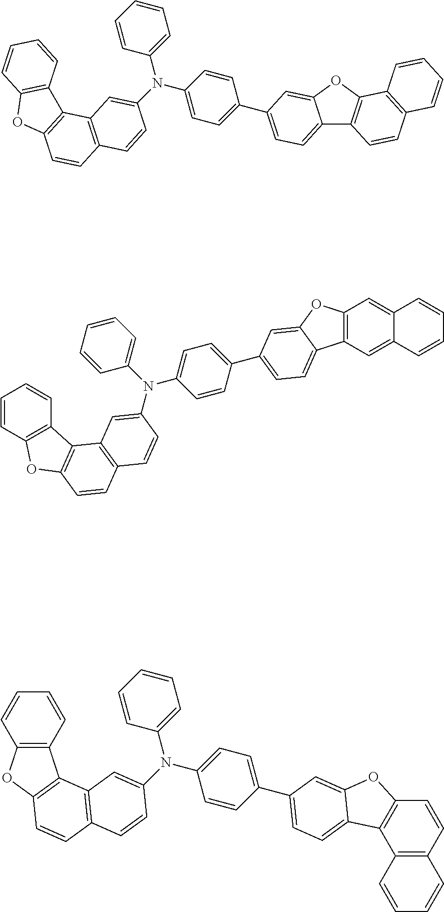

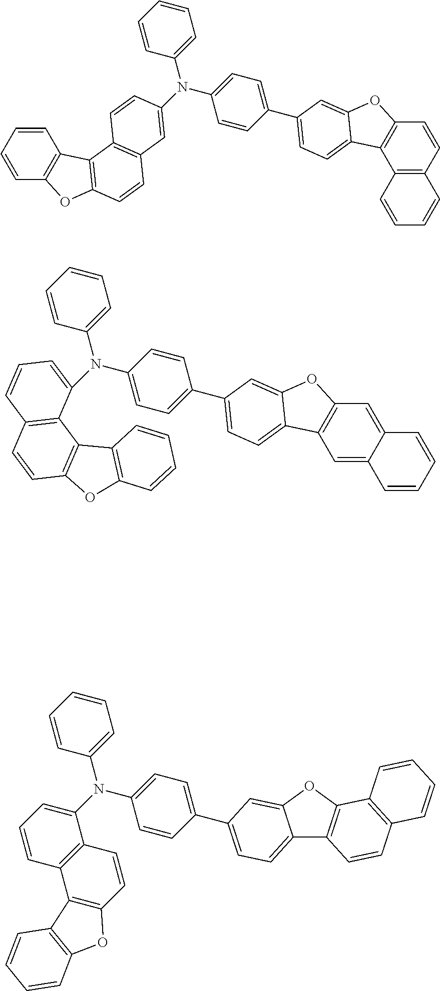

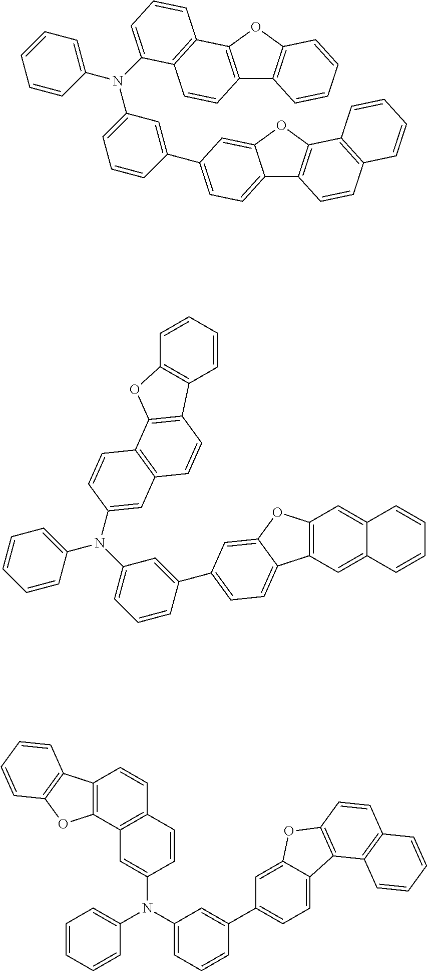

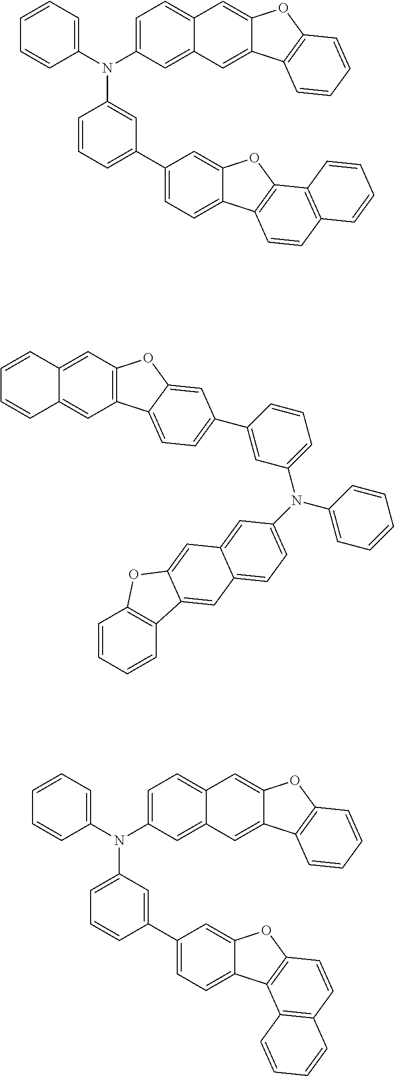

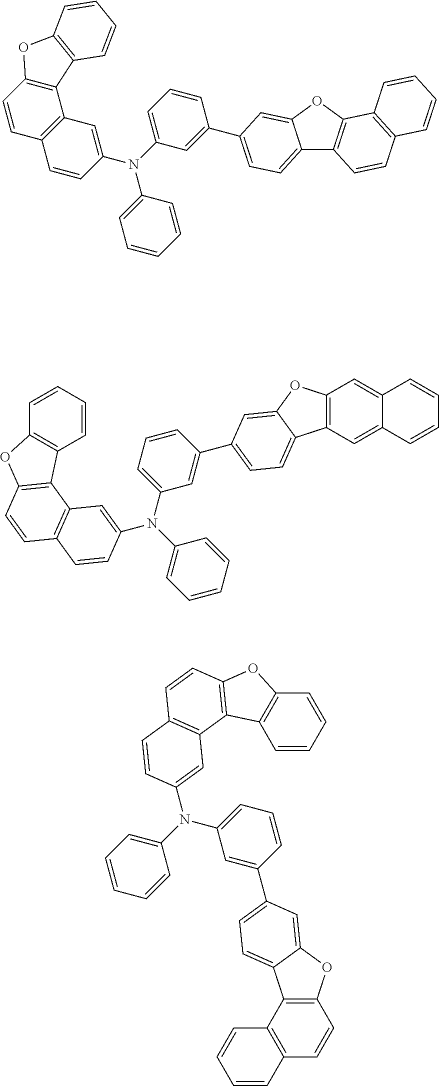

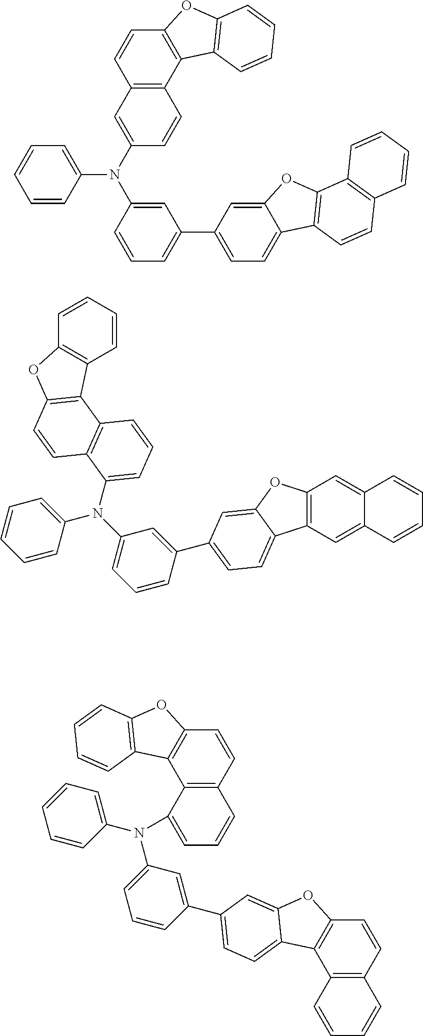

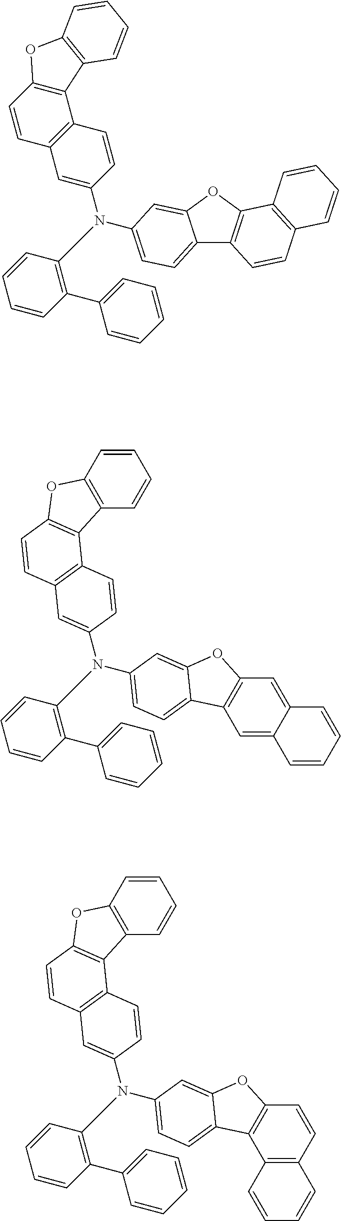

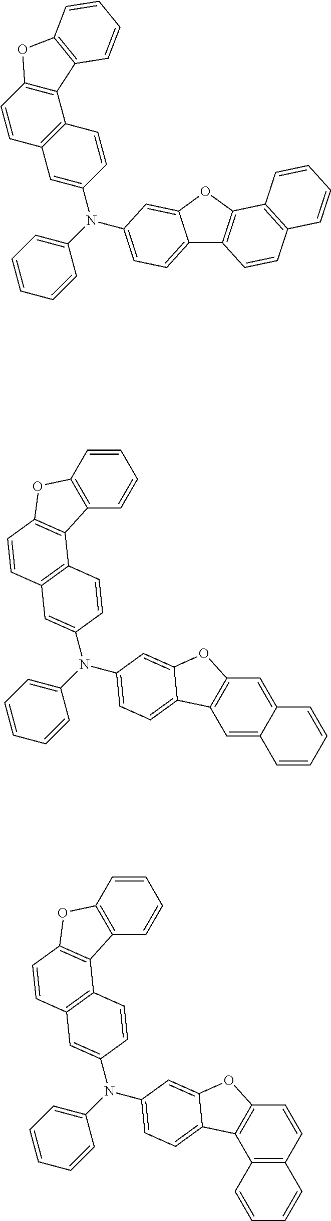

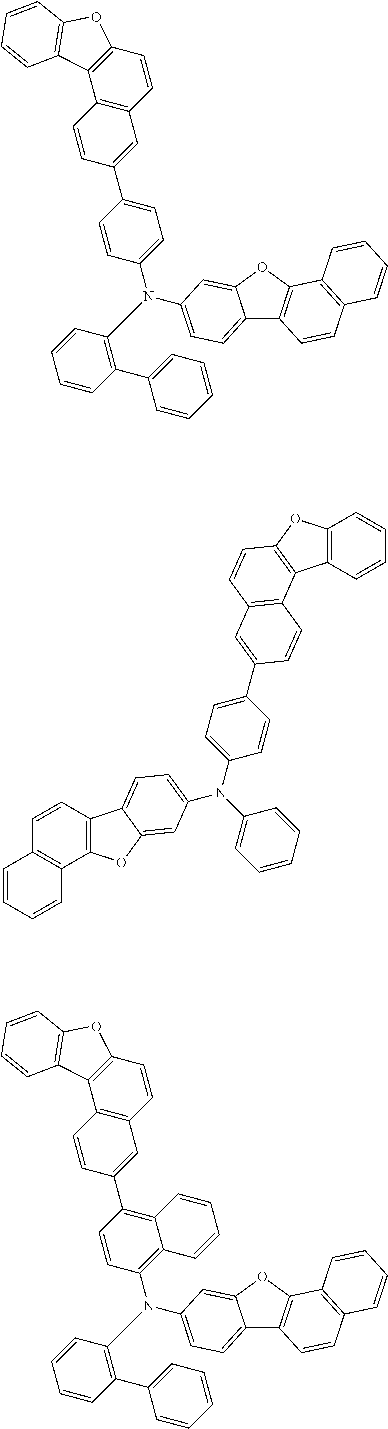

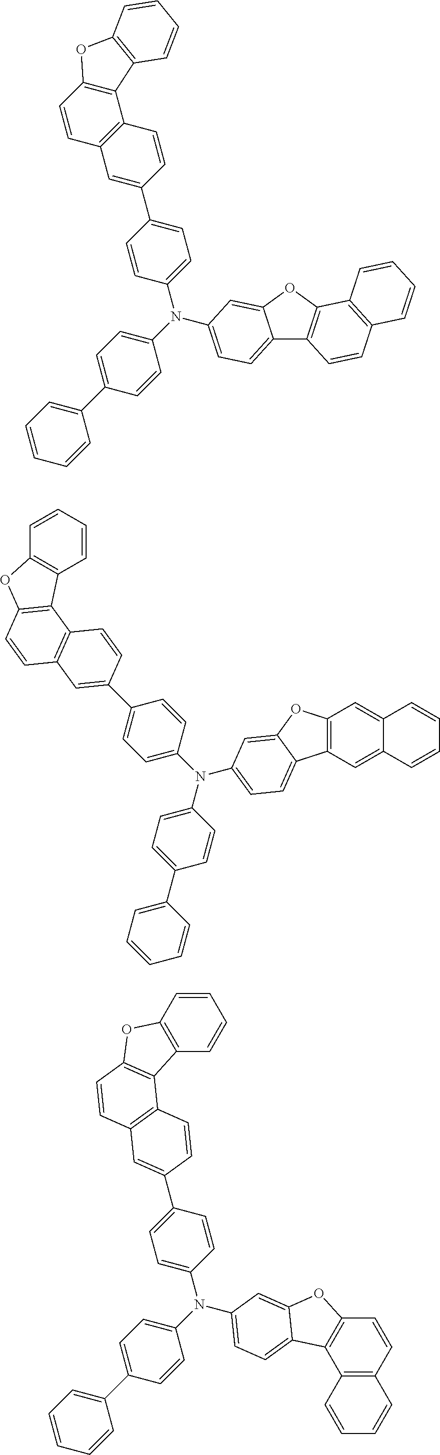

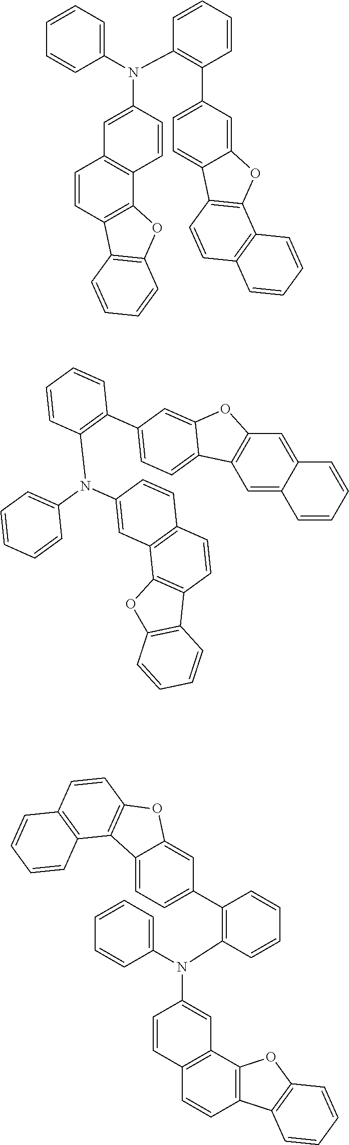

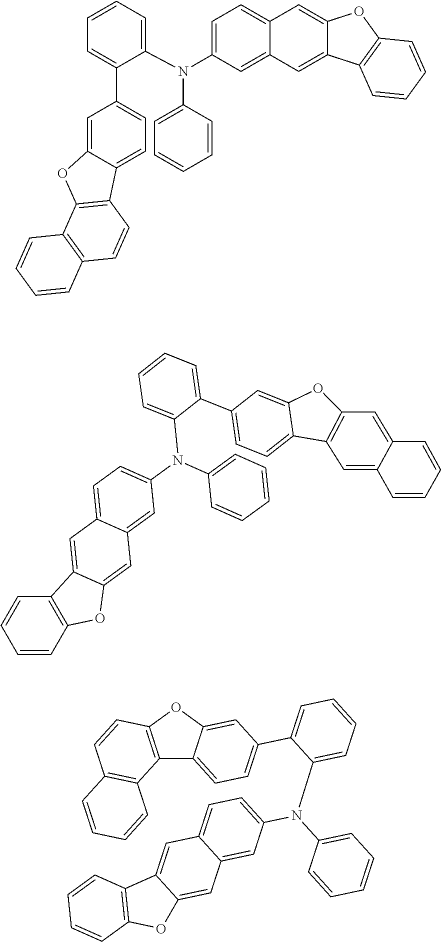

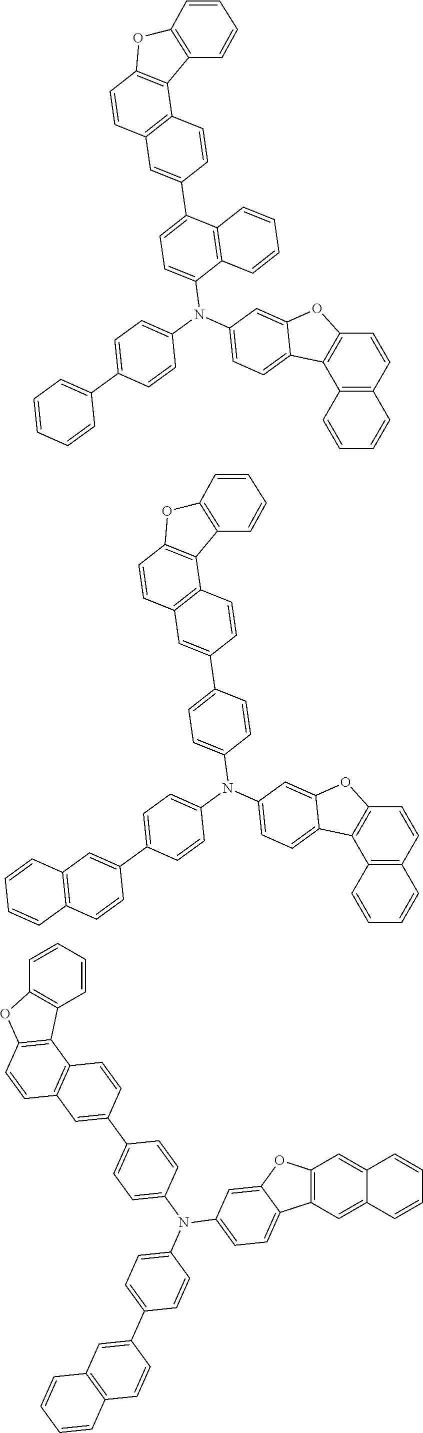

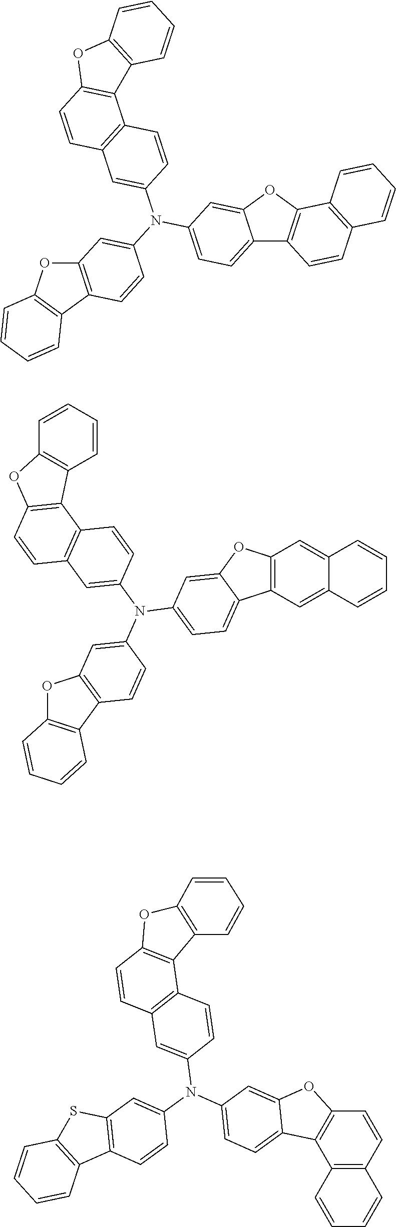

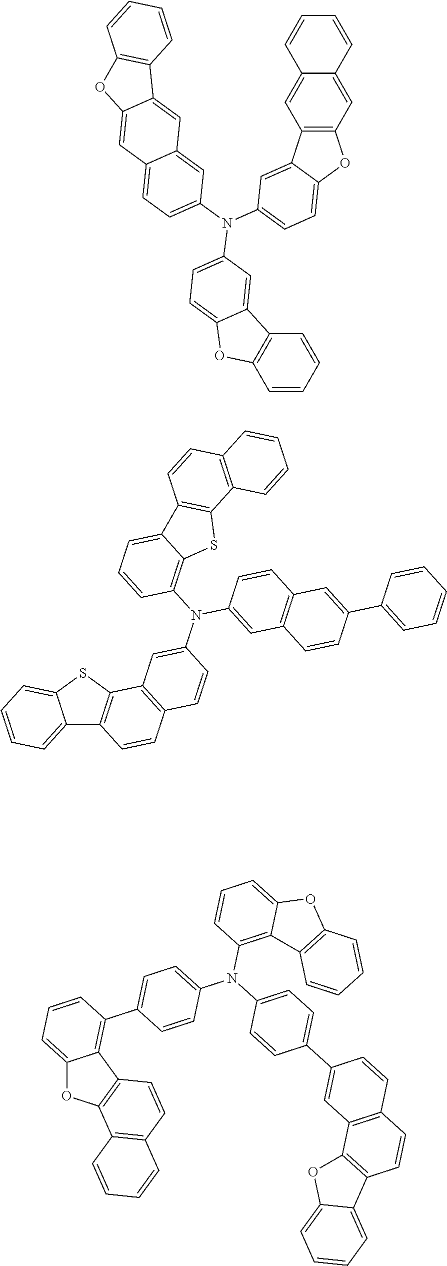

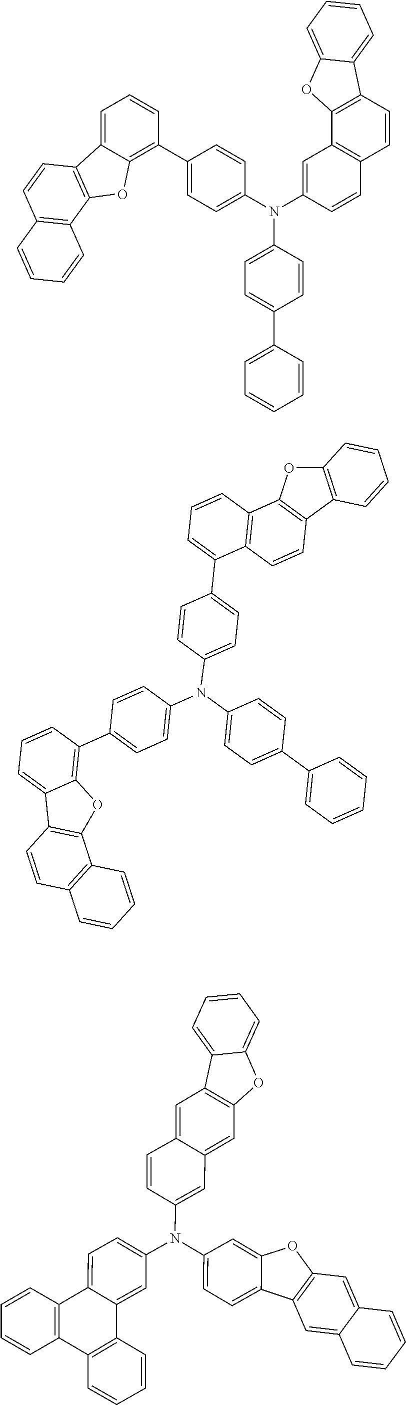

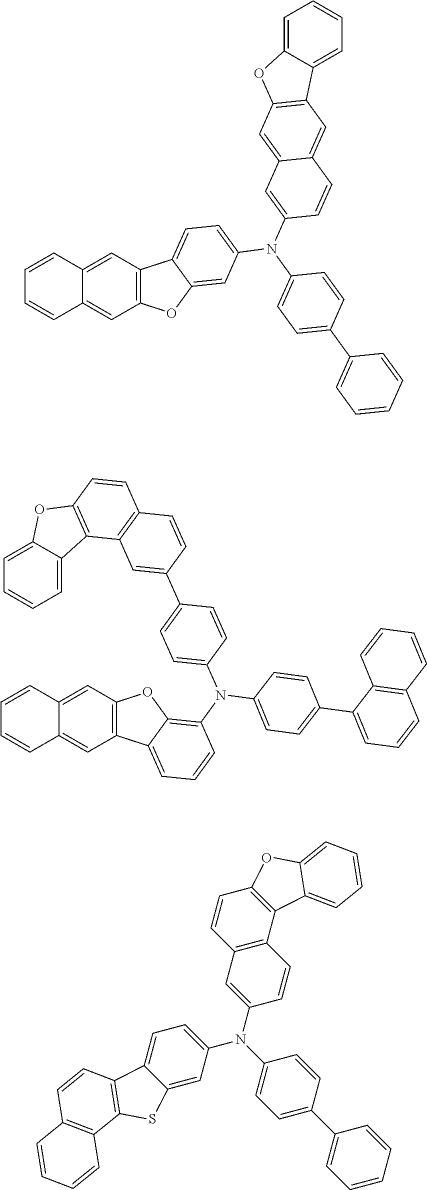

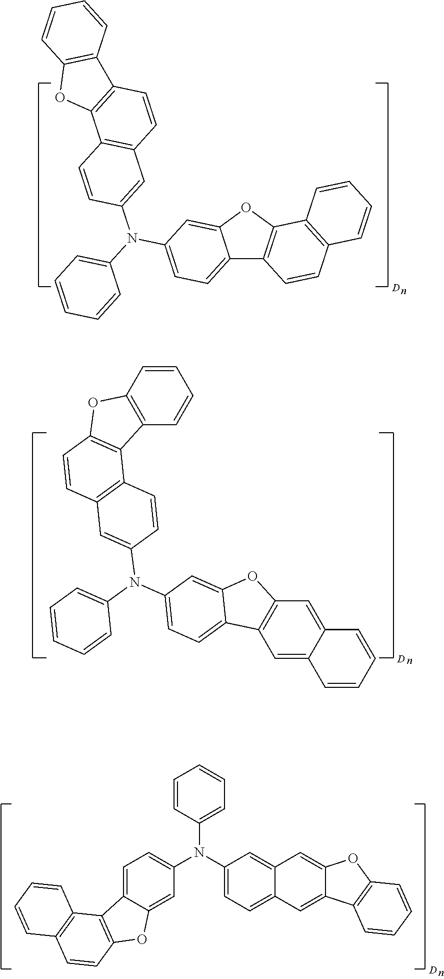

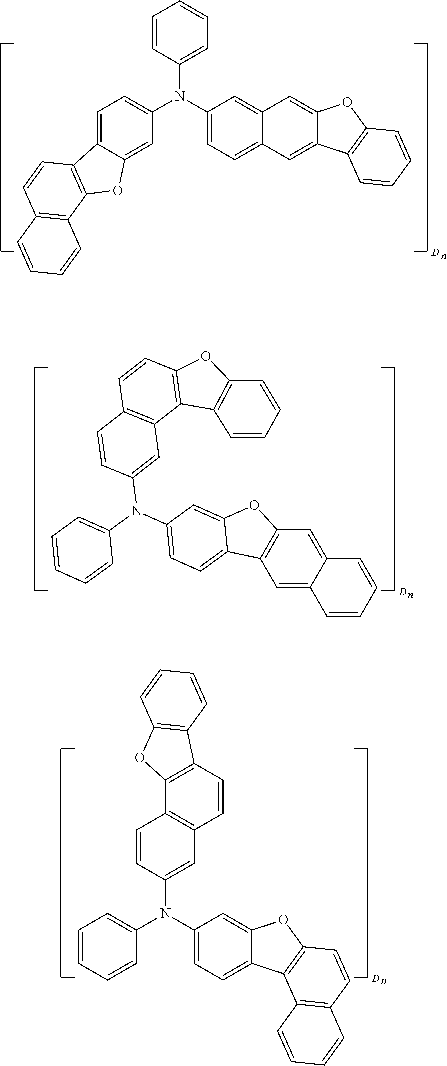

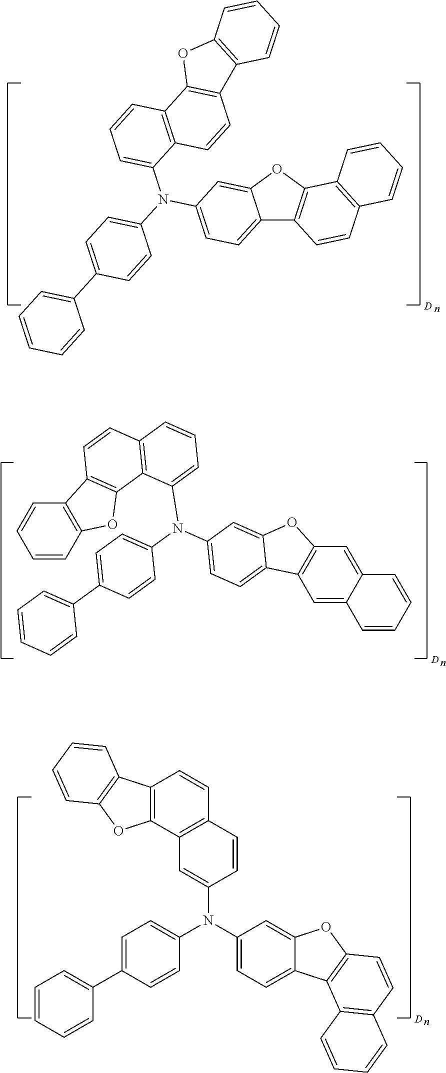

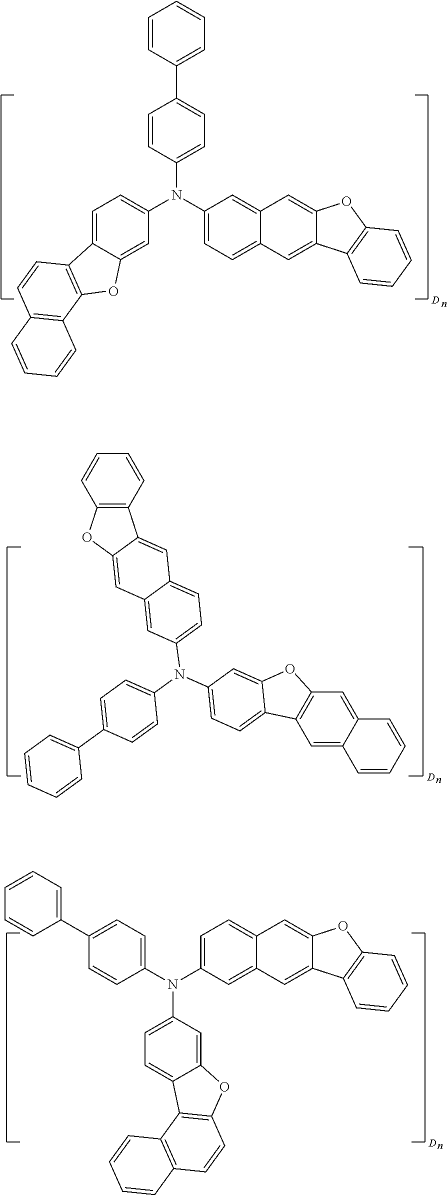

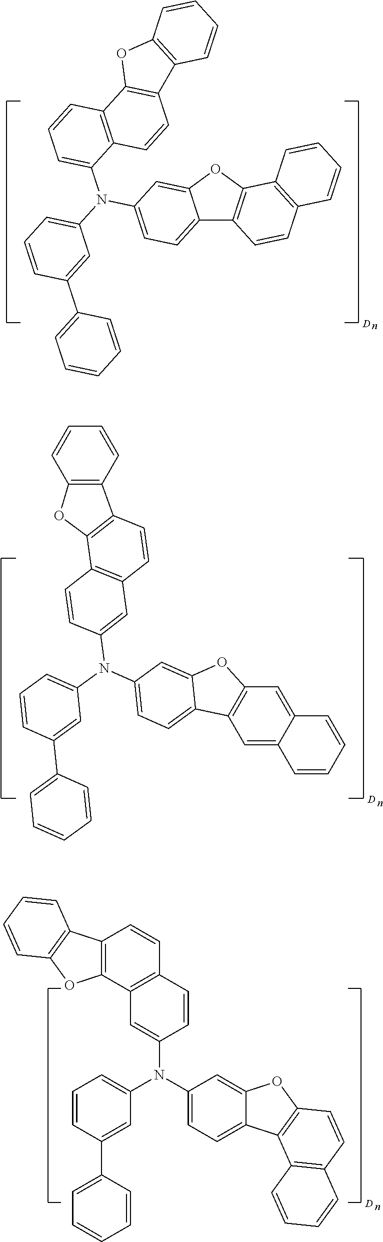

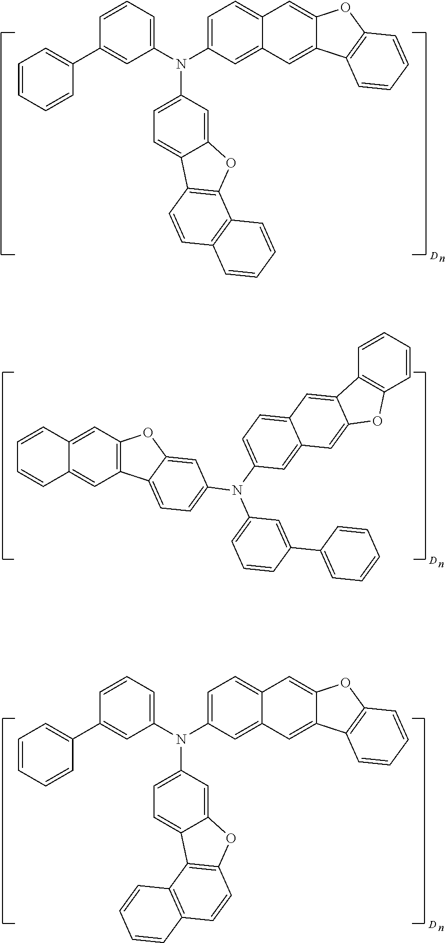

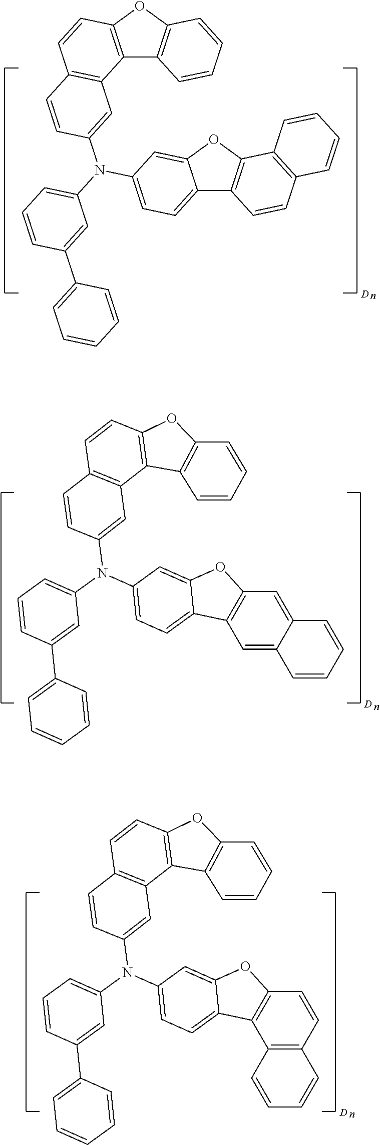

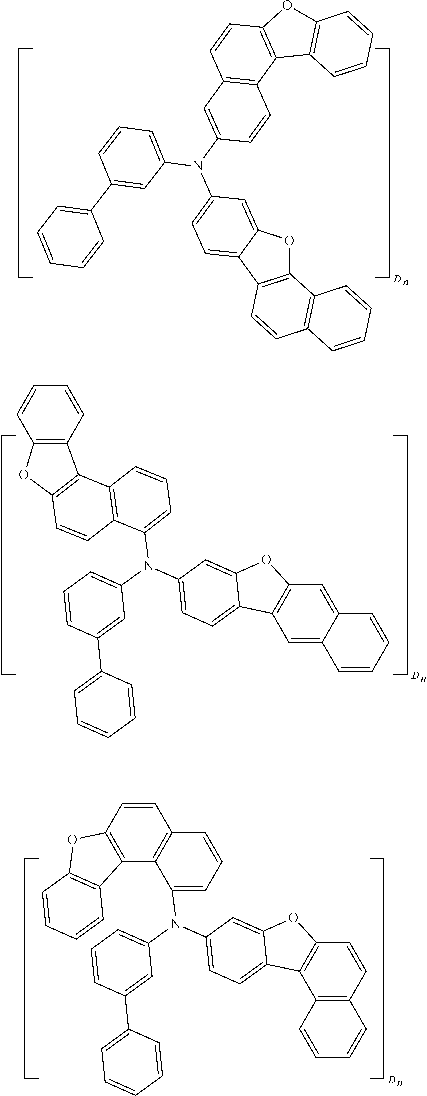

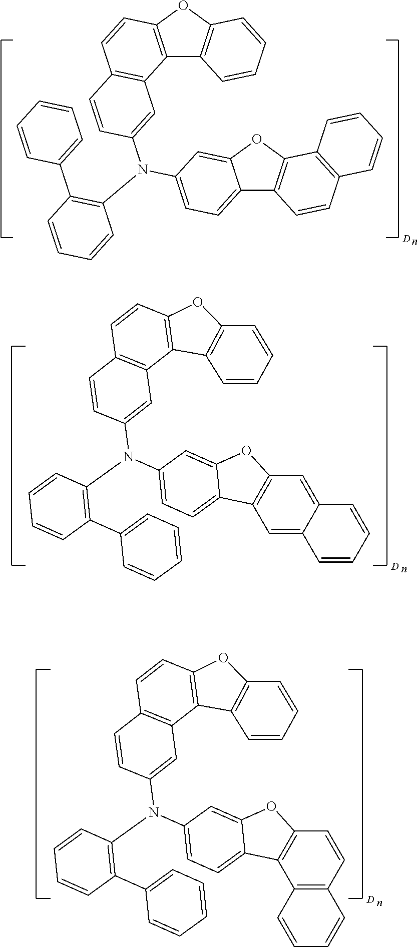

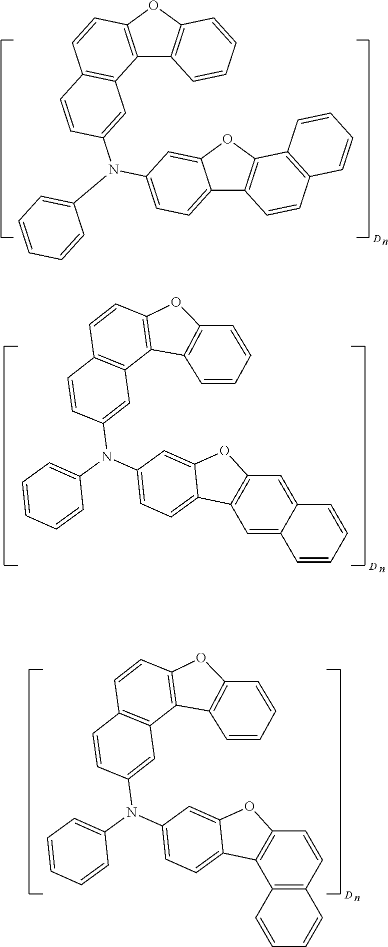

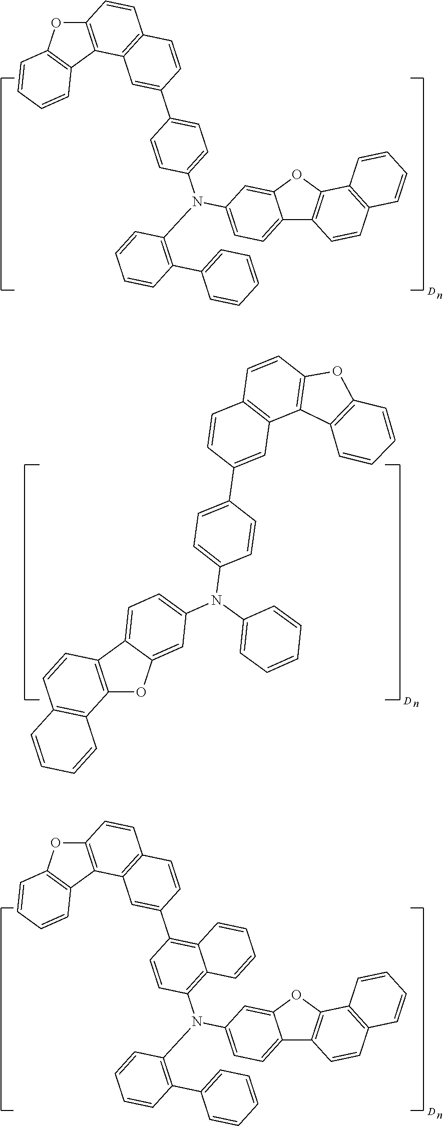

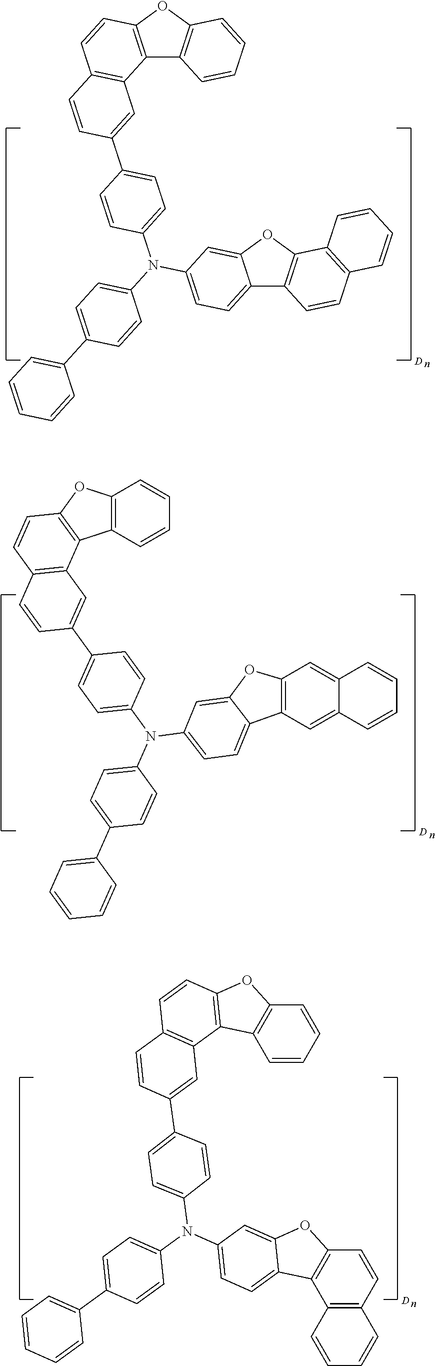

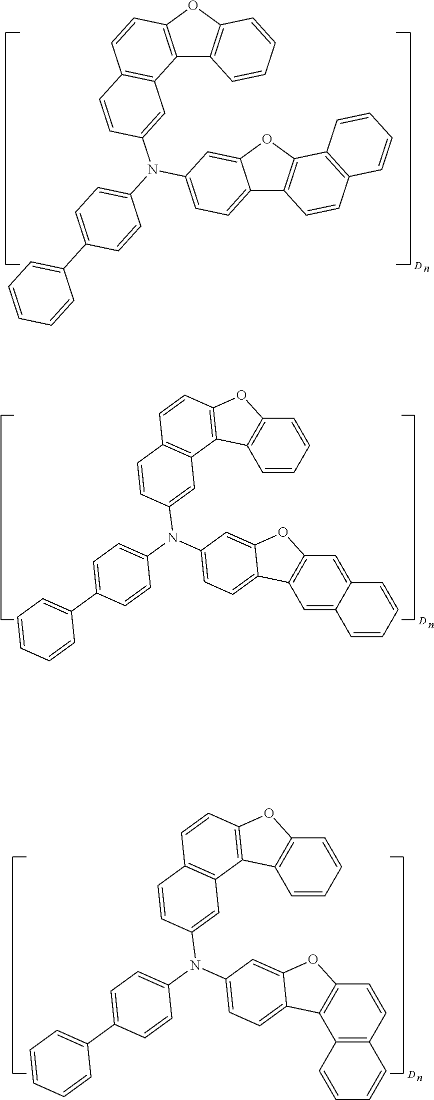

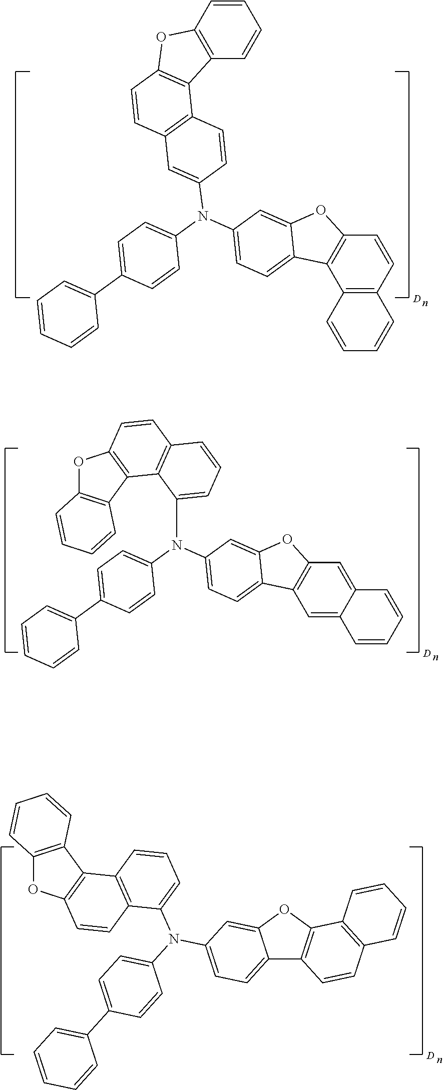

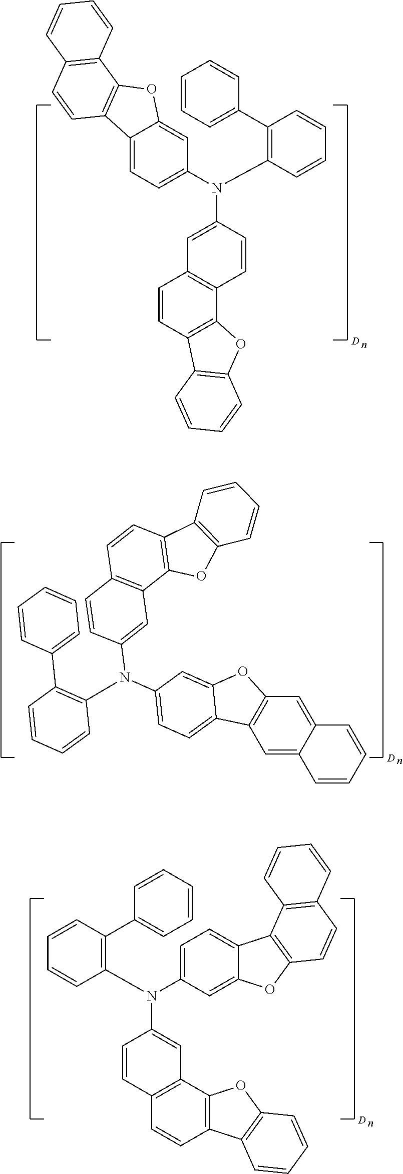

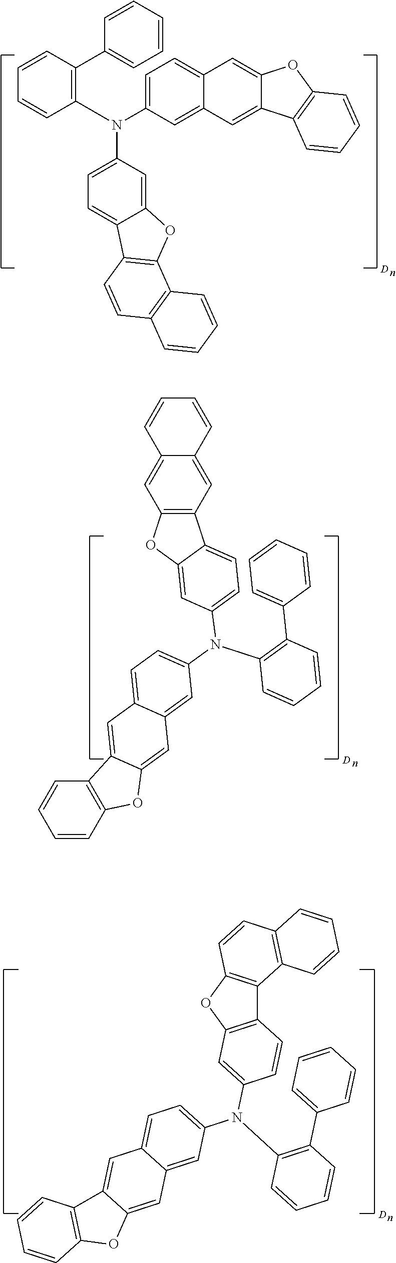

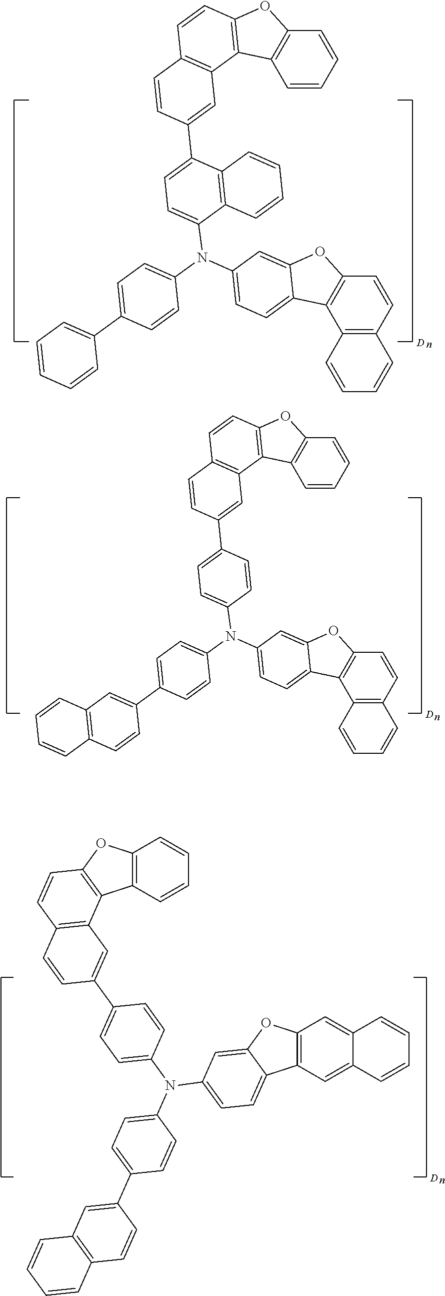

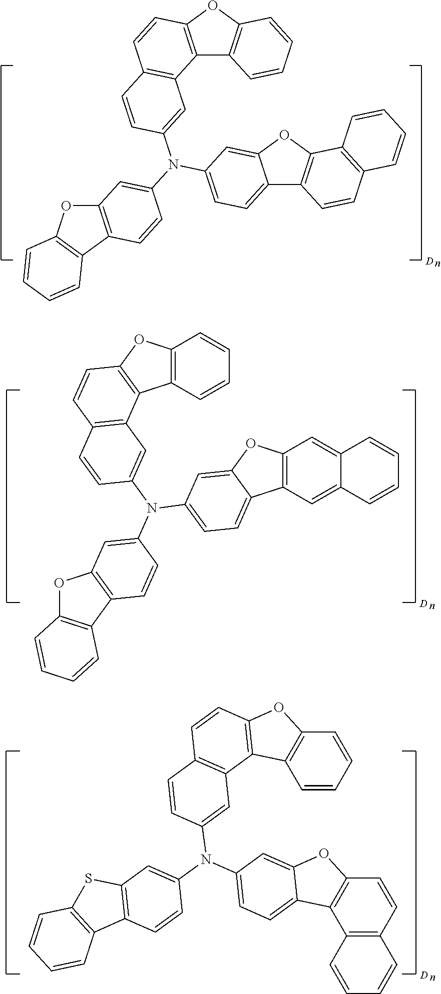

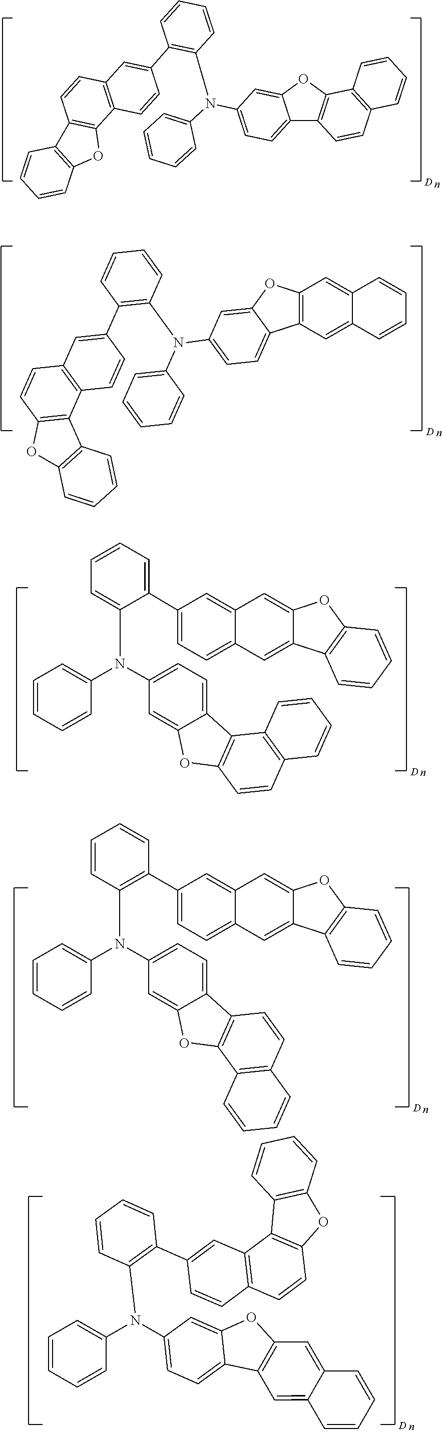

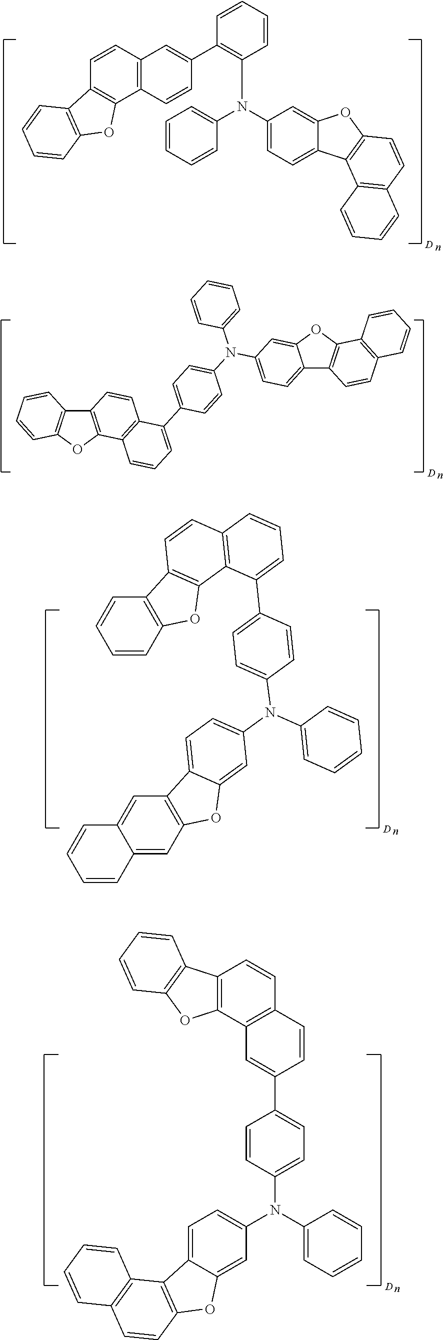

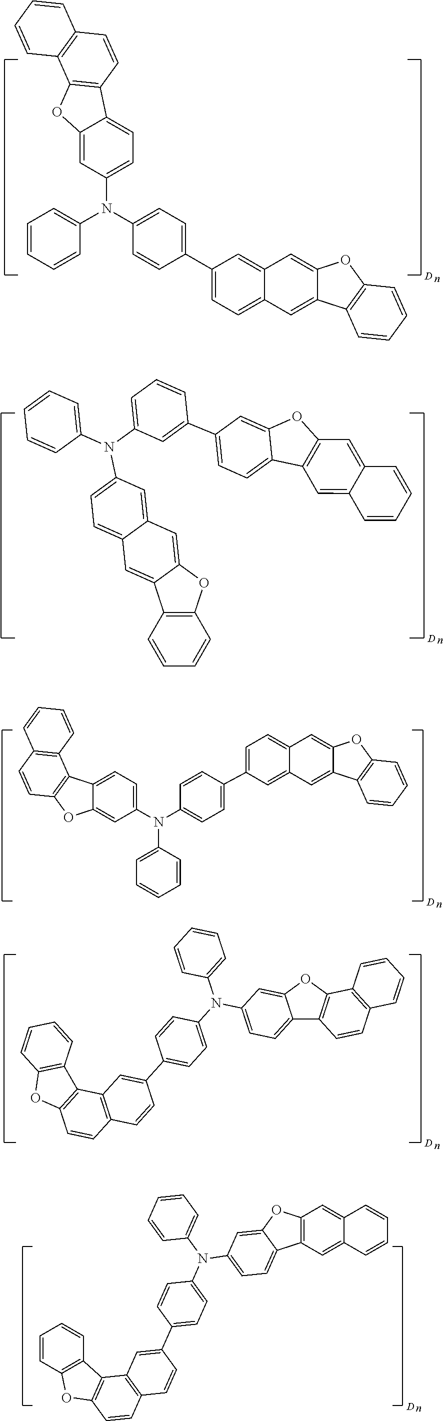

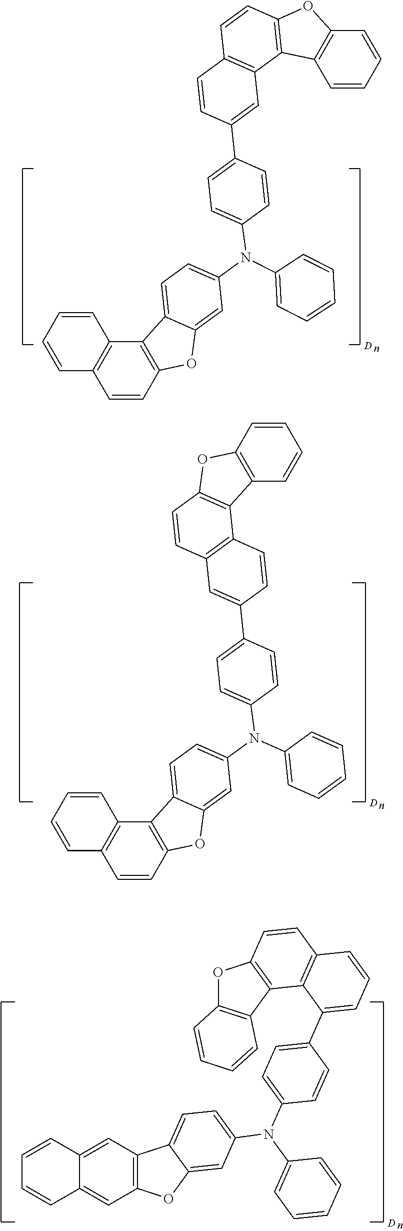

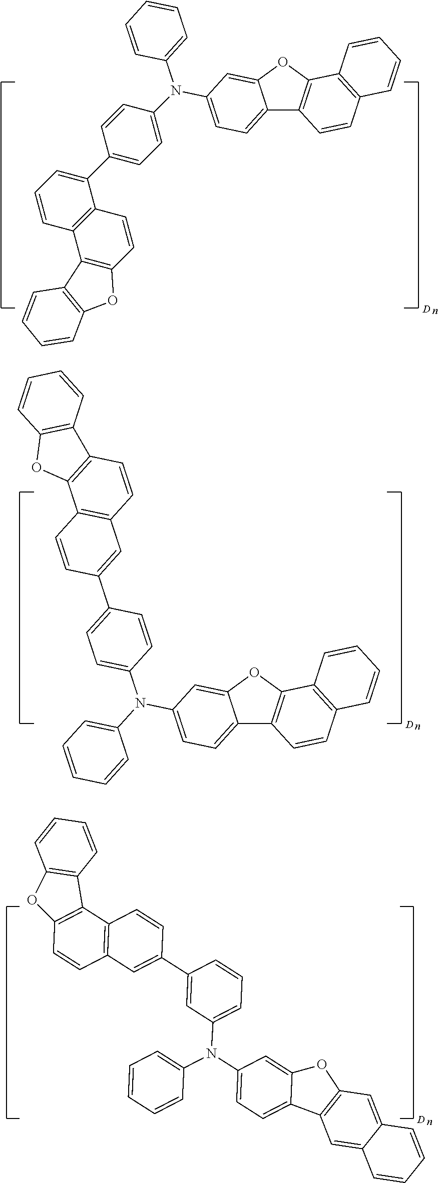

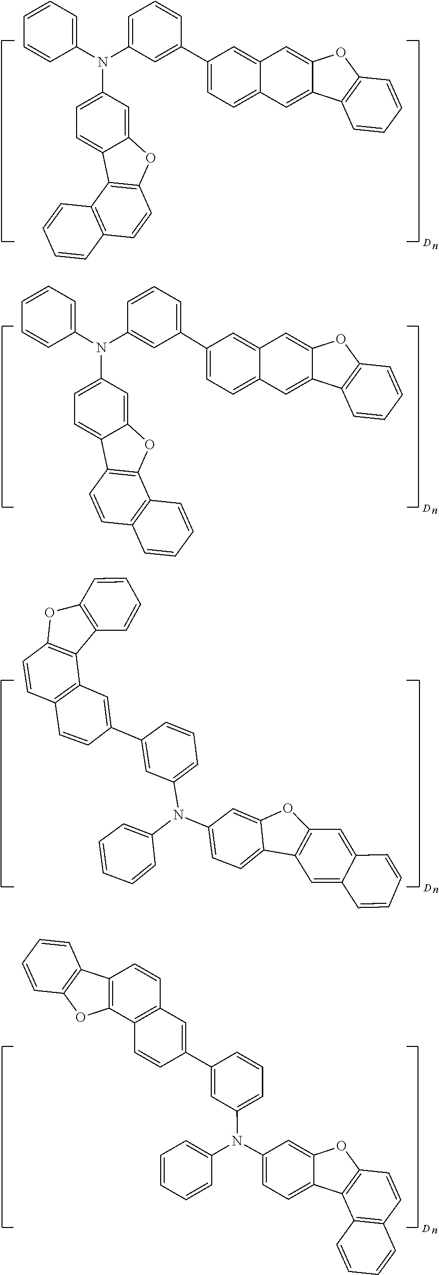

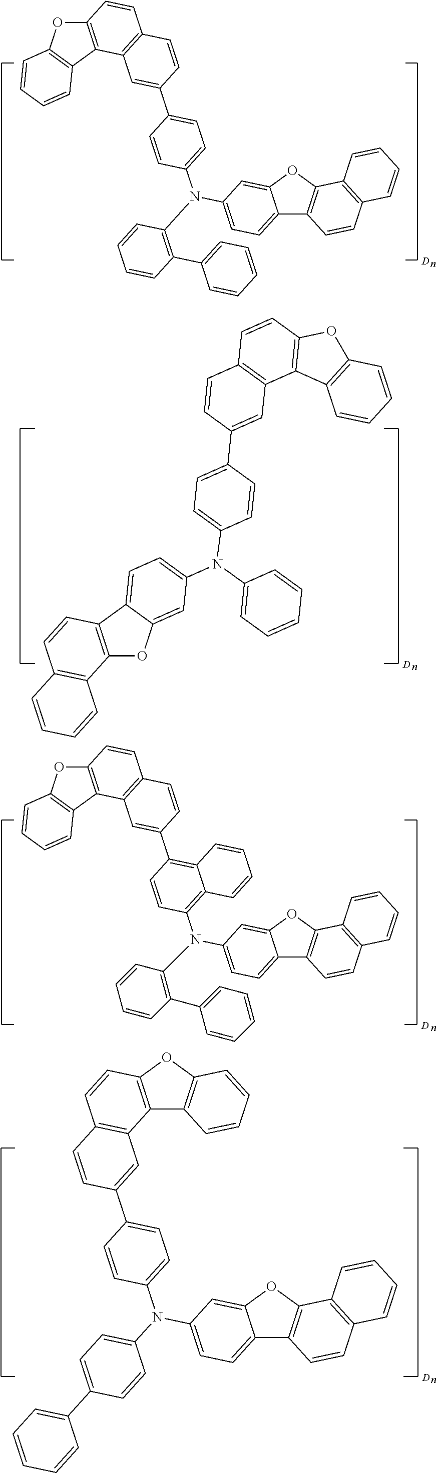

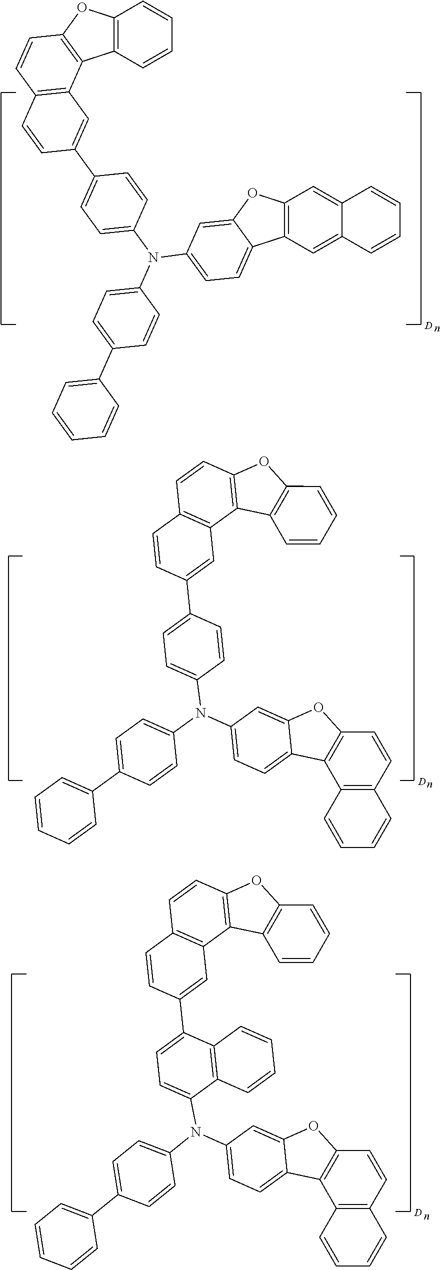

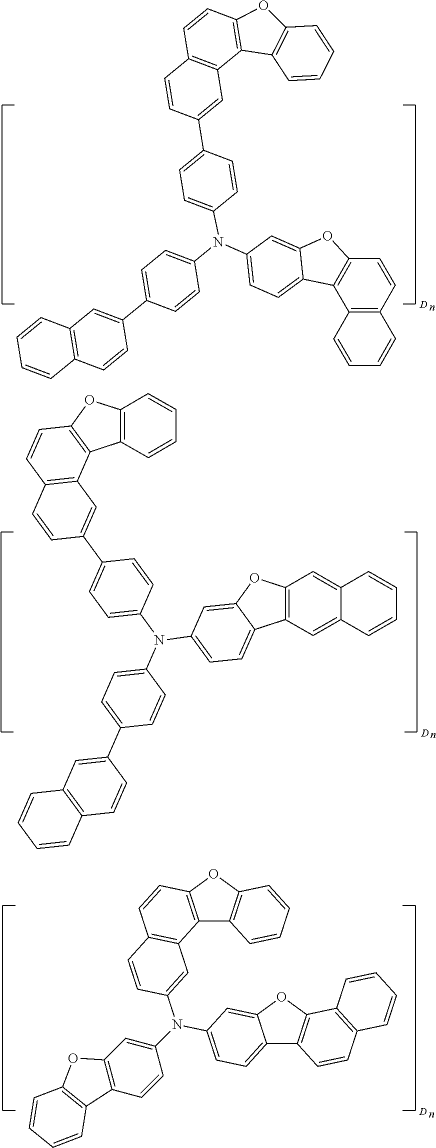

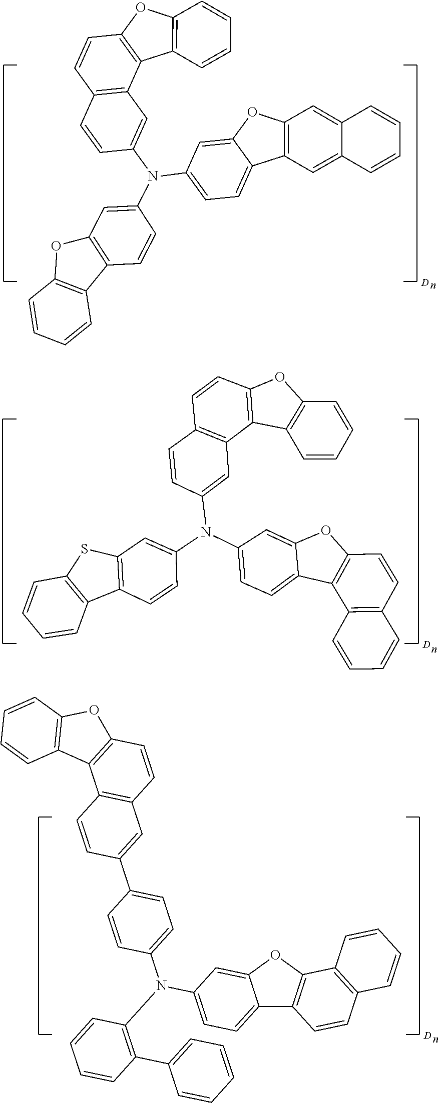

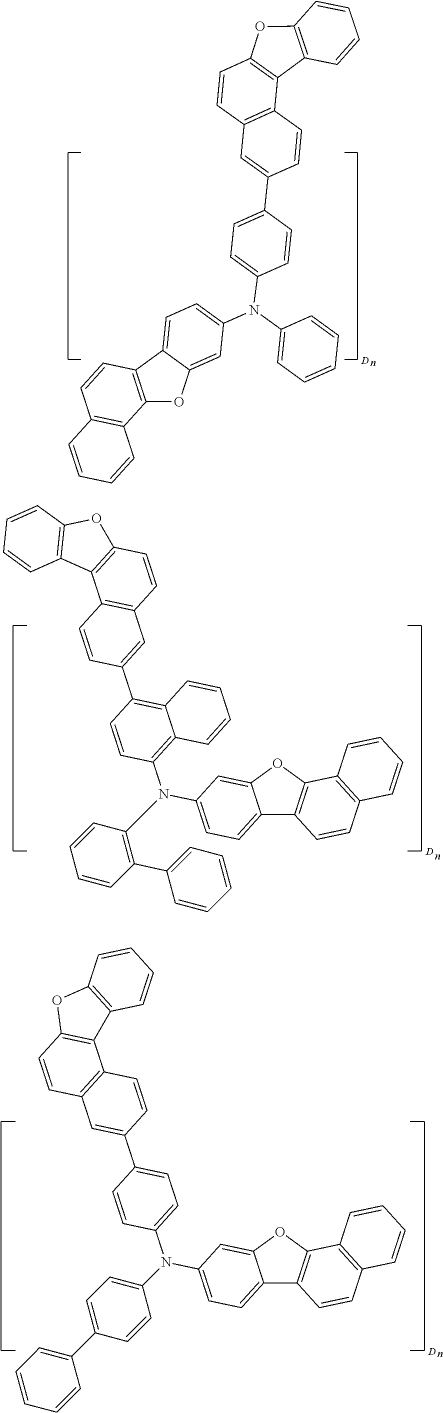

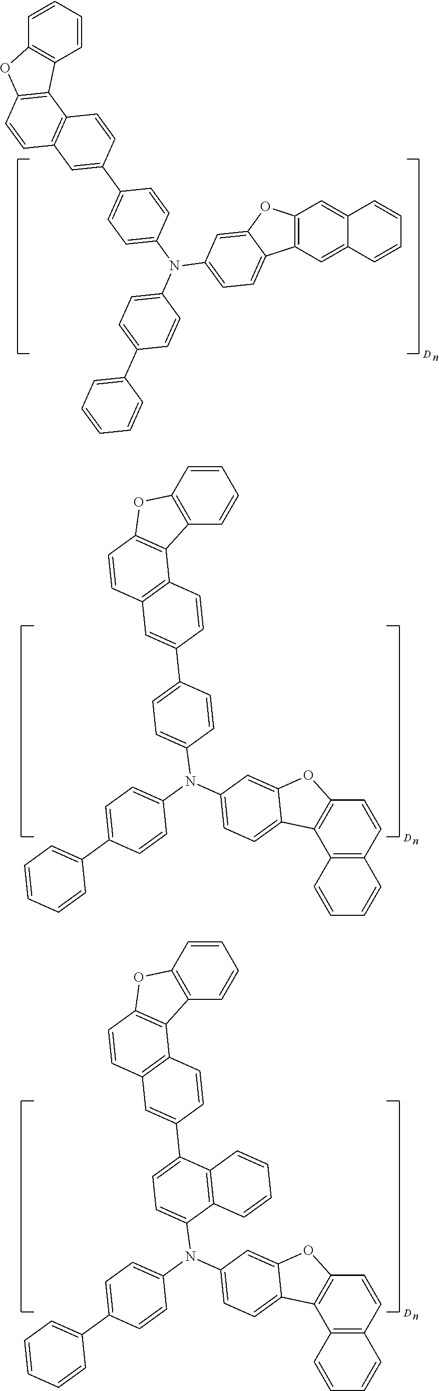

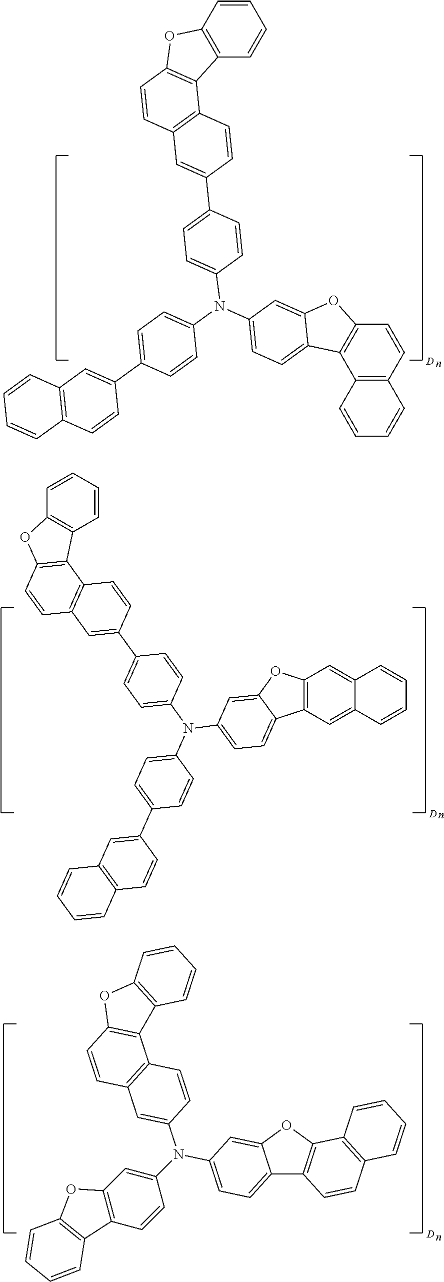

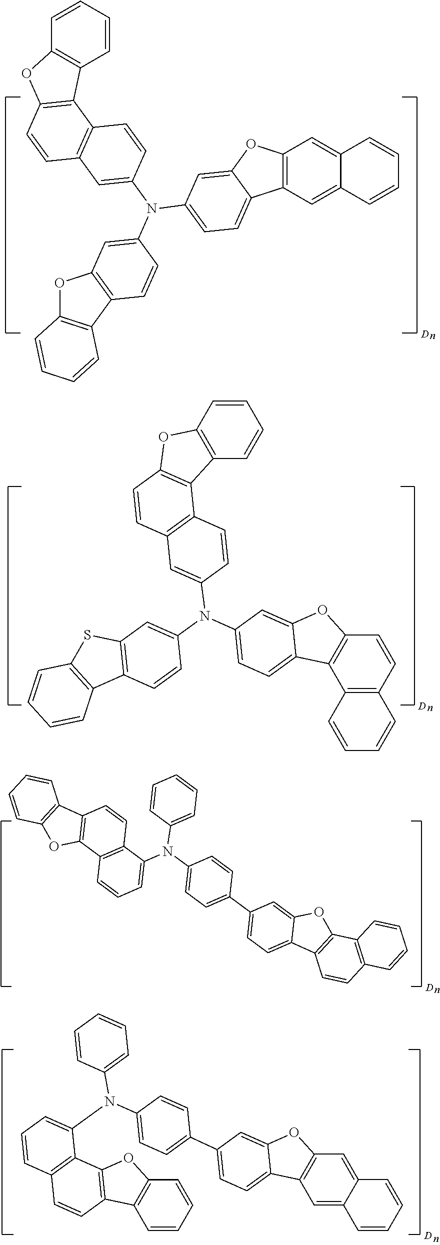

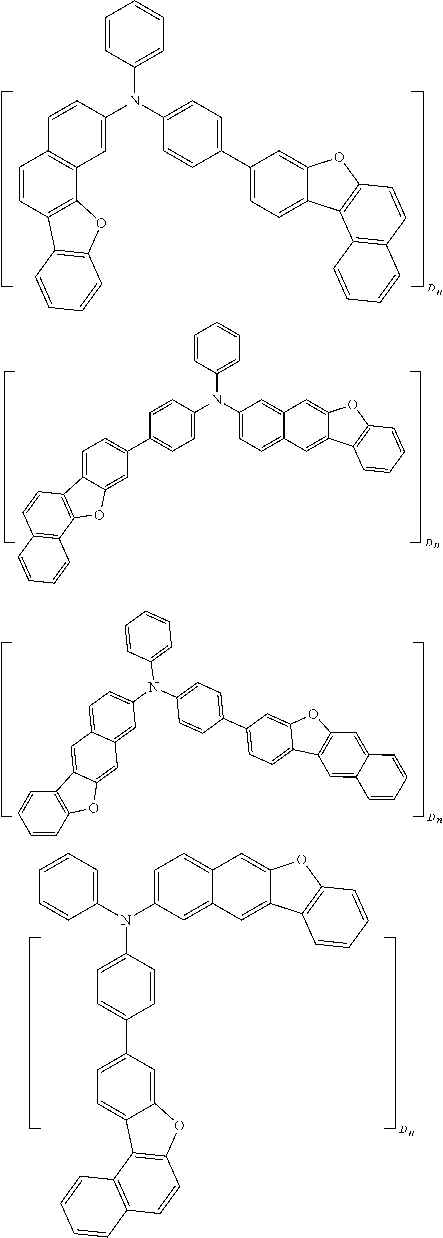

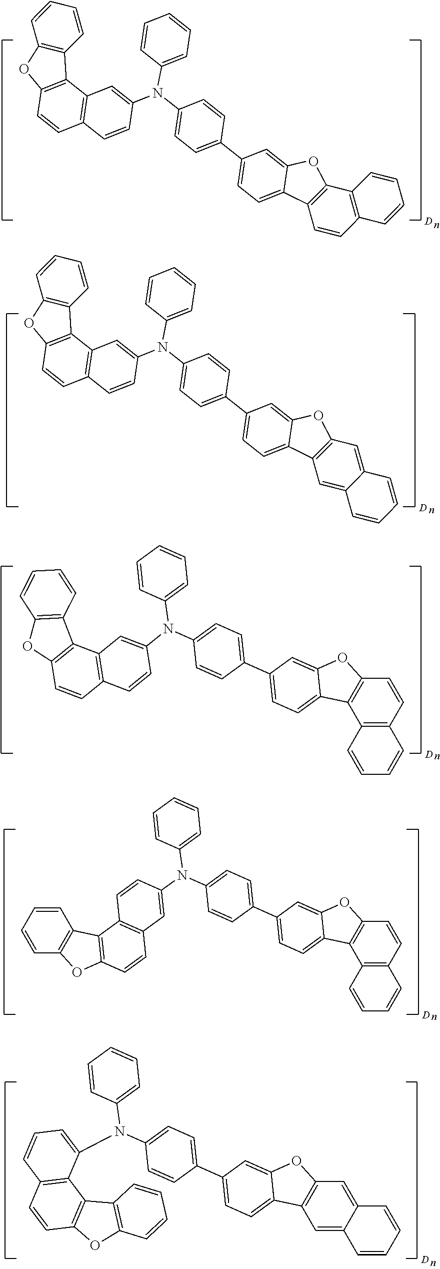

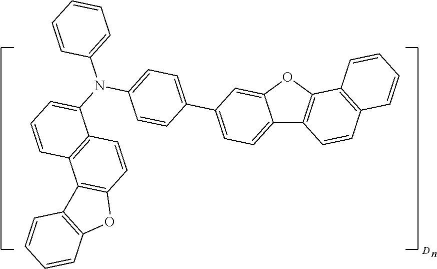

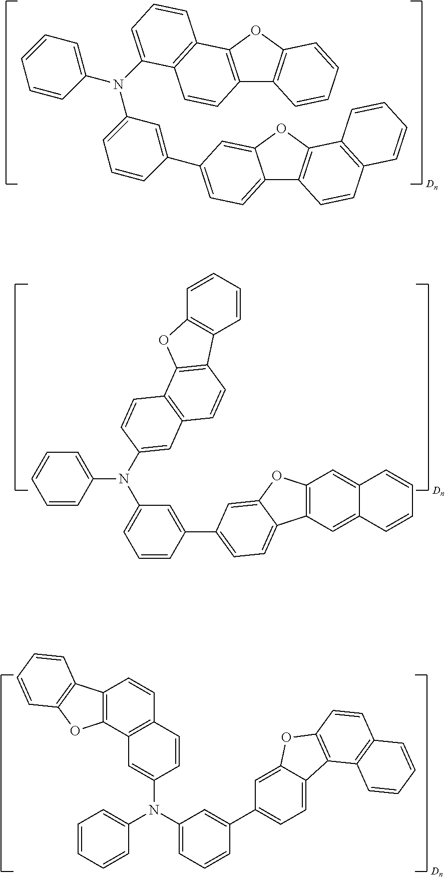

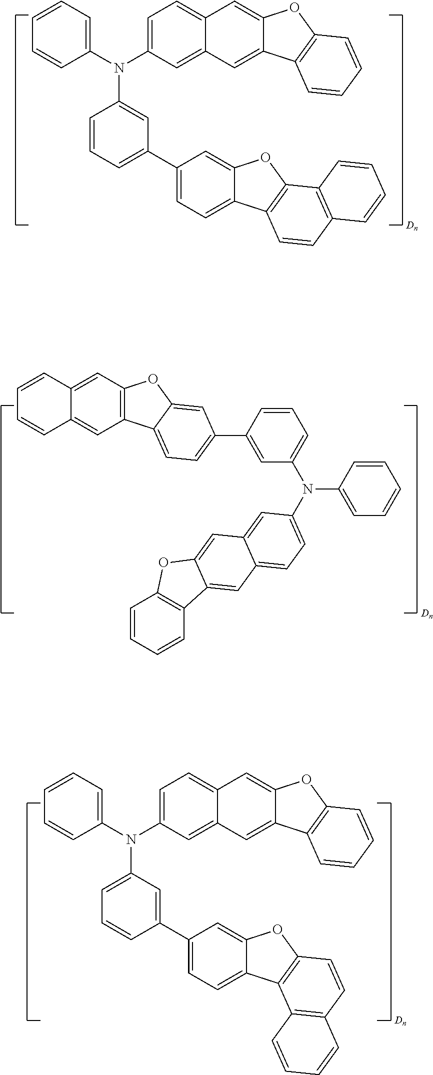

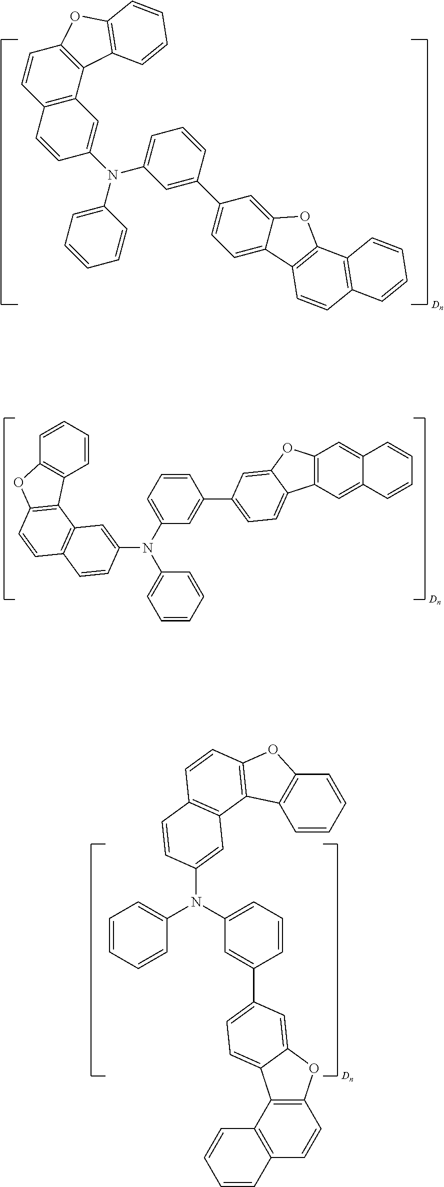

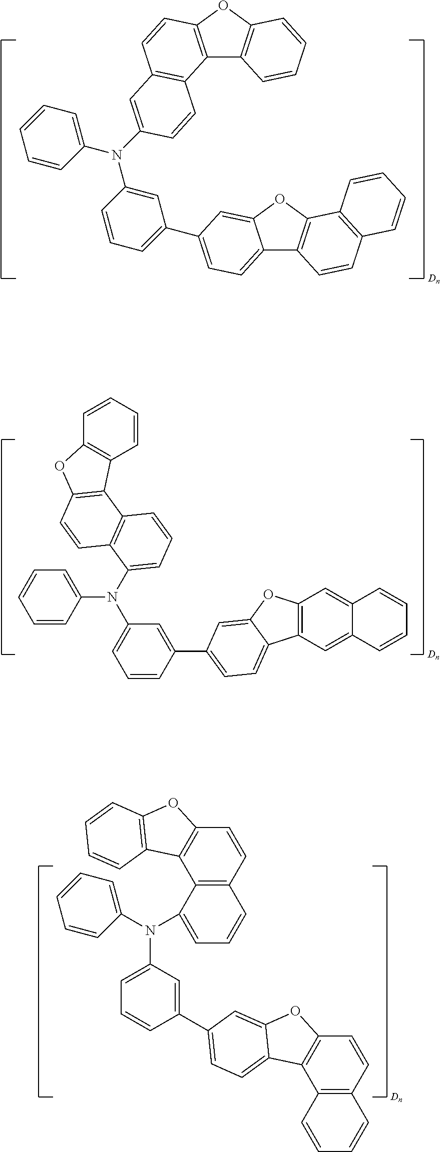

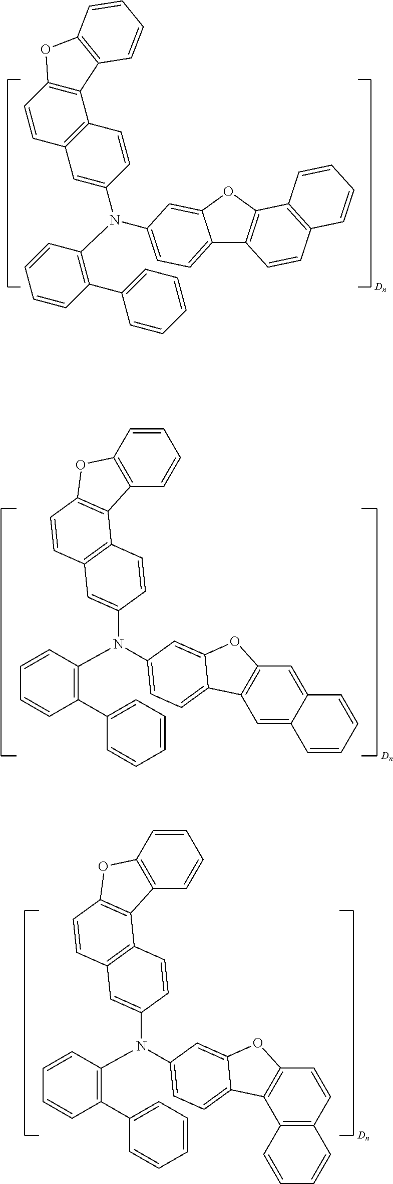

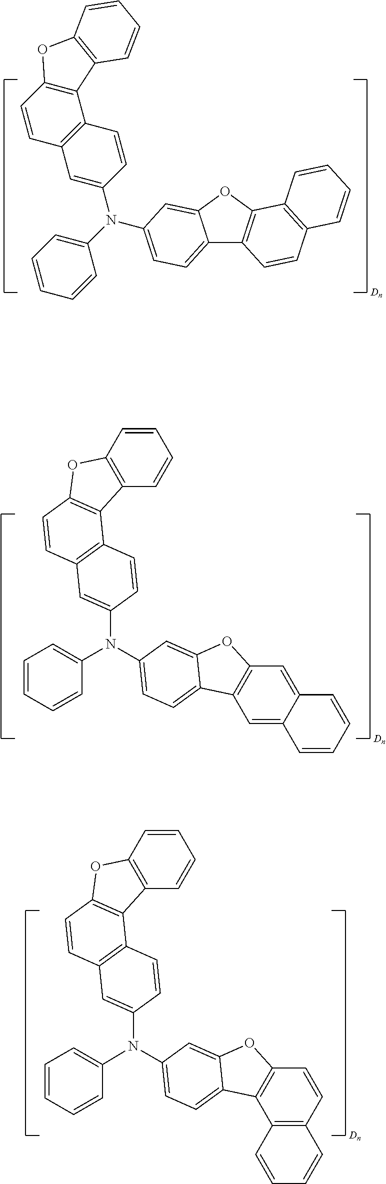

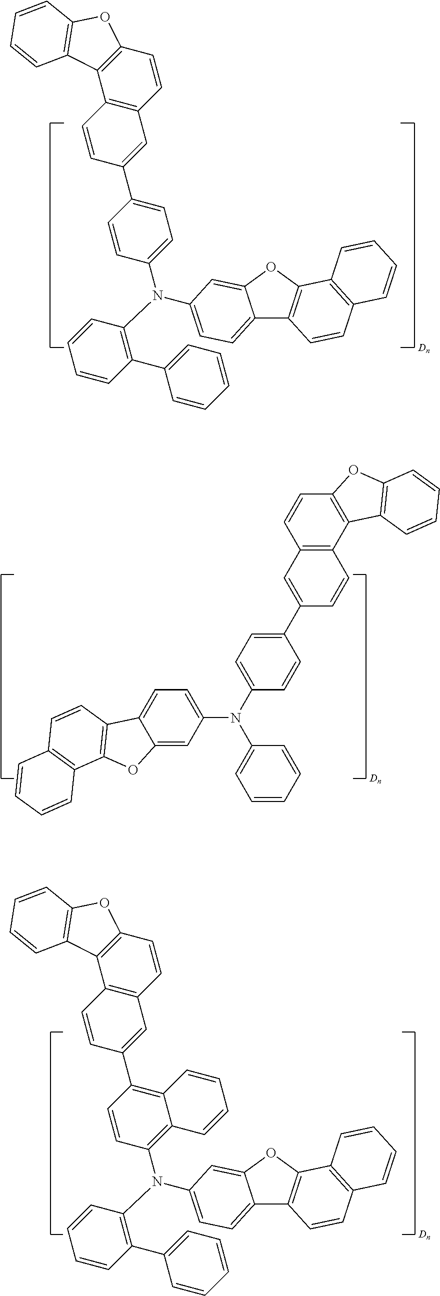

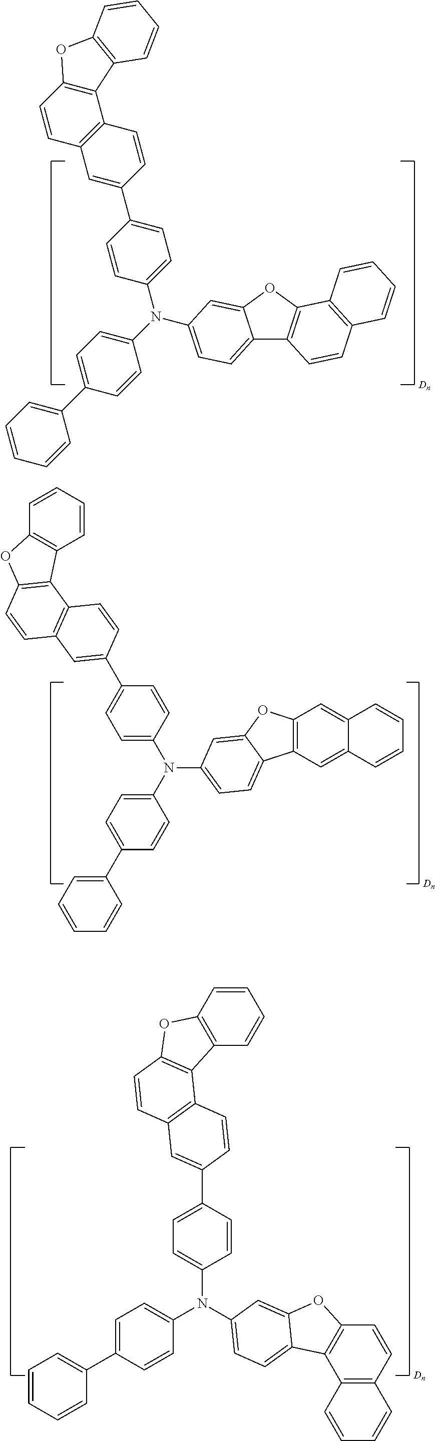

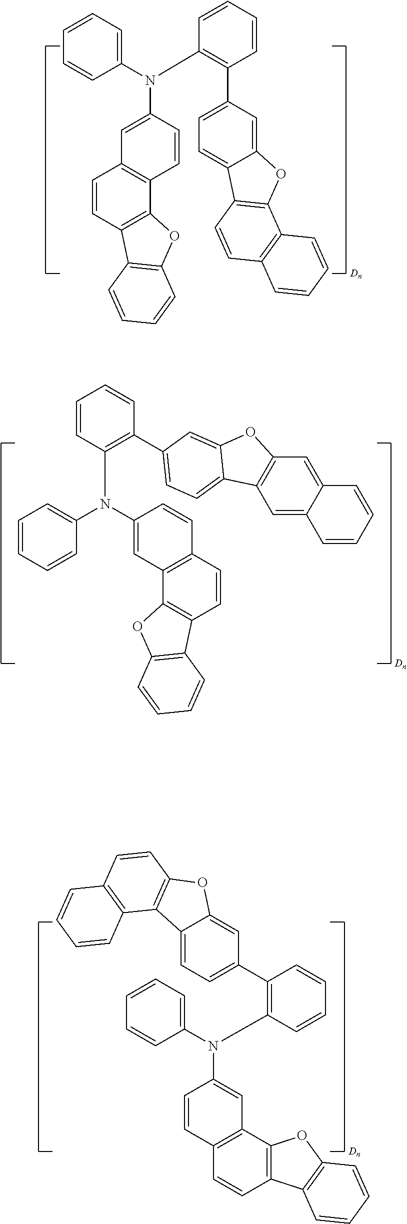

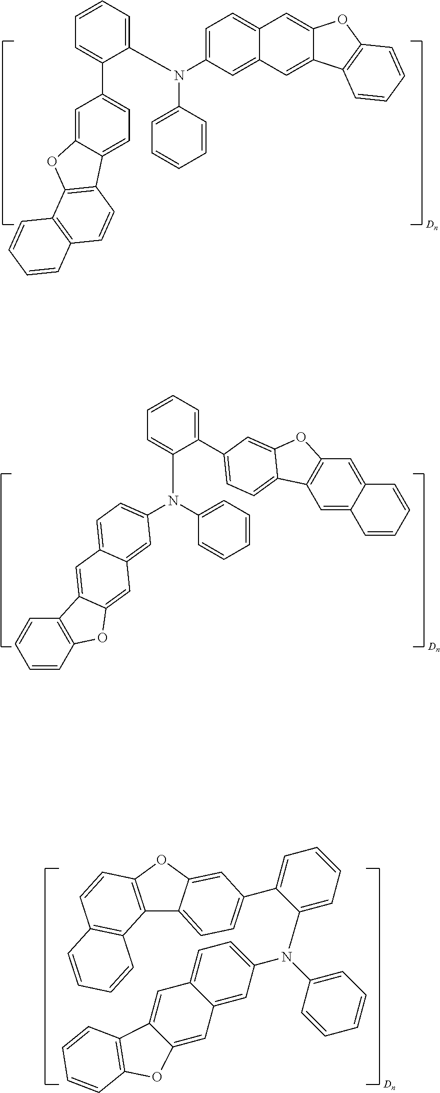

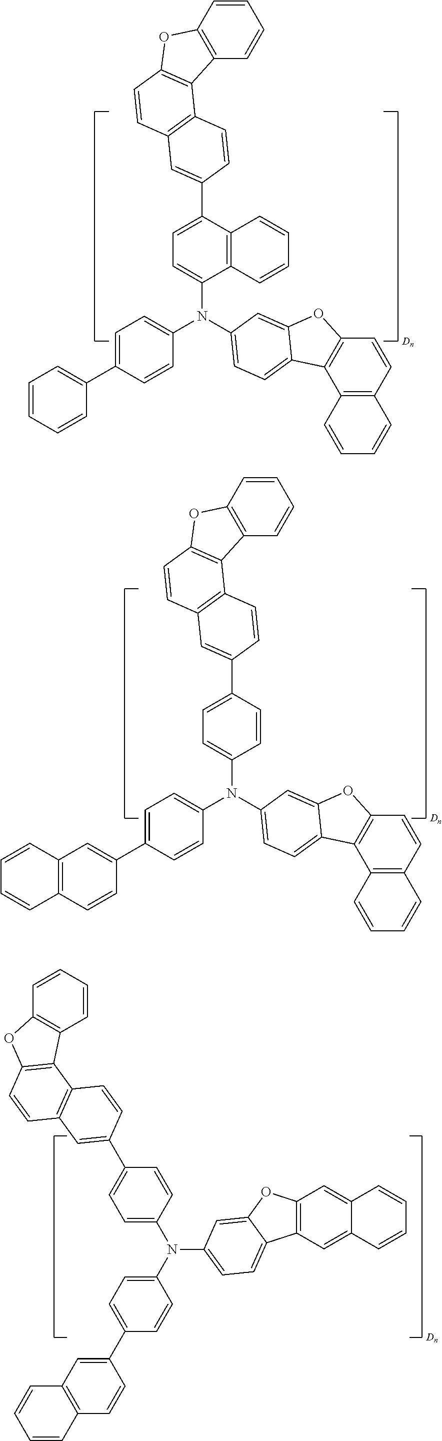

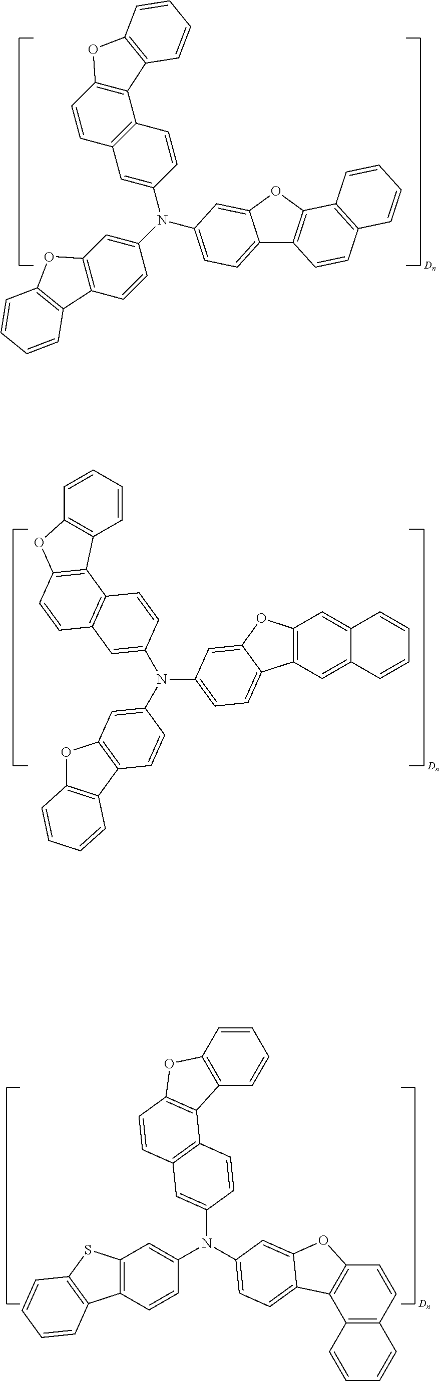

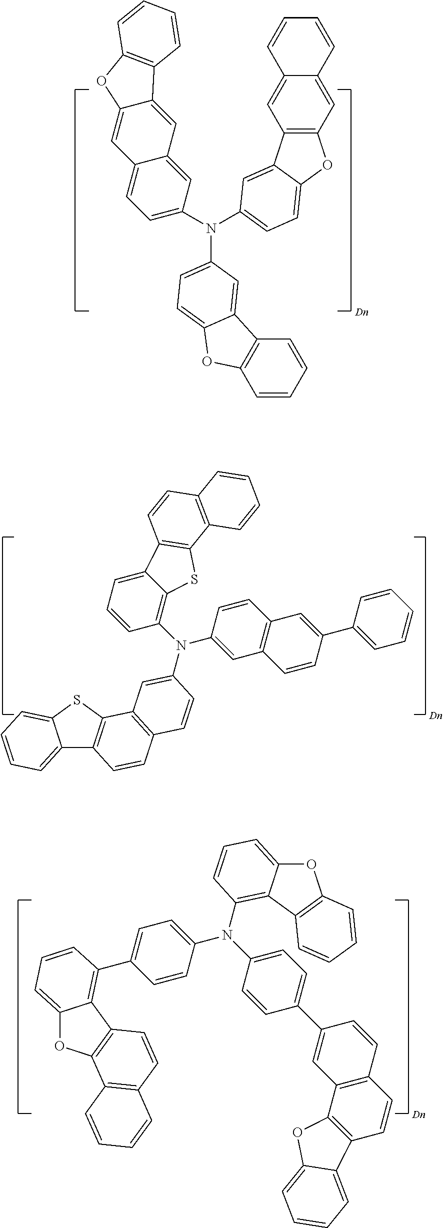

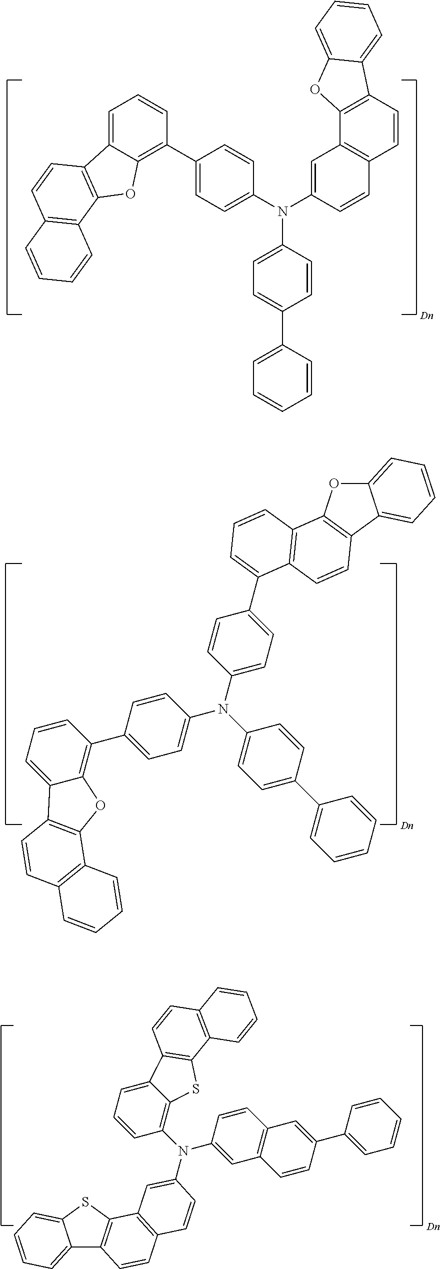

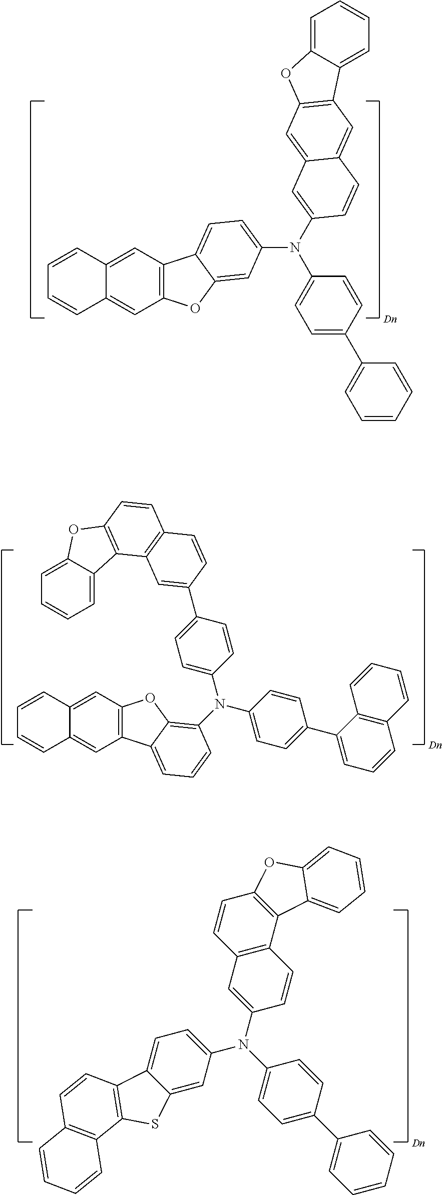

wherein,
Dn means that n hydrogens have been replaced by deuterium,
wherein n is an integer of 1 or more,

Meanwhile, the compound represented by Chemical Formula 1 can be prepared by a preparation method as shown in the following Reaction Scheme 1 as an example:

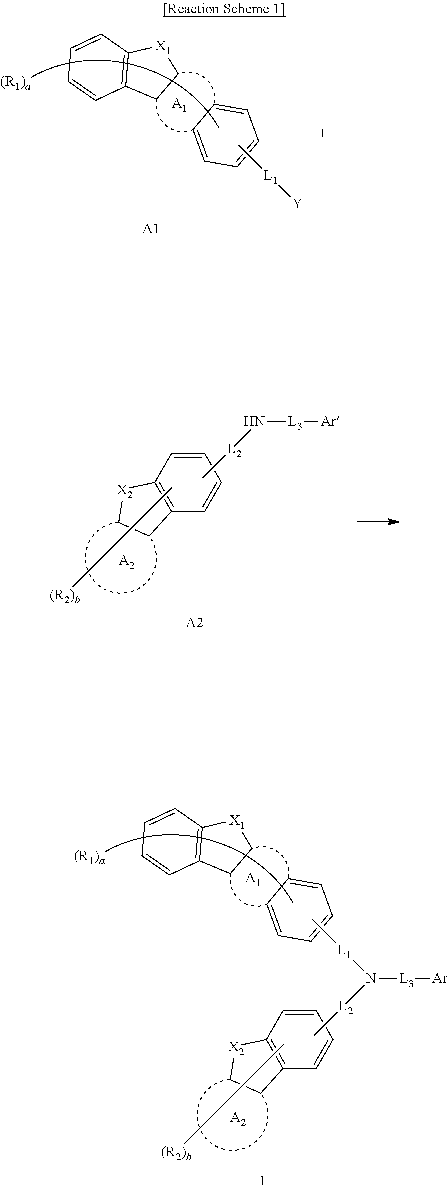

In Reaction Scheme 1, Y is halogen, preferably bromo, or chloro, and the definitions of other substituents are the same as described above.

Specifically, the compound may be prepared by an amine substitution reaction of the starting materials A1 and A2. Such an amine substitution reaction is preferably carried out in the presence of a palladium catalyst and a base, and a reactive group for the amine substitution reaction can be appropriately changed.

### (Organic Light Emitting Device)

Meanwhile, according to the present disclosure, there is provided an organic light emitting device comprising a compound represented by Chemical Formula 1. In one example, the present disclosure provides an organic light emitting device comprising: a first electrode; a second electrode that is provided opposite to the first electrode; and one or more organic material layers that are provided between the first electrode and the second electrode, wherein one or more layers of the organic material layers includes the compound represented by Chemical Formula 1.

Wherein, the organic material layers comprising the compound represented by Chemical Formula 1 may be a hole transport layer, an electron blocking layer, or a light emitting layer.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, a light emitting layer, an electron transport layer, an electron injection layer and the like as the organic material layer. However, the structure of the organic light emitting device is not limited thereto, and it may include a smaller number of organic layers.

In one embodiment, the organic material layer may include a light emitting layer, wherein the organic material layers comprising the compound may be a light emitting layer.

In another embodiment, the organic material layer may include a hole injection layer, a hole transport layer, a light emitting layer, and an electron injection and transport layer, wherein the organic material layer comprising the compound may be a hole transport layer, an electron blocking layer, or a light emitting layer.

In yet another embodiment, the organic material layer may include a hole injection layer, a hole transport layer, an electron blocking layer, a light emitting layer, and an electron injection and transport layer, wherein the organic material layer comprising the compound may be a hole transport layer, an electron blocking layer, or a light emitting layer.

In yet another embodiment, the organic material layer may include a hole injection layer, a hole transport layer, an electron blocking layer, a light emitting layer, a hole blocking layer, and an electron injection and transport layer, wherein the organic material layer comprising the compound may be a hole transport layer, an electron blocking layer, or a light emitting layer.

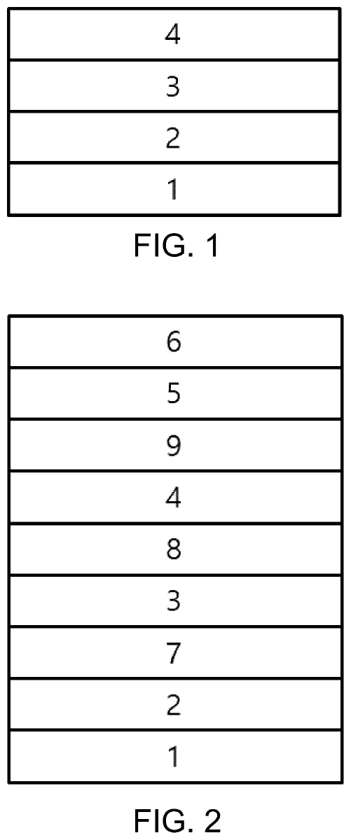

Further, the organic light emitting device according to the present disclosure may be a normal type organic light emitting device in which an anode, one or more organic material layers and a cathode are sequentially stacked on a substrate, wherein the first electrode is an anode, and the second electrode is a cathode. Further, the organic light emitting device according to the present disclosure may be an inverted type organic light emitting device in which a cathode, one or more organic material layers and an anode are sequentially stacked on a substrate, wherein the first electrode is a cathode and the second electrode is an anode. For example, the structure of the organic light emitting device according to one embodiment of the present disclosure is illustrated in FIGS. 1 and 2.

FIG. 1 shows an example of an organic light emitting device comprising a substrate 1, an anode 2, a hole transport layer 3, a light emitting layer 4, an electron injection and transport layer 5, and a cathode 6. In such a structure, the compound represented by Chemical Formula 1 may be included in the hole injection layer, or the light emitting layer.

FIG. 2 shows an example of an organic light emitting device comprising a substrate 1, an anode 2, a hole injection layer 7, a hole transport layer 3, an electron blocking layer 8, a light emitting layer 4, a hole blocking layer 9, an electron injection and transport layer 5, and a cathode 6. In such a structure, the compound represented by Chemical Formula 1 may be included in the hole transport layer, the electron blocking layer, or the light emitting layer.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Further, the hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include the compound represented by Chemical Formula 1, metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. The hole transport material includes the compound represented by Chemical Formula 1, or an arylamine-based organic material, a conductive polymer, a block copolymer in which a conjugate portion and a non-conjugate portion are present together, and the like, but are not limited thereto.

Further, the electron blocking layer (electron suppression layer) refers to a layer which is formed on the hole transport layer, preferably provided in contact with the light emitting layer, and serves to adjust the hole mobility, prevent excessive movement of electrons, and increase the probabilities of hole-electron coupling, thereby improving the efficiency of the organic light emitting device. The electron blocking layer includes an electron blocking material, and examples of such electron blocking material may include the compound represented by Chemical Formula 1 or an arylamine-based organic material, and the like, but is not limited thereto.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq3); a carbazole-based compound; a dimerized styryl compound; BAlq; a 10-hydroxybenzoquinoline-metal compound; a benzoxazole, benzthiazole and benzimidazole-based compound; a poly(p-phenylenevinylene) (PPV)-based polymer; a spiro compound; polyfluorene, lubrene, and the like, but are not limited thereto.

Further, the light emitting layer may include a host material and a dopant material. The compound represented by Chemical Formula 1 can be used as such a host material. Further, the host material may further include a fused aromatic ring derivative, a heterocycle-containing compound or the like in addition to the compound represented by Chemical Formula 1. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The hole blocking layer refers to a layer which is formed on the light emitting layer, and preferably, is provided in contact with the light emitting layer, and thus severs to control electron mobility, to prevent excessive movement of holes, and to increase the probabilities of hole-electron bonding, thereby improving the efficiency of the organic light emitting device. The hole blocking layer includes a hole blocking material, and as an example of such hole blocking material, a compound into which an electron-withdrawing group is introduced, such as azine derivatives including triazine; triazole derivatives; oxadiazole derivatives; phenanthroline derivatives; phosphine oxide derivatives can be used, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline; a complex including Alq3; an organic radical compound; a hydroxyflavone-metal complex, a triazine derivative, and the like, but are not limited thereto. Alternatively, it may be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer may also be formed as a separate layer such as an electron injection layer and an electron transport layer. In such a case, the electron transport layer is formed on the light emitting layer or the hole blocking layer, and the above-mentioned electron injection and transport material may be used as the electron transport material included in the electron transport layer. In addition, the electron injection layer is formed on the electron transport layer, and examples of the electron injection material included in the electron injection layer include LiF, NaCl, CsF, Li₂O, BaO, fluorenone, anthraquinodimethane, diphenoquinone, thiopyran dioxide, oxazole, oxadiazole, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

The metal complex compound includes 8-hydroxyquinolinato lithium, bis(8-hydroxyquinolinato) zinc, bis(8-hydroxyquinolinato) copper, bis(8-hydroxyquinolinato) manganese, tris(8-hydroxyquinolinato)aluminum, tris(2-methyl-8-hydroxyquinolinato)aluminum, tris(8-hydroxyquinolinato) gallium, bis(10-hydroxybenzo[h]quinolinato) beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but is not limited thereto.

The organic light emitting device according to the present disclosure may be a bottom emission type device, a top emission type device, or a double side emission type device, and in particular, it may be a bottom emission type light emitting device that requires relatively high luminous efficiency.

In addition, the compound represented by Chemical Formula 1 may be included in an organic solar cell or an organic transistor in addition to an organic light emitting device.

### Synthesis Example

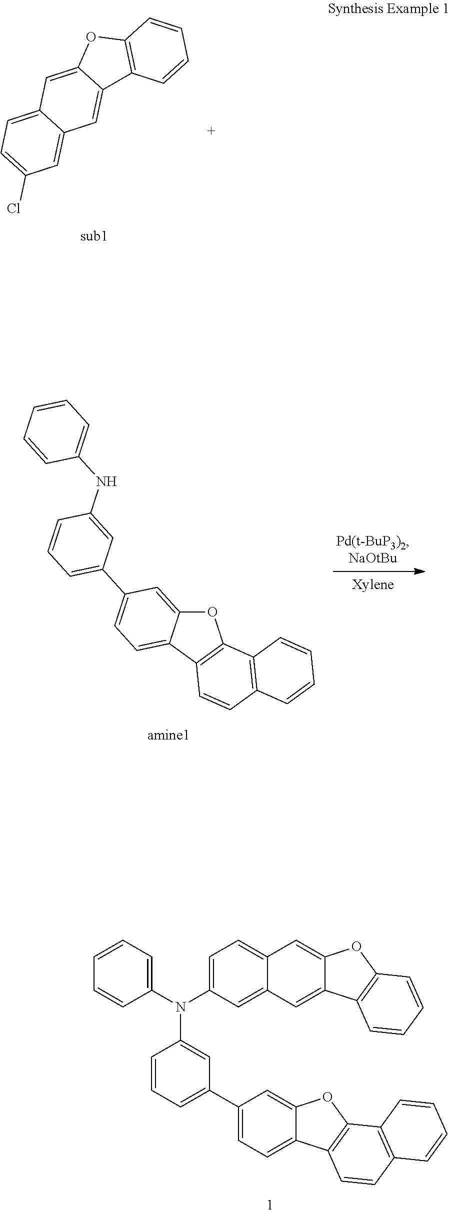 [SMILES: Clc1ccc2cc3oc4ccccc4c3cc2c1 ; c1ccc(Nc2cccc(-c3ccc4c(c3)oc3c5ccccc5ccc43)c2)cc1 ; c1ccc(N(c2cccc(-c3ccc4c(c3)oc3c5ccccc5ccc43)c2)c2ccc3cc4oc5ccccc5c4cc3c2)cc1]

sub1 (15 g, 59.4 mmol), amine1 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 24.6 g of Compound 1 (yield: 69%, MS: [M+H]⁺=603).

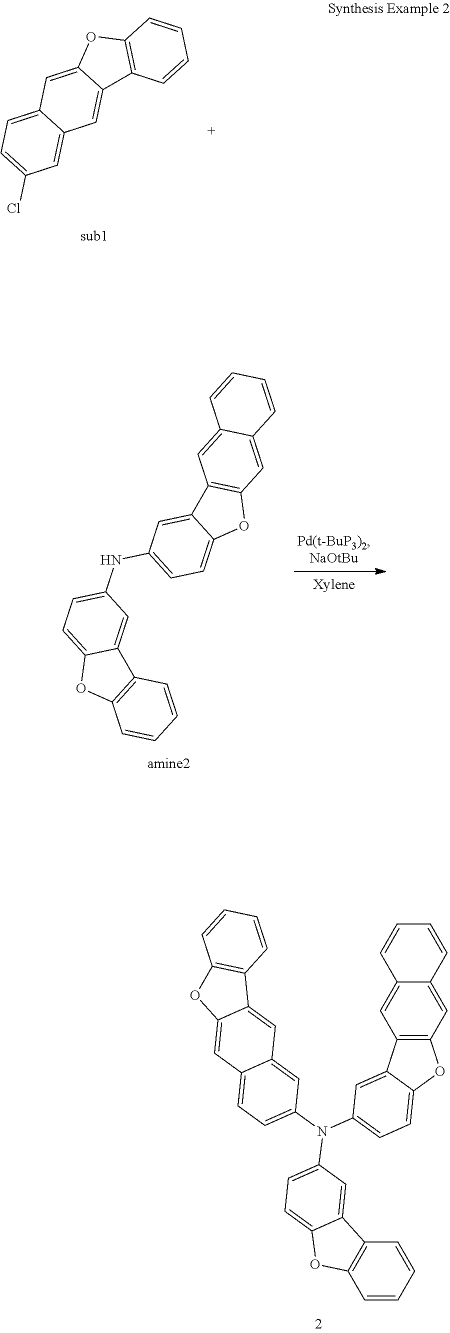 [SMILES: Clc1ccc2cc3oc4ccccc4c3cc2c1 ; c1ccc2cc3c(cc2c1)oc1ccc(Nc2ccc4oc5ccccc5c4c2)cc13 ; c1ccc2cc3c(cc2c1)oc1ccc(N(c2ccc4cc5oc6ccccc6c5cc4c2)c2ccc4oc5ccccc5c4c2)cc13]

sub1 (15 g, 59.4 mmol), amine2 (24.9 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 24.9 g of Compound 2 (yield: 68%, MS: [M+H]⁺=617).

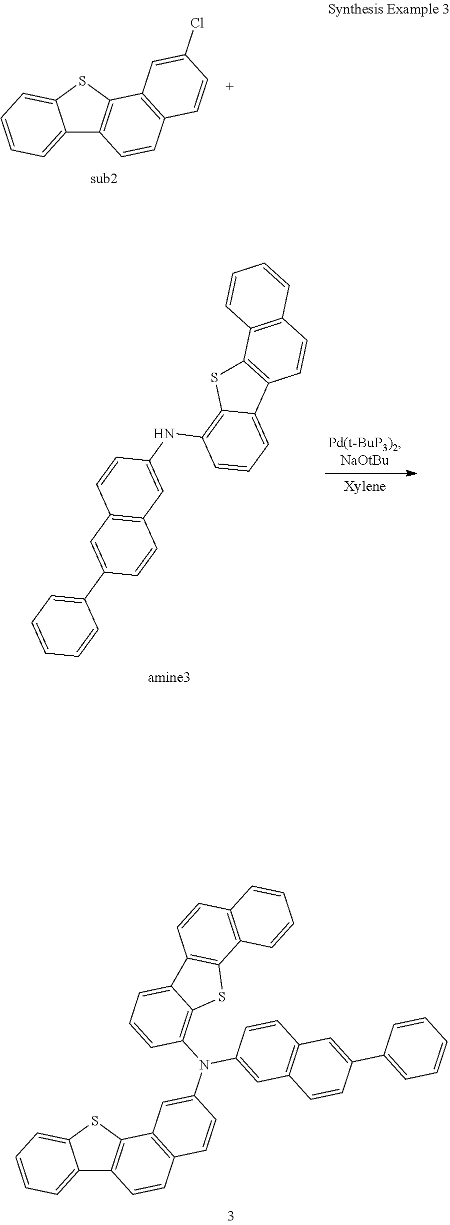 [SMILES: Clc1ccc2ccc3c4ccccc4sc3c2c1 ; c1ccc(-c2ccc3cc(Nc4cccc5c4sc4c6ccccc6ccc54)ccc3c2)cc1 ; c1ccc(-c2ccc3cc(N(c4ccc5ccc6c7ccccc7sc6c5c4)c4cccc5c4sc4c6ccccc6ccc54)ccc3c2)cc1]

sub2 (15 g, 55.8 mmol), amine3 (26.5 g, 58.6 mmol), and sodium tert-butoxide (8 g, 83.7 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 21.8 g of Compound 3 (yield: 57%, MS: [M+H]⁺=685).

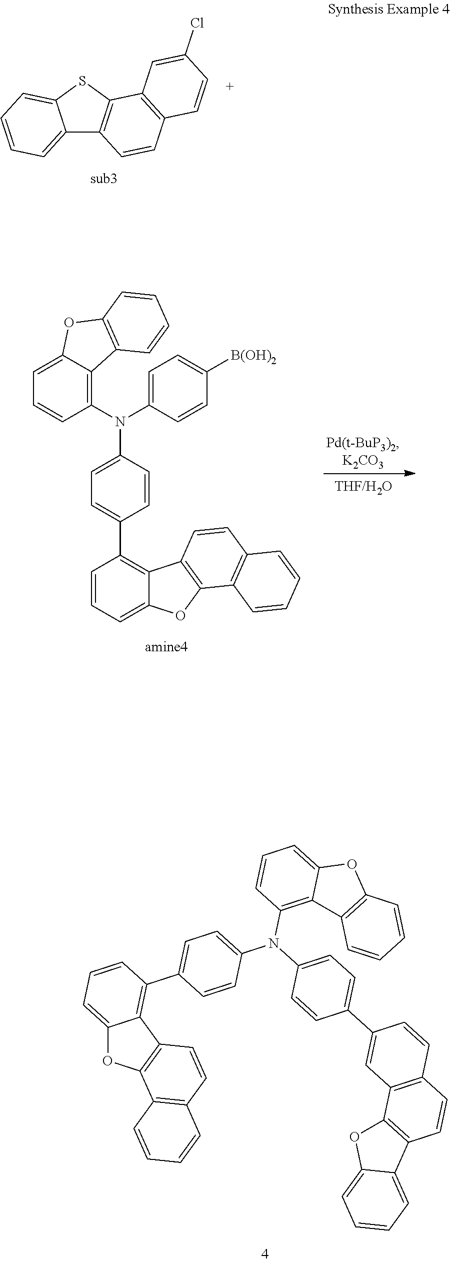 [SMILES: Clc1ccc2ccc3c4ccccc4sc3c2c1 ; OB(O)c1ccc(N(c2ccc(-c3cccc4oc5c6ccccc6ccc5c34)cc2)c2cccc3oc4ccccc4c23)cc1 ; c1ccc2c(c1)ccc1c2oc2cccc(-c3ccc(N(c4ccc(-c5ccc6ccc7c8ccccc8oc7c6c5)cc4)c4cccc5oc6ccccc6c45)cc3)c21]

Sub3 (15 g, 59.4 mmol) and amine4 (37.1 g, 62.3 mmol) were added to 300 ml of THF, and the mixture was stirred and refluxed. Then, potassium carbonate (24.6 g, 178.1 mmol) was dissolved in 100 ml of water, added thereto, sufficiently stirred, and then bis(tri-tert-butylphosphine)palladium(0) (0.3 g, 0.6 mmol) was added. After the reaction for 5 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 32.8 g of Compound 4 (yield: 72%, MS: [M+H]⁺=769).

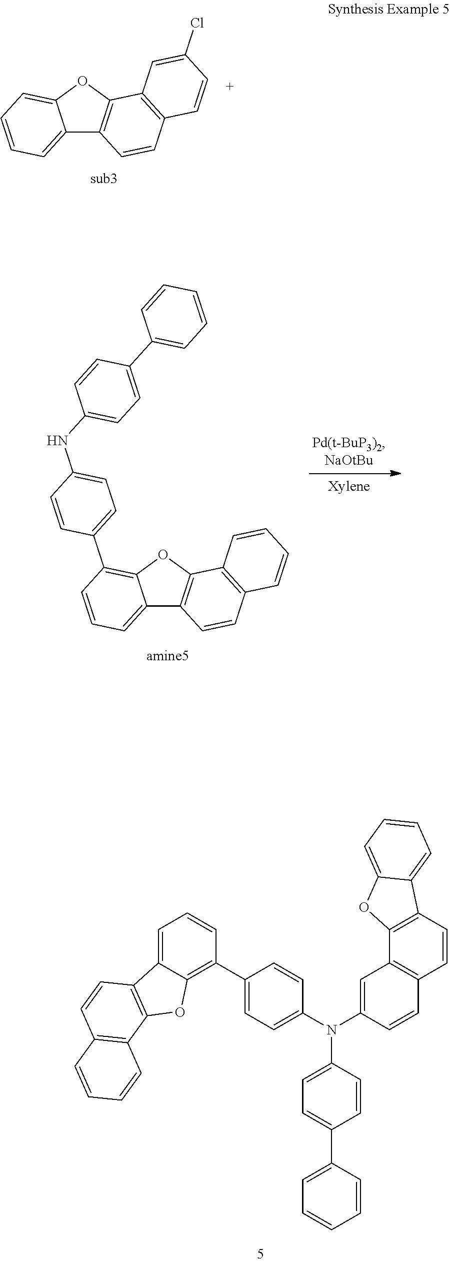 [SMILES: Clc1ccc2ccc3c4ccccc4oc3c2c1 ; c1ccc(-c2ccc(Nc3ccc(-c4cccc5c4oc4c6ccccc6ccc54)cc3)cc2)cc1 ; c1ccc(-c2ccc(N(c3ccc(-c4cccc5c4oc4c6ccccc6ccc54)cc3)c3ccc4ccc5c6ccccc6oc5c4c3)cc2)cc1]

sub3 (15 g, 59.4 mmol), amine5 (28.8 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 25.3 g of Compound 5 (yield: 63%, MS: [M+H]⁺=679).

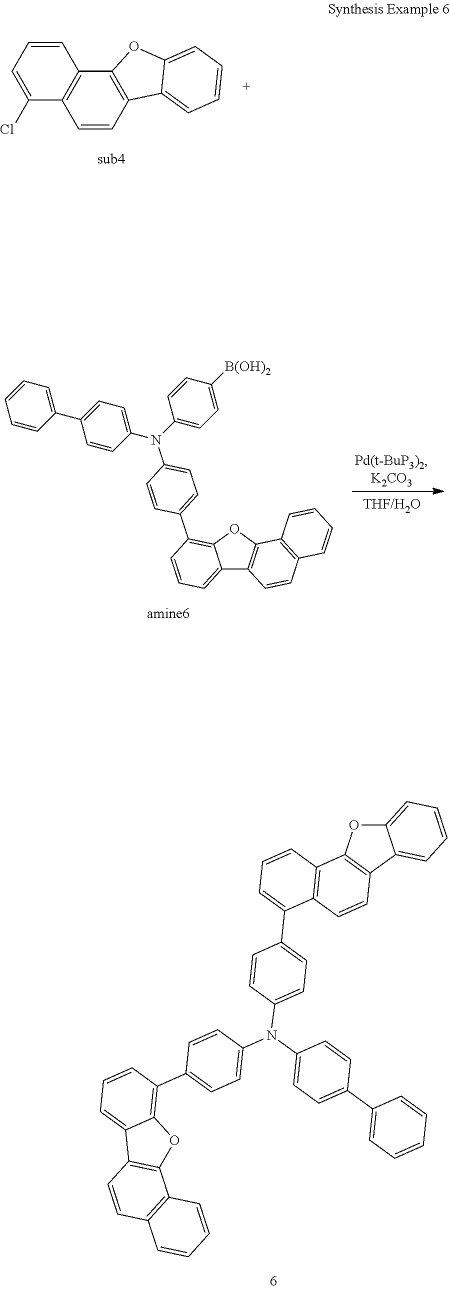 [SMILES: Clc1cccc2c1ccc1c3ccccc3oc21 ; OB(O)c1ccc(N(c2ccc(-c3ccccc3)cc2)c2ccc(-c3cccc4c3oc3c5ccccc5ccc43)cc2)cc1 ; c1ccc(-c2ccc(N(c3ccc(-c4cccc5c4ccc4c6ccccc6oc54)cc3)c3ccc(-c4cccc5c4oc4c6ccccc6ccc54)cc3)cc2)cc1]

Sub4 (15 g, 59.4 mmol) and amine6 (36.2 g, 62.3 mmol) were added to 300 ml of THF, and the mixture was stirred and refluxed. Then, potassium carbonate (24.6 g, 178.1 mmol) was dissolved in 100 ml of water, added thereto, sufficiently stirred, and then bis(tri-tert-butylphosphine)palladium(0) (0.3 g, 0.6 mmol) was added. After the reaction for 4 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 31.8 g of Compound 6 (yield: 71%, MS: [M+H]⁺=755).

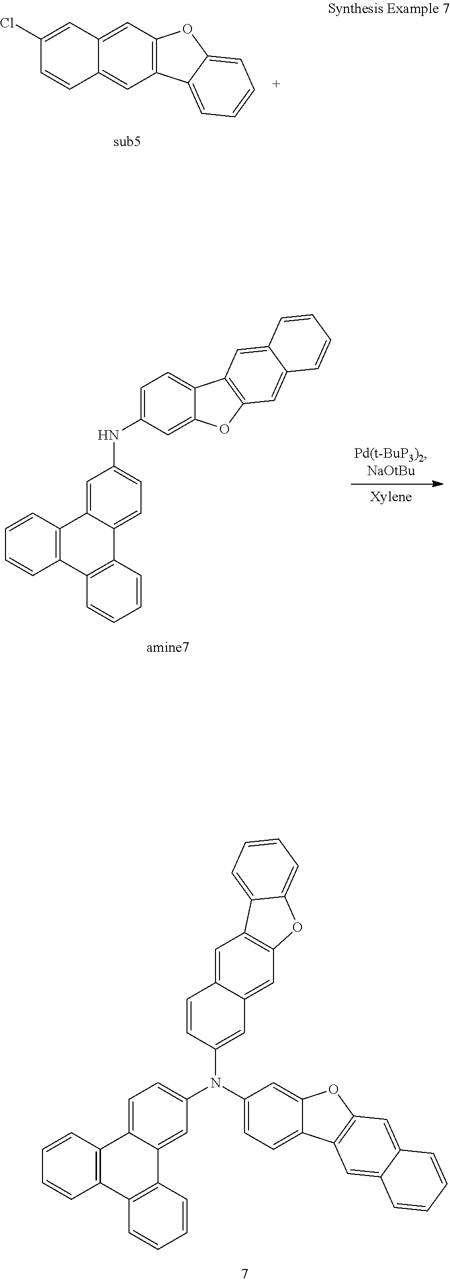 [SMILES: Clc1ccc2cc3c(cc2c1)oc1ccccc13 ; c1ccc2cc3c(cc2c1)oc1cc(Nc2ccc4c5ccccc5c5ccccc5c4c2)ccc13 ; c1ccc2cc3c(cc2c1)oc1cc(N(c2ccc4cc5c(cc4c2)oc2ccccc25)c2ccc4c5ccccc5c5ccccc5c4c2)ccc13]

sub5 (15 g, 59.4 mmol), amine7 (28.6 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 20.9 g of Compound 7 (yield: 52%, MS: [M+H]⁺=677).

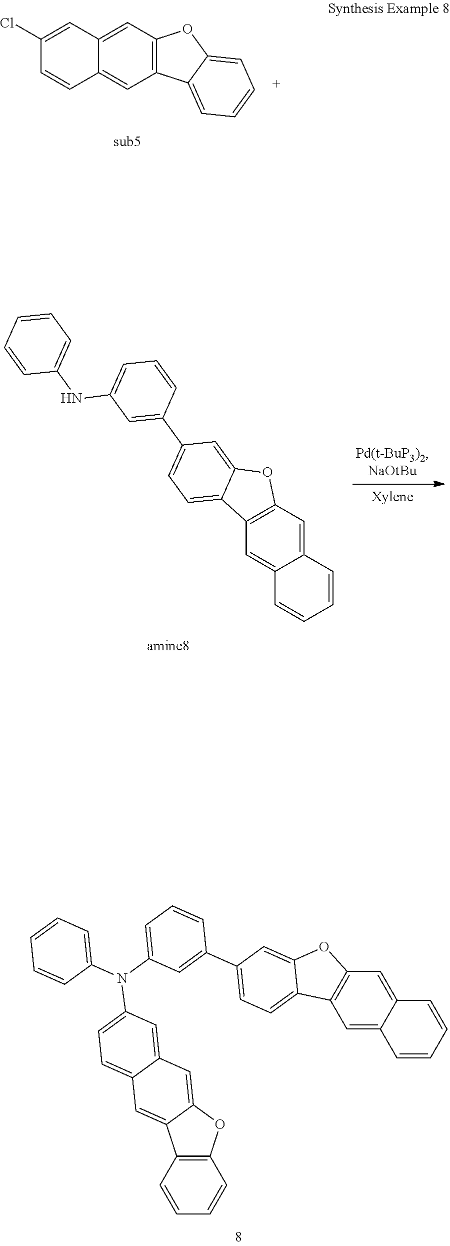 [SMILES: Clc1ccc2cc3c(cc2c1)oc1ccccc13 ; c1ccc(Nc2cccc(-c3ccc4c(c3)oc3cc5ccccc5cc34)c2)cc1 ; c1ccc(N(c2cccc(-c3ccc4c(c3)oc3cc5ccccc5cc34)c2)c2ccc3cc4c(cc3c2)oc2ccccc24)cc1]

sub5 (15 g, 59.4 mmol), amine8 (24 g, 62.3 mmol), and sodium tert-butoxide (8.6 g, 89 mmol) to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 25 g of Compound 8 (yield: 70%, MS: [M+H]⁺=603).

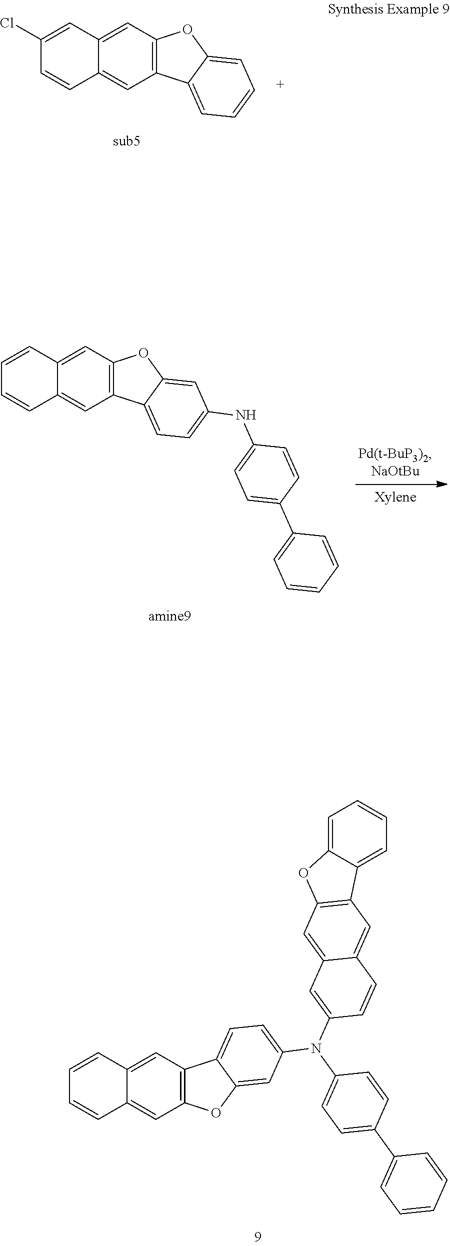 [SMILES: Clc1ccc2cc3c(cc2c1)oc1ccccc13 ; c1ccc(-c2ccc(Nc3ccc4c(c3)oc3cc5ccccc5cc34)cc2)cc1 ; c1ccc(-c2ccc(N(c3ccc4cc5c(cc4c3)oc3ccccc35)c3ccc4c(c3)oc3cc5ccccc5cc34)cc2)cc1]

sub5 (15 g, 59.4 mmol), amine9 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 20.4 g of Compound 9 (yield: 57%, MS: [M+H]⁺=603).

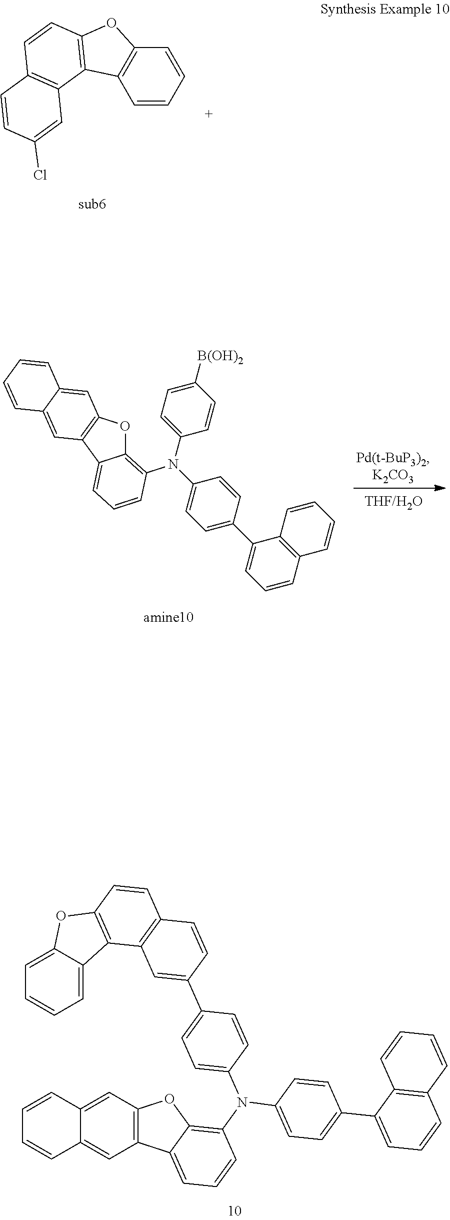 [SMILES: Clc1ccc2ccc3oc4ccccc4c3c2c1 ; OB(O)c1ccc(N(c2ccc(-c3cccc4ccccc34)cc2)c2cccc3c2oc2cc4ccccc4cc23)cc1 ; c1ccc2cc3c(cc2c1)oc1c(N(c2ccc(-c4ccc5ccc6oc7ccccc7c6c5c4)cc2)c2ccc(-c4cccc5ccccc45)cc2)cccc13]

Sub6 (15 g, 59.4 mmol) and amine10 (34.6 g, 62.3 mmol) were added to 300 ml of THF, and the mixture was stirred and refluxed. Then, potassium carbonate (24.6 g, 178.1 mmol) was dissolved in 100 ml of water, added thereto, sufficiently stirred, and then bis(tri-tert-butylphosphine)palladium(0) (0.3 g, 0.6 mmol) was added. After the reaction for 6 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 26.4 g of Compound 10 (yield: 61%, MS: [M+H]⁺=729).

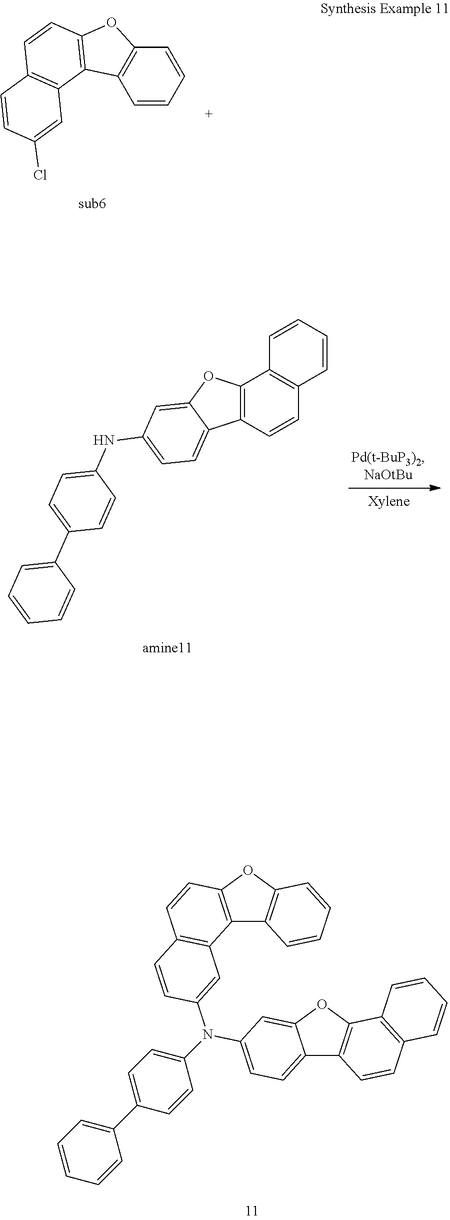 [SMILES: Clc1ccc2ccc3oc4ccccc4c3c2c1 ; c1ccc(-c2ccc(Nc3ccc4c(c3)oc3c5ccccc5ccc43)cc2)cc1 ; c1ccc(-c2ccc(N(c3ccc4c(c3)oc3c5ccccc5ccc43)c3ccc4ccc5oc6ccccc6c5c4c3)cc2)cc1]

sub6 (15 g, 59.4 mmol), amine11 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 18.9 g of Compound 11 (yield: 53%, MS: [M+H]⁺=603).

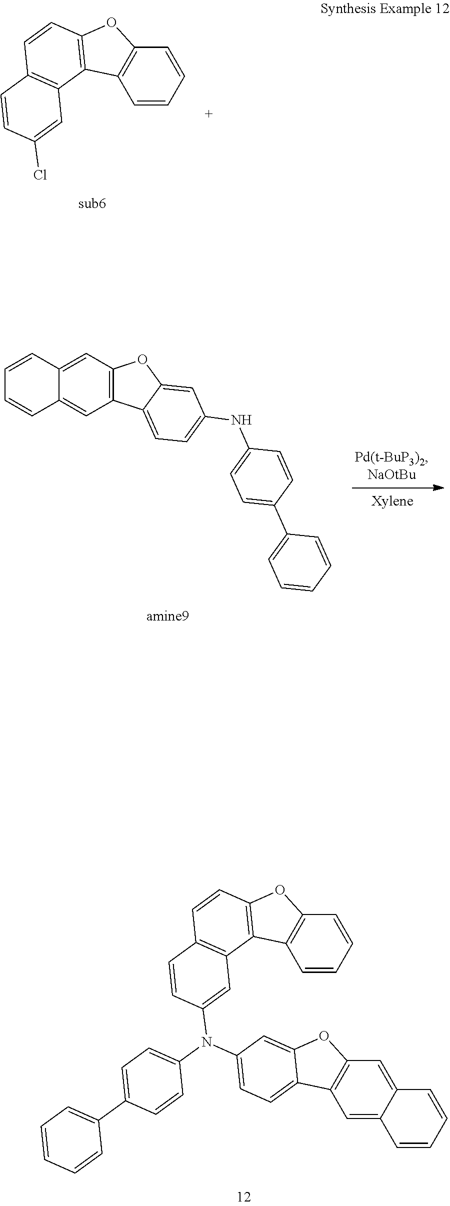 [SMILES: Clc1ccc2ccc3oc4ccccc4c3c2c1 ; c1ccc(-c2ccc(Nc3ccc4c(c3)oc3cc5ccccc5cc34)cc2)cc1 ; c1ccc(-c2ccc(N(c3ccc4c(c3)oc3cc5ccccc5cc34)c3ccc4ccc5oc6ccccc6c5c4c3)cc2)cc1]

sub6 (15 g, 59.4 mmol), amine9 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.9 g of Compound 12 (yield: 64%, MS: [M+H]⁺=603).

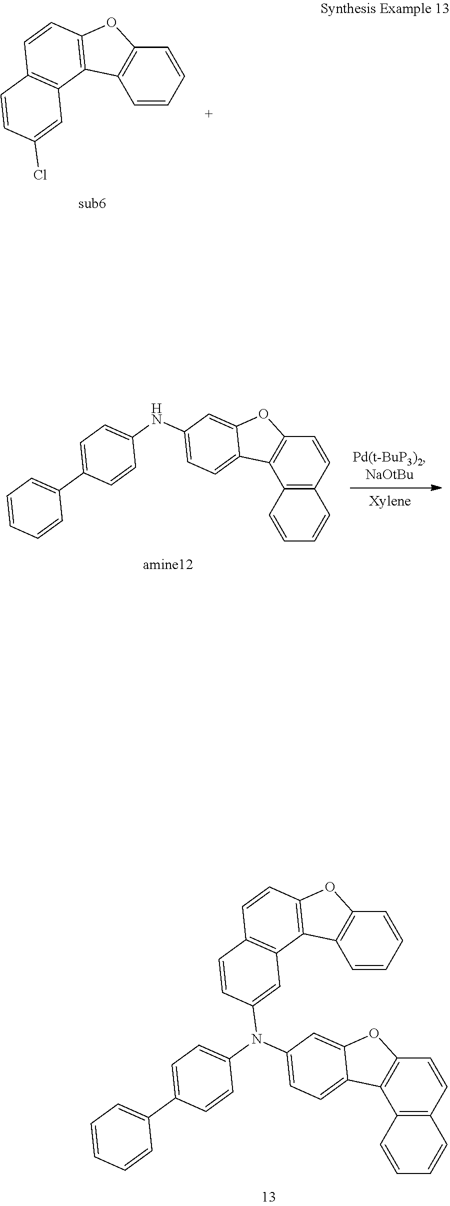 [SMILES: Clc1ccc2ccc3oc4ccccc4c3c2c1 ; c1ccc(-c2ccc(Nc3ccc4c(c3)oc3ccc5ccccc5c34)cc2)cc1 ; c1ccc(-c2ccc(N(c3ccc4c(c3)oc3ccc5ccccc5c34)c3ccc4ccc5oc6ccccc6c5c4c3)cc2)cc1]

sub6 (15 g, 59.4 mmol), amine12 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.5 g of Compound 13 (yield: 63%, MS: [M+H]⁺=603).

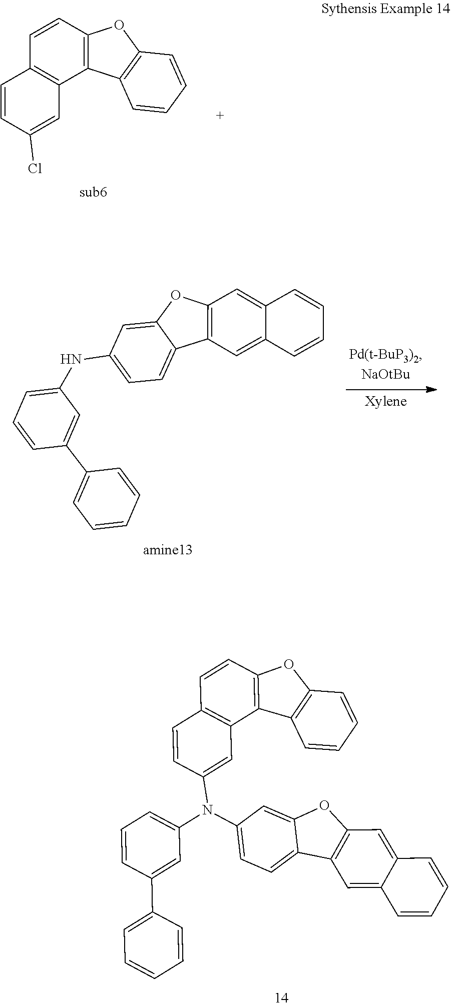 [SMILES: Clc1ccc2ccc3oc4ccccc4c3c2c1 ; c1ccc(-c2cccc(Nc3ccc4c(c3)oc3cc5ccccc5cc34)c2)cc1 ; c1ccc(-c2cccc(N(c3ccc4c(c3)oc3cc5ccccc5cc34)c3ccc4ccc5oc6ccccc6c5c4c3)c2)cc1]

sub6 (15 g, 59.4 mmol), amine13 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 24.6 g of Compound 14 (yield: 69%, MS: [M+H]⁺=603).

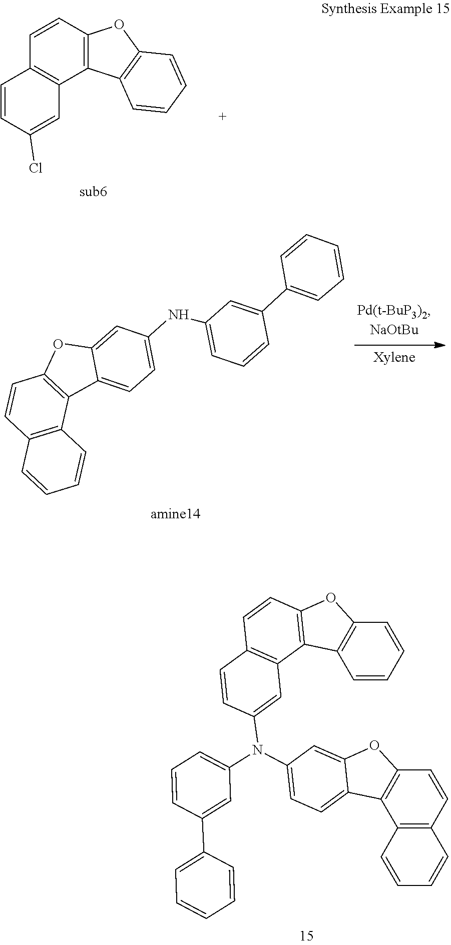 [SMILES: Clc1ccc2ccc3oc4ccccc4c3c2c1 ; c1ccc(-c2cccc(Nc3ccc4c(c3)oc3ccc5ccccc5c34)c2)cc1 ; c1ccc(-c2cccc(N(c3ccc4c(c3)oc3ccc5ccccc5c34)c3ccc4ccc5oc6ccccc6c5c4c3)c2)cc1]

sub6 (15 g, 59.4 mmol), amine14 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 21.1 g of Compound 15 (yield: 59%, MS: [M+H]⁺=603).

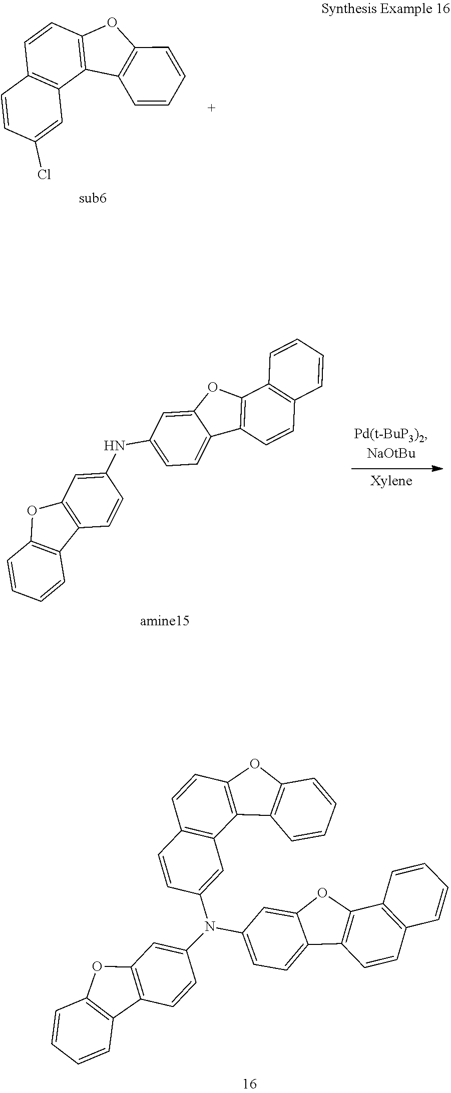 [SMILES: Clc1ccc2ccc3oc4ccccc4c3c2c1 ; c1ccc2c(c1)ccc1c3ccc(Nc4ccc5c(c4)oc4ccccc45)cc3oc21 ; c1ccc2c(c1)ccc1c3ccc(N(c4ccc5c(c4)oc4ccccc45)c4ccc5ccc6oc7ccccc7c6c5c4)cc3oc21]

sub6 (15 g, 59.4 mmol), amine15 (24.9 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19.4 g of Compound 16 (yield: 53%, MS: [M+H]⁺=617).

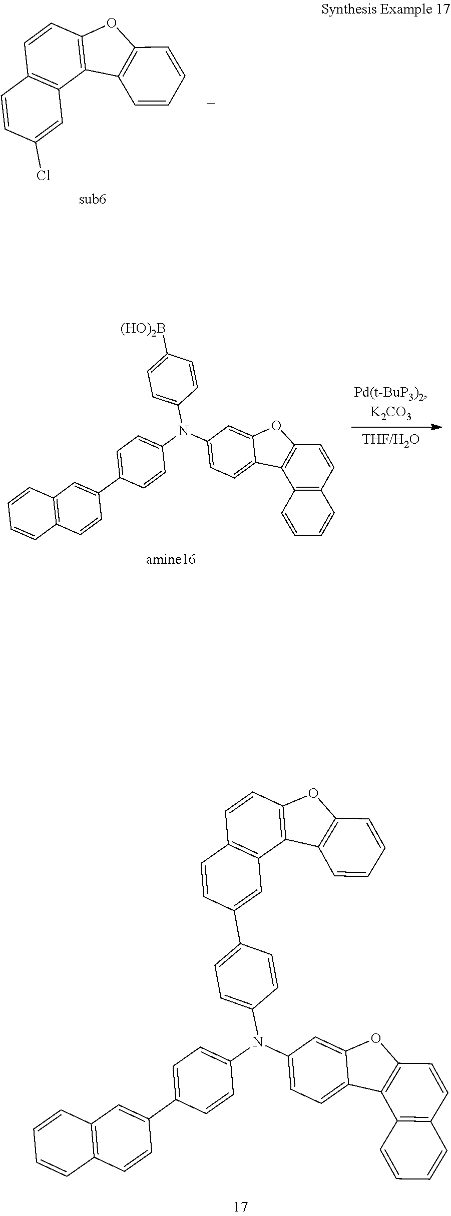 [SMILES: Clc1ccc2ccc3oc4ccccc4c3c2c1 ; OB(O)c1ccc(N(c2ccc(-c3ccc4ccccc4c3)cc2)c2ccc3c(c2)oc2ccc4ccccc4c23)cc1 ; c1ccc2cc(-c3ccc(N(c4ccc(-c5ccc6ccc7oc8ccccc8c7c6c5)cc4)c4ccc5c(c4)oc4ccc6ccccc6c45)cc3)ccc2c1]

Sub6 (15 g, 59.4 mmol) and amine16 (31.5 g, 62.3 mmol) were added to 300 ml of THF, and the mixture was stirred and refluxed. Then, potassium carbonate (24.6 g, 178.1 mmol) was dissolved in 100 ml of water, added thereto, sufficiently stirred, and then bis(tri-tert-butylphosphine)palladium(0) (0.3 g, 0.6 mmol) was added. After the reaction for 6 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 29.8 g of Compound 17 (yield: 69%, MS: [M+H]⁺=729).

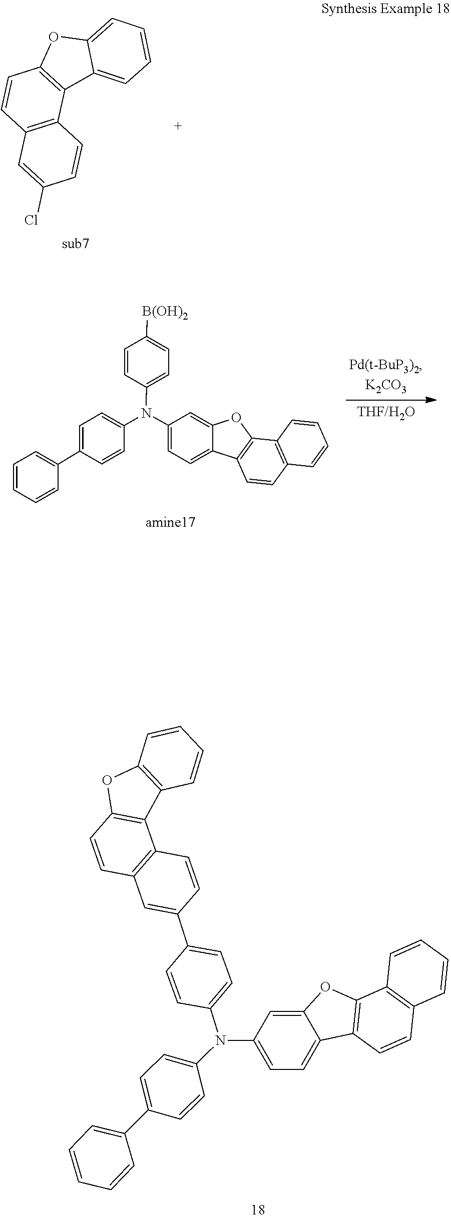 [SMILES: Clc1ccc2c(ccc3oc4ccccc4c32)c1 ; OB(O)c1ccc(N(c2ccc(-c3ccccc3)cc2)c2ccc3c(c2)oc2c4ccccc4ccc32)cc1 ; c1ccc(-c2ccc(N(c3ccc(-c4ccc5c(ccc6oc7ccccc7c65)c4)cc3)c3ccc4c(c3)oc3c5ccccc5ccc43)cc2)cc1]

Sub7 (15 g, 59.4 mmol) and amine17 (37.1 g, 62.3 mmol) were added to 300 ml of THF, and the mixture was stirred and refluxed. Then, potassium carbonate (24.6 g, 178.1 mmol) was dissolved in 100 ml of water, added thereto, sufficiently stirred, and then bis(tri-tert-butylphosphine)palladium(0) (0.3 g, 0.6 mmol) was added. After the reaction for 2 hours, the reaction mixture was cooled to room temperature, the organic layer and the aqueous layer were separated, and the organic layer was distilled. This was dissolved again in chloroform and washed twice with water. The organic layer was then separated, anhydrous magnesium sulfate was added thereto, stirred, then filtered, and the filtrate was distilled under reduced pressure. The concentrated compound was purified by a silica gel column chromatography to prepare 30.6 g of Compound 18 (yield: 76%, MS: [M+H]⁺=679).

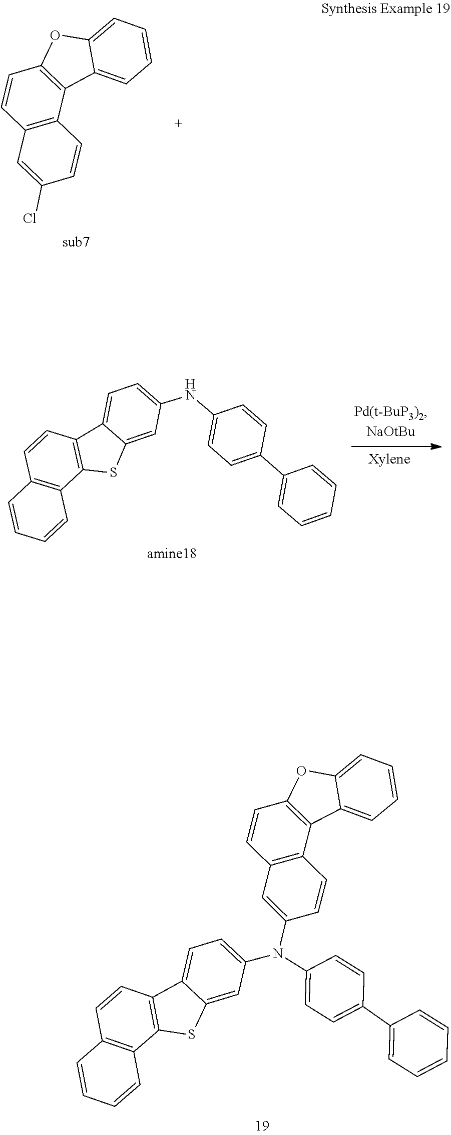 [SMILES: Clc1ccc2c(ccc3oc4ccccc4c32)c1 ; c1ccc(-c2ccc(Nc3ccc4c(c3)sc3c5ccccc5ccc43)cc2)cc1 ; c1ccc(-c2ccc(N(c3ccc4c(ccc5oc6ccccc6c54)c3)c3ccc4c(c3)sc3c5ccccc5ccc43)cc2)cc1]

sub7 (15 g, 59.4 mmol), amine18 (25 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 22.7 g of Compound 19 (yield: 62%, MS: [M+H]⁺=619).

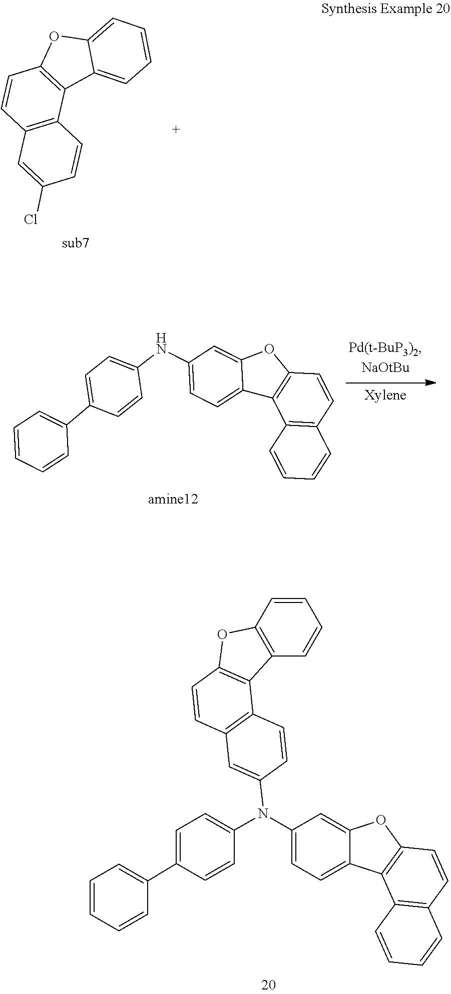 [SMILES: Clc1ccc2c(ccc3oc4ccccc4c32)c1 ; c1ccc(-c2ccc(Nc3ccc4c(c3)oc3ccc5ccccc5c34)cc2)cc1 ; c1ccc(-c2ccc(N(c3ccc4c(ccc5oc6ccccc6c54)c3)c3ccc4c(c3)oc3ccc5ccccc5c34)cc2)cc1]

sub7 (15 g, 59.4 mmol), amine12 (24 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 17.9 g of Compound 20 (yield: 50%, MS: [M+H]⁺=603).

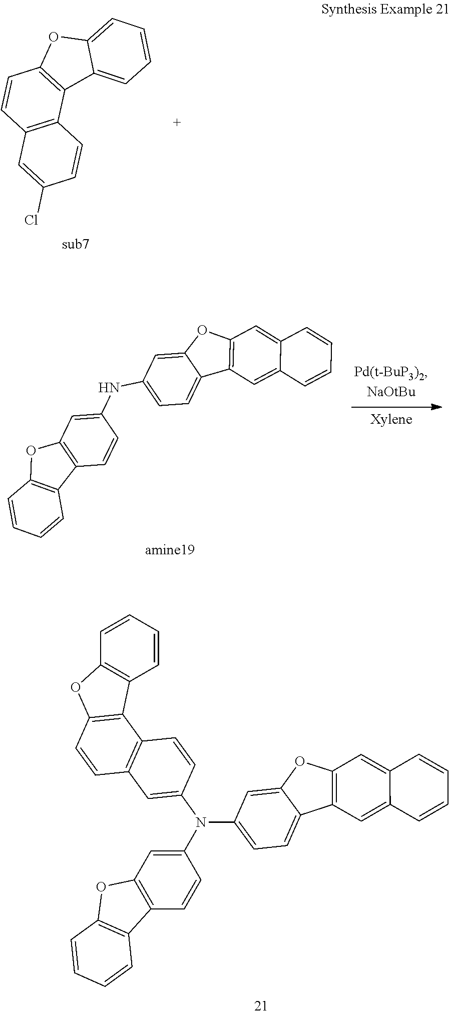 [SMILES: Clc1ccc2c(ccc3oc4ccccc4c32)c1 ; c1ccc2cc3c(cc2c1)oc1cc(Nc2ccc4c(c2)oc2ccccc24)ccc13 ; c1ccc2cc3c(cc2c1)oc1cc(N(c2ccc4c(ccc5oc6ccccc6c54)c2)c2ccc4c(c2)oc2ccccc24)ccc13]

sub7 (15 g, 59.4 mmol), amine19 (24.9 g, 62.3 mmol), and sodium tert-butoxide (8.6 g, 89 mmol) were added to 300 ml of xylene under a nitrogen atmosphere, and the mixture was stirred and refluxed. Then, bis(tri-tert-butylphosphine)palladium(0) (0.3 g, 0.6 mmol) was added thereto. When the reaction was terminated after 5 hours, the reaction mixture was cooled to room temperature, and the solvent was removed under reduced pressure. Then, the compound was completely dissolved again in chloroform, washed twice with water, and then the organic layer was separated, treated with anhydrous magnesium sulfate and then filtered, and the filtrate was distilled under reduced pressure. The concentrated compound was purified by a silica gel column chromatography to prepare 19 g of Compound 21 (yield: 52%, MS: [M+H]⁺=617).

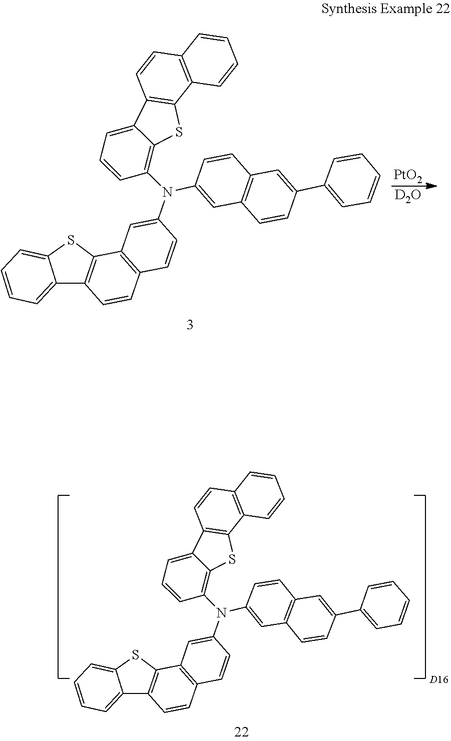

Compound 3 (10 g, 14.6 mmol), PtO₂ (1 g, 4.4 mmol), and D₂O (73 ml) were added to a shaker tube, and then the tube was sealed, and heated at 250° C. and 600 psi for 12 hours. When the reaction was terminated, chloroform was added thereto, and the reaction solution was transferred to a separatory funnel, and extracted. The extract was dried over MgSO₄ and concentrated, and then the sample was purified by silica gel column chromatography to prepare 5.1 g of Compound 22 (yield: 50%, M.W.=701).

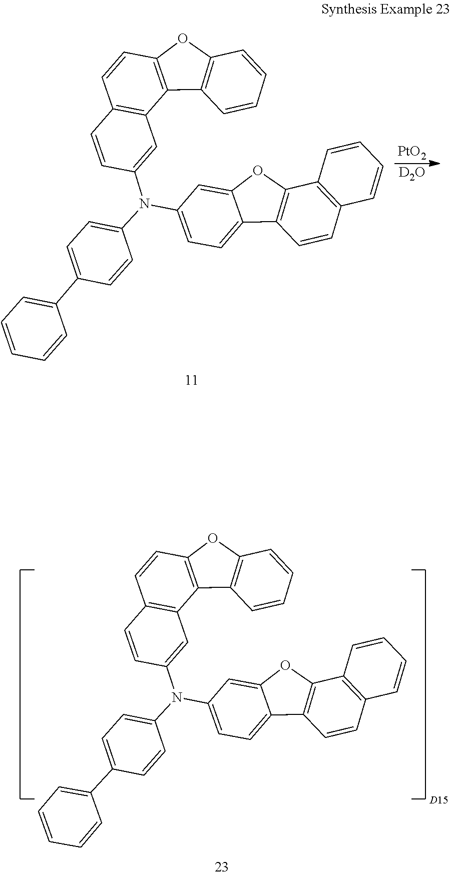

Compound 11 (10 g, 16.6 mmol), PtO₂ (1.1 g, 5 mmol), and D₂O (83 ml) were added to a shaker tube, and then the tube was sealed, and heated at 250° C. and 600 psi for 12 hours. When the reaction was terminated, chloroform was added thereto, and the reaction solution was transferred to a separatory funnel, and extracted. The extract was dried over MgSO₄ and concentrated, and then the sample was purified by silica gel column chromatography to prepare 4.9 g of Compound 23 (yield: 48%, M.W.=618).

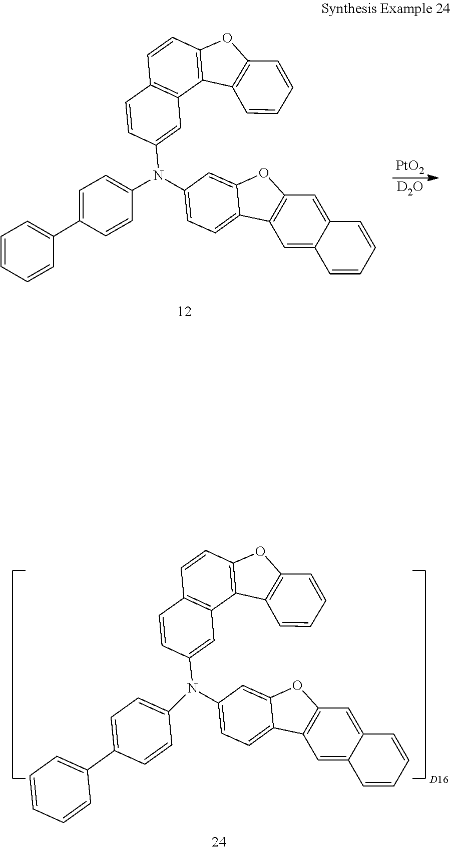

Compound 12 (10 g, 16.6 mmol), PtO₂ (1.1 g, 5 mmol) and D₂O (83 ml) were added to a shaker tube, and then the tube was sealed, and heated at 250° C. and 600 psi for 12 hours. When the reaction was terminated, chloroform was added thereto, and the reaction solution was transferred to a separatory funnel, and extracted. The extract was dried over MgSO₄ and concentrated, and then the sample was purified by silica gel column chromatography to prepare 5.4 g of Compound 24 (yield: 53%, M.W.=619).

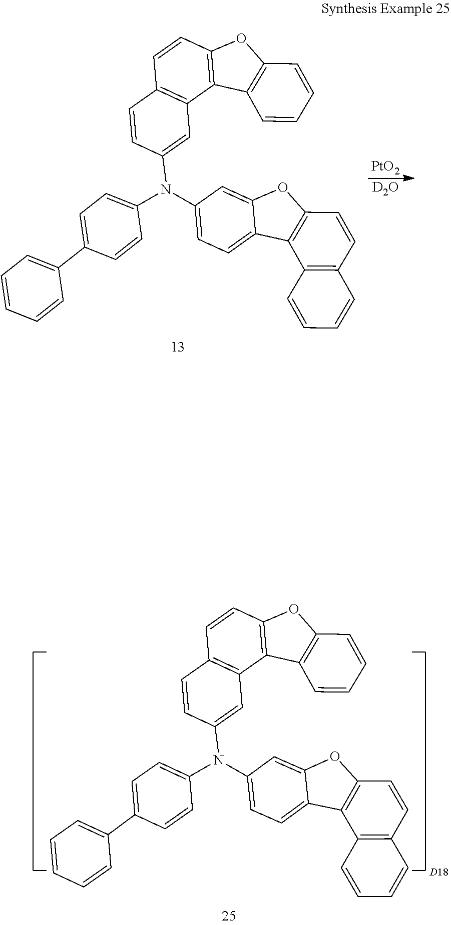

Compound 13 (10 g, 16.6 mmol), PtO₂ (1.1 g, 5 mmol), and D₂O (83 ml) were added to a shaker tube, and then the tube was sealed, and heated at 250° C. and 600 psi for 12 hours. When the reaction was terminated, chloroform was added thereto, and the reaction solution was transferred to a separatory funnel, and extracted. The extract was dried over MgSO₄ and concentrated, and then the sample was purified by silica gel column chromatography to prepare 5 g of Compound 25 (yield: 49%, M.W.=621).

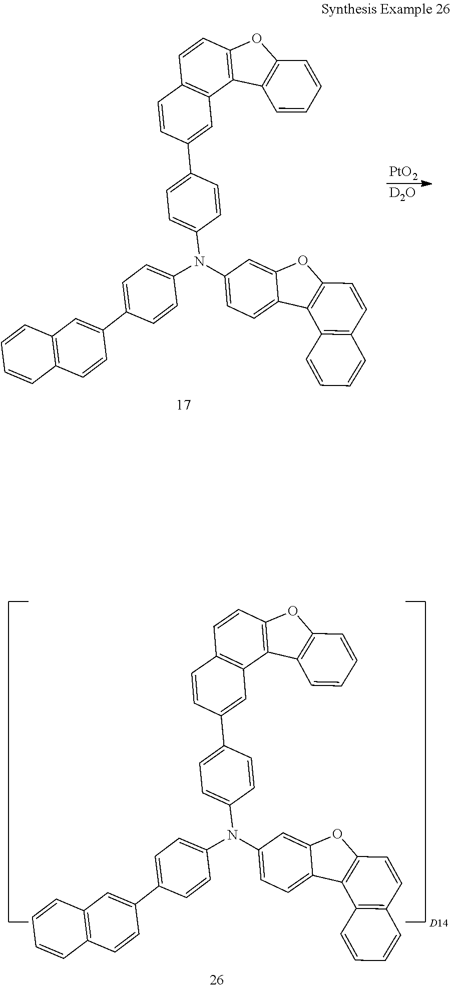

Compound 17 (10 g, 13.7 mmol), PtO₂ (0.9 g, 4.1 mmol), and D₂O (69 ml) were added to a shaker tube, and then the tube was sealed, and heated at 250° C. and 600 psi for 12 hours. When the reaction was terminated, chloroform was added thereto, and the reaction solution was transferred to a separatory funnel, and extracted. The extract was dried over MgSO₄ and concentrated, and then the sample was purified by silica gel column chromatography to prepare 4.9 g of Compound 26 (yield: 48%, M.W.=743).

### EXAMPLE AND COMPARATIVE EXAMPLE

### Example 1

A glass substrate on which ITO (indium tin oxide) was coated as a thin film to a thickness of 1,000 Å was put into distilled water in which a detergent was dissolved, and ultrasonically cleaned. A product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HI-1 was formed to a thickness of 1150 Å as a hole injection layer, but the following compound PD was p-doped at a concentration of 1.5%. The following compound HT-1 was vacuum deposited on the hole injection layer to form a hole transport layer with a layer thickness of 800 Å. Then, the following compound EB-1 was vacuum deposited on the hole transport layer to a layer thickness of 150 Å to form an electron blocking layer (electron suppression layer).

Then, the following Compound RH-1 as the first host compound, Compound 1 prepared in Synthesis Example 1 as the second host compound, and the following compound RD were vacuum deposited at a weight ratio of 49:49:2 on the EB-1 deposited layer to form a red light emitting layer with a thickness of 400 Å.

The following Compound HB-1 was vacuum deposited on the light emitting layer to a layer thickness of 30 Å to form a hole blocking layer. Then, the following Compound ET-1 and the following Compound LiQ were vacuum deposited at a weight ratio of 2:1 on the hole blocking layer to form an electron injection and transport layer with a film thickness of 300 Å. Lithium fluoride (LIF) and aluminum were sequentially deposited to have a thickness of 12 Å and 1,000 Å, respectively, on the electron injection and transport layer, thereby forming a cathode.

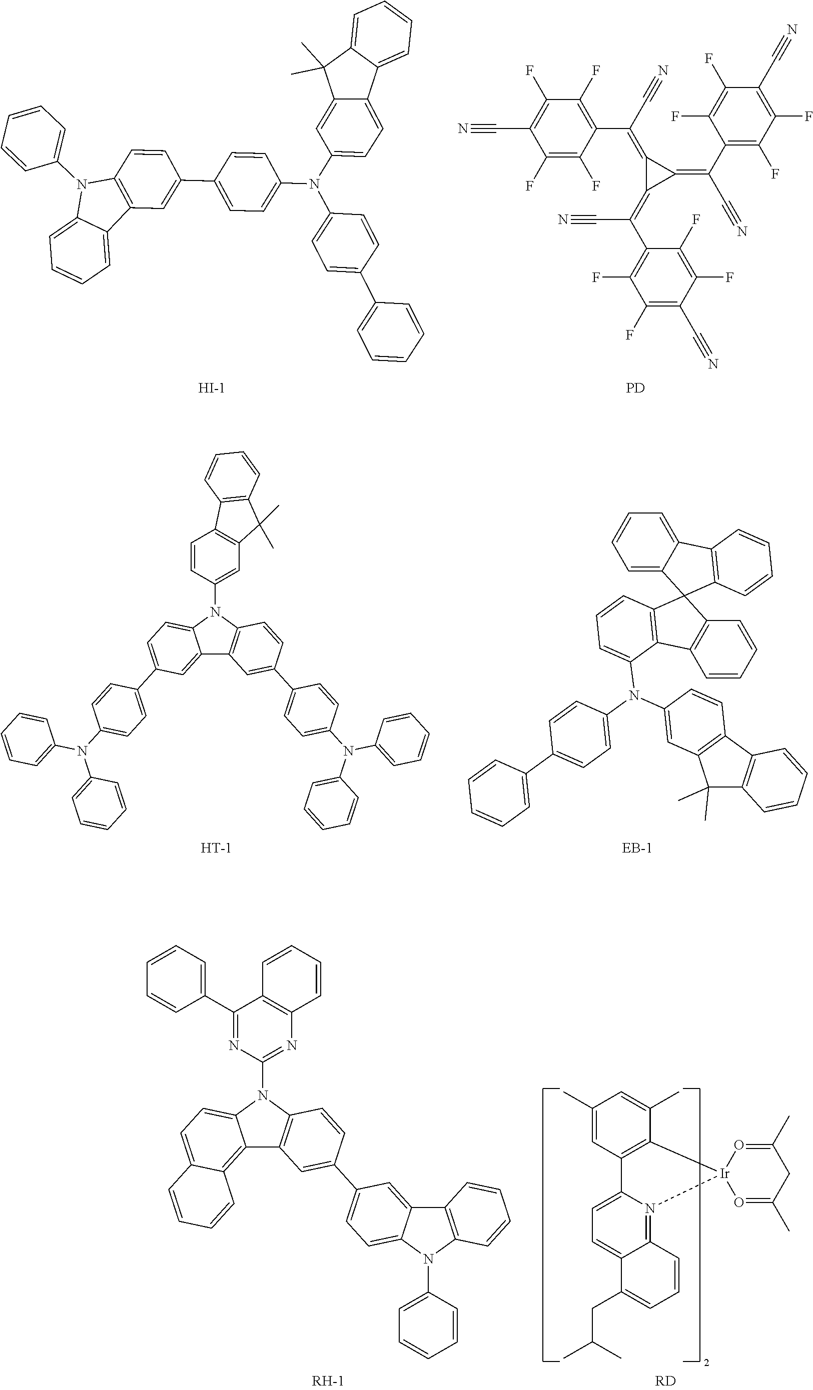

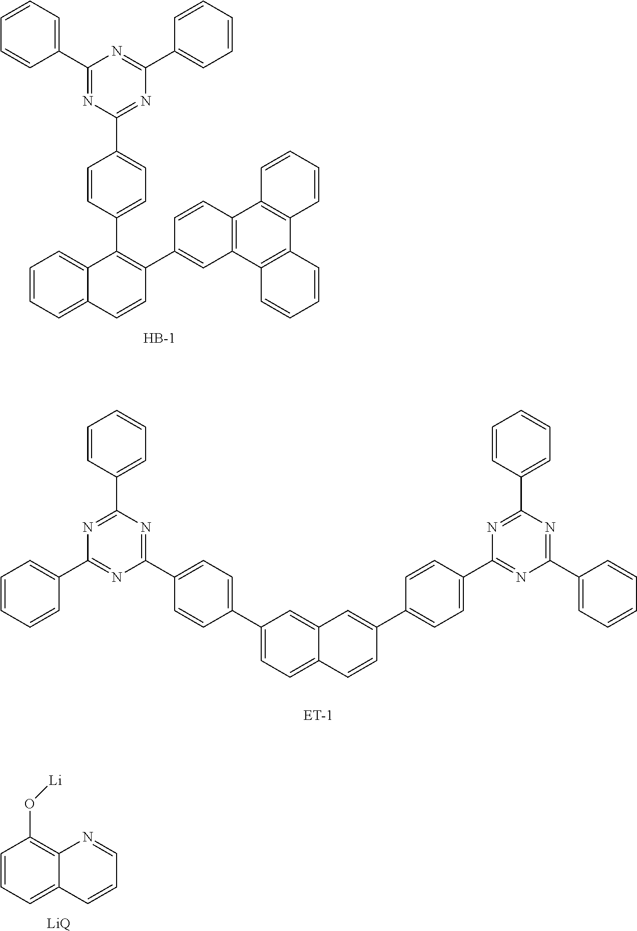

In the above-mentioned processes, the vapor deposition rate of the organic material was maintained at 0.4˜0.7 Å/sec, the deposition rates of lithium fluoride and aluminum of the cathode were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Examples 2 to 26

The organic light emitting device was manufactured in the same manner as in Example 1, except that the compounds listed in Table 1 below were used instead of Compound 1 as the second host compound of the light emitting layer.

### Comparative Examples 1 to 9

The organic light emitting device was manufactured in the same manner as in Example 1, except that the compounds C1 to C9 listed in Table 1 below were used instead of Compound 1 as the second host compound of the light emitting layer. Herein, the compounds C1 to C9 are summarized as follows.

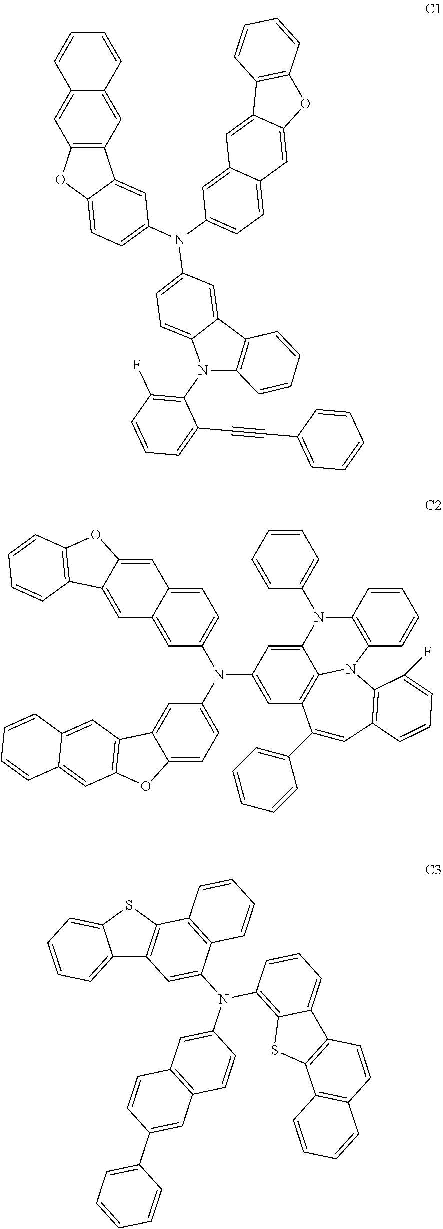 [SMILES: Fc1cccc(C#Cc2ccccc2)c1-n1c2ccccc2c2cc(N(c3ccc4cc5oc6ccccc6c5cc4c3)c3ccc4oc5cc6ccccc6cc5c4c3)ccc21 ; Fc1cccc2c1N1c3ccccc3N(c3ccccc3)c3cc(N(c4ccc5cc6oc7ccccc7c6cc5c4)c4ccc5oc6cc7ccccc7cc6c5c4)cc(c31)C(c1ccccc1)=C2 ; c1ccc(-c2ccc3cc(N(c4cc5c6ccccc6sc5c5ccccc45)c4cccc5c4sc4c6ccccc6ccc54)ccc3c2)cc1]

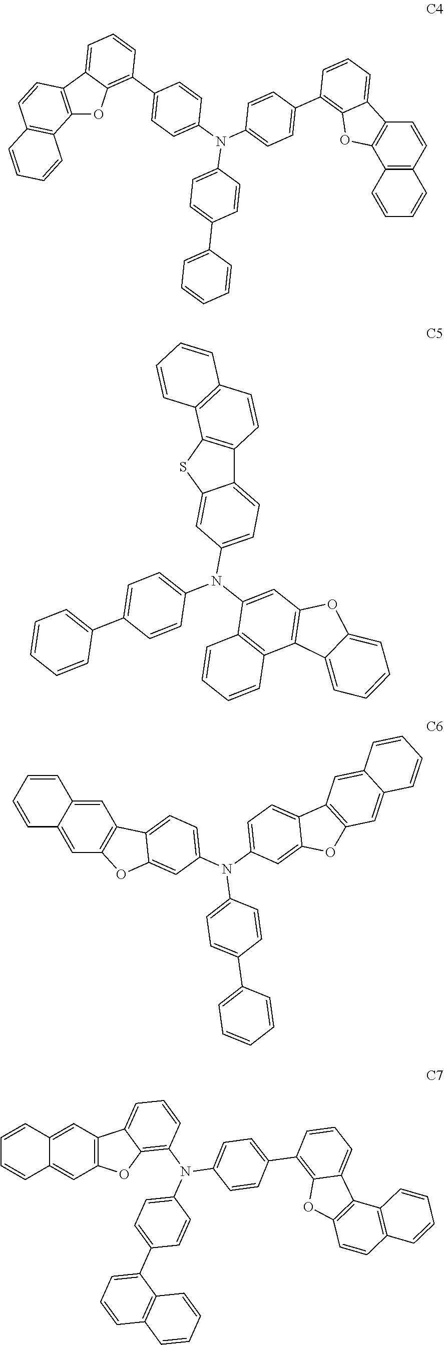 [SMILES: c1ccc(-c2ccc(N(c3ccc(-c4cccc5c4oc4c6ccccc6ccc54)cc3)c3ccc(-c4cccc5c4oc4c6ccccc6ccc54)cc3)cc2)cc1 ; c1ccc(-c2ccc(N(c3ccc4c(c3)sc3c5ccccc5ccc43)c3cc4oc5ccccc5c4c4ccccc34)cc2)cc1 ; c1ccc(-c2ccc(N(c3ccc4c(c3)oc3cc5ccccc5cc34)c3ccc4c(c3)oc3cc5ccccc5cc34)cc2)cc1 ; c1ccc2cc3c(cc2c1)oc1c(N(c2ccc(-c4cccc5ccccc45)cc2)c2ccc(-c4cccc5c4oc4ccc6ccccc6c45)cc2)cccc13]

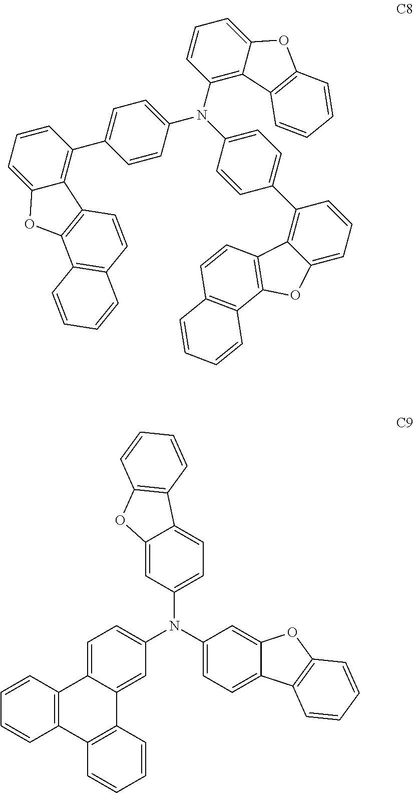 [SMILES: c1ccc2c(c1)ccc1c2oc2cccc(-c3ccc(N(c4ccc(-c5cccc6oc7c8ccccc8ccc7c56)cc4)c4cccc5oc6ccccc6c45)cc3)c21 ; c1ccc2c(c1)oc1cc(N(c3ccc4c(c3)oc3ccccc34)c3ccc4c5ccccc5c5ccccc5c4c3)ccc12]

### Experimental Example

The voltage, efficiency and lifetime were measured by applying a current to the organic light emitting devices manufactured in the Examples 1 to 26 and Comparative Examples 1 to 9, and the results are shown in Table 1 below. T95 means the time required for the luminance to be reduced to 95% of the initial luminance.

TABLE 1

| | | Voltage | Efficiency | Lifetime |
|---|---|---|---|---|
| | Second | (V) | (Cd/A) | (h) |
| | host | (@10 | (@10 | (LT95 at |
| Category | compound | mA/cm²) | mA/cm²) | 50 mA/cm²) |
| Comparative | C1 | 6.9 | 9.7 | 16 |
| Example 1 | | | | |
| Comparative | C2 | 7.1 | 11.7 | 20 |
| Example 2 | | | | |
| Comparative | C3 | 6.4 | 15.8 | 78 |
| Example 3 | | | | |
| Comparative | C4 | 6.7 | 16.5 | 82 |
| Example 4 | | | | |
| Comparative | C5 | 6.7 | 16.9 | 87 |
| Example 5 | | | | |
| Comparative | C6 | 6.3 | 17.7 | 89 |
| Example 6 | | | | |
| Comparative | C7 | 6.4 | 16.9 | 87 |
| Example 7 | | | | |
| Comparative | C8 | 6.5 | 16.2 | 81 |
| Example 8 | | | | |
| Comparative | C9 | 6.7 | 12.7 | 34 |
| Example 9 | | | | |
| Example 1 | Compound | 5.0 | 21.1 | 155 |
| | 1 | | | |
| Example 2 | Compound | 4.9 | 21.3 | 157 |
| | 2 | | | |
| Example 3 | Compound | 4.8 | 22.3 | 158 |
| | 3 | | | |
| Example 4 | Compound | 4.9 | 22.3 | 160 |
| | 4 | | | |
| Example 5 | Compound | 4.9 | 22.5 | 161 |
| | 5 | | | |
| Example 6 | Compound | 4.7 | 23.5 | 155 |
| | 6 | | | |
| Example 7 | Compound | 4.9 | 21.6 | 158 |
| | 7 | | | |
| Example 8 | Compound | 4.8 | 23.7 | 161 |
| | 8 | | | |
| Example 9 | Compound | 4.7 | 24.2 | 165 |
| | 9 | | | |
| Example 10 | Compound | 4.8 | 23.7 | 162 |
| | 10 | | | |
| Example 11 | Compound | 4.3 | 25.0 | 181 |
| | 11 | | | |
| Example 12 | Compound | 4.1 | 25.3 | 186 |
| | 12 | | | |
| Example 13 | Compound | 4.0 | 25.7 | 192 |
| | 13 | | | |
| Example 14 | Compound | 4.3 | 24.8 | 176 |
| | 14 | | | |
| Example 15 | Compound | 4.3 | 25.2 | 178 |
| | 15 | | | |
| Example 16 | Compound | 4.5 | 24.5 | 174 |
| | 16 | | | |
| Example 17 | Compound | 4.6 | 24.1 | 175 |
| | 17 | | | |
| Example 18 | Compound | 4.6 | 23.3 | 170 |
| | 18 | | | |
| Example 19 | Compound | 4.9 | 23.4 | 168 |
| | 19 | | | |
| Example 20 | Compound | 4.5 | 24.9 | 175 |
| | 20 | | | |
| Example 21 | Compound | 4.5 | 24.4 | 171 |
| | 21 | | | |
| Example 22 | Compound | 4.8 | 22.9 | 178 |
| | 22 | | | |
| Example 23 | Compound | 4.3 | 25.0 | 196 |
| | 23 | | | |
| Example 24 | Compound | 4.1 | 25.3 | 198 |
| | 24 | | | |
| Example 25 | Compound | 4.0 | 25.7 | 208 |
| | 25 | | | |
| Example 26 | Compound | 4.6 | 24.1 | 193 |
| | 26 | | | |

As shown in Table 1, it could be confirmed that the organic light emitting devices of Examples using the compound represented by Chemical Formula 1 as the host material of the light emitting layer exhibit excellent performance in terms of efficiency and lifetime as compared to the organic light emitting devices of Comparative Examples using a compound having a structure different from that.

As set forth above, it could be confirmed that the compounds of the present disclosure exhibit superior properties in terms of efficiency and lifetime depending on the position and type of the substituent.

| | [Description of Symbols] |
|---|---|
| 1: | substrate |
| 2: | anode |
| 3: | hole transport layer |
| 4: | light emitting layer |
| 5: | electron injection and transport layer |
| 6: | cathode |
| 7: | hole injection layer |
| 8: | electron blocking layer |
| 9: | hole blocking layer |

1. A compound represented by the following Chemical Formula 1: 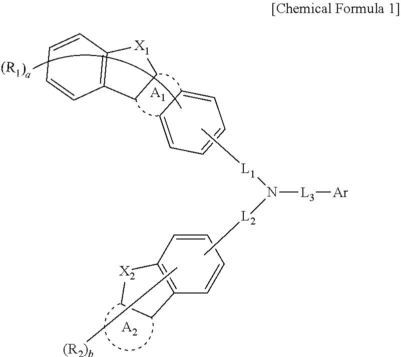
wherein in Chemical Formula 1,
X₁ and X₂ are each independently O or S,
A₁ is a benzene ring fused with adjacent pentagonal and benzene rings,
A₂ is a naphthalene ring fused with an adjacent pentagonal ring,
L₁ to L₃ are each independently a single bond; or a substituted or unsubstituted C₆₋₆₀ arylene,
Ar is a substituted or unsubstituted C₆₋₆₀ aryl; a substituted or unsubstituted C₂₋₆₀ heteroaryl containing a heteroatom of O or S; or a substituted or unsubstituted C₂₋₆₀ heteroaryl containing heteroatom of N and at least one selected of O and S,
R₁ and R₂ are each independently hydrogen; deuterium; halogen; cyano; or a substituted or unsubstituted C₁₋₆₀ alkyl, and
a and b are each independently an integer of 0 to 9.

2. The compound of claim 1, wherein:
both X₁ and X₂ are O; or
both X₁ and X₂ are S; or
X₁ is O, and X₂ is S; or
X₁ is S, and X₂ is O.

3. The compound of claim 1, wherein: 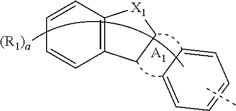
is any one of the substituents represented by the following Chemical Formulas 2a to 2l: 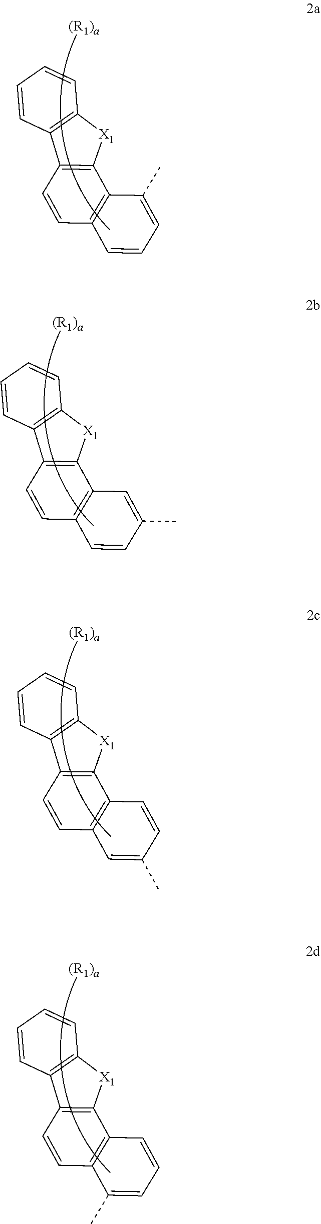 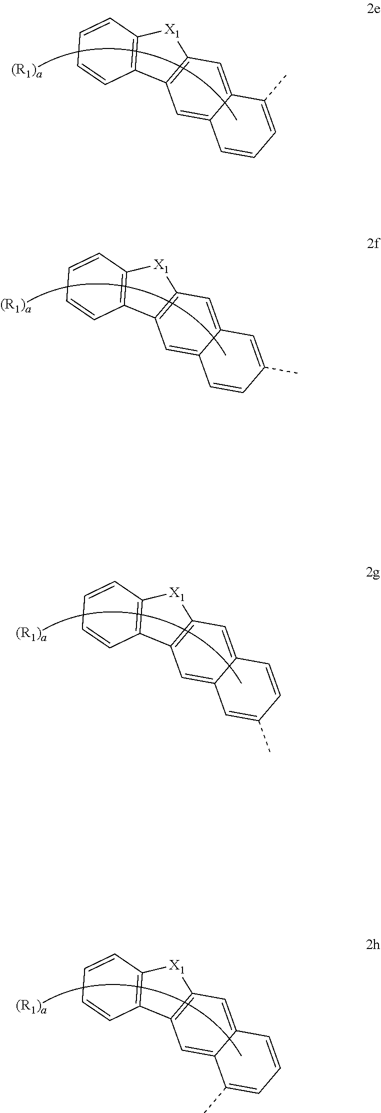 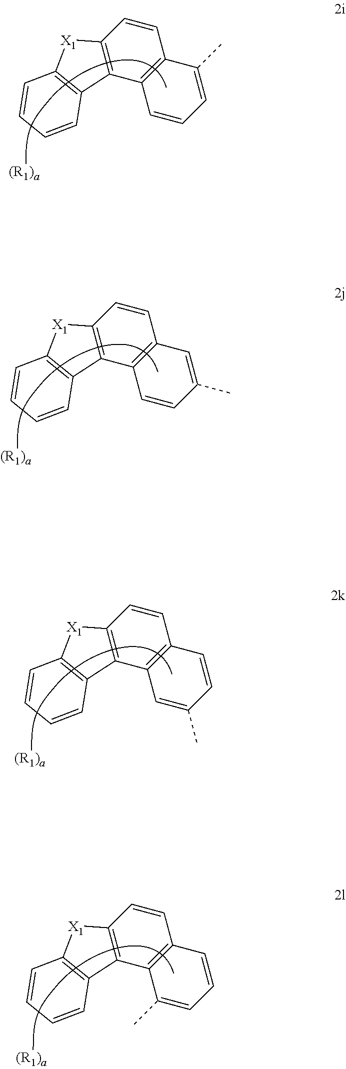
wherein in Chemical Formulas 2a to 2l,
X₁, R₁ and a are as defined in claim 1.

4. The compound of claim 1, wherein: 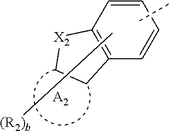
is any one of the substituents represented by the following Chemical Formulas 3a to 3l: 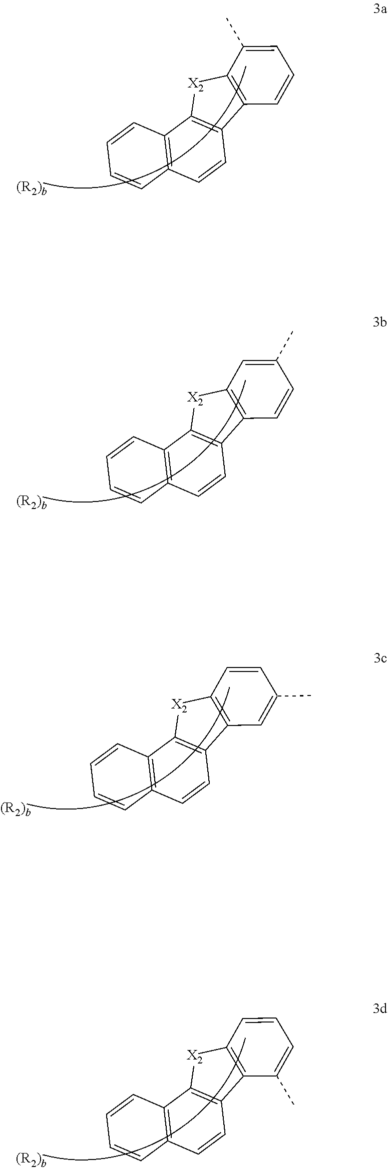 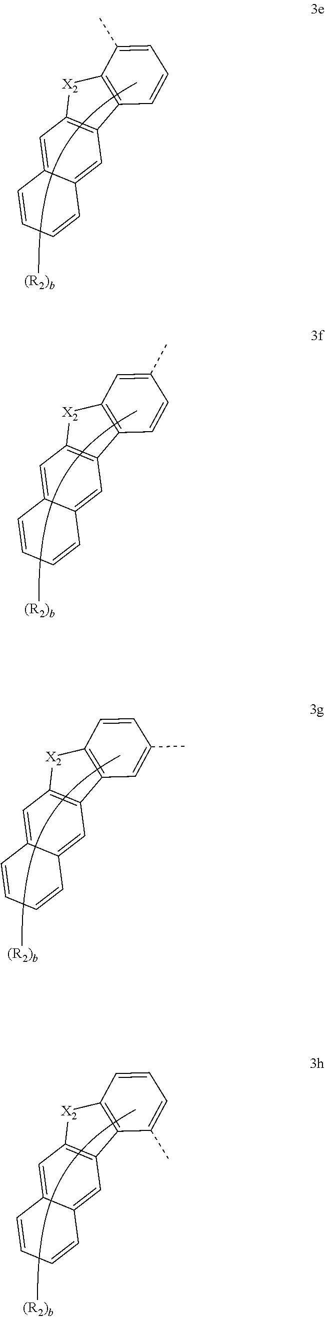 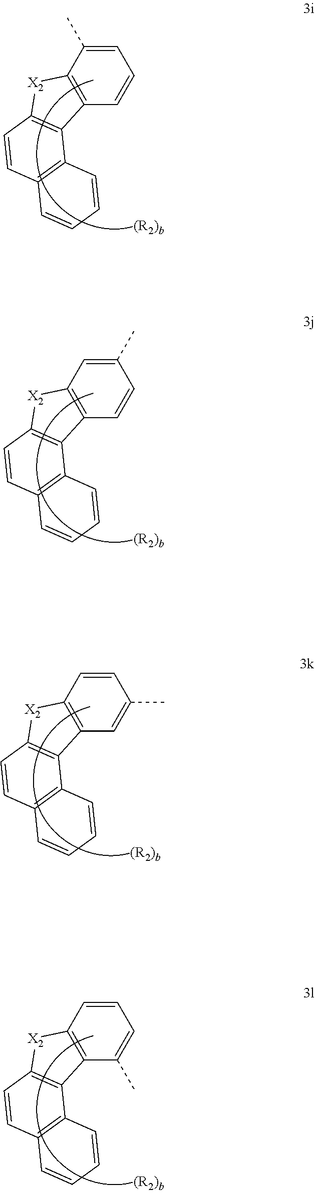
wherein in Chemical Formulas 3a to 3l,
X₂, R₂ and bare each independently as defined in claim 1.

5. The compound of claim 1, wherein:
L₁ and L₂ are each independently a single bond, or a substituted or unsubstituted C₆₋₁₂ arylene.

6. The compound of claim 5, wherein:
both L₁ and L₂ are a single bond; or
L₁ is a single bond, and L₂ is a substituted or unsubstituted C₆₋₁₂ arylene; or
L₁ is a substituted or unsubstituted C₆₋₁₂ arylene, and L₂ is a single bond.

7. The compound of claim 1, wherein:
L₃ is a single bond, or a substituted or unsubstituted phenylene,

8. The compound of claim 1, wherein:
Ar is a substituted or unsubstituted C₆₋₂₀ aryl, or a substituted or unsubstituted C₂₋₂₀ heteroaryl containing a heteroatom of O or S.

9. The compound of claim 1, wherein:
Ar is phenyl, biphenylyl, terphenylyl, naphthyl, phenanthryl, triphenylenyl, dibenzofuranyl, dibenzothiophenyl, benzonaphthofuranyl, or benzonaphthothiophenyl,
wherein the Ar is unsubstituted, or substituted with one or more substituents selected from the group consisting of deuterium, halogen, cyano, phenyl substituted or unsubstituted with deuterium, biphenylyl substituted or unsubstituted with deuterium, and naphthyl substituted or unsubstituted with deuterium.

10. The compound of claim 1, wherein:
R₁ and R₂ are hydrogen, or deuterium.

11. The compound of claim 1, wherein:
the compound is represented by any one of the following Chemical Formulas 1-1 to 1-9: 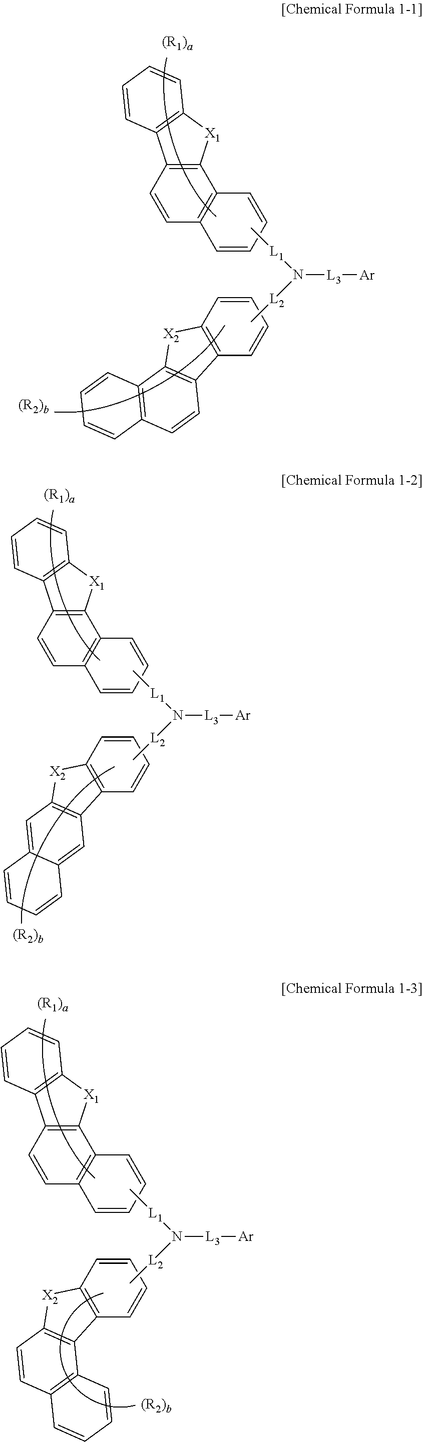 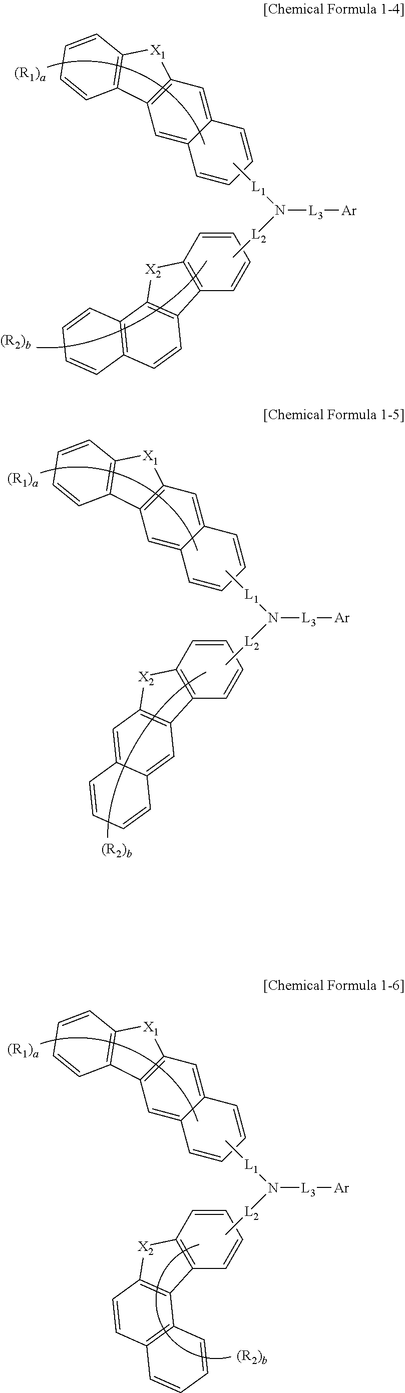 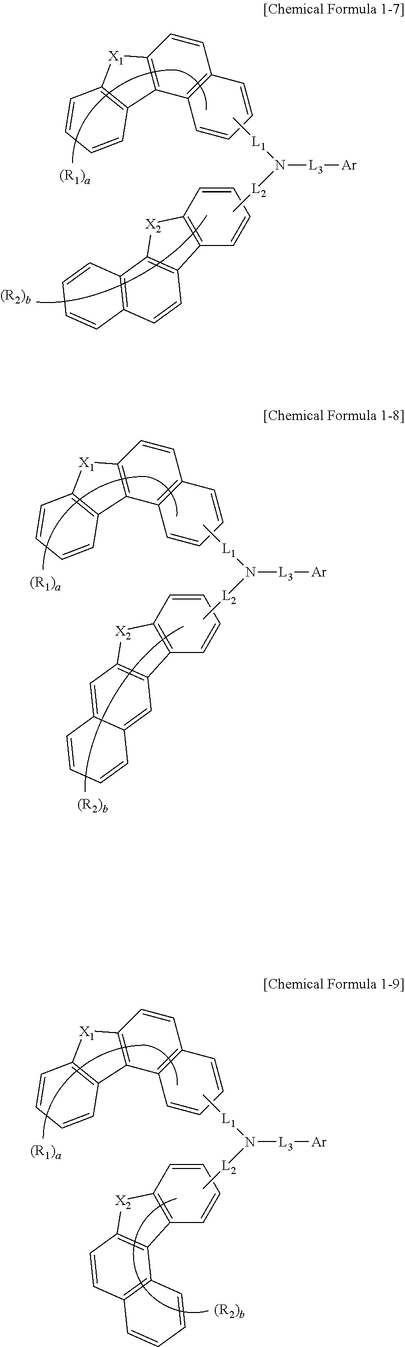
wherein in Chemical Formulas 1-1 to 1-9,
X₁, X₂, L₁ to L₃, Ar, R₁, R₂, a and b are as defined in claim 1.

12. The compound of claim 1, wherein:
the compound is any one compound selected from among the following compounds: 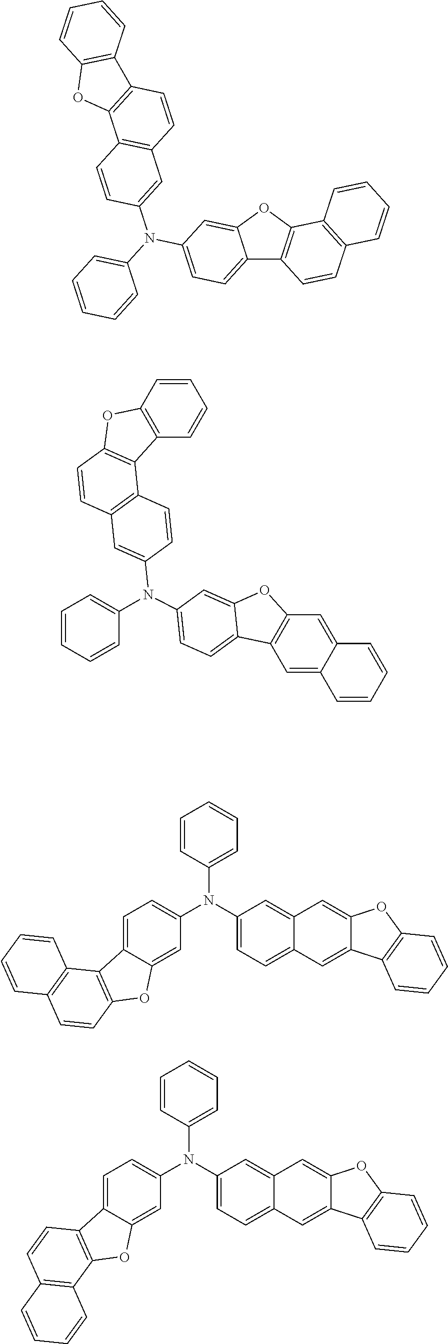 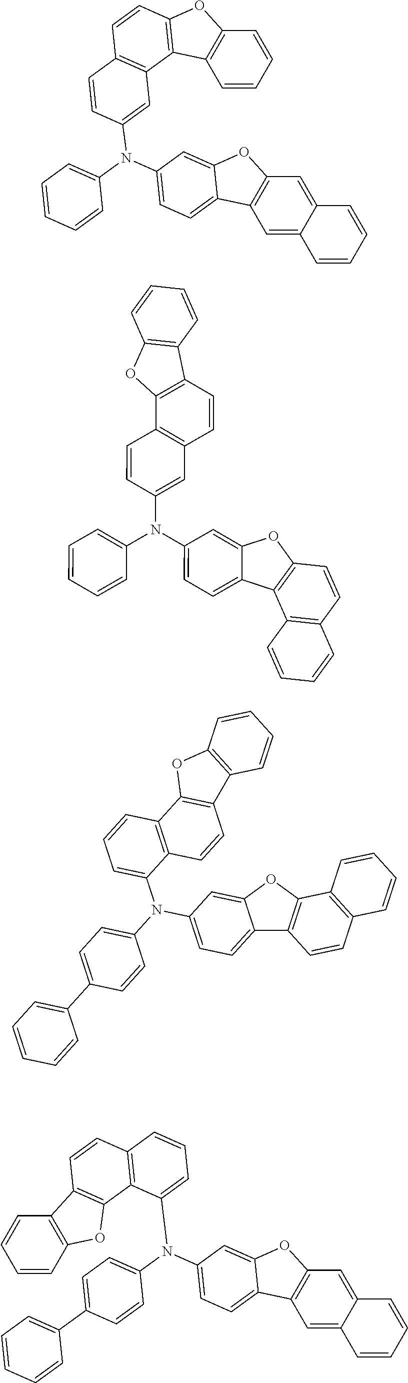 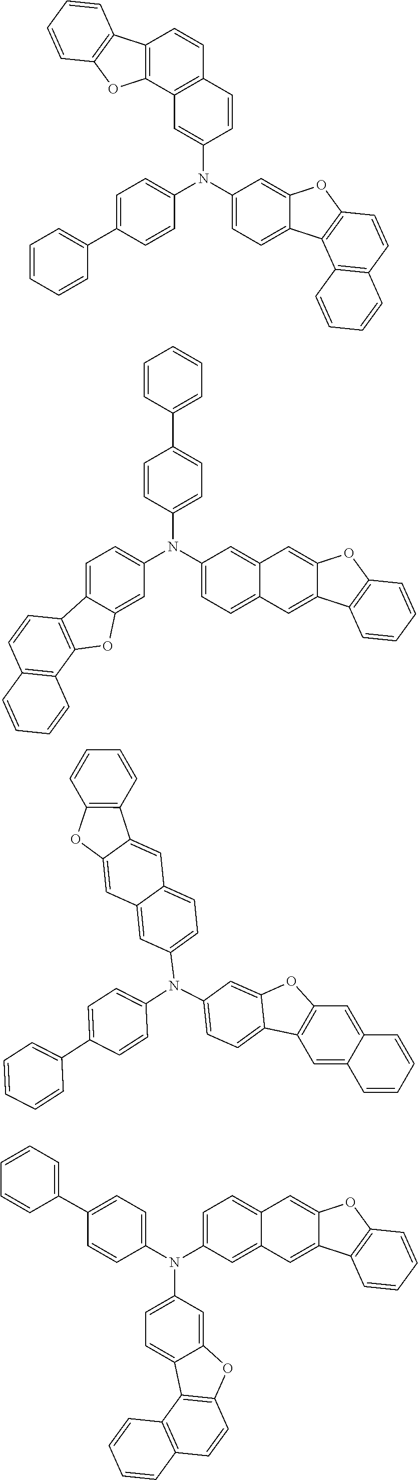 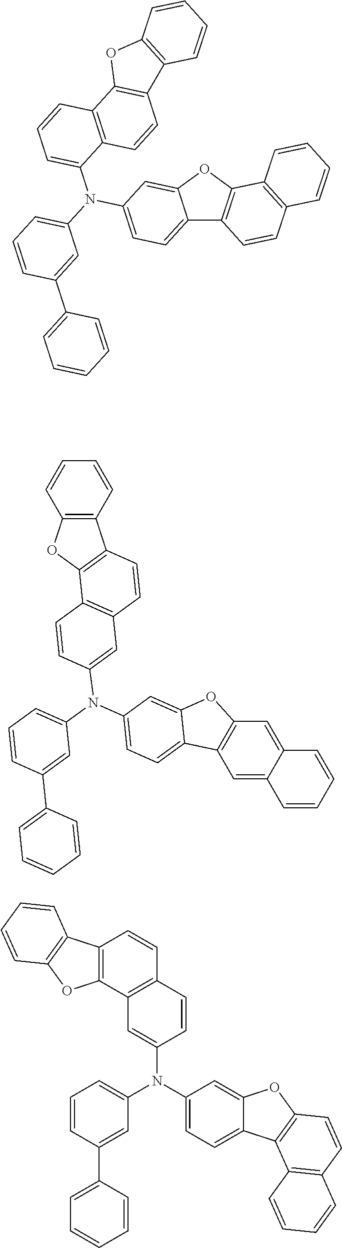 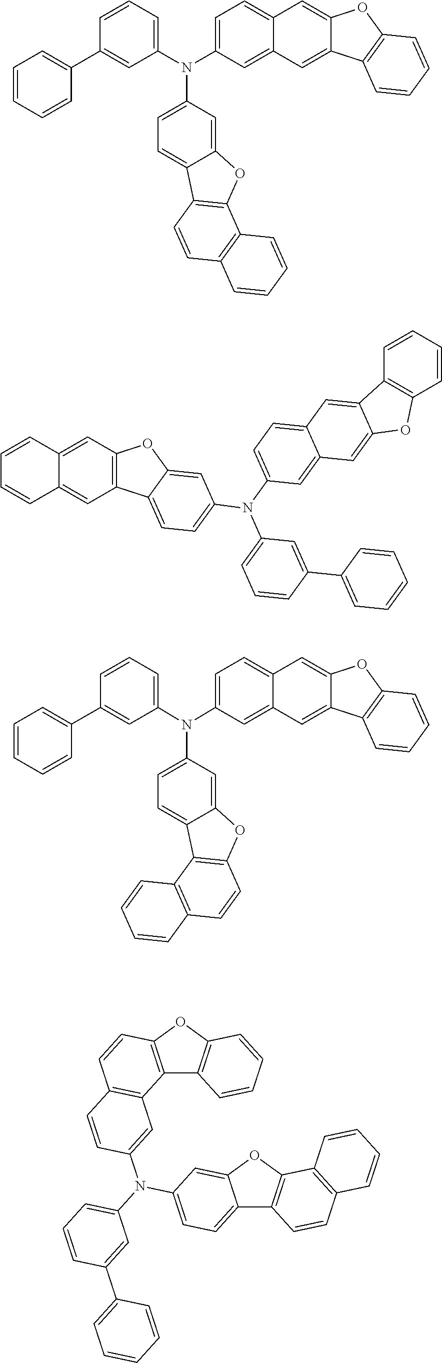 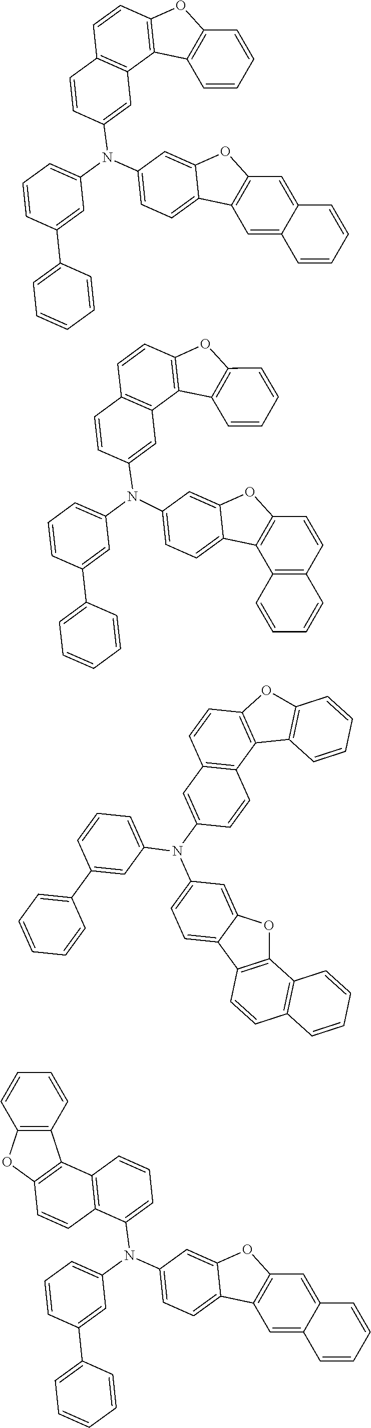 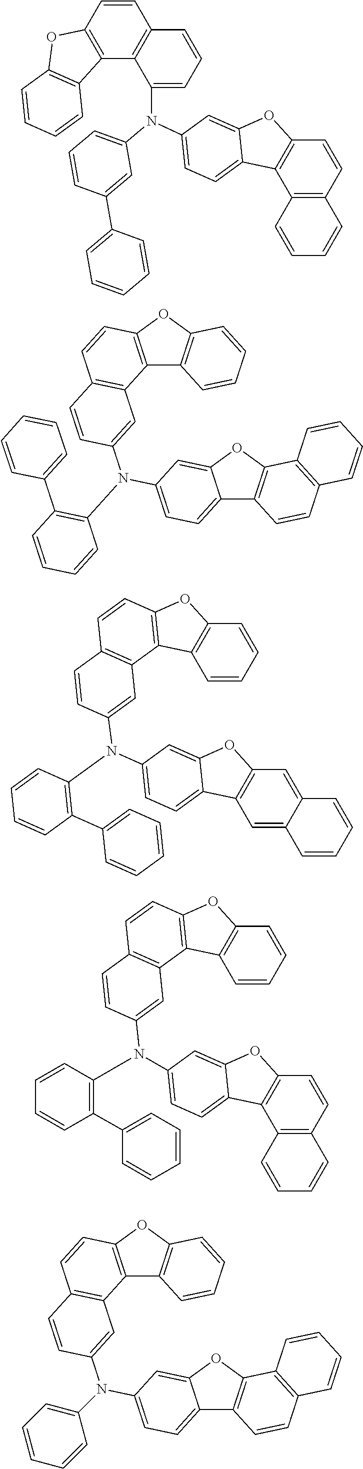 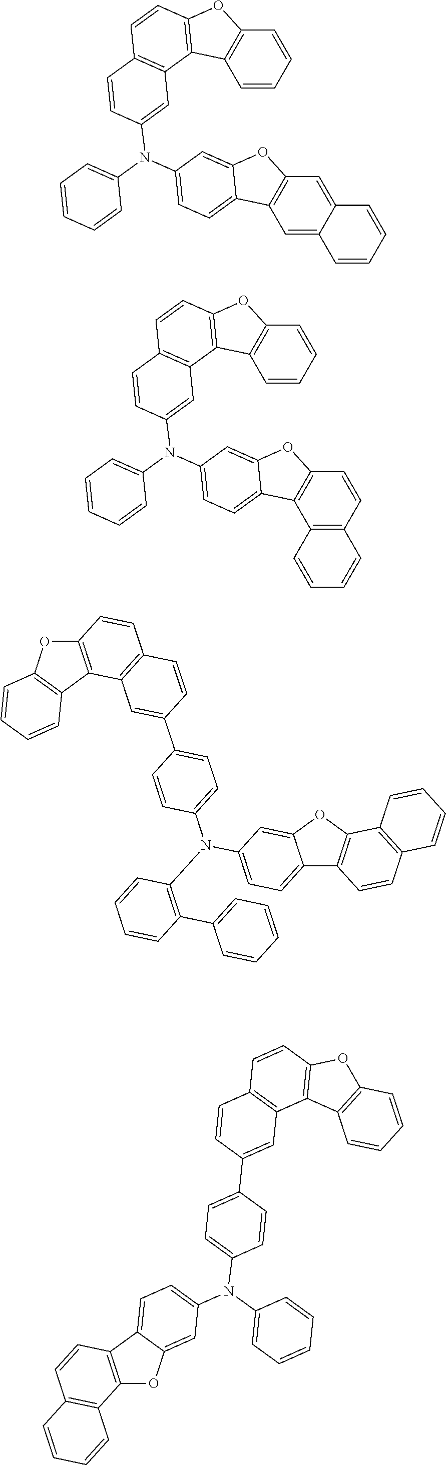 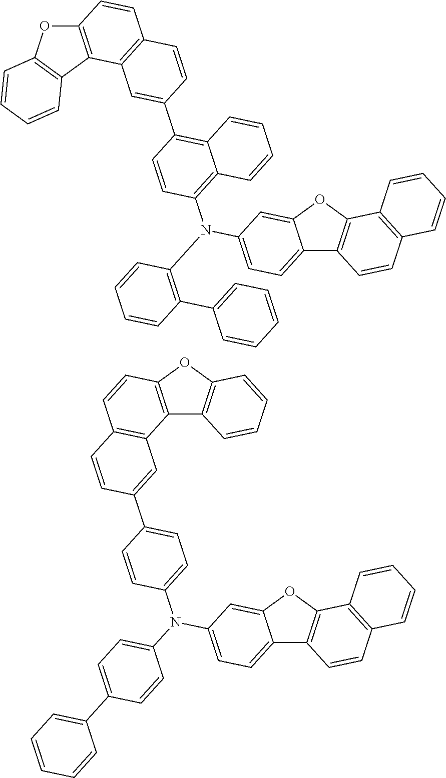 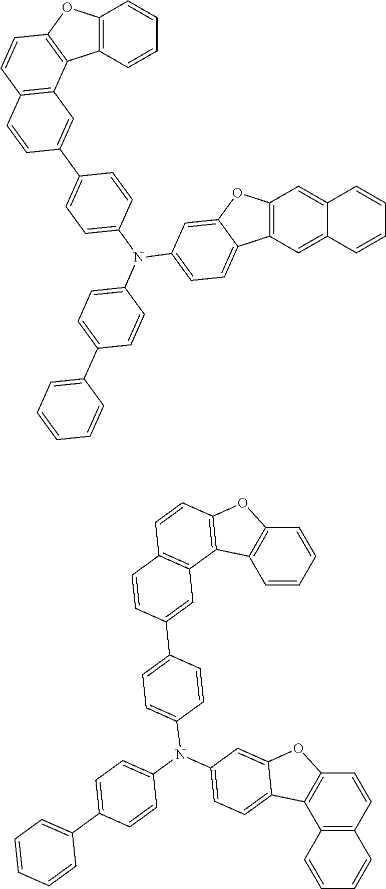 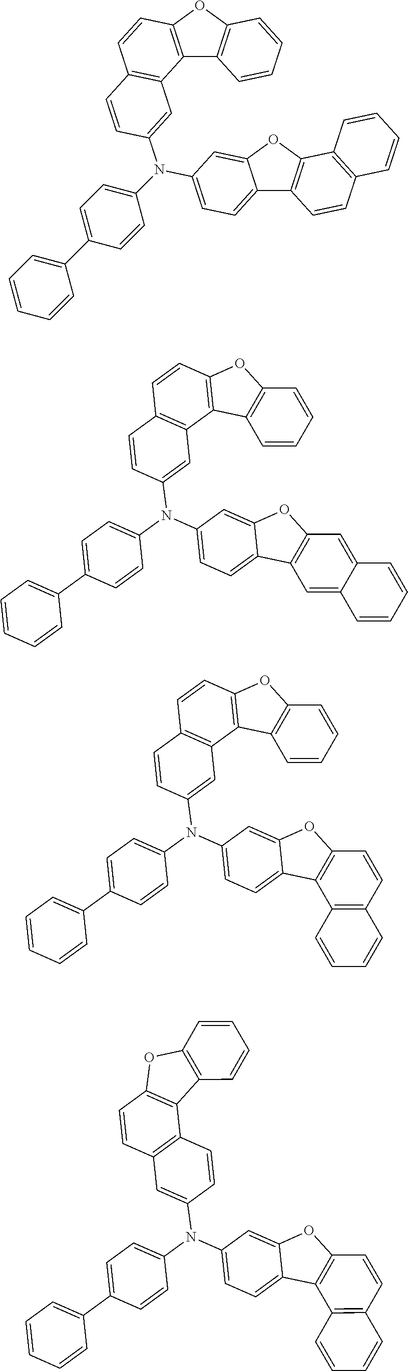 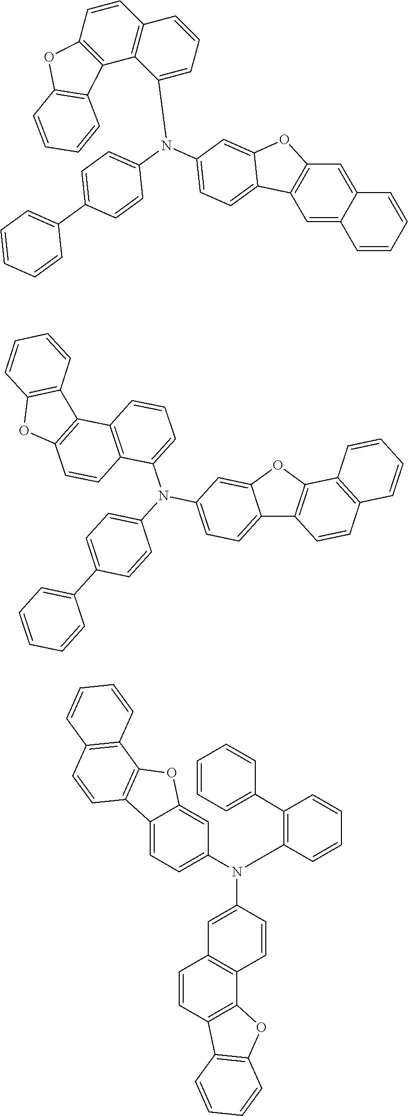 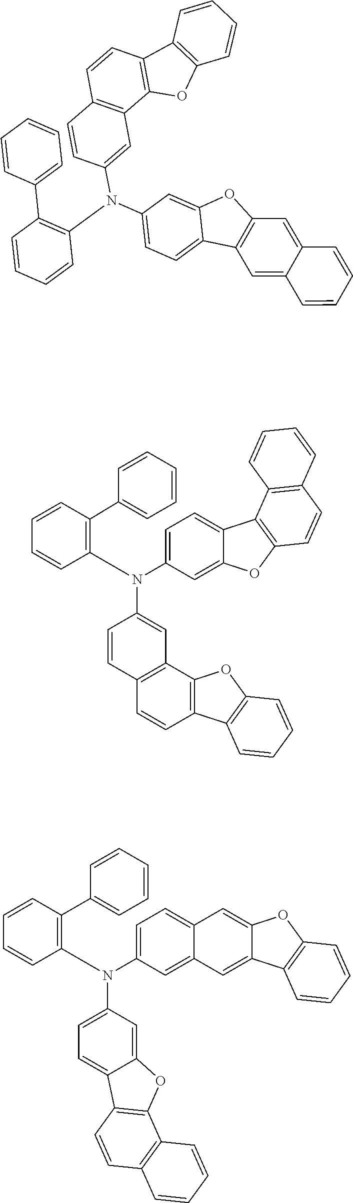 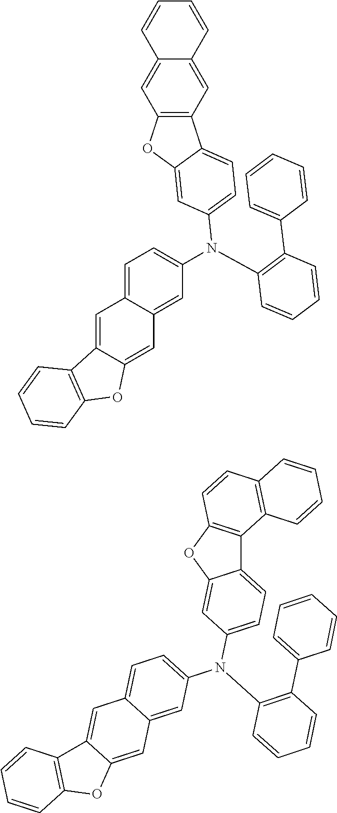 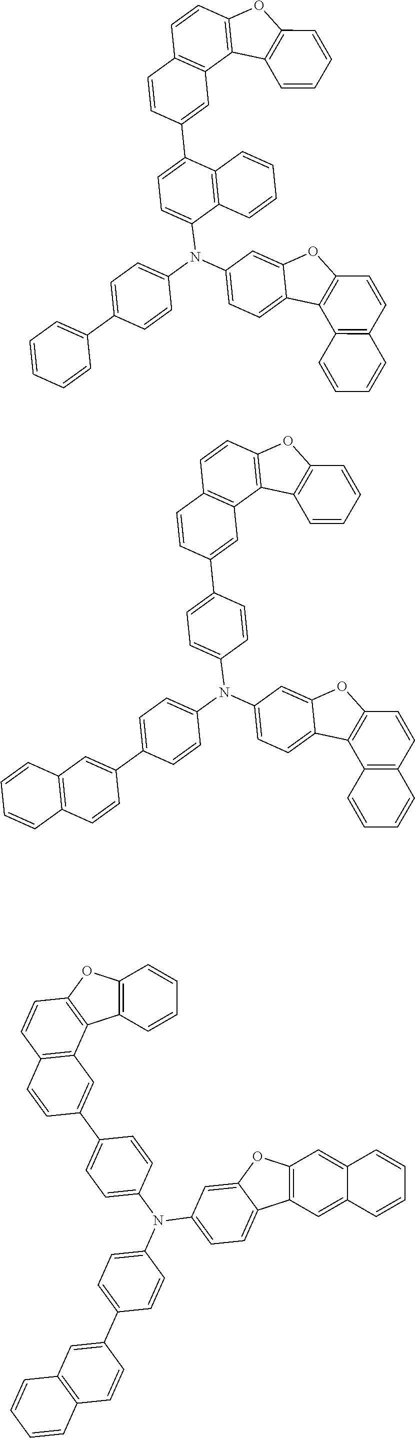 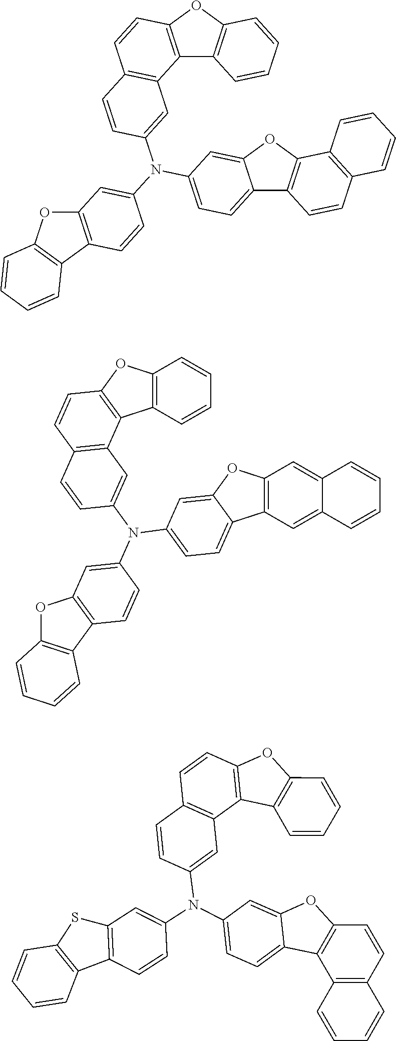 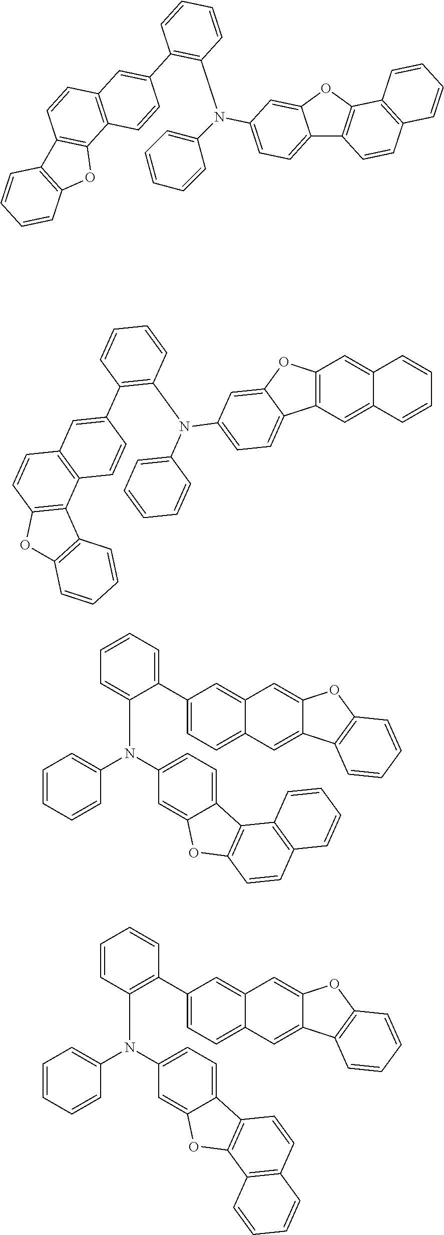 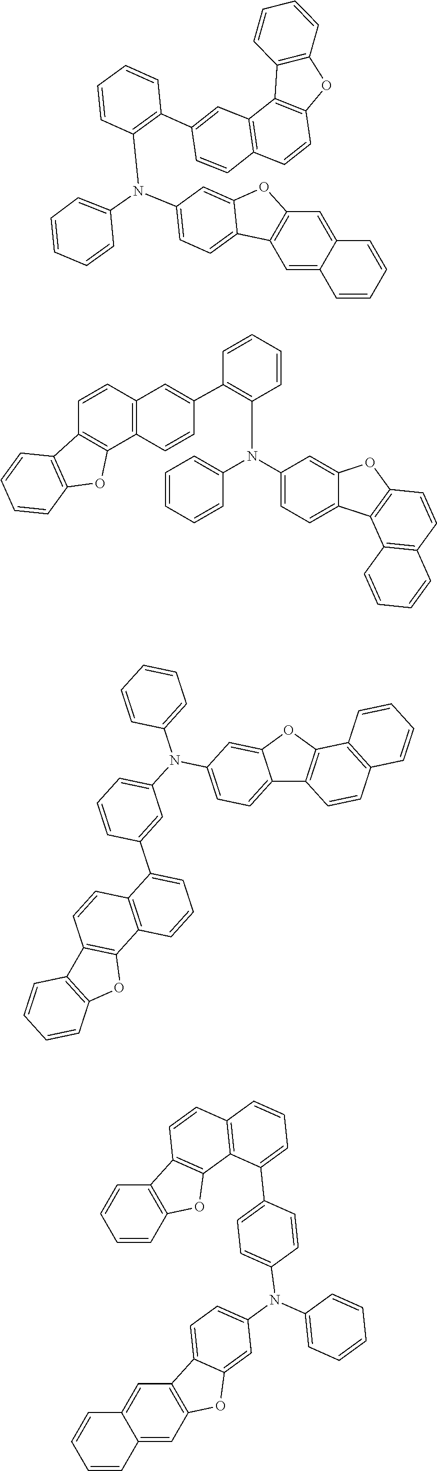 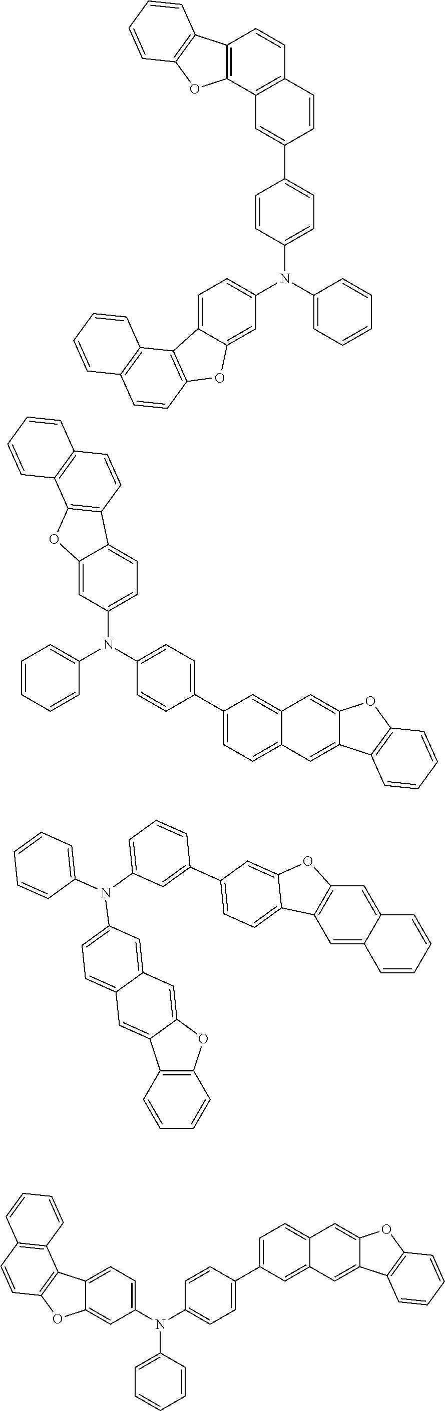 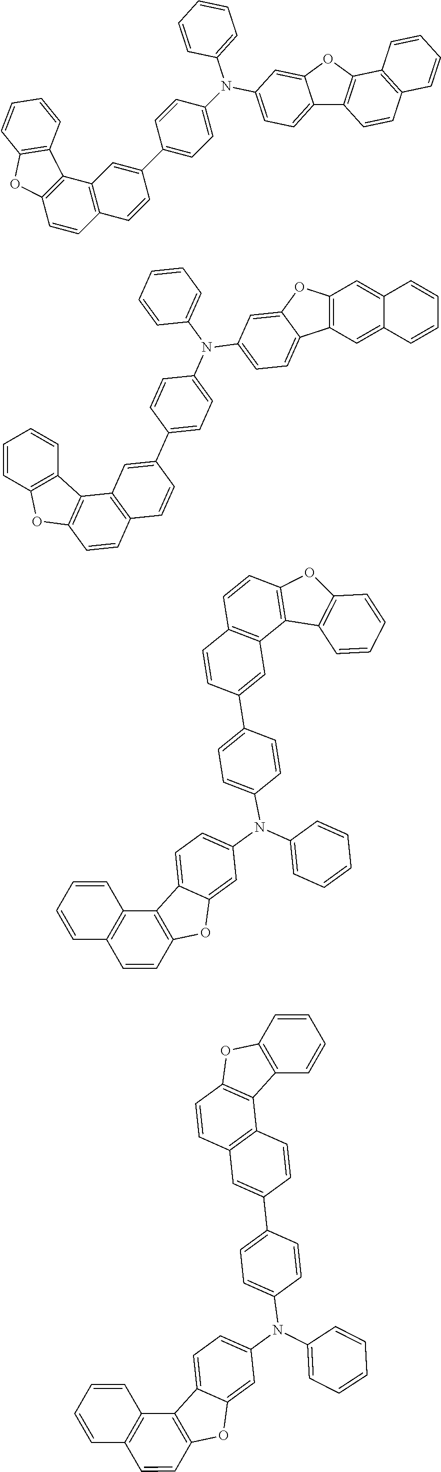 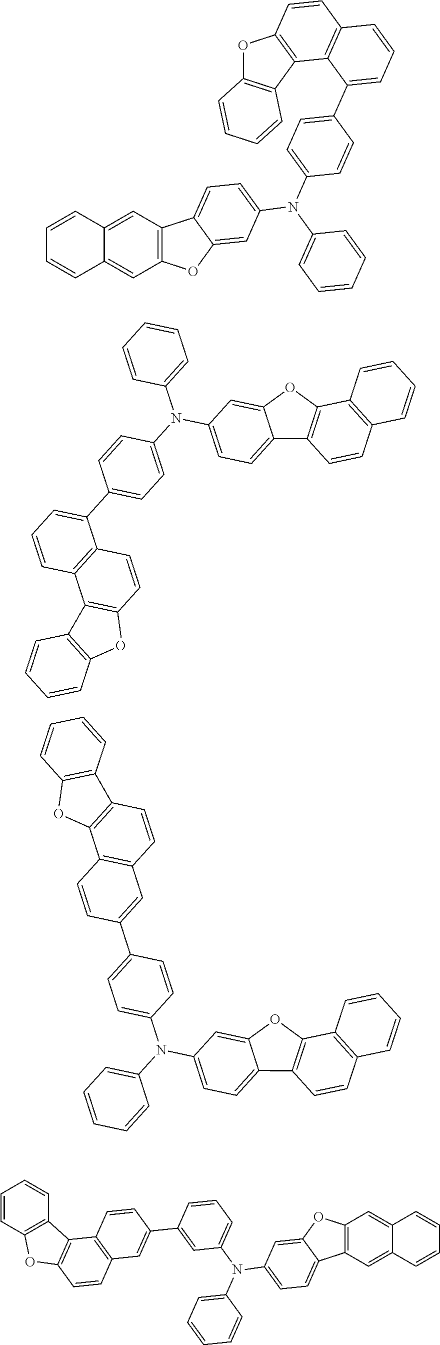 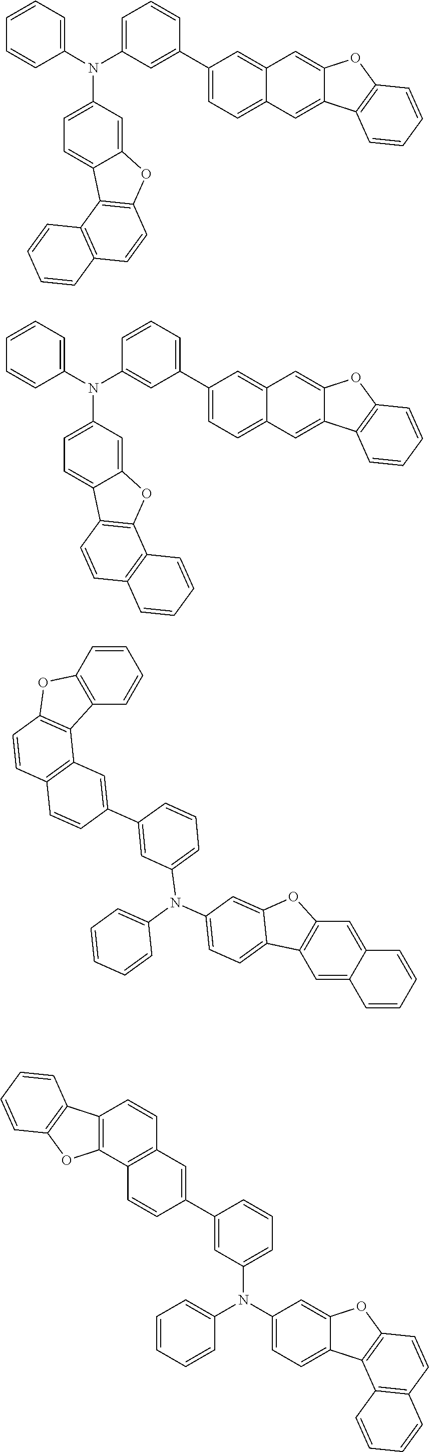 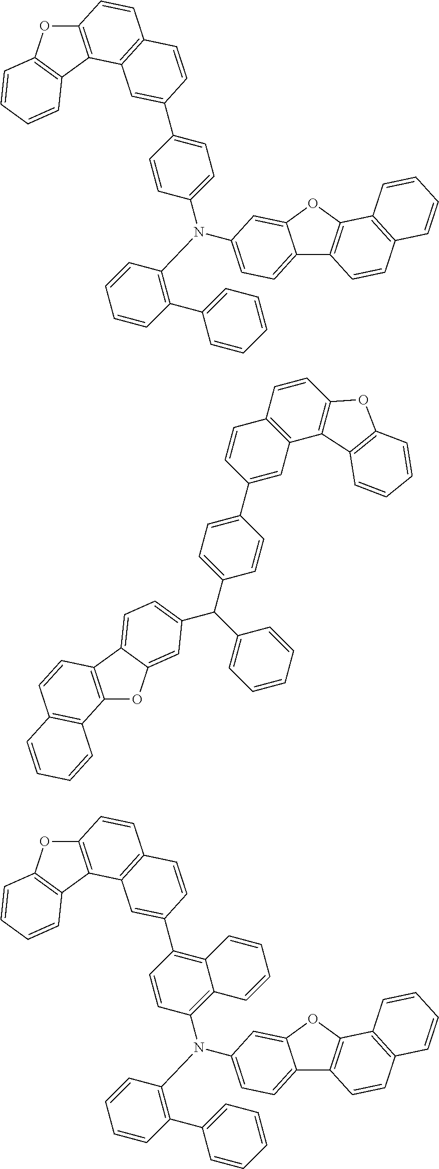 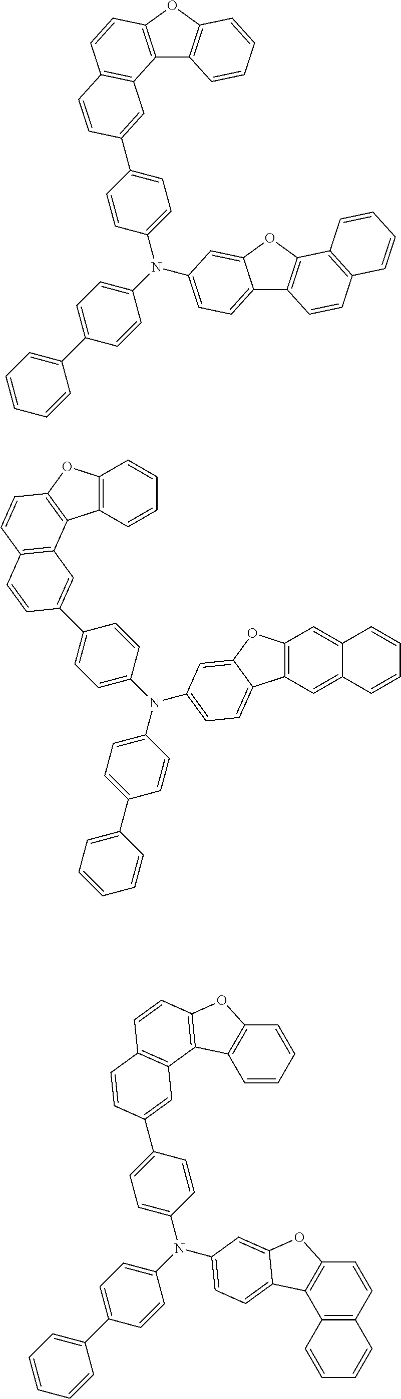 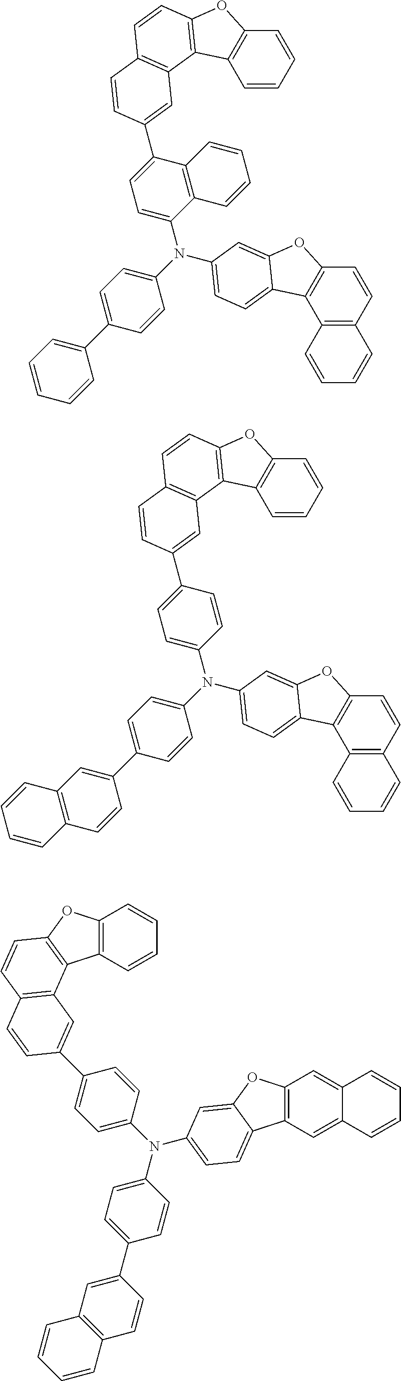 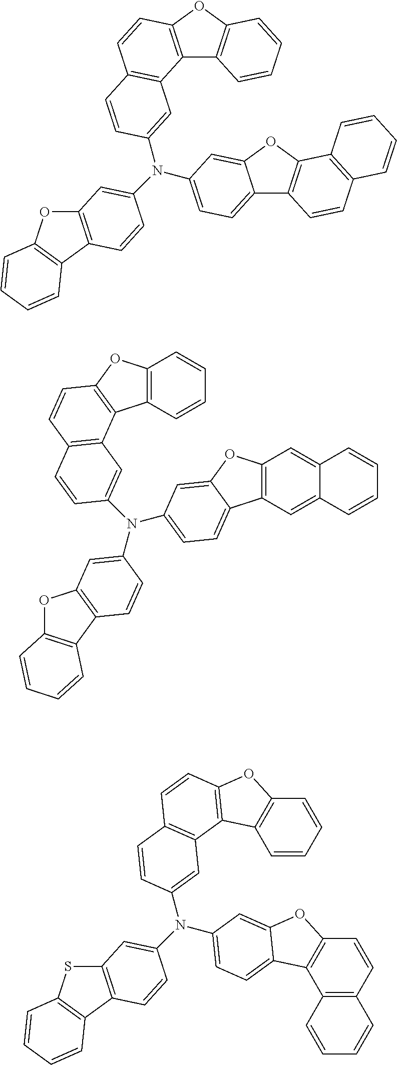 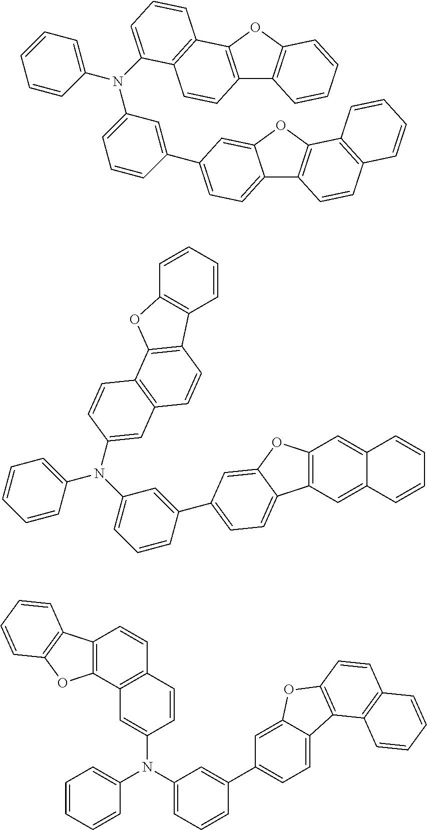 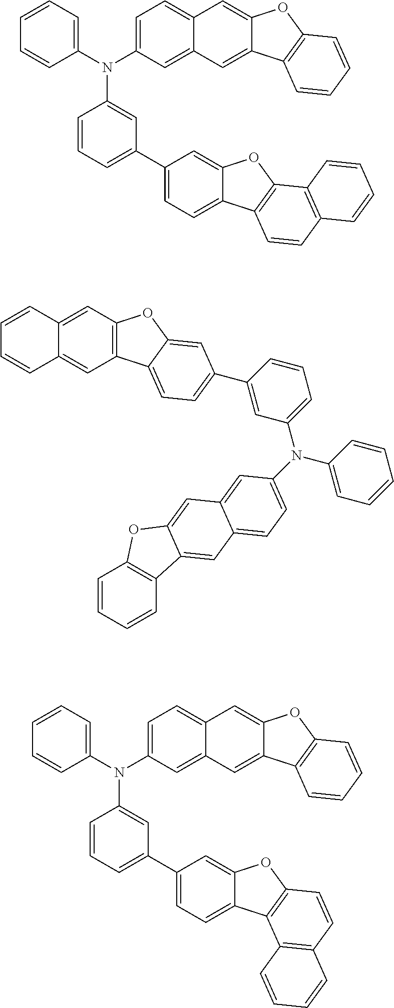 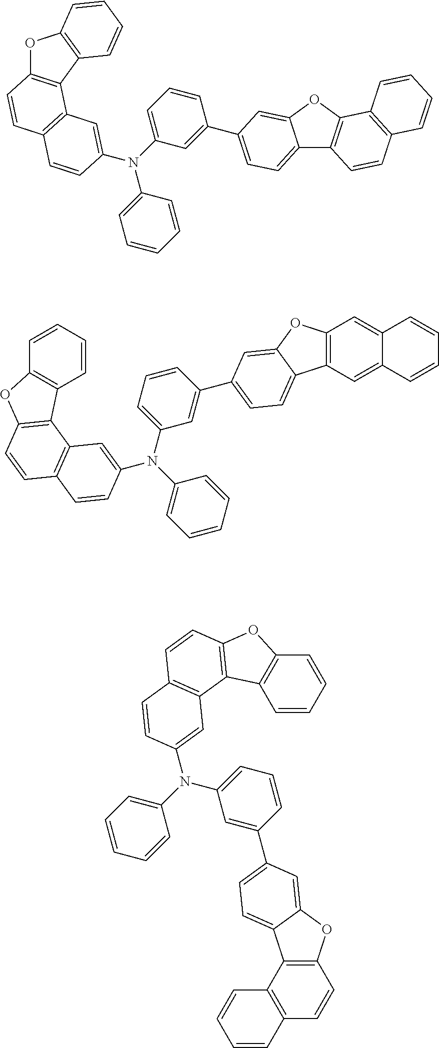 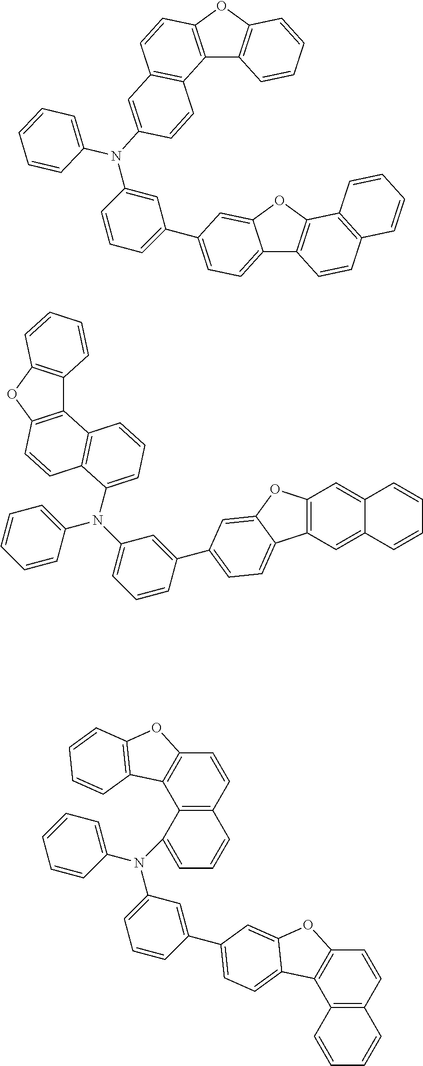 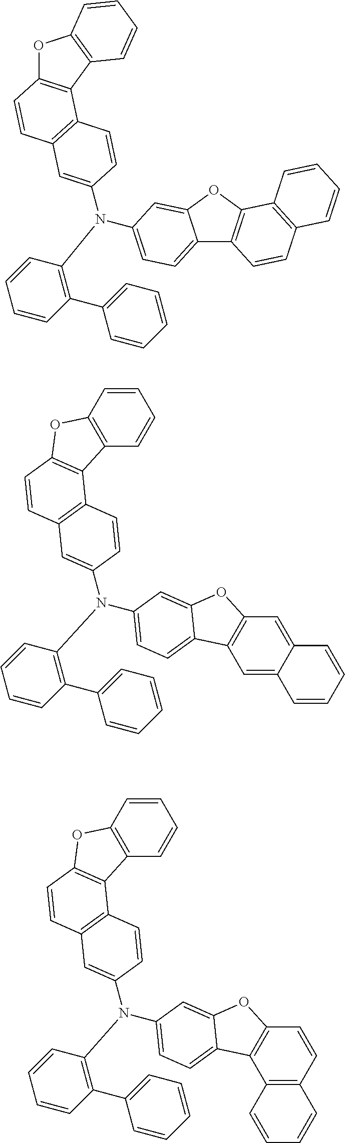 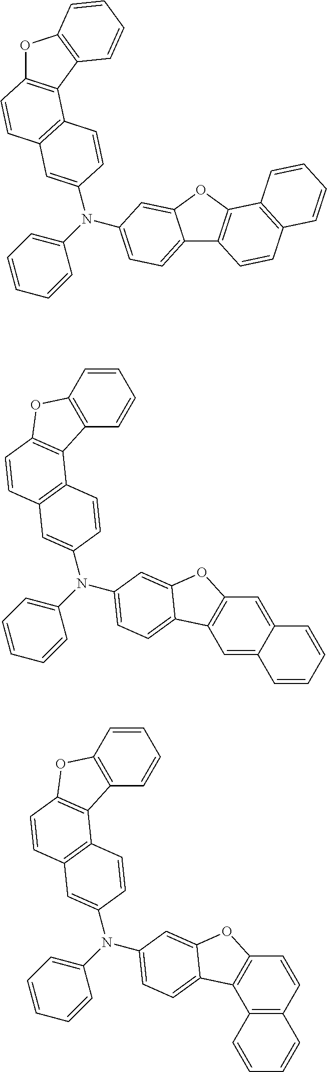 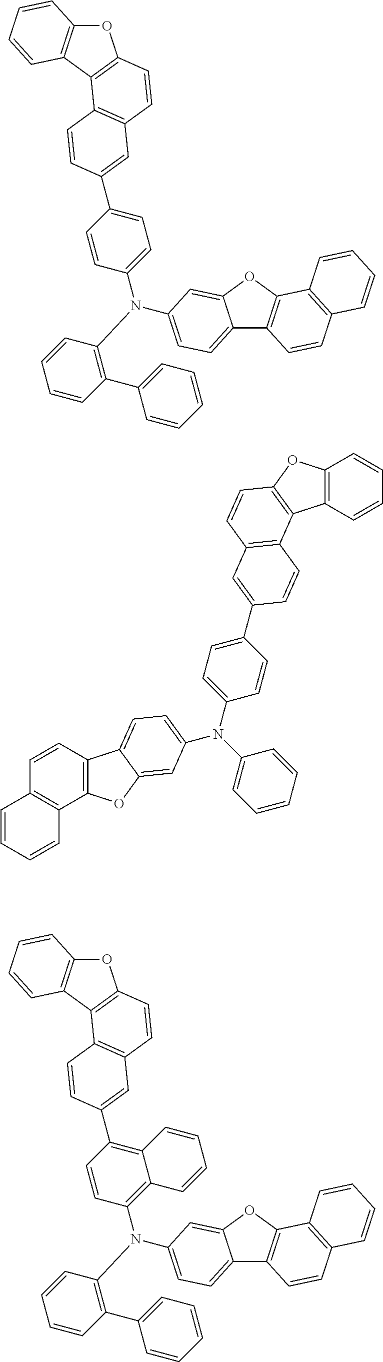 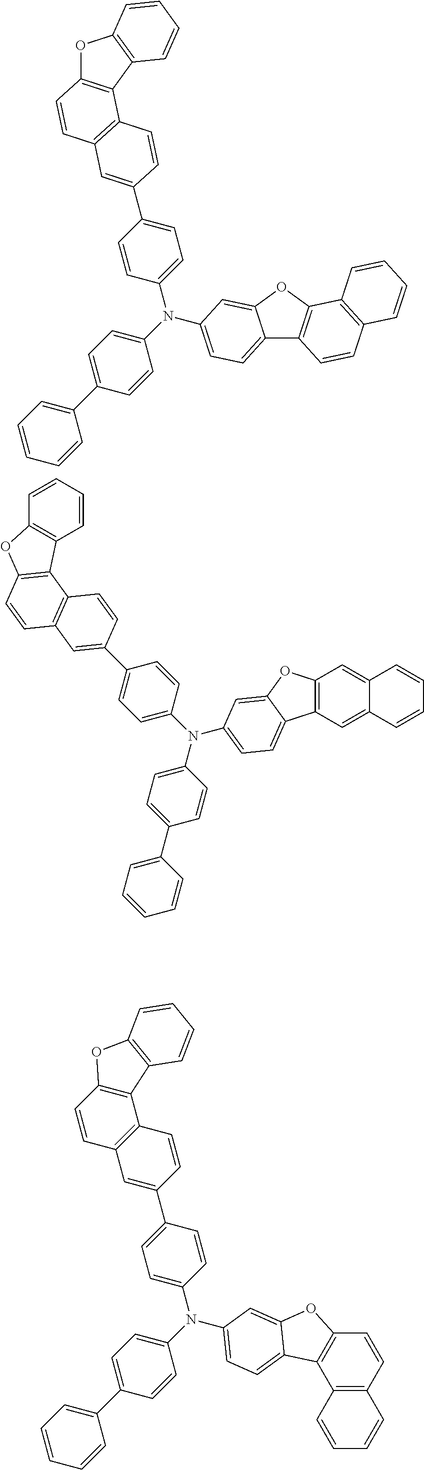 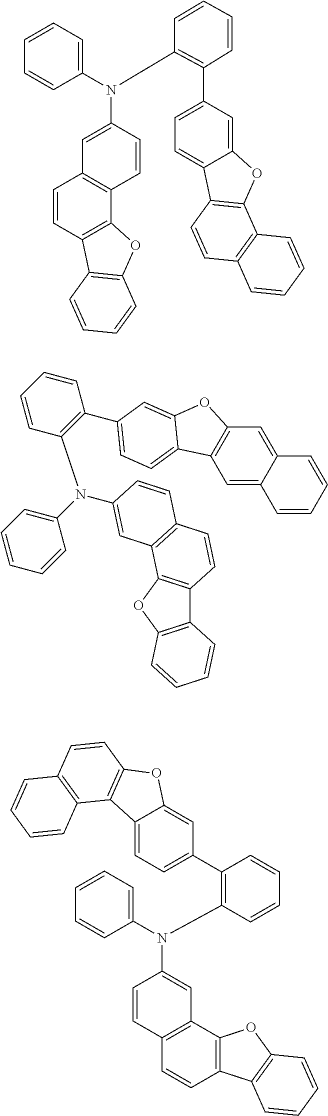 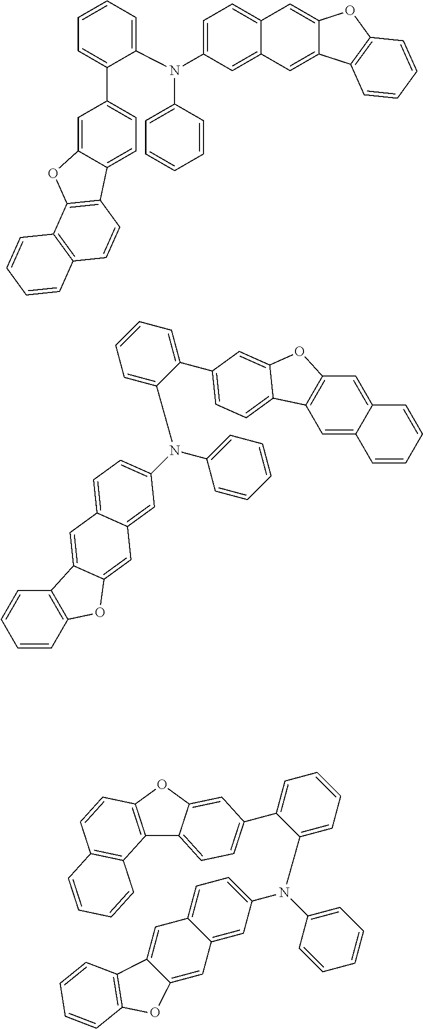 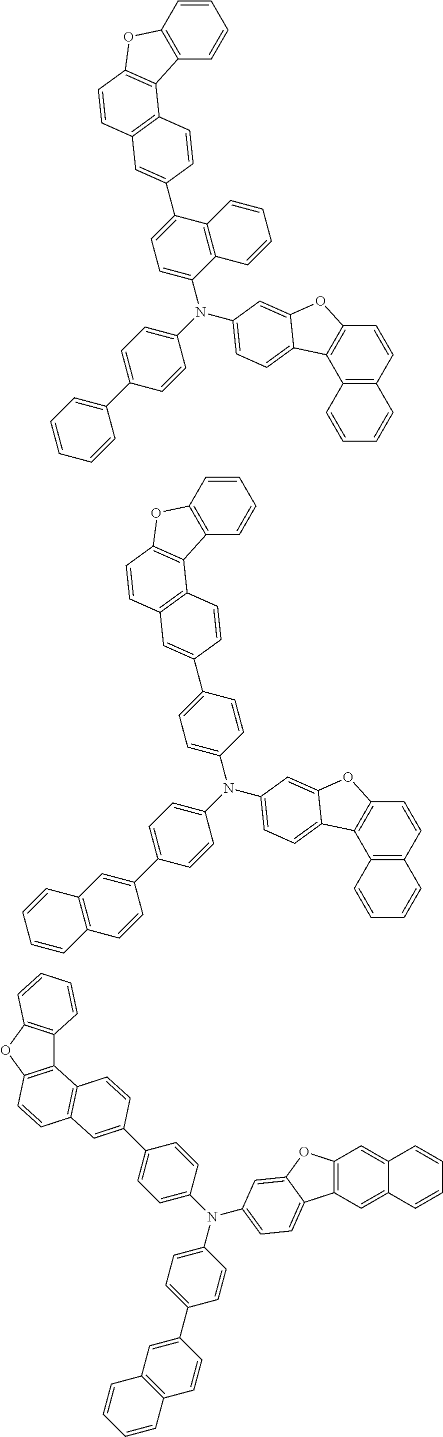 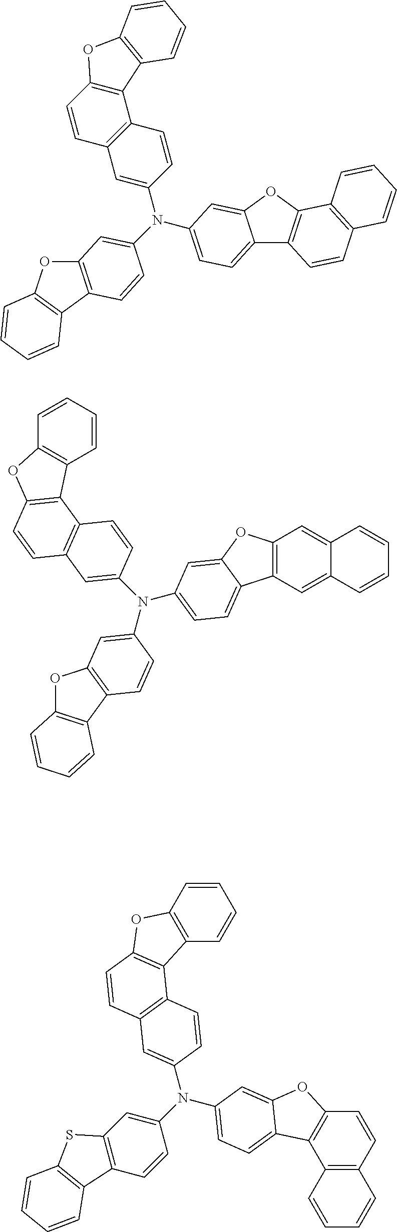 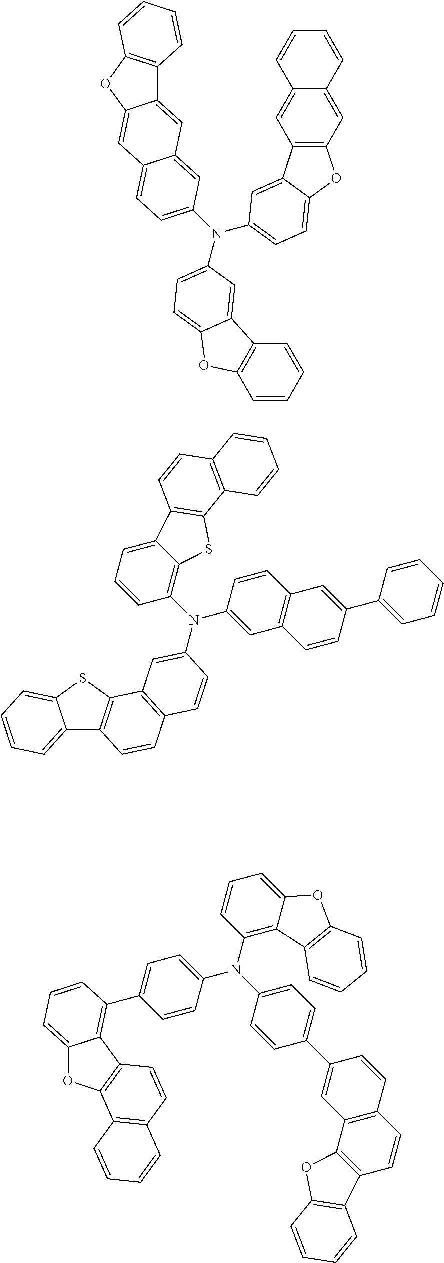 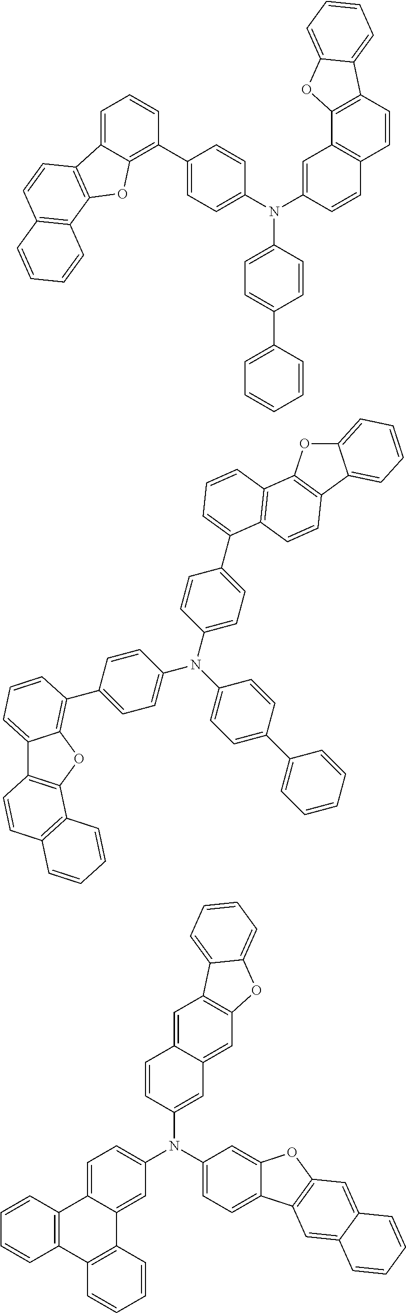 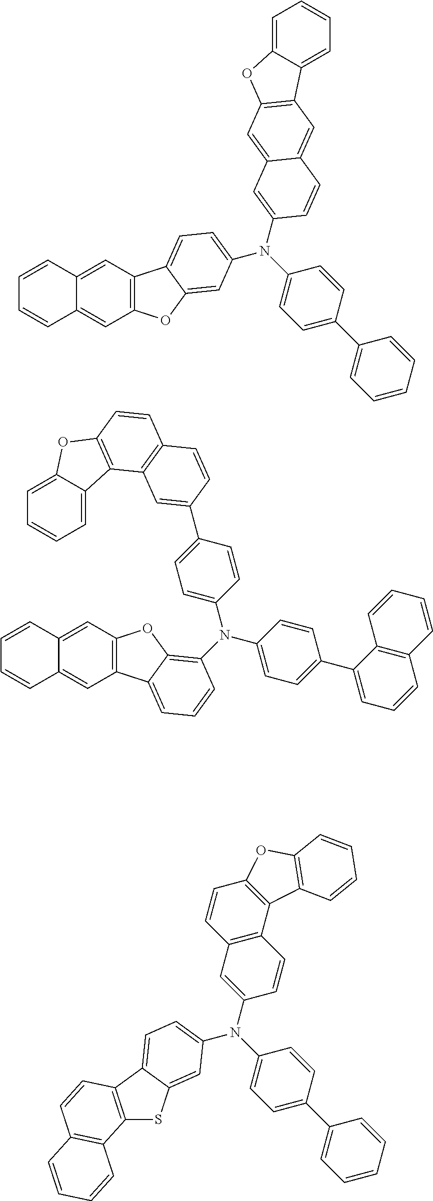 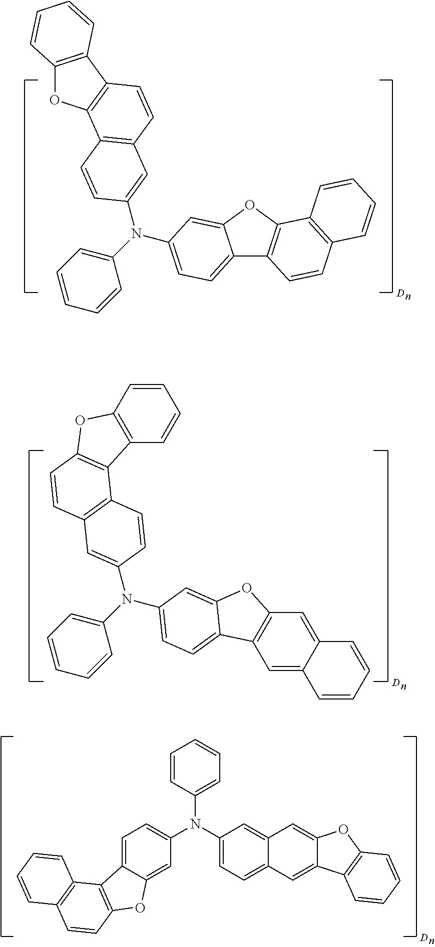 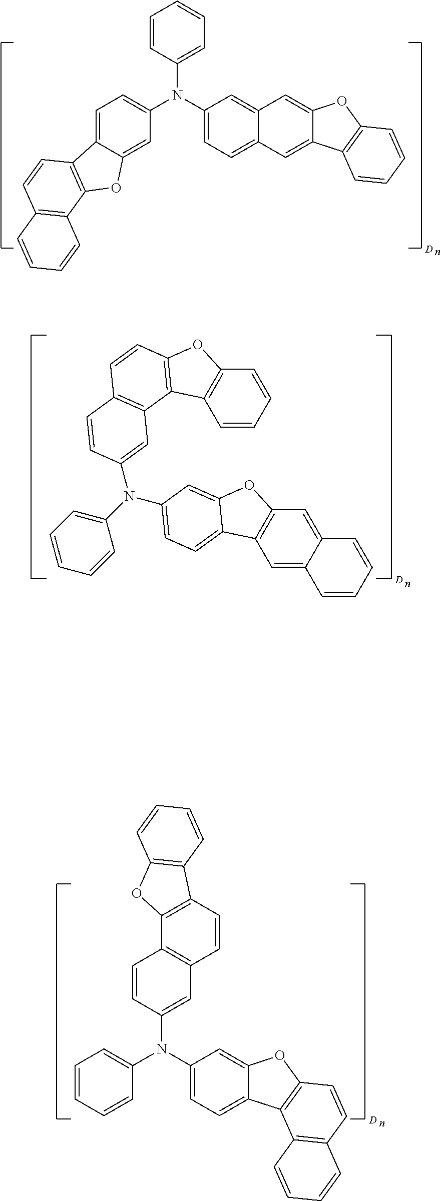 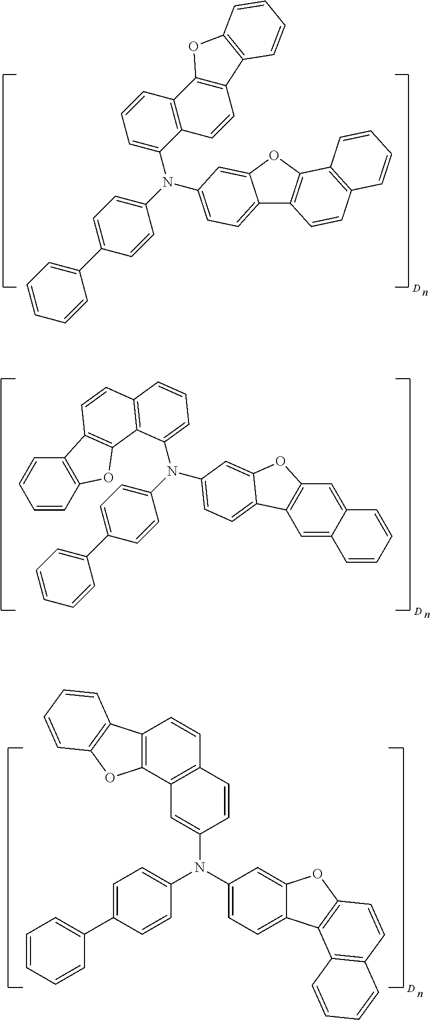 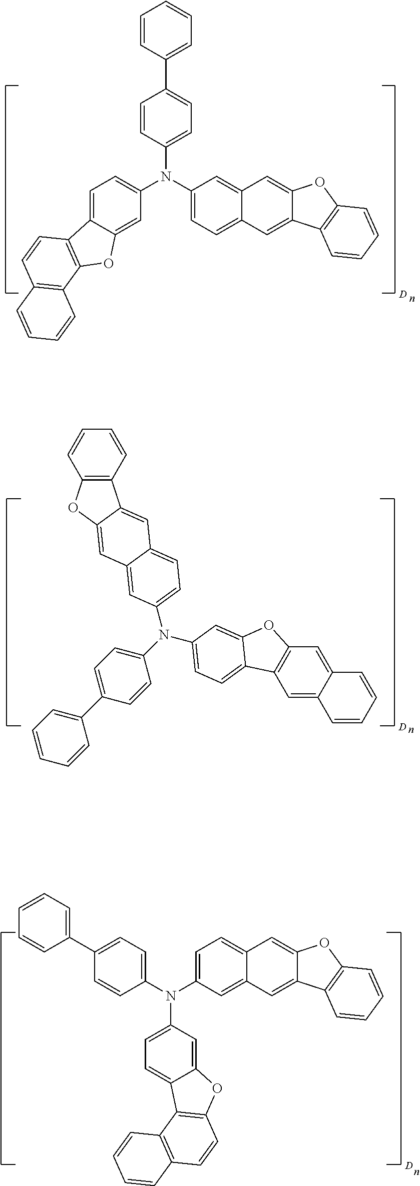 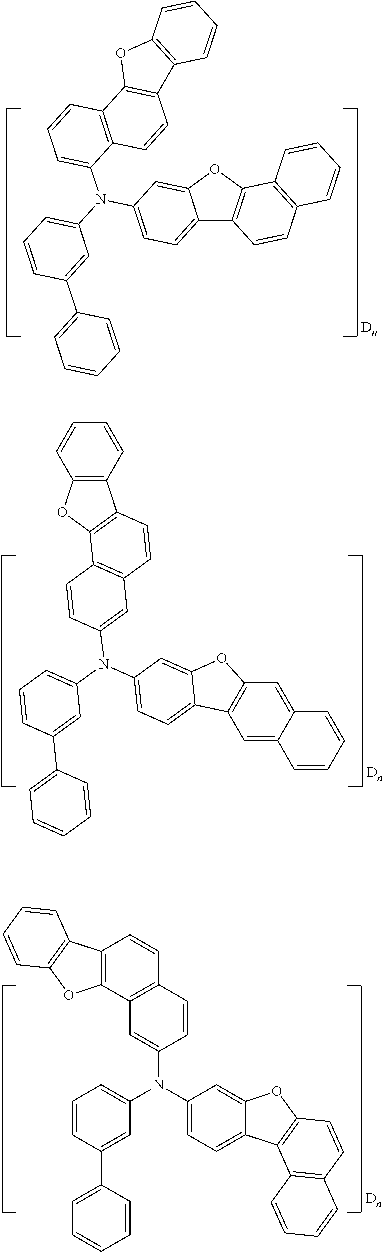 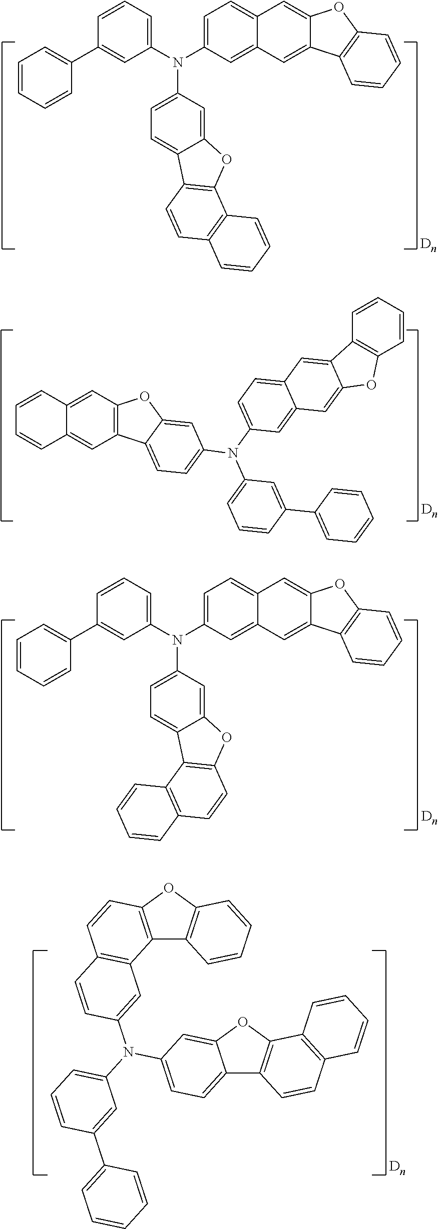 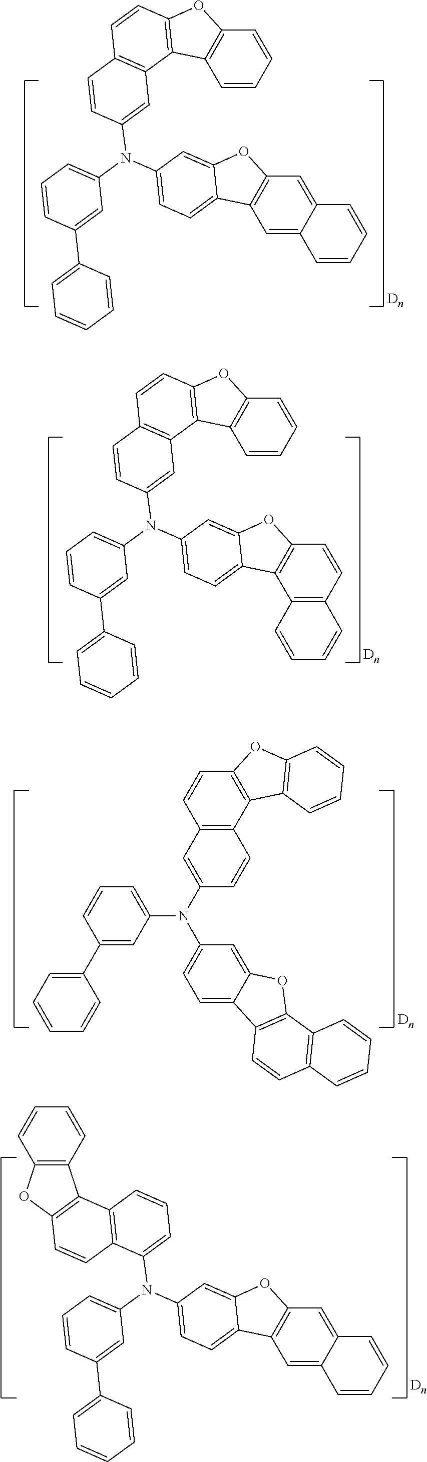 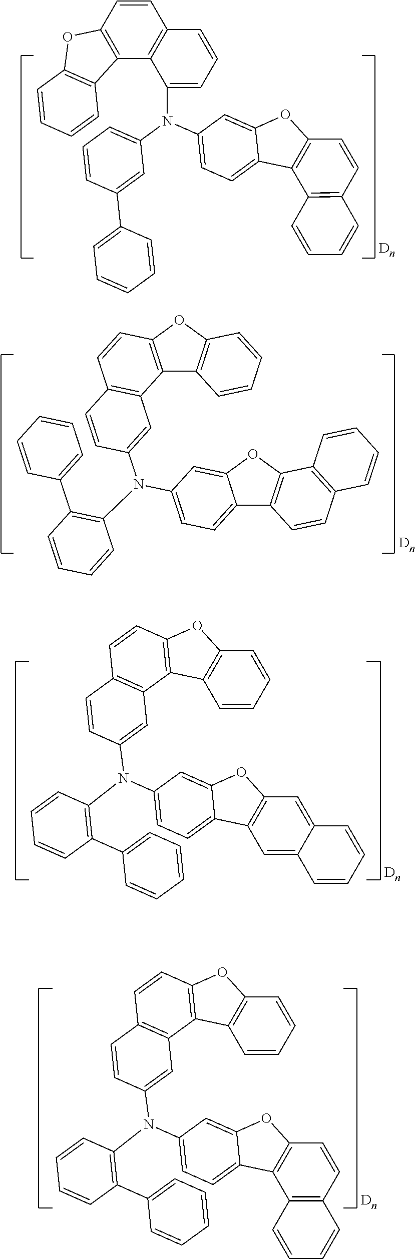 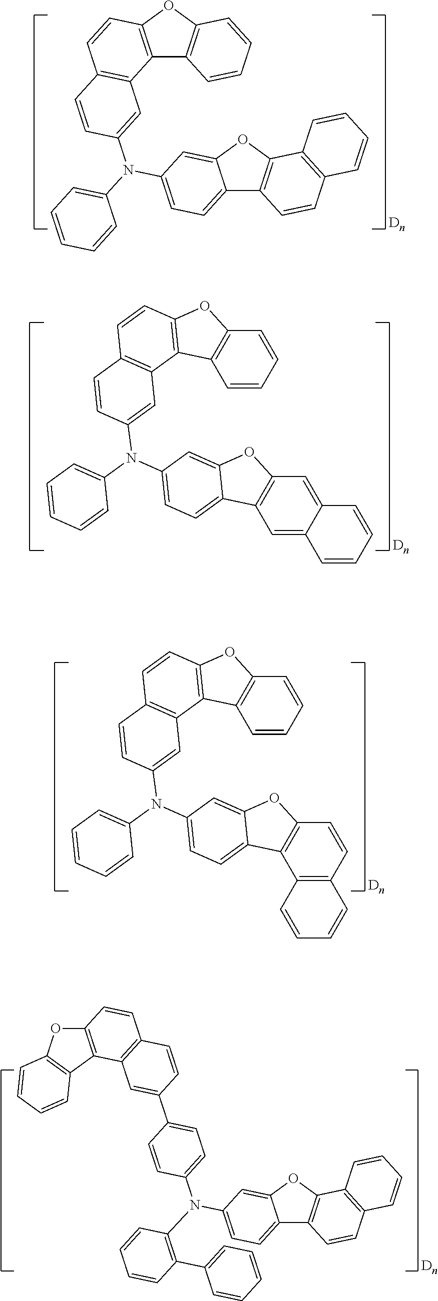 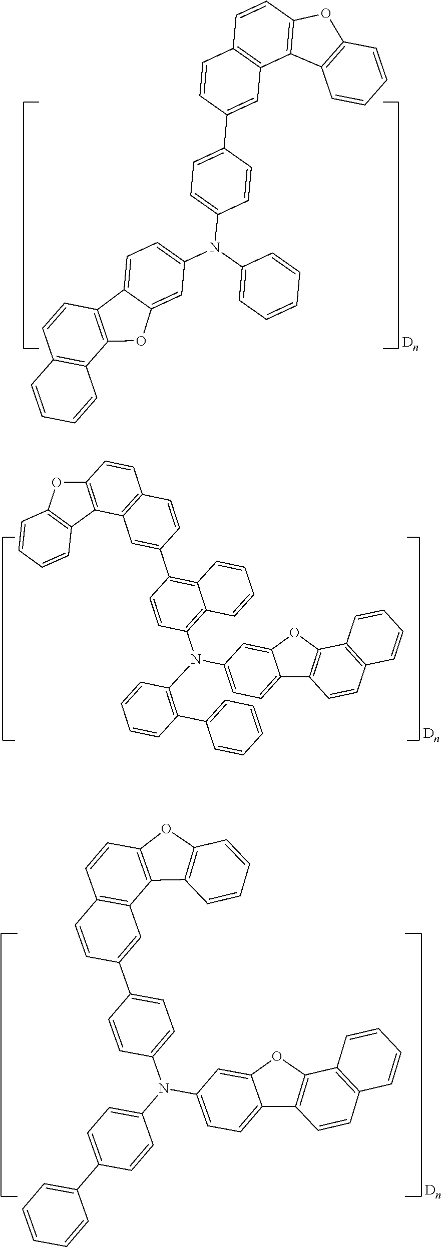 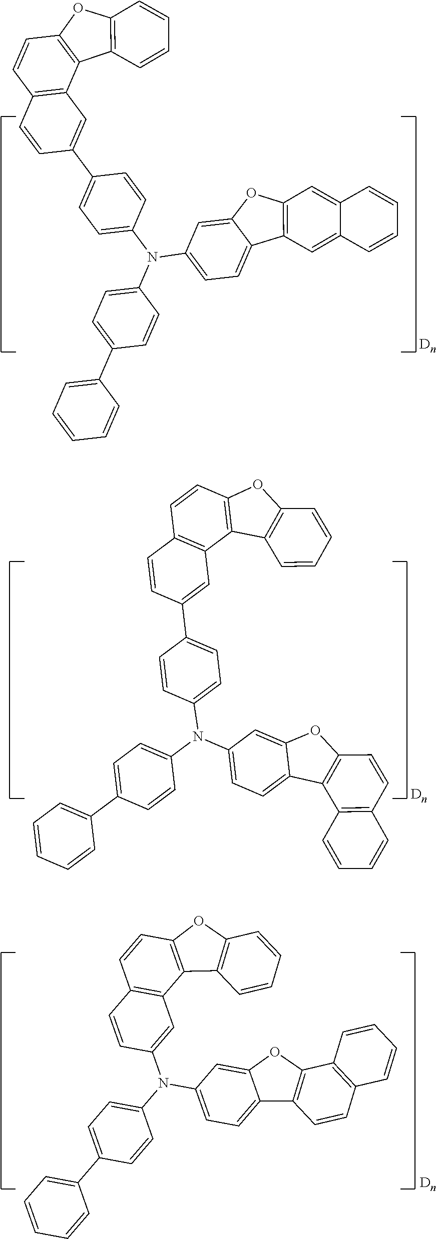 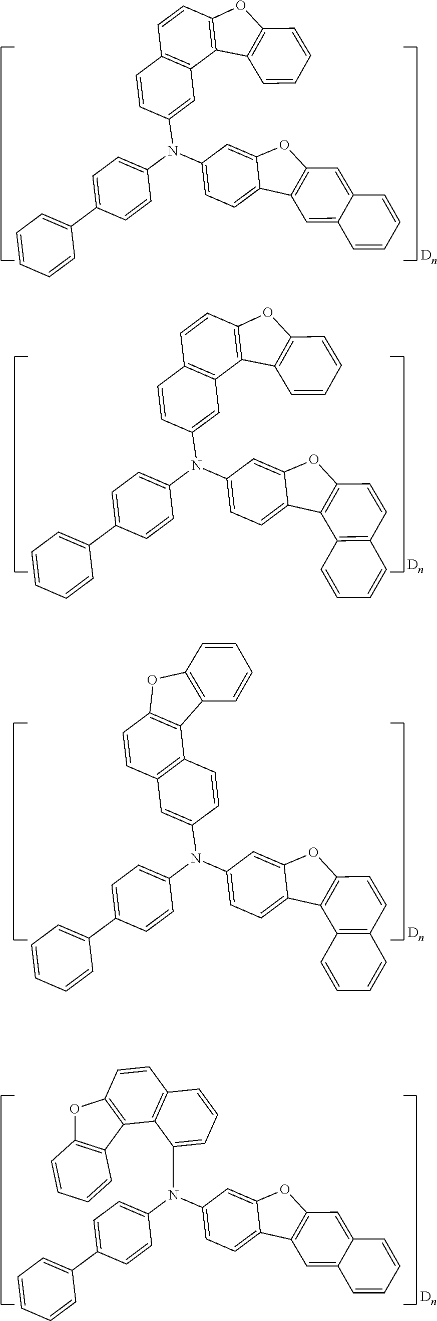 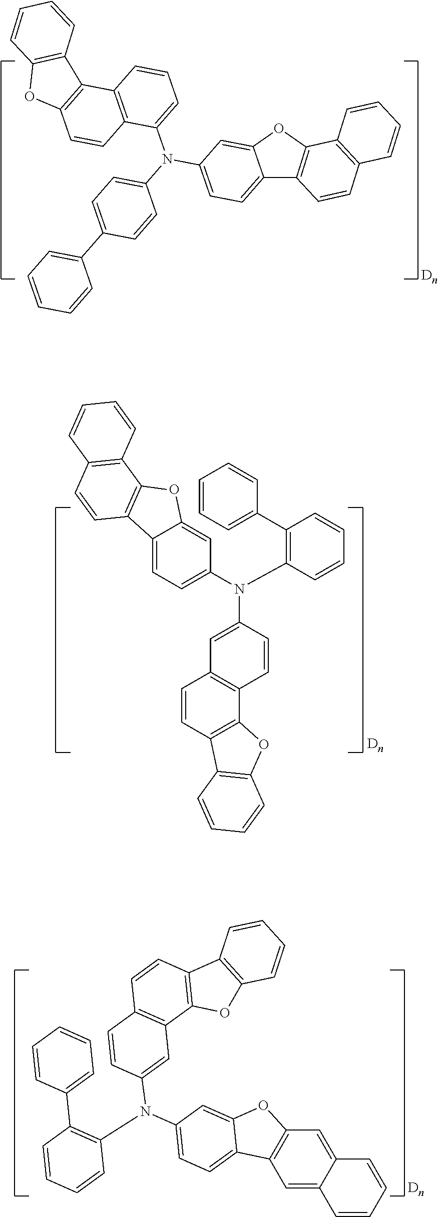 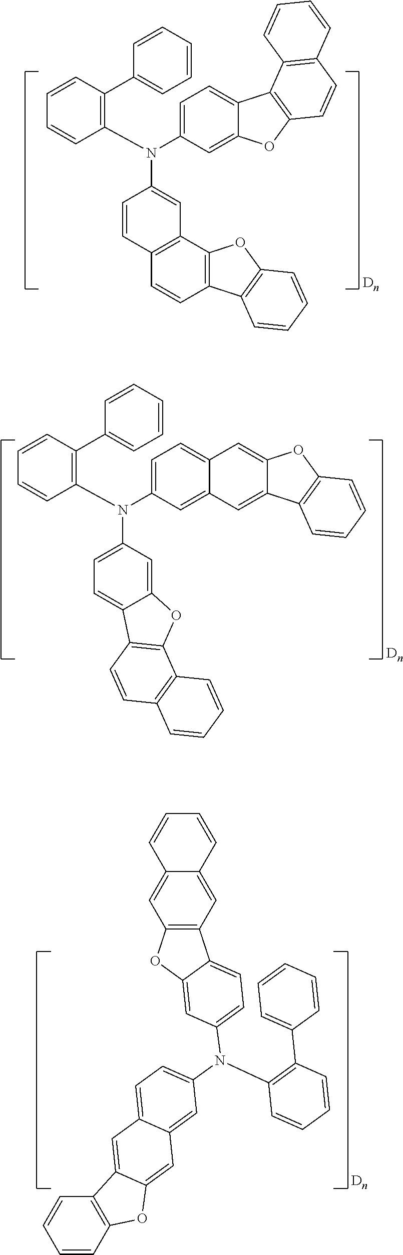 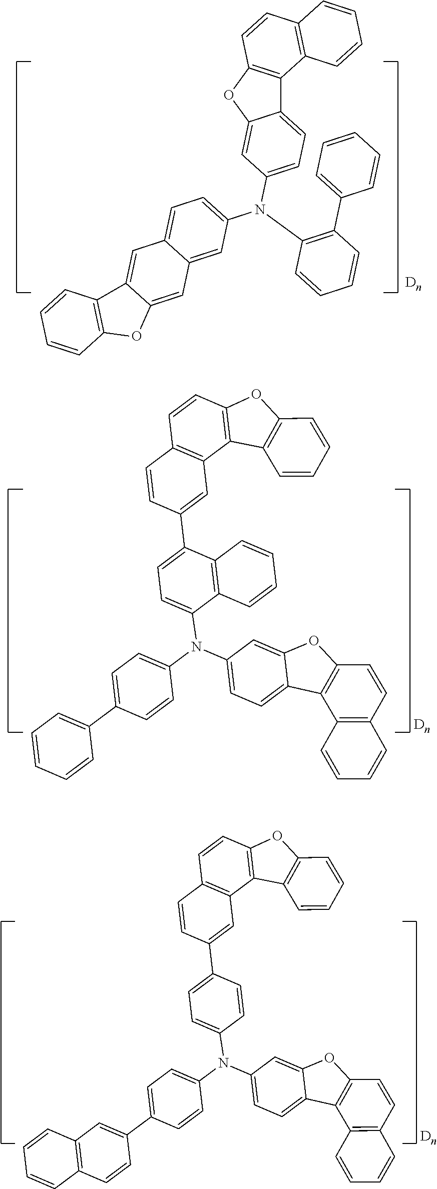 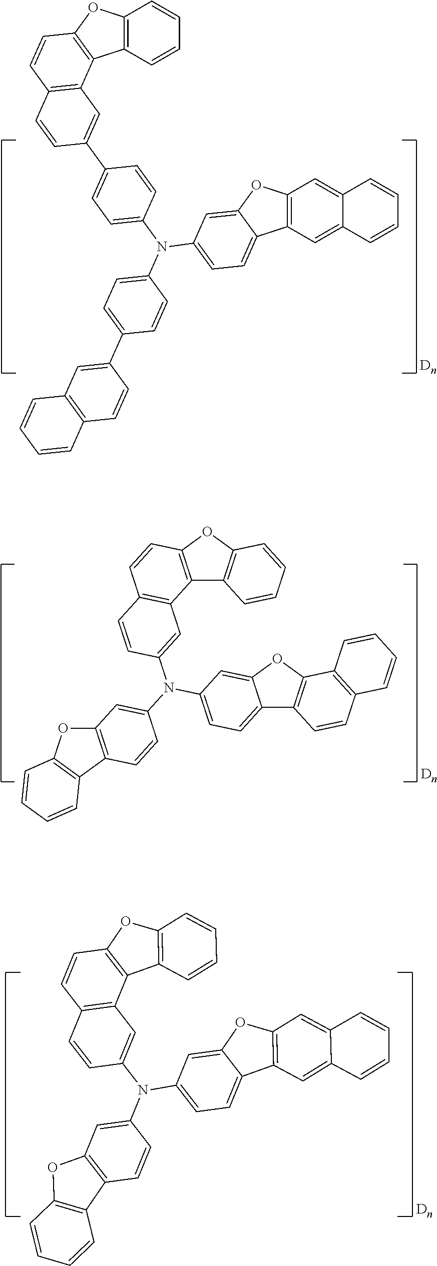 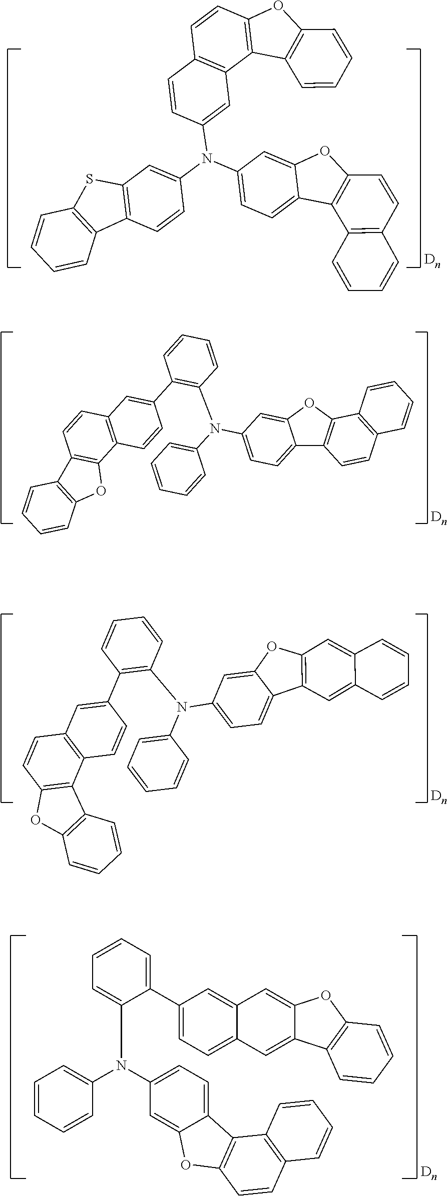 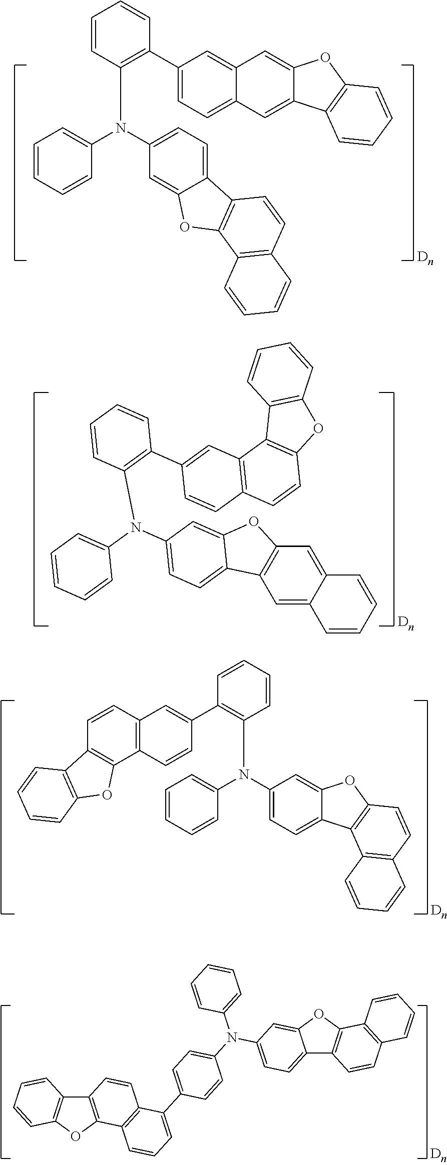 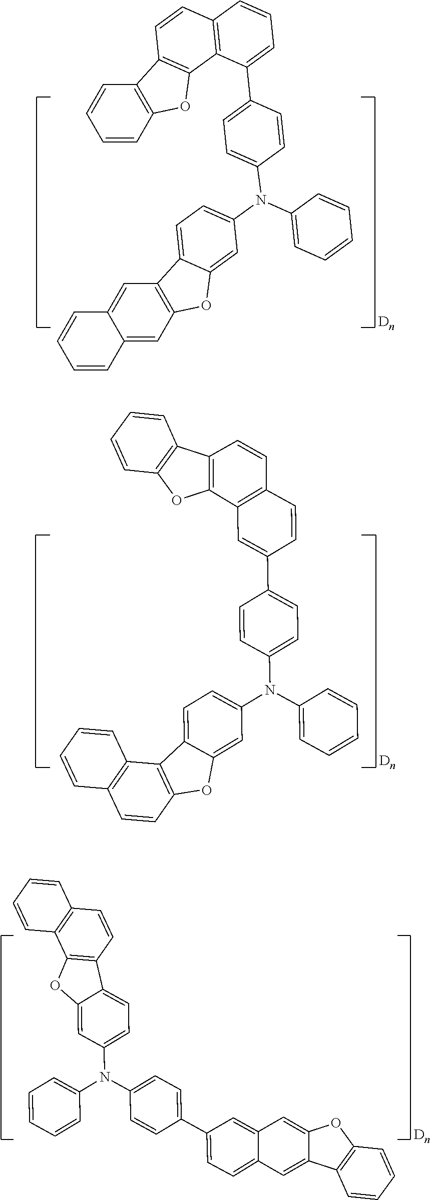 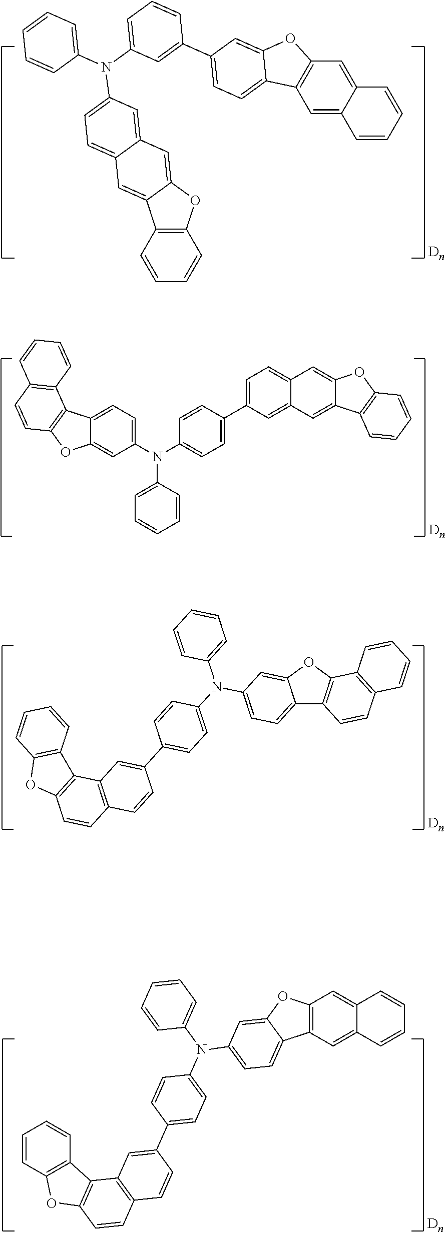 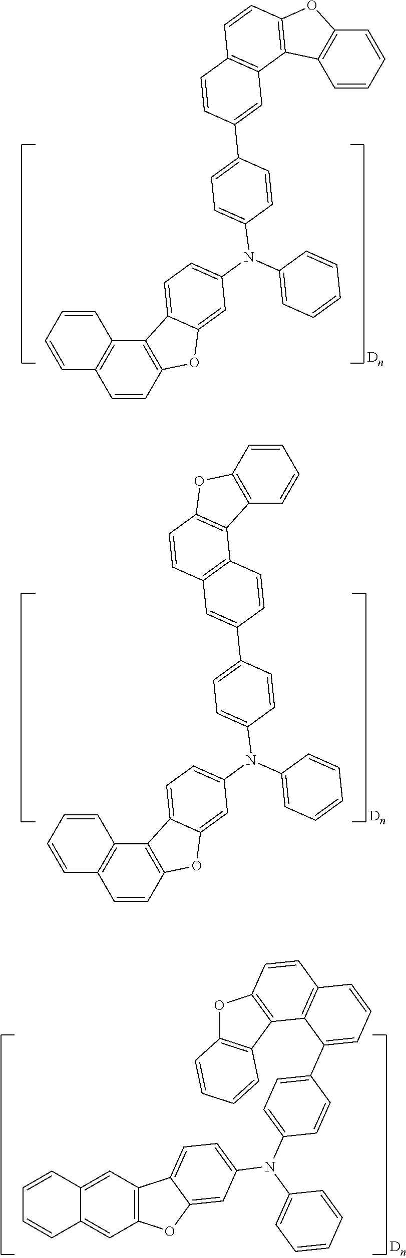 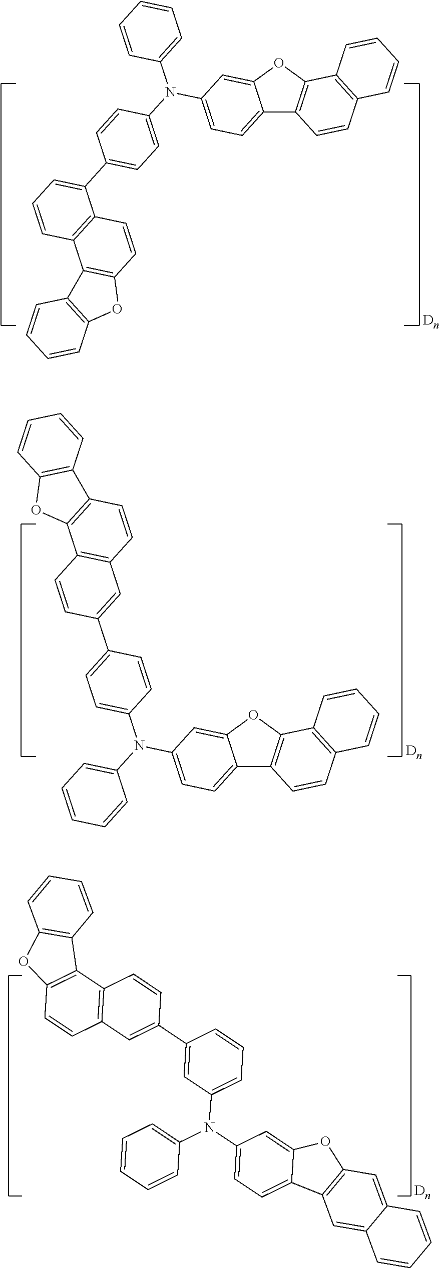 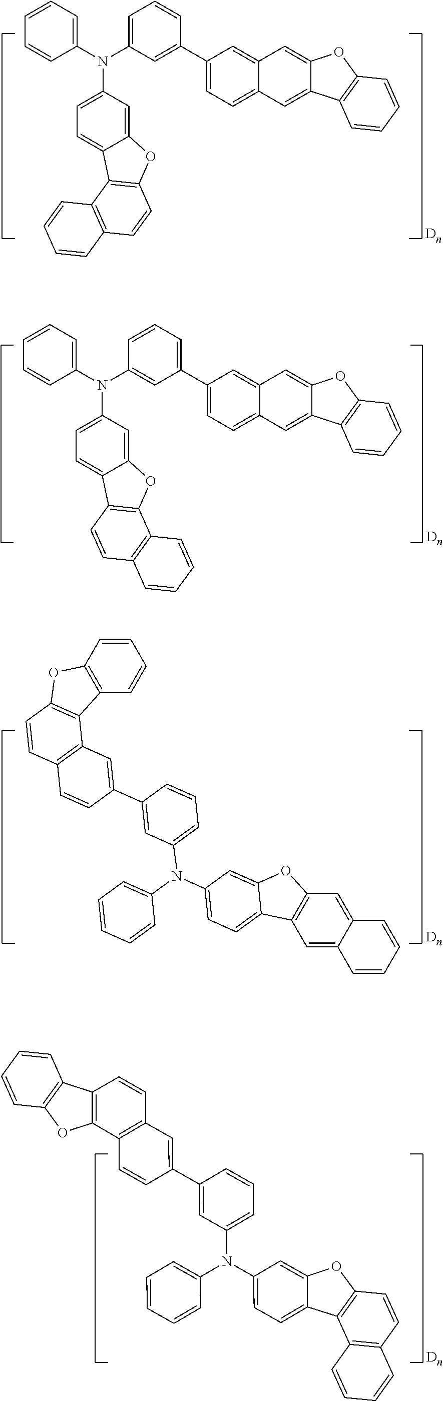 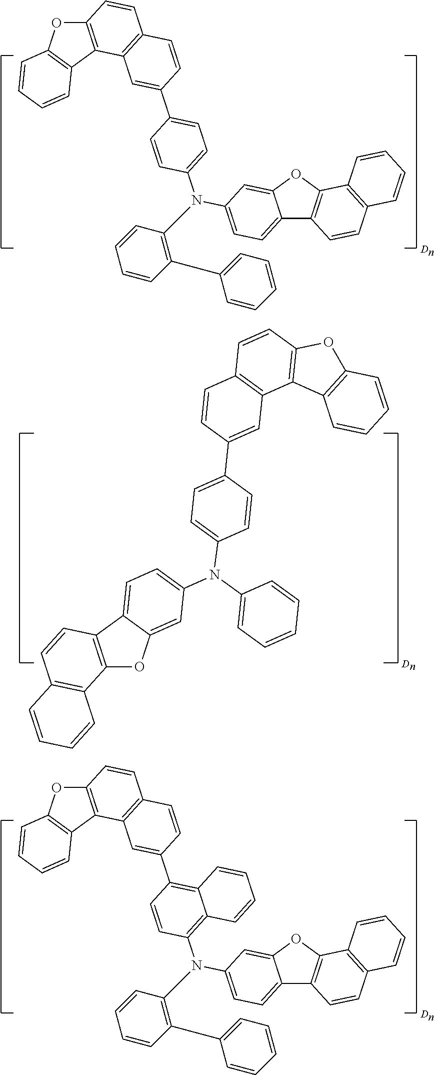 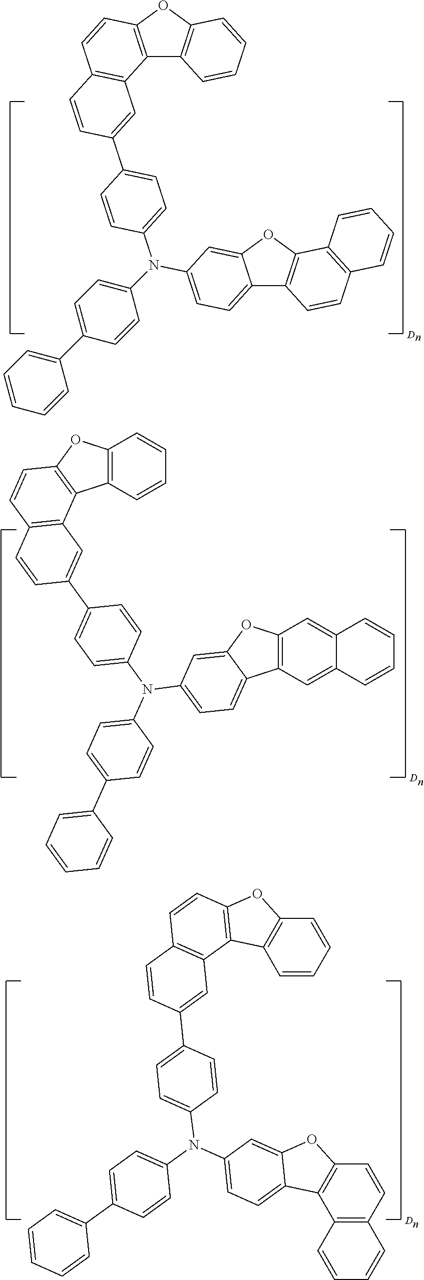 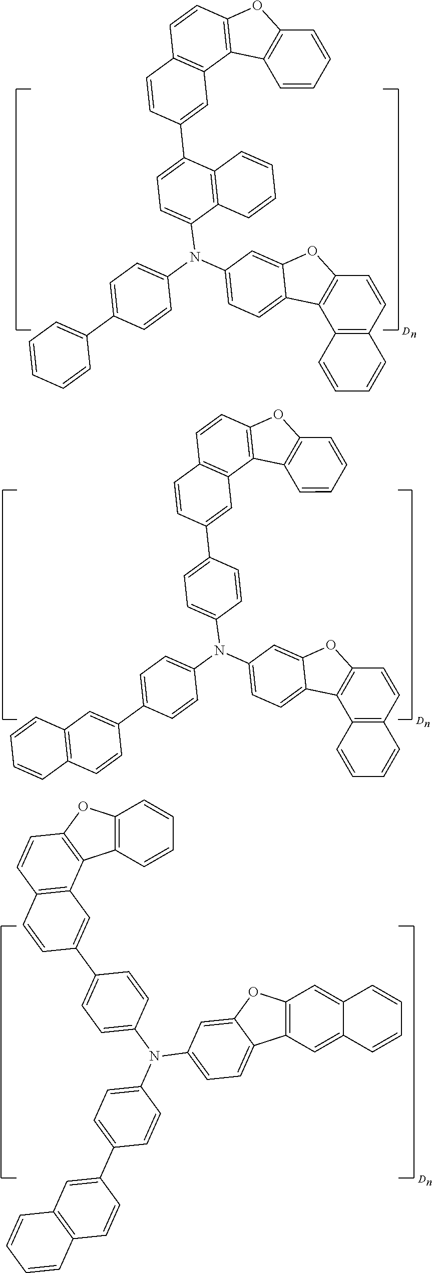 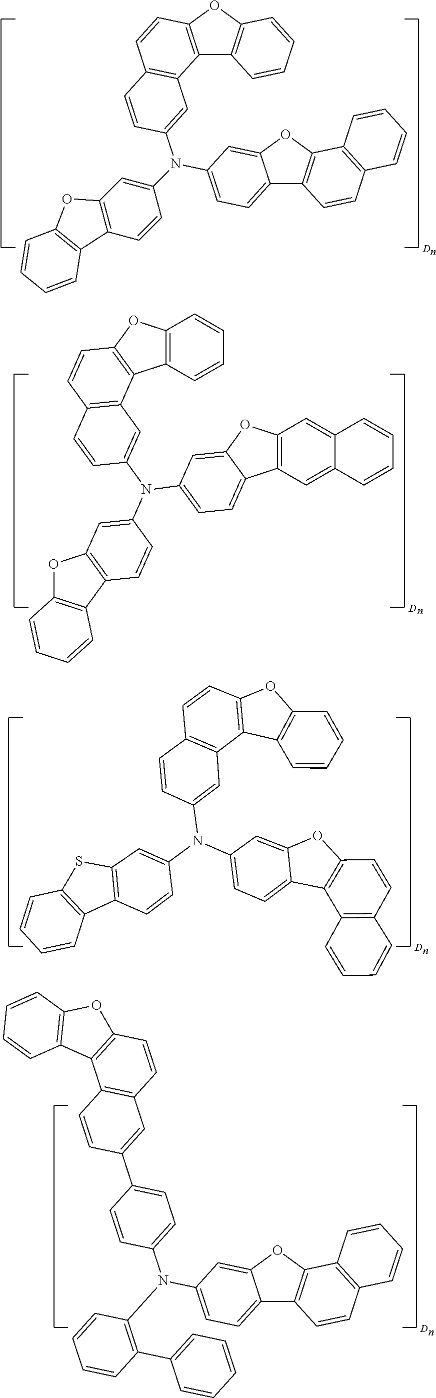 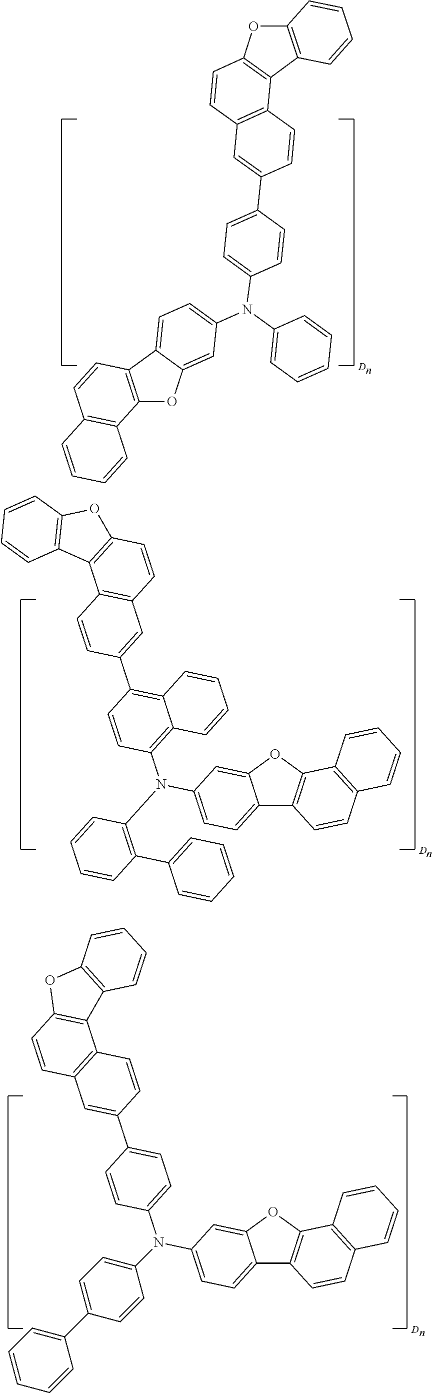 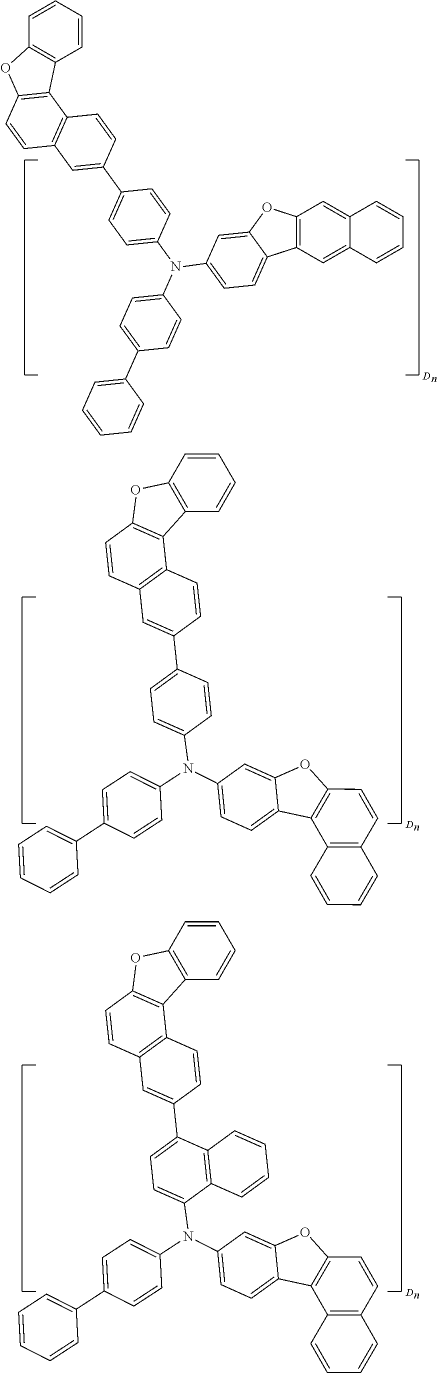 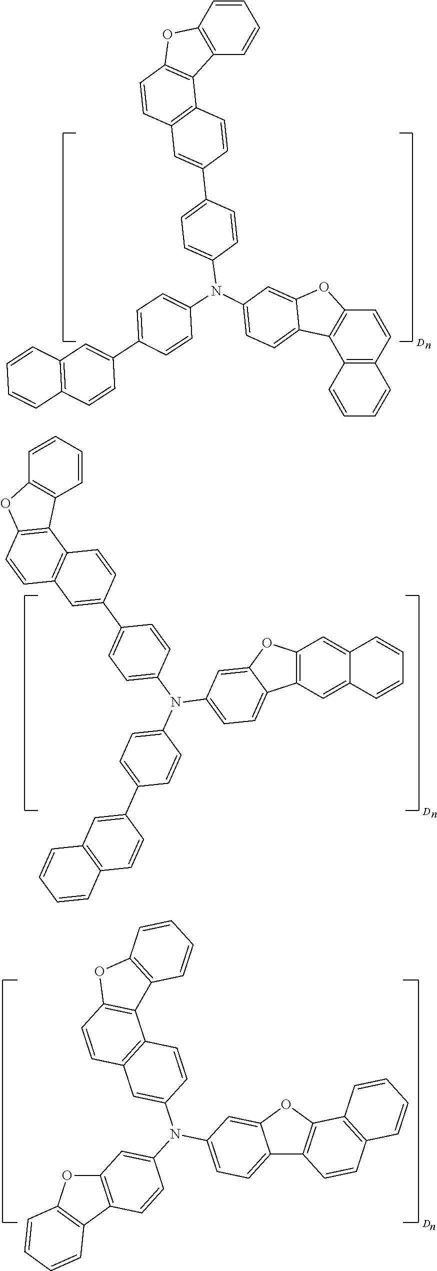 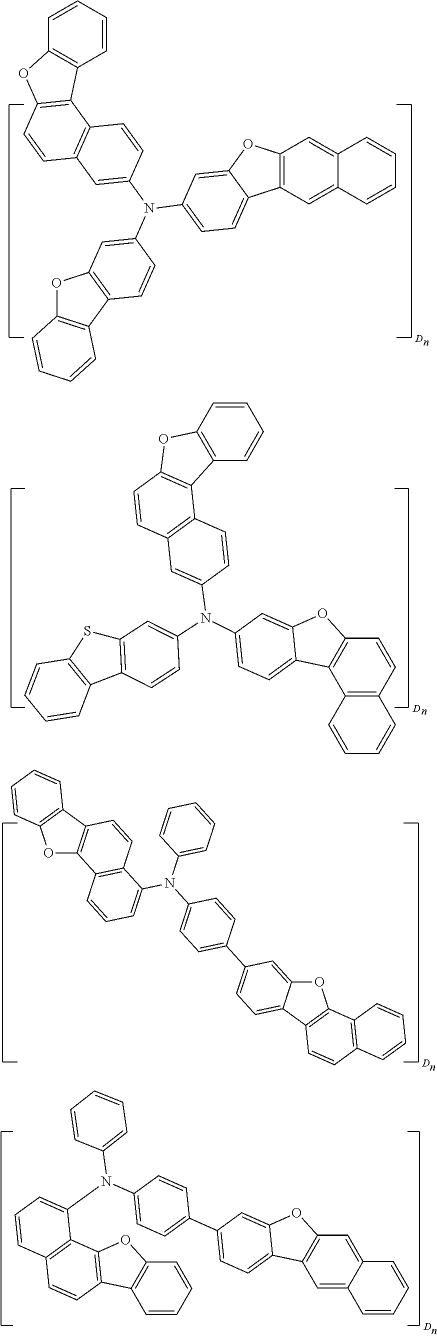 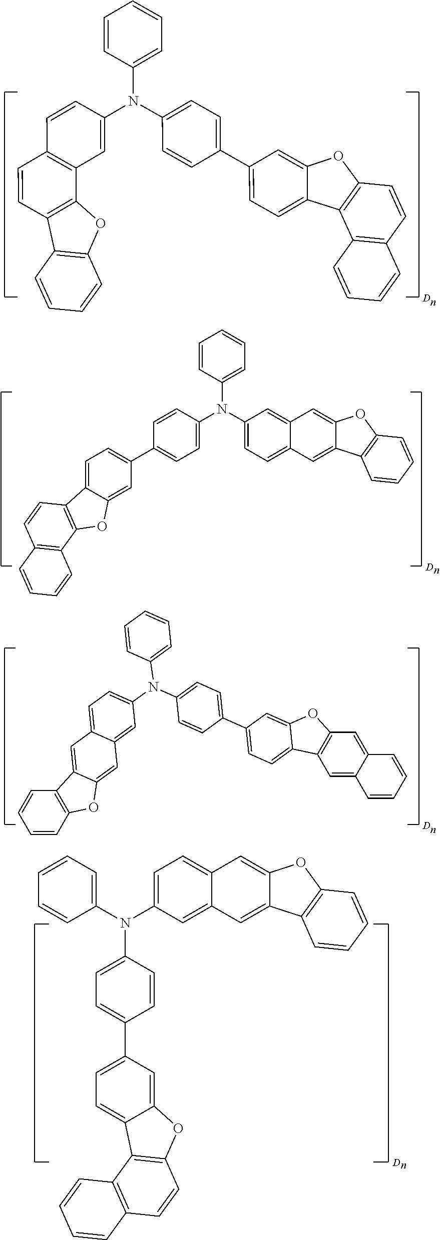 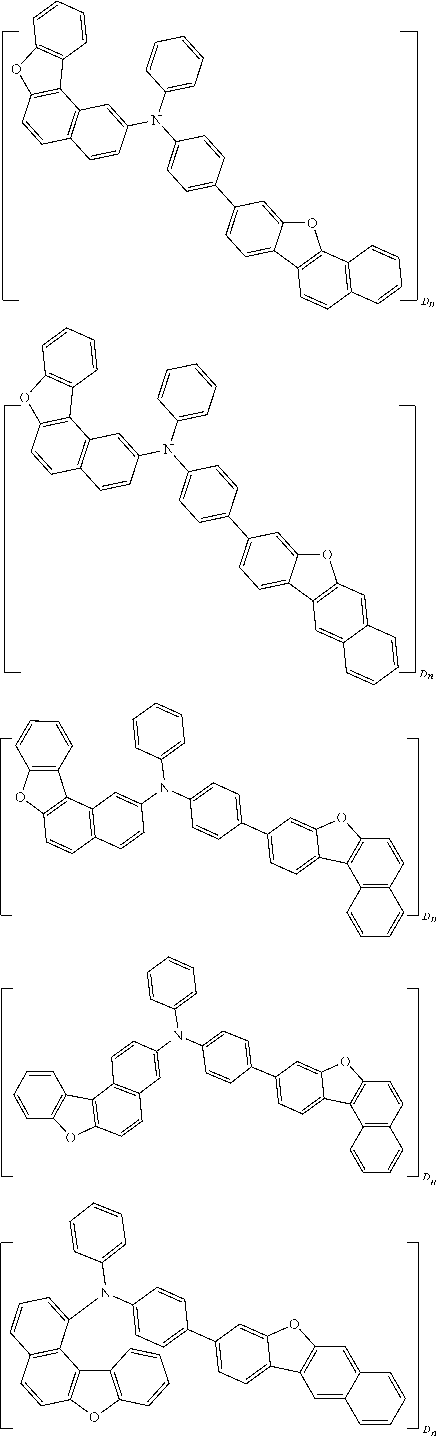 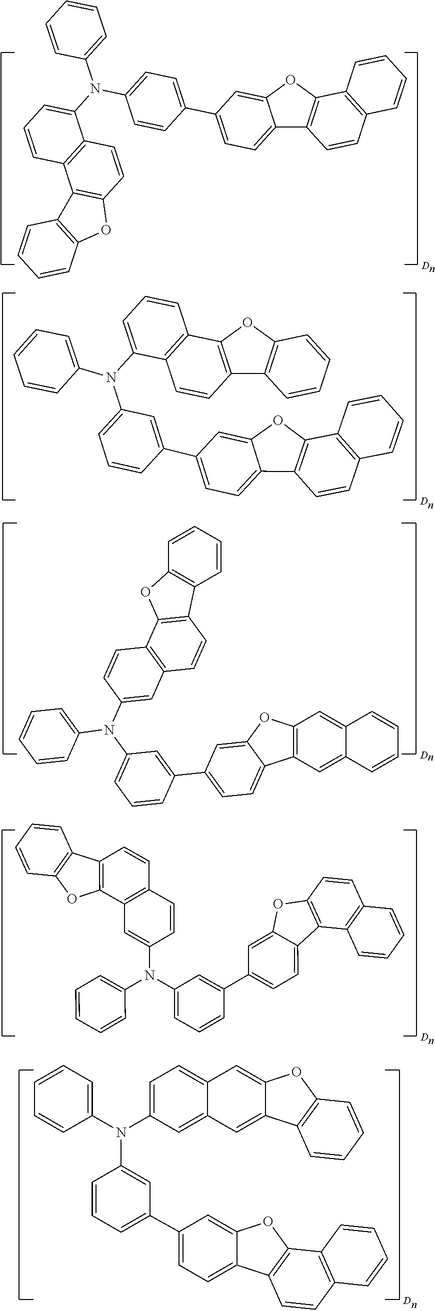 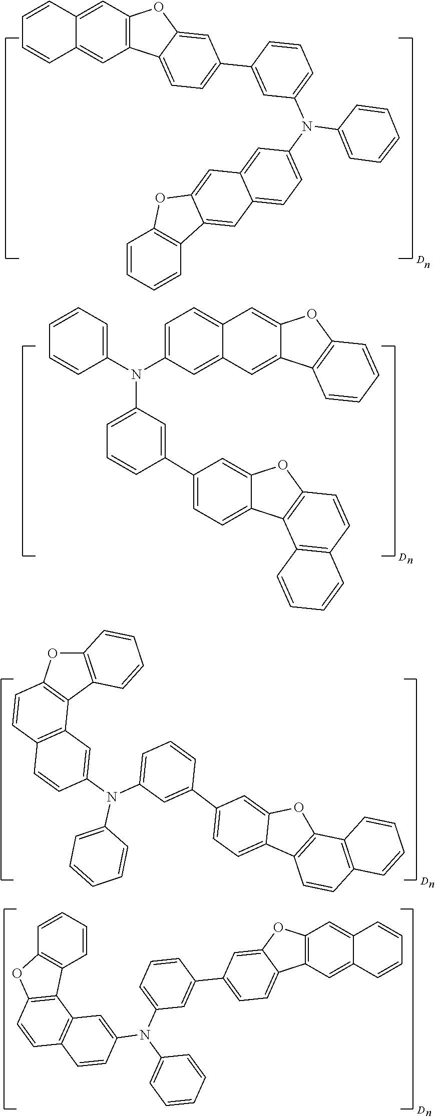 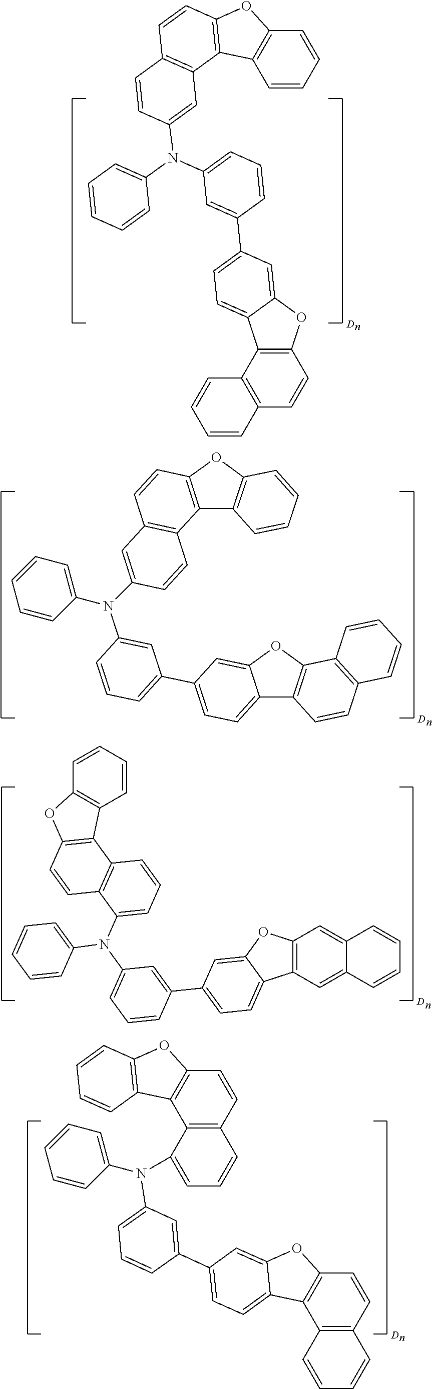 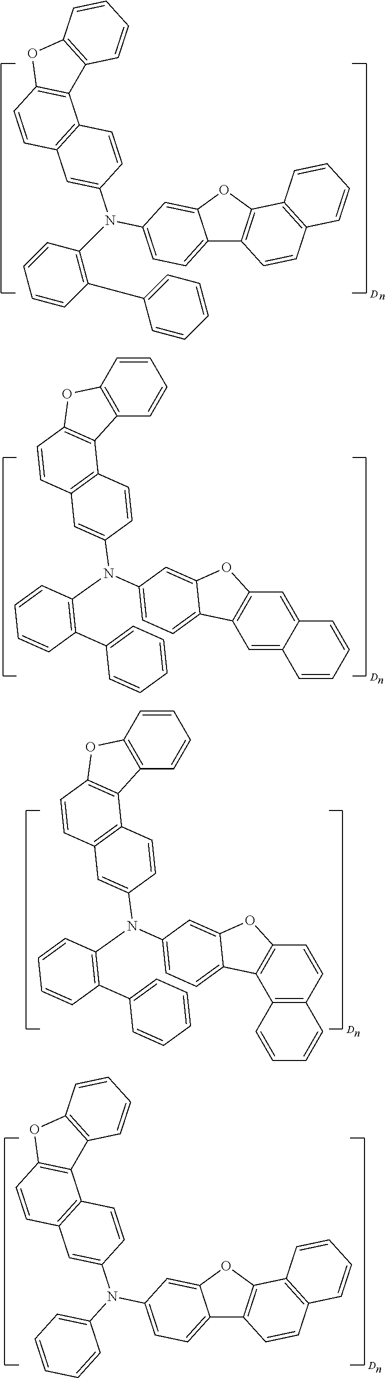 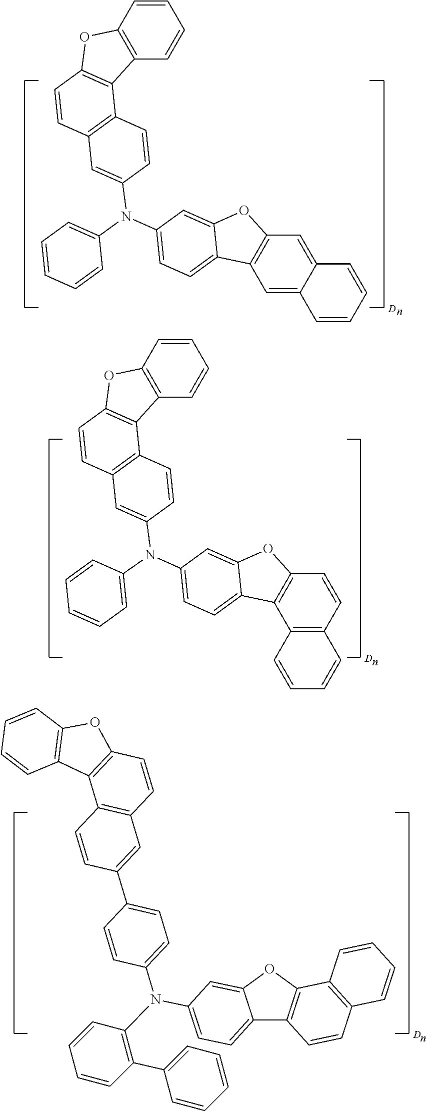 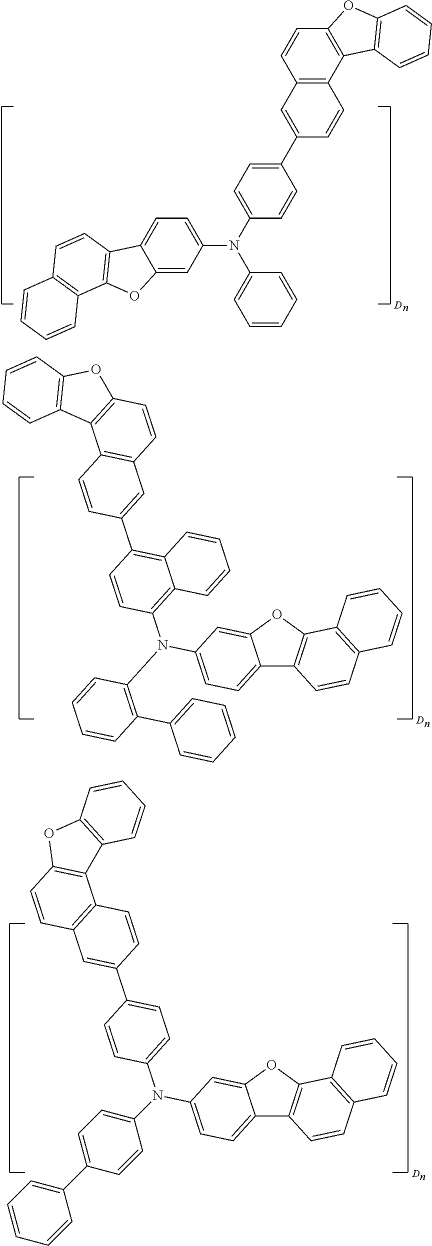 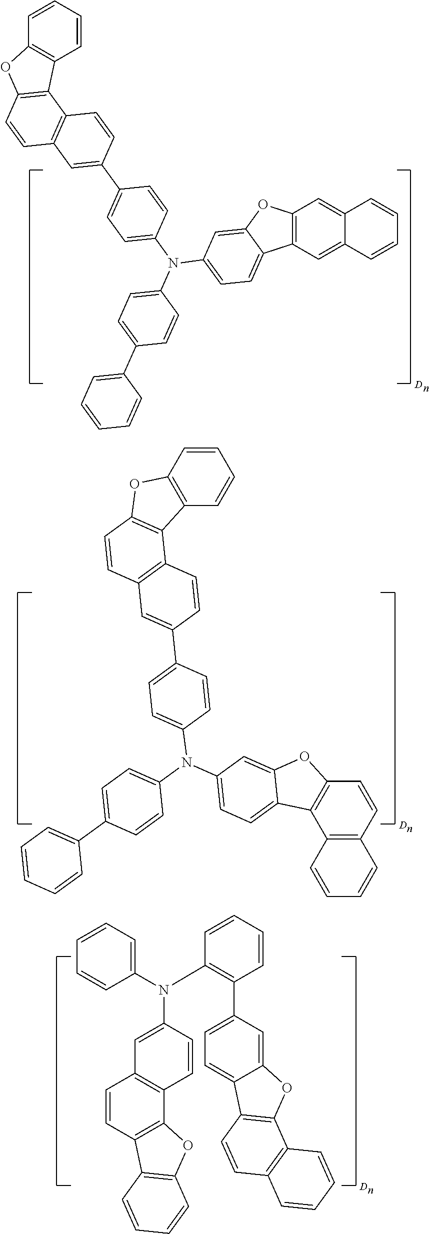 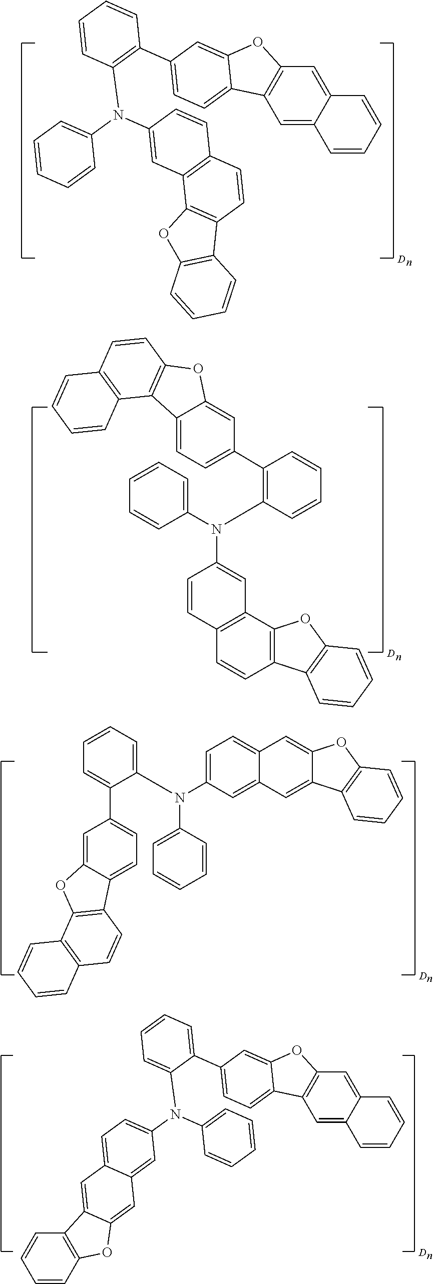 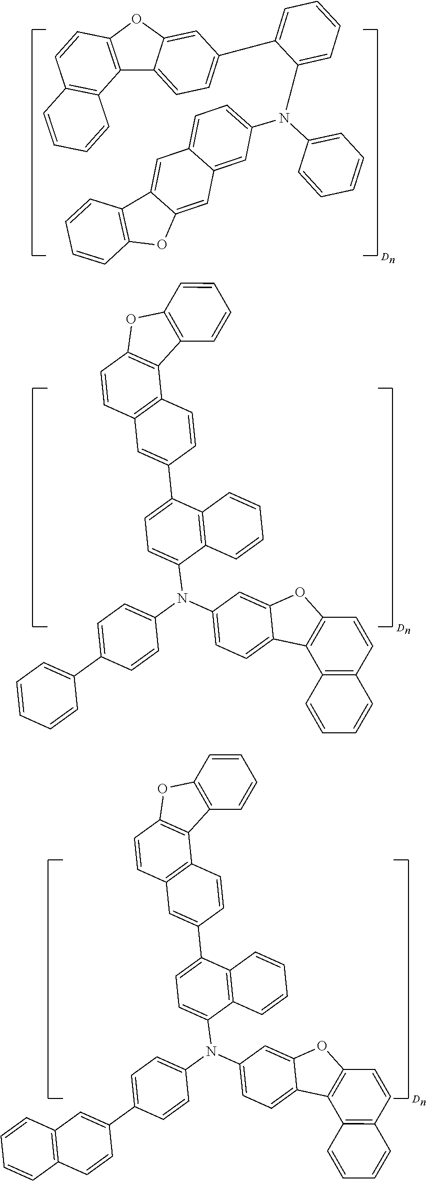 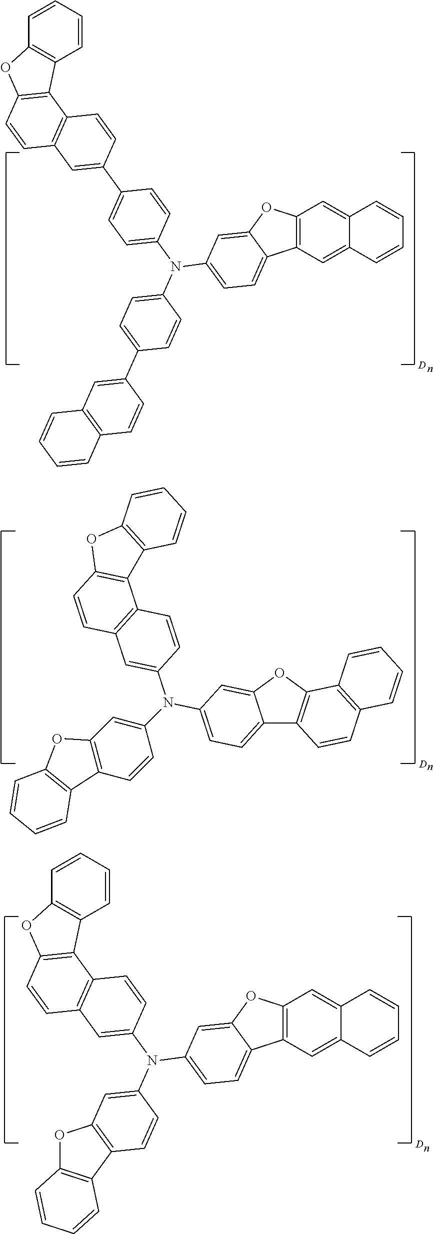 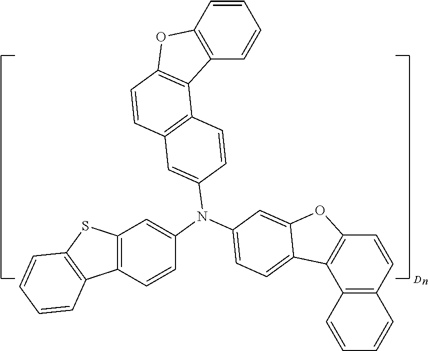 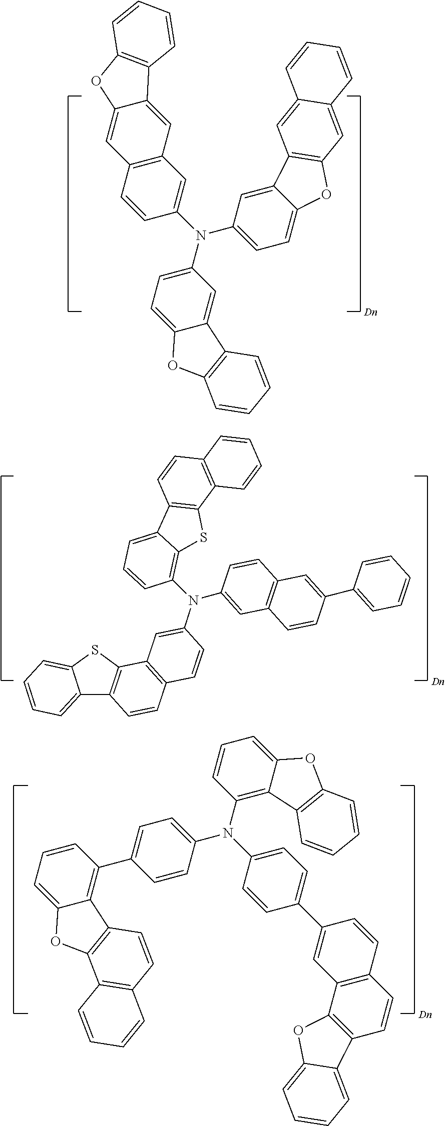 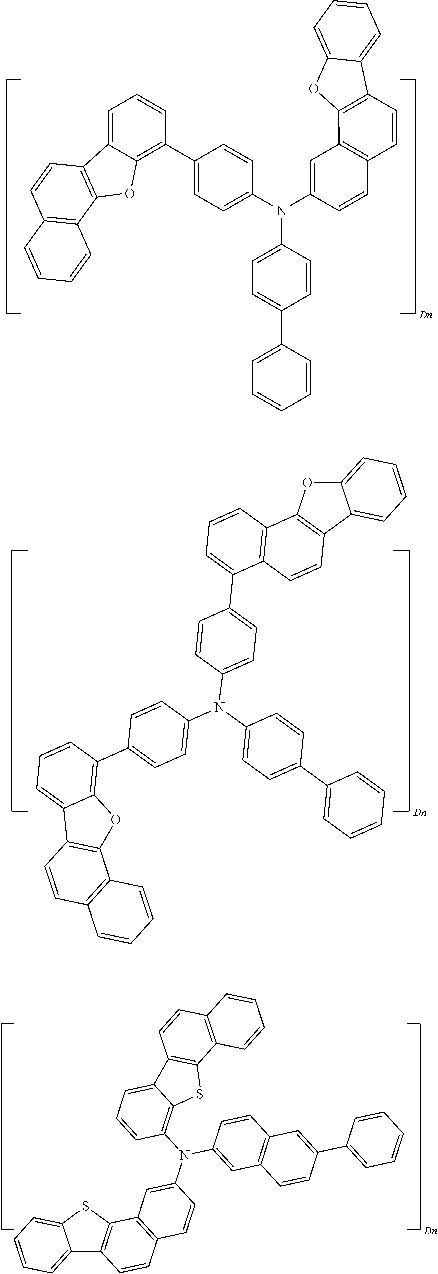 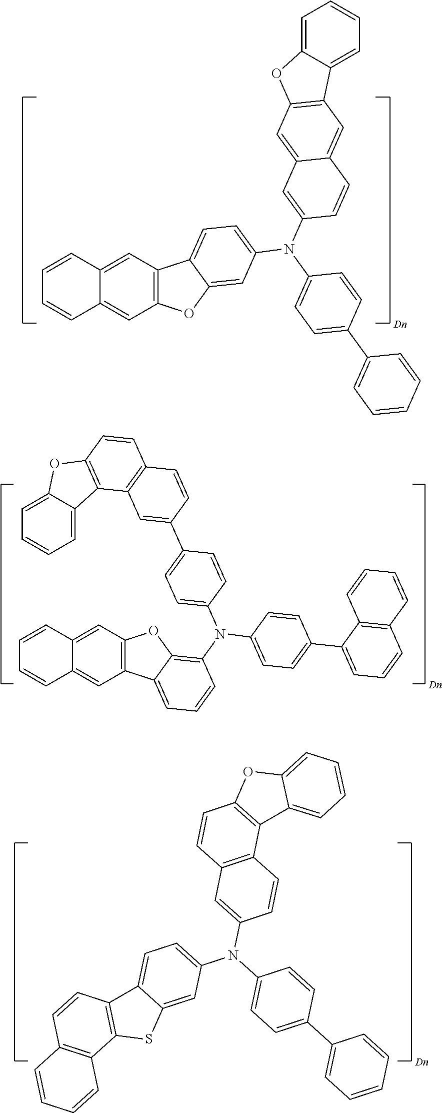
wherein,
Dn means that n hydrogens have been replaced by deuterium, wherein n is an integer of 1 or more.

13. An organic light emitting device comprising: a first electrode; a second electrode that is provided opposite to the first electrode; and one or more organic material layers that are provided between the first electrode and the second electrode, wherein one or more layers of the organic material layers comprises the compound of claim 1.

14. The organic light emitting device of claim 13, wherein:
the organic material layers comprising the compound are a hole transport layer, an electron blocking layer, or a light emitting layer.
